(19) United States
(12) Patent Application Publication
(10) Pub. No.: US 2025/0154156 A1
(43) Pub. Date: May 15, 2025

(54) QUINAZOLINE DERIVATIVE AS KRAS G12C MUTATION INHIBITOR

(71) Applicant: Shenzhen Forward Pharmaceuticals Co., Limited, Shenzhen, Guangdong (CN)

(72) Inventors: Chaochun ZHANG, Shenzhen, Guangdong (CN); Xuan YANG, Shenzhen, Guangdong (CN); Kai GAO, Shenzhen, Guangdong (CN); Chenggang ZHU, Shenzhen, Guangdong (CN); Liming BAO, Shenzhen, Guangdong (CN); Chaole CHEN, Shenzhen, Guangdong (CN); Xiangyan MIN, Shenzhen, Guangdong (CN); Qianwen WANG, Shenzhen, Guangdong (CN); Chunniu WANG, Shenzhen, Guangdong (CN); Liangliang XU, Shenzhen, Guangdong (CN)

(21) Appl. No.: 18/838,136

(22) PCT Filed: Feb. 13, 2023
(86) PCT No.: PCT/CN2023/075571

§ 371 Date: Sep. 24, 2024

(30) Foreign Application Priority Data
Feb. 14, 2022 (CN) 202210135494.9
Mar. 10, 2022 (CN) 202210234267.1

(51) Int. Cl. C07D 487/04; A61K 31/517; A61P 35/00

(52) U.S. Cl. CPC C07D 487/04; A61K 31/517; A61P 35/00

(57) ABSTRACT

Provided are a quinazoline derivative of formula (II) and a preparation method therefor, the quinazoline derivative being used as a KRAS G12C mutation inhibitor. The KRAS G12C mutation inhibitor can more efficiently inhibit KRAS G12C mutation, prevents drug-resistant mutation, and has good metabolic stability.

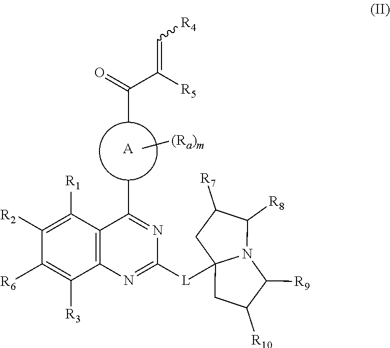

### TECHNICAL FIELD

The present invention provides a class of quinazoline derivatives as KRAS G12C mutation inhibitors and a preparation method therefor. The KRAS G12C small molecule inhibitor can be used for treating diseases caused by KRAS G12C mutation.

### BACKGROUND ART

Kirsten rat sarcoma viral oncogene homolog (KRAS), a membrane-bound protein located on the inner side of a cell membrane, functions similarly to a molecular switch of a GTPase. It regulates a signaling pathway in the cell via KRAS protein by regulating the mutual transformation between guanosine diphosphate (GDP) and guanosine triphosphate (GTP). When KRAS mutation occurs and leads to abnormality in the encoded protein, the mutant protein will activate a downstream related signaling pathway, leading to excessive activation in cells such as proliferation, differentiation and survival, which in turn causes the occurrence and spread of tumors.

Due to its structural characteristics, KRAS protein lacks a suitable target on the surface for drug action, so KRAS has been regarded as an undruggable target for a long time, and the related drug research progress is slow. Among numerous mutations in KRAS protein, KRAS G12C mutation is widely prevalent in human cancers. There is an urgent need to develop small molecule drugs specifically targeting KRAS G12C, and the prospect is broad.

In the prior art, there are known compounds with KRAS G12C mutation inhibitory activity, including AMG510 and MRTX849, the structures of which are as shown below; however, these compounds can not achieve ideal KRAS G12C mutation inhibitory activity. Furthermore, from the current ongoing clinical trials, it has been found that KRAS G12C patients who received MRTX849 treatment successively developed drug-resistant mutations such as Y96C, R68S, H95Q, and Q99L. This adds a lot of difficulty to the development of novel effective inhibitors against drug-resistant KRAS G12C, and no inhibitors capable of inhibiting drug-resistant mutations have ever been developed in the prior art. In addition, the KRAS G12C mutation inhibitors in the prior art also have the problem of poor metabolic stability. Therefore, there is an urgent need in the art to develop a compound capable of inhibiting KRAS G12C mutation more efficiently, preventing drug-resistant mutations and improving metabolic stability.

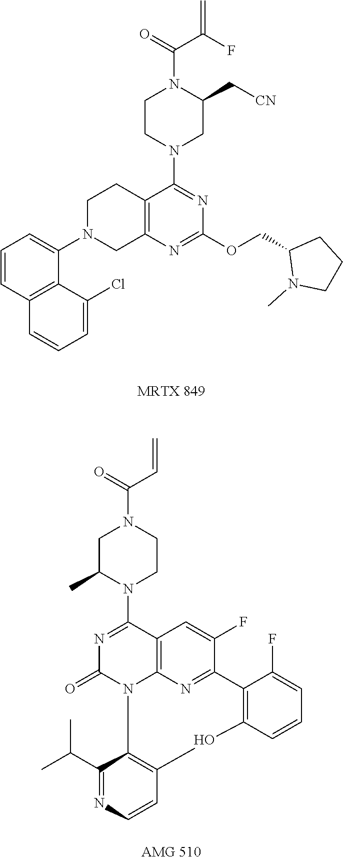

### SUMMARY OF THE INVENTION

In one aspect, the present invention provides a compound of formula (II) or a pharmaceutically acceptable salt or stereoisomer thereof.

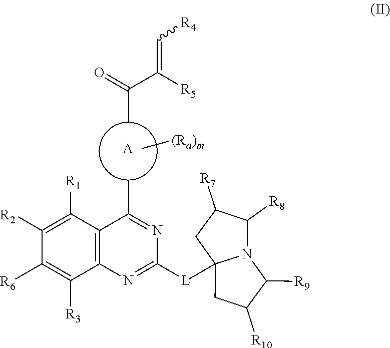

wherein
L is selected from a bond, oxygen, sulfur, —NH—, —(CH₂)ₙ—, —O(CH₂)ₙ—, —S(CH₂)ₙ—, —NH(CH₂)ₙ—, —(CH₂)ₙNH—, —(CH₂)ₙO—, —(CH₂)ₙS—, —(CH₂)ₙC(═O)—, —C(═O)O(CH₂)ₙ—, —OC(═O)(CH₂)ₙ—, —C(═O)(CH₂)ₙ—, —C(═O)NH(CH₂)ₙ—, and —NHC(═O)(CH₂)ₙ—; preferably —OCH₂—, —NHCH₂—, or —NHC(═O)—;
ring A is selected from substituted or unsubstituted, saturated or unsaturated, N-containing cyclic, spiro or bridged C₄₋₁₄ compounds containing at least additionally one heteroatom selected from O, S, or N;
R₁, R₂, and R₃ are each independently selected from hydrogen, deuterium, cyano, amino, hydroxyl, halogen (such as —F, —Cl, or —Br), nitro, alkyl, alkenyl, alkynyl, alkoxy, C₁₋₃ haloalkyl, C₁₋₃ haloalkoxy (such as —OCF₃), and deuterated alkyl, wherein the amino, alkyl, alkenyl, alkynyl, alkoxy, haloalkyl, haloalkoxy, and deuterated alkyl can be optionally further substituted;
R₄ is selected from hydrogen, halogen, C₁₋₆ alkyl, C₂₋₆ alkynyl, carboxyl-substituted C₁₋₆ alkyl, amino-substituted C₁₋₆ alkyl, hydroxyl-substituted C₁₋₆ alkyl, C₁₋₆ haloalkyl, cycloalkyl, C₁₋₄ alkylene-C₂₋₆ heterocycloalkyl, C₂₋₆ heterocycloalkyl, aryl, heteroaryl, C₁₋₃ alkylamino-substituted C₁₋₄ alkyl, and C₁₋₃ alkylamino-substituted C₁₋₈ alkoxy;
R₅ is selected from halogen or haloalkyl;
R₆ is selected from C₆₋₁₀ aryl (preferably naphthyl), C₅₋₉ heteroaryl, C₆₋₁₀ aryl-C₁₋₆ alkylene, and C₅₋₁₀ heteroaryl-C₁₋₆ alkylene, wherein the alkyl, aryl, heteroaryl, or heterocyclyl is optionally substituted with one or more substituents independently selected from halogen, cyano, amino, hydroxyl, nitro, C₁₋₆ alkyl, C₁₋₆ alkoxy, C₁₋₆ alkylamino, C₂₋₄ alkynyl, C₂₋₄ alkenyl, C₁₋₄ alkylcyano, C₃₋₆ cycloalkyl, di-C₁₋₆ alkylamino, C₁₋₆ haloalkyl, aminoacyl, C₁₋₆ alkylaminoacyl, or di-C₁₋₆ alkylaminoacyl;
R₇, R₈, R₉, and R₁₀ are each selected from hydrogen, —NH₂, —OH, —C(═O), —CN, —NO₂, halogen, —COOH, C₁₋₁₂ alkyl, C₂₋₆ alkenyl, C₂₋₆ alkynyl, C₁₋₁₂ haloalkyl, C₃₋₁₂ cycloalkyl, C₃₋₁₄ heterocycloalkyl, C₆₋₁₀ aryl, C₅₋₉ heteroaryl, —(CH₂)ₙOC(═O)—C₆₋₁₀ aryl, —(CH₂)ₙOC(═O)—C₅₋₉ heteroaryl, —(CH₂)ₙOC(═O)—C₃₋₁₄ heterocycloalkyl, —(CH₂)ₙOC(═O)N(R₁)₂, —N(R₁₁)₂, —NHC(═NH)NH₂, —NR₁₁C(═O)—C₆₋₁₀ aryl, —NR₁₁C(═O)—C₅₋₉ heteroaryl, —NR₁₁C(═O)—C₃₋₁₄ heterocycloalkyl, —C(═O)N(R₁₁)₂, —(CH₂)ₙNHC(═O)—C₆₋₁₀ aryl, —(CH₂)ₙNHC(═O)—C₅₋₉ heteroaryl, and —(CH₂)ₙNHC(═O)—C₃₋₁₄ heterocycloalkyl, wherein the alkyl, amino, aryl, heteroaryl, cycloalkyl, or heterocyclyl is optionally substituted with one or more substituents independently selected from C₁₋₆ alkyl, C₁₋₆ alkoxy, C₁₋₆ haloalkyl, C₁₋₆ heterocycloalkyl, —NH₂, —OH, —C(═O), —CN, —NO₂, halogen, and —COOH;
m and n are each independently 0, 1, 2, or 3; and
R₁₁ is selected from C₁₋₃ alkyl, C₁₋₃ alkoxy, C₁₋₃ haloalkyl, and hydroxyl.

In a specific solution, L is selected from —O(CH₂)ₙ—, —S(CH₂)ₙ—, —NH(CH₂)ₙ—; preferably, L is selected from —OCH₂—, —NHCH₂—; more preferably, L is —OCH₂—.

In a specific solution, ring A is an optionally substituted 3- to 14-membered heterocyclyl, wherein preferably, the heterocyclyl contains only two nitrogen atoms as ring atoms; Rₐ is selected from C₁₋₆ alkyl (such as methyl or ethyl), C₂₋₆ alkenyl, C₂₋₆ alkynyl, halogen, —C(═O)H, —CN, —NH₂, —COOH, —OH, C₁₋₆ alkylamino, C₁₋₆ cyanoalkyl, C₁₋₆ carboxyalkyl, C₁₋₆ hydroxyalkyl, C₁₋₆ nitroalkyl, C₁₋₆ aminoalkyl, C₁₋₆ haloalkyl, C₁₋₆ alkoxy, and di-C₁₋₆ alkylamino; and m is 0, 1, or 2.

In a preferred solution, when ring A is unsubstituted (i.e., m=0), it is selected from the following groups:

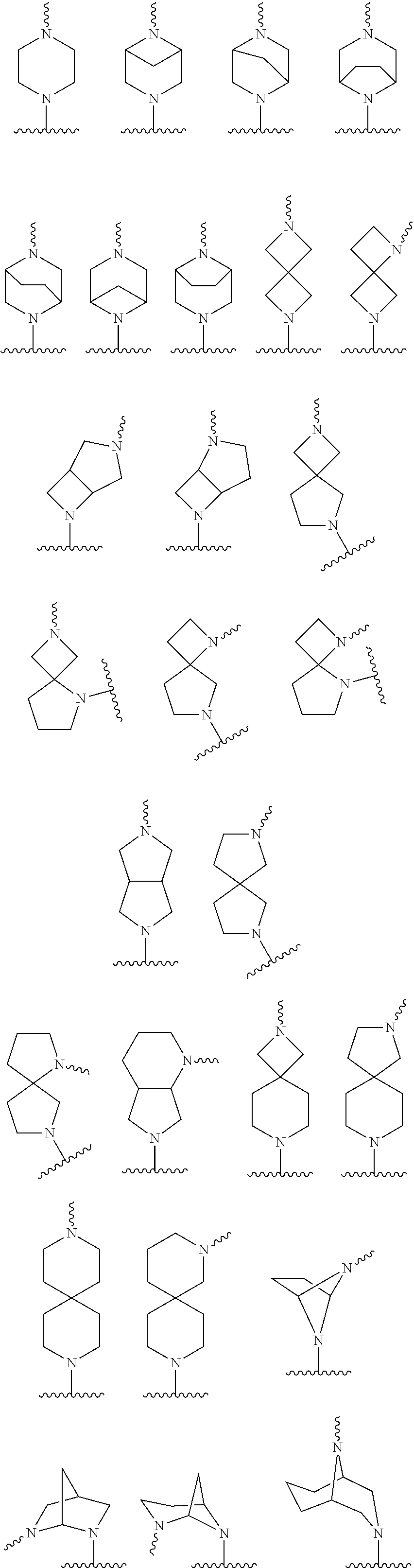

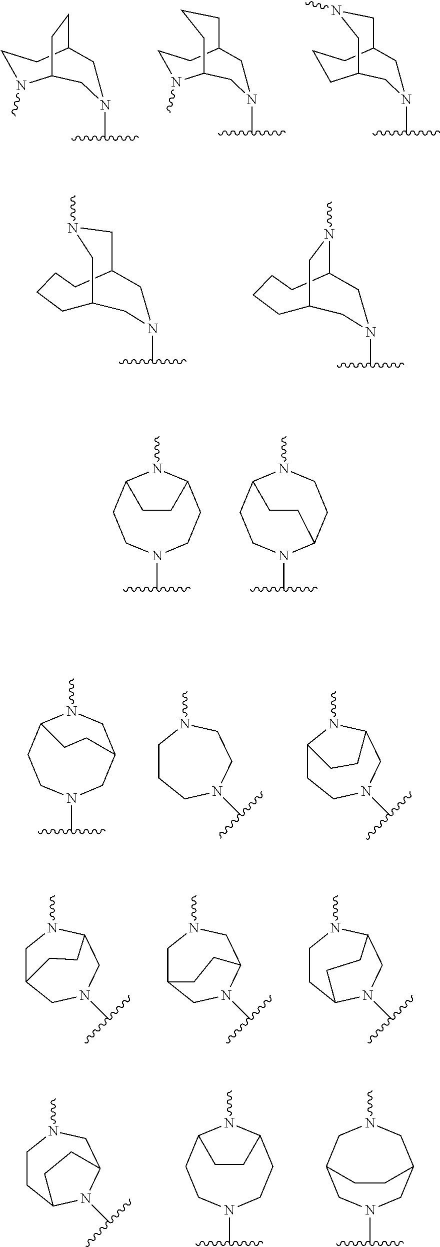

In some embodiments, when ring A is substituted with substituent Rₐ (that is, m is not 0), Rₐ is selected from hydrogen, halogen, halogen, hydroxyl, C₁₋₆ alkyl, C₂₋₆ alkenyl, C₂₋₆ alkynyl, C₁₋₆ haloalkyl, hydroxyl-substituted C₁₋₆ alkyl, carboxyl-substituted C₁₋₆ alkyl, amino-substituted C₁₋₆ alkyl, cyano-substituted C₁₋₆ alkyl, nitro-substituted C₁₋₆ alkyl, and C₁₋₆ alkoxy; more preferably, Rₐ is cyano-substituted C₁₋₆ alkyl, particularly preferably —CH₂CN.

Therefore, when there is a substitution with group Rₐ, ring A is selected from:

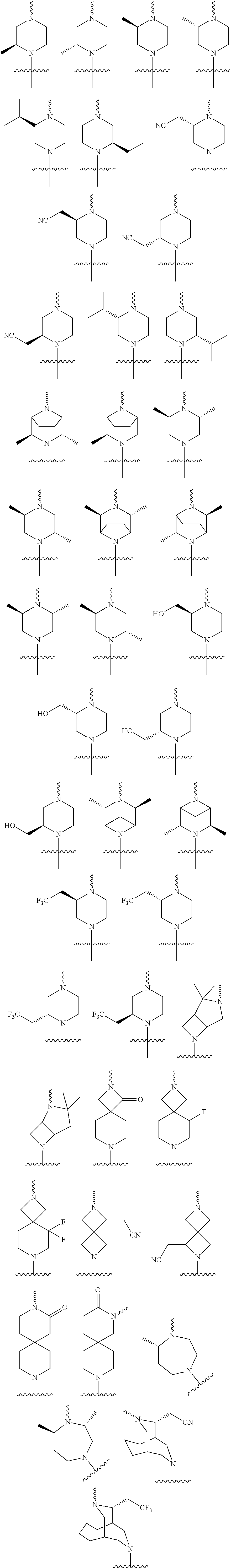

Particularly preferably, ring A is:

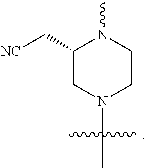

In a specific solution, R₁, R₂, and R₃ are each independently selected from hydrogen and halogen (preferably fluorine, chlorine, and more preferably fluorine). In a preferred solution, R₁ is hydrogen, R₂ is selected from hydrogen, fluorine, and chlorine, and R₃ is fluorine.

In a specific solution, R₄ is selected form H, halogen, C₁₋₆ alkyl, C₂₋₆ alkynyl, carboxyl-substituted C₁₋₄ alkyl, amino-substituted C₁₋₆ alkyl, hydroxyl-substituted C₁₋₆ alkyl, C₁₋₆ haloalkyl, C₁₋₆ cycloalkyl, C₁₋₄ alkylene-C₂₋₆ heterocycloalkyl, C₂₋₆ heterocycloalkyl, aryl, heteroaryl, C₁₋₃ alkylamino-substituted C₁₋₄ alkyl, and C₁₋₃ alkylamino-substituted C₁₋₈ alkoxy.

In a preferred solution, R₄ is selected from the following groups: H,

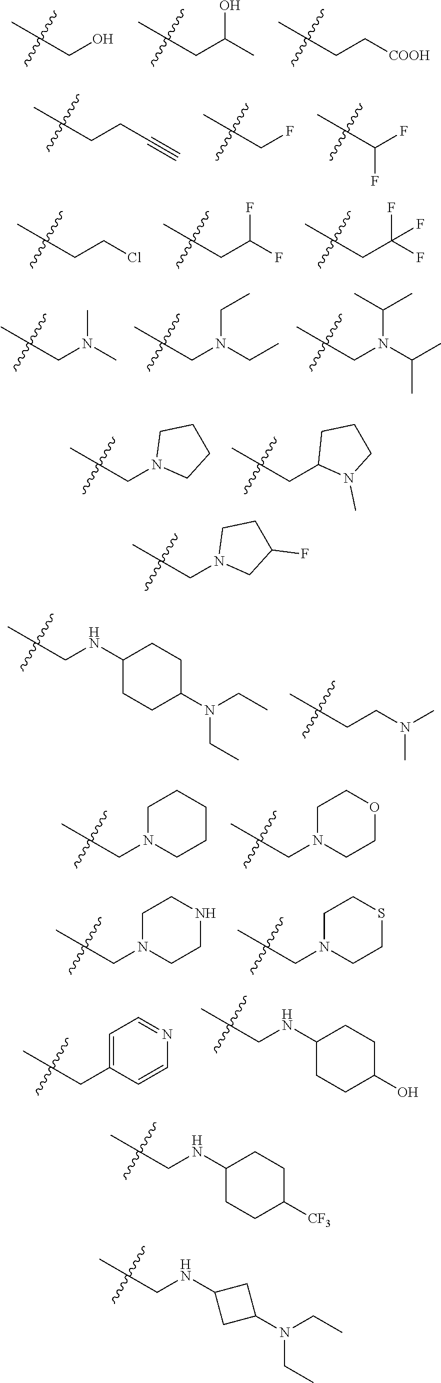

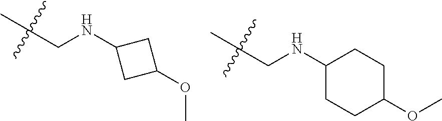

more preferably, R₄ is H.

In a specific solution, R₅ is halogen, preferably fluorine.

In a specific solution, R₆ is an optionally substituted C₆₋₁₀ aryl or C₅₋₁₀ heteroaryl. Where R₆ is substituted, the substituent thereon is selected from a C₁₋₆ hydrocarbon group (e.g., a saturated C₁₋₆ hydrocarbon group, such as C₁₋₆ alkyl, such as methyl and ethyl; or an unsaturated C₂₋₆ hydrocarbon group, such as C₂₋₆ alkenyl or C₂₋₆ alkynyl, such as ethynyl or vinyl), C₃₋₆ cycloalkyl (such as cyclopropyl), C₁₋₆ alkoxy (such as methoxy or ethoxy), —NH₂, —OH, halogen (such as —F, —Cl, —Br, and —I), C₁₋₆ haloalkyl (e.g., —CF₃), and —CN.

In a preferred solution, R₆ is selected from the following groups:

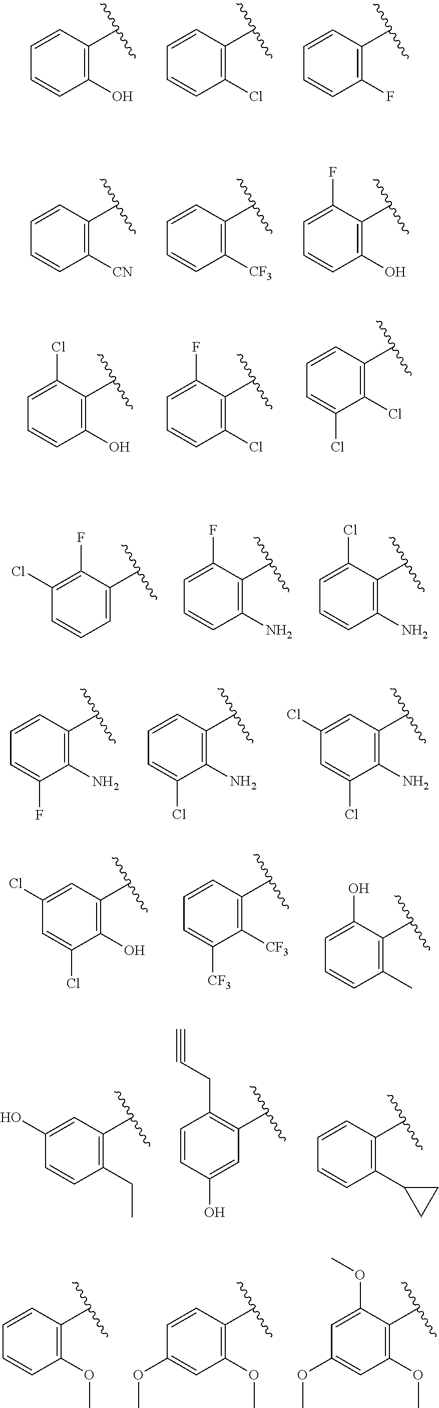

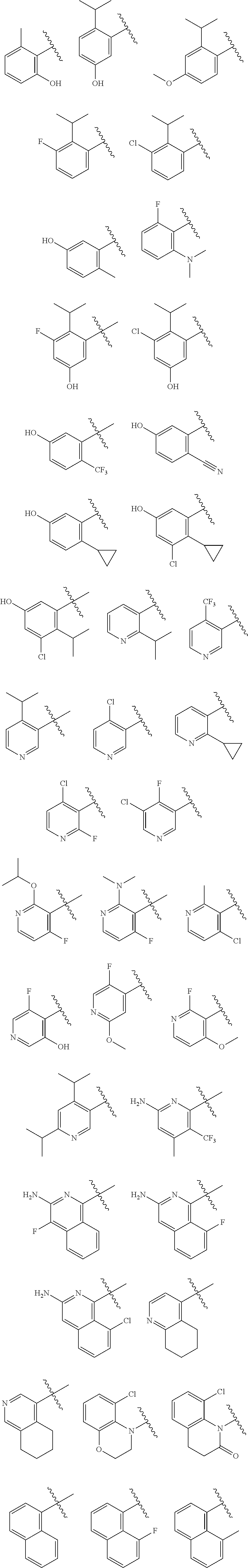

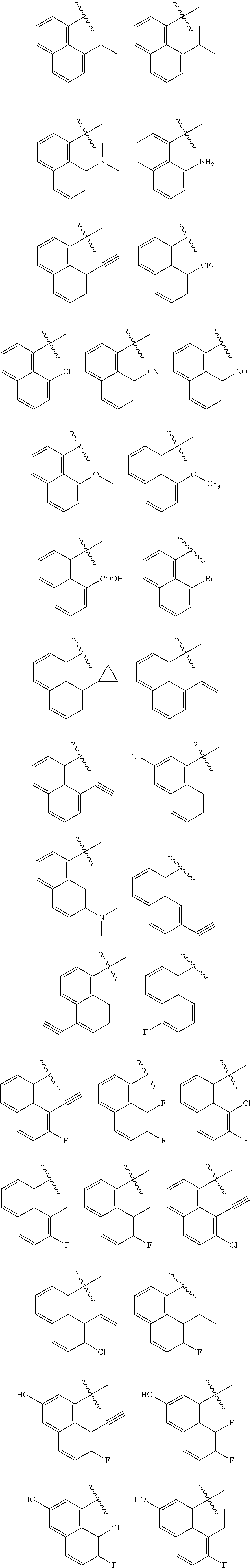

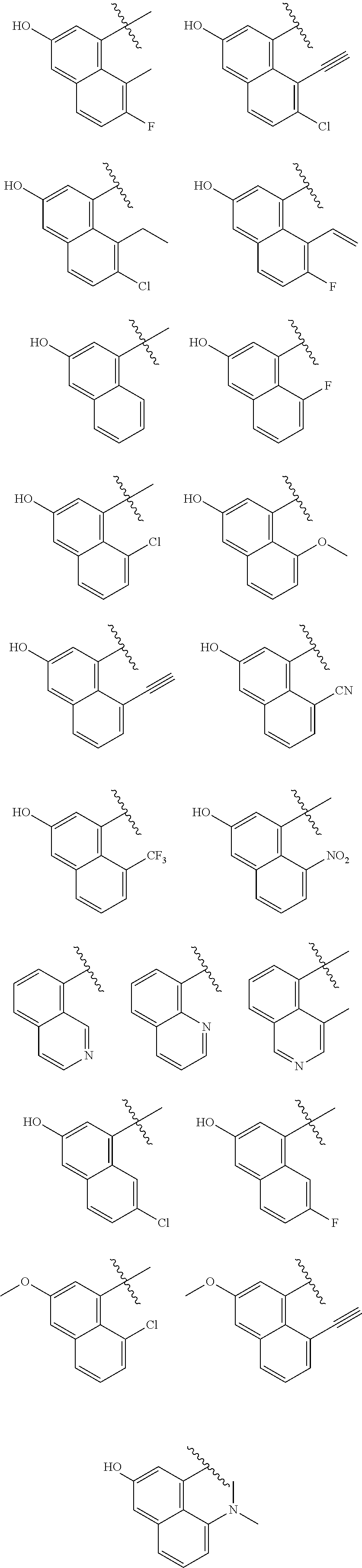

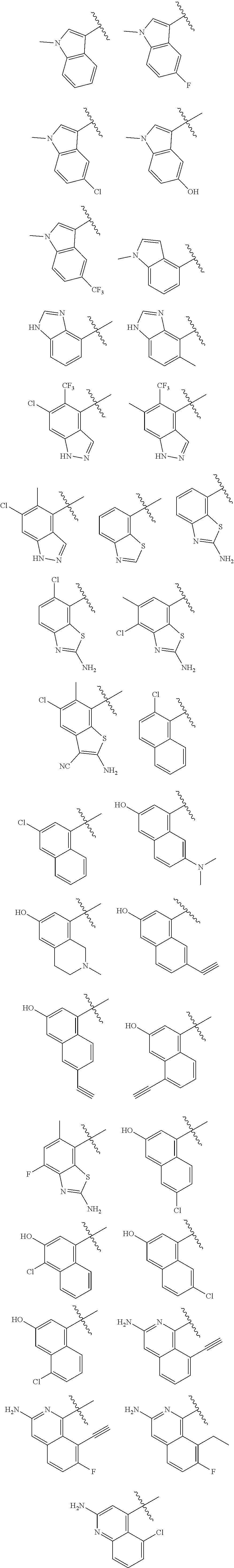

In a preferred solution, R₆ is naphthyl substituted with a group selected from halogen (especially fluorine and chlorine), cyano, hydroxyl, amino, and C₂₋₆ alkynyl (especially ethynyl). More preferably, R₆ is selected from the following groups.

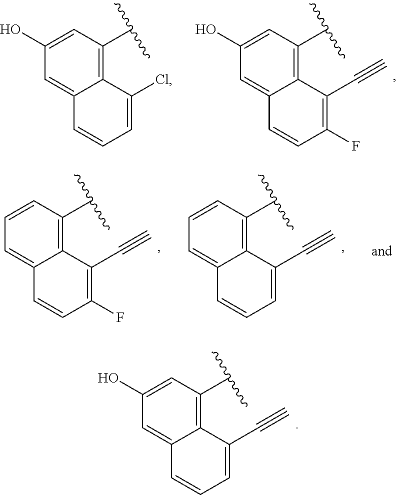

In a specific solution, R₇, R₈, R₉ and R₁₀ are H, halogen, C₁₋₆ alkyl, C₂₋₆ alkynyl, carboxyl-substituted C₁₋₄ alkyl, amino-substituted C₁₋₆ alkyl, hydroxyl-substituted C₁₋₆ alkyl, C₁₋₆ haloalkyl, cycloalkyl, —C₁₋₄ alkylene-C₂₋₆ heterocycloalkyl, C₂₋₆ heterocycloalkyl, aryl, heteroaryl, and C₁₋₃ alkylamino-substituted C₁₋₄ alkyl.

In a preferred solution, R₇ is hydrogen or halogen (preferably fluorine), and R₅, R₉, and R₁₀ are hydrogen.

In another specific solution, the first atom to which the “ [symbol] ” in the moiety

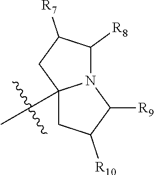

in formula (II) is connected (i.e., the atom in L that is connected to this moiety) is a carbon atom, and this moiety together with this carbon atom constitutes a group selected from the following:

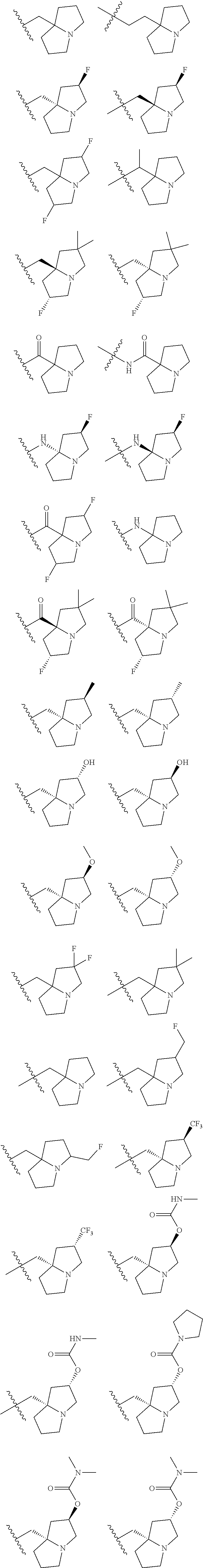

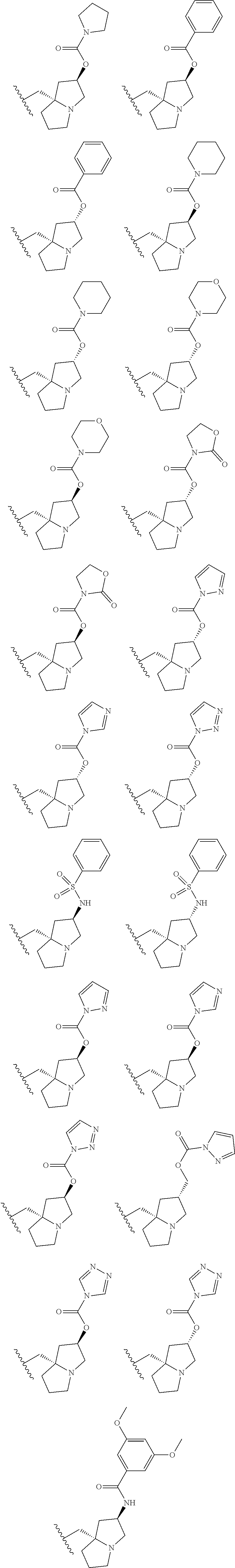

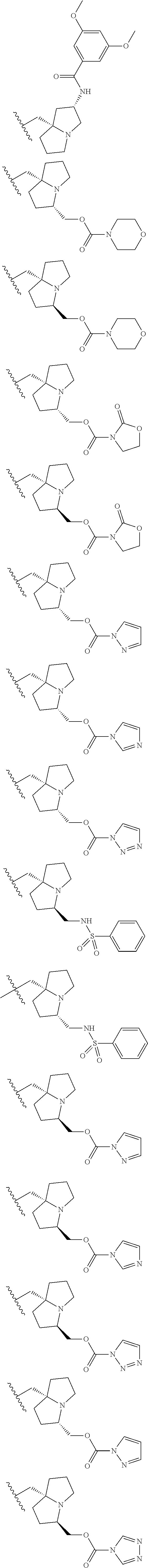

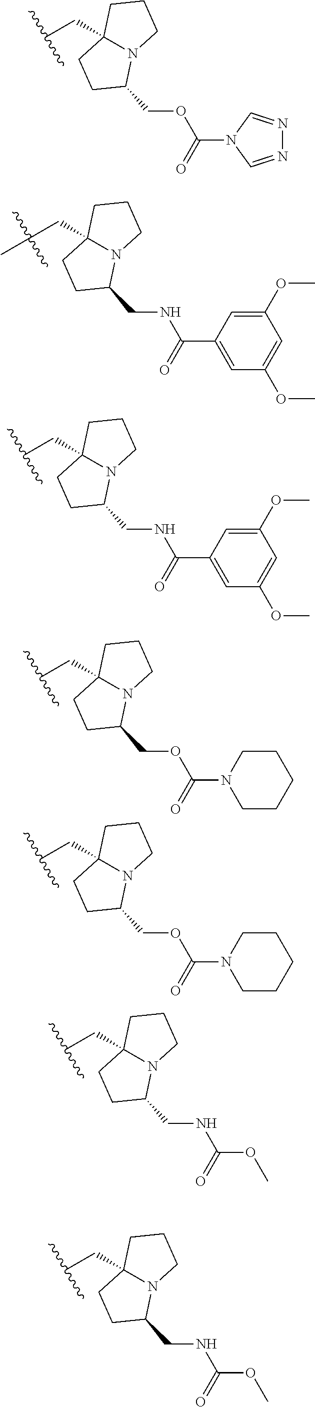

More preferably, this moiety, together with the carbon atom, constitutes pyrrolizinylmethyl optionally substituted with halogen (preferably fluorine). Further preferably, the moiety together with the carbon atom constitutes a group selected from:

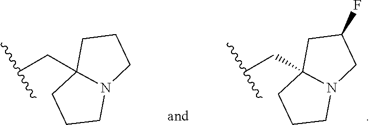 [SMILES: *CC12CCCN1CCC2 ; *C[C@@]12CCCN1C[C@H](F)C2]

### DETAILED DESCRIPTION OF EMBODIMENTS

In a preferred aspect of the present invention, there is provided a compound of formula (II-A0) or formula (II-A) or a pharmaceutically acceptable salt or stereoisomer thereof:

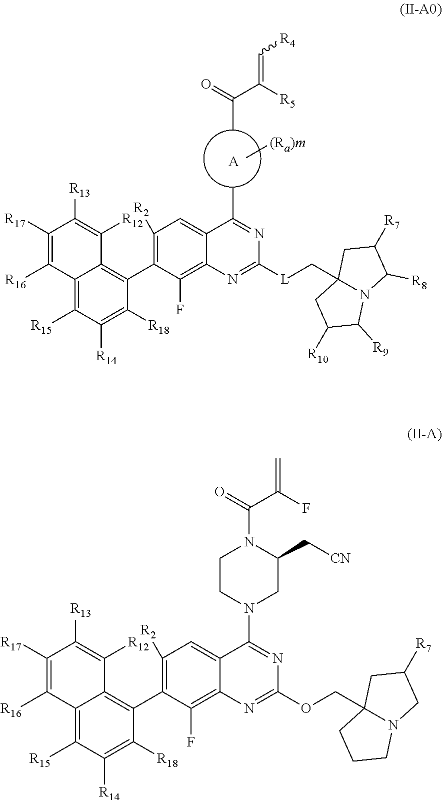

wherein R₂ has the definition given above and is preferably hydrogen, fluorine, or chlorine; and R₇ has the definition given above and is preferably hydrogen or fluorine. R₁₂ to R₁₈ are each independently selected from hydrogen, halogen (especially fluorine and chlorine), cyano, hydroxyl, amino, C₁₋₆ alkyl (especially methyl and ethyl), and C₂₋₆ alkynyl (especially ethynyl).

In a preferred aspect of the present invention, there is provided a compound of formula (II-B0) or formula (II-B) or a pharmaceutically acceptable salt or stereoisomer thereof:

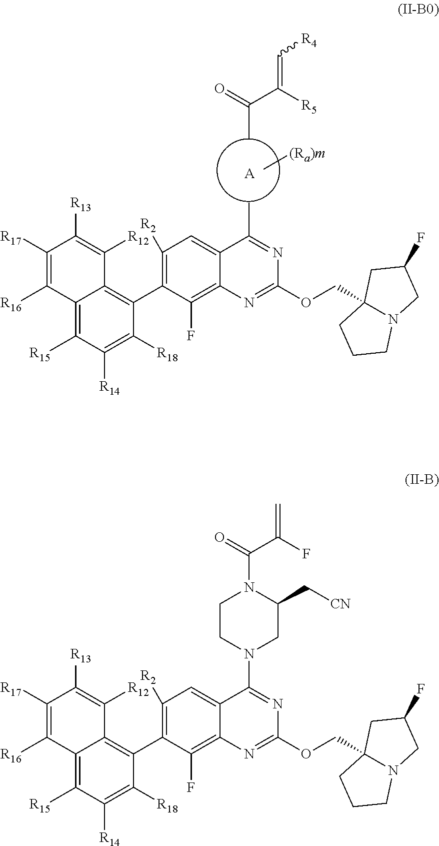

wherein R₂ and R₁₂ to R₁₈ have the definitions given above.

In addition, the inventors of the present invention have unexpectedly found that when R₆ in the compound of the present invention is 1-naphthyl and R₁₂ in the 8-position of the naphthyl is ethynyl (and also optionally, R₁₄ in the 3-position is hydroxyl), or R₁₂ in the 8-position of the naphthyl is chlorine and also preferably R₁₄ in the 3-position is hydroxyl, drug-resistant mutation can be significantly inhibited while ensuring excellent KRAS G12C mutation inhibitory activity and metabolic stability. If there is no specific substituent mentioned above or the substitution position is inconsistent with the above, excellent drug-resistant mutation inhibition may not be achieved.

Therefore, in a preferred technical aspect of the present invention, there is provided a compound of formula (II-C) or a pharmaceutically acceptable salt or stereoisomer thereof:

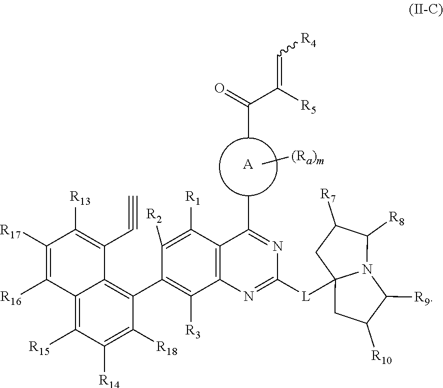

In a preferred technical solution of this aspect, R₁, R₃-R₅, R₈-R₁₀, A, L, Rₐ, and m in the compound of general formula (II-C) are defined as specific groups or values to obtain a compound of general formula (II-C1); and R₁₄ in the compound of formula (II-C1) is further defined as hydroxyl to obtain a compound of formula (II-C2):

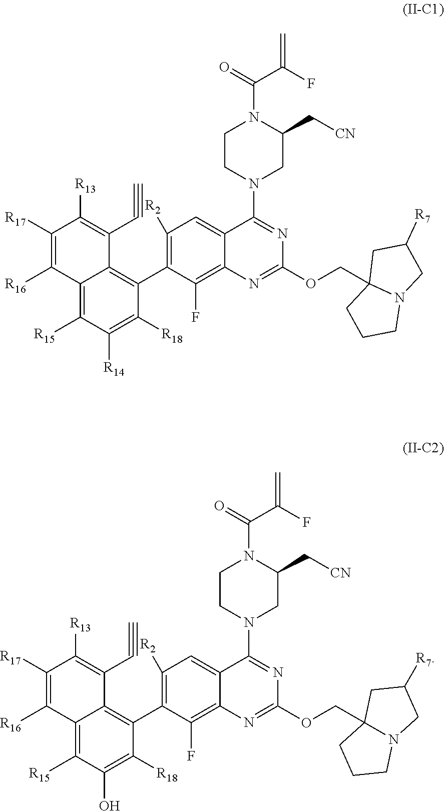

R₁₃ to R₁₈ are each independently selected from hydrogen, halogen (especially fluorine and chlorine), cyano, hydroxyl, amino, C₁-6 alkyl (especially ethyl), C₂₋₆ alkynyl (especially ethynyl), preferably hydrogen, fluorine, chlorine, cyano, methyl, ethyl, hydroxyl, and amino; and R₇ is preferably hydrogen or fluorine, and the other groups also each have the definitions given above.

The compounds of formula (II-C1) and formula (II-C2) further have a better KRAS G12C mutation inhibitory activity, drug-resistant mutation inhibitory effect, and/or metabolic stability.

In another preferred technical aspect of the present invention, there are provided compounds of formula (II-D) and formula (II-D0) or pharmaceutically acceptable salts or stereoisomers thereof:

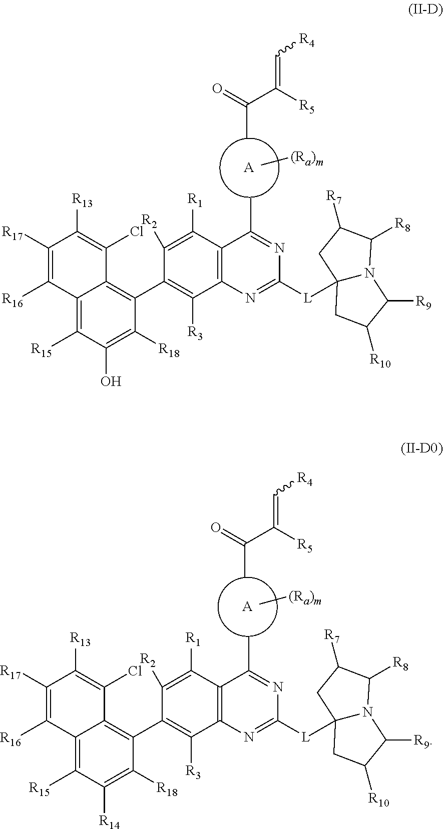

In a preferred technical solution of this aspect, R₁, R₃-R₅, R₈-R₁₀, R₁₄, A, L, Rₐ, and m in the compound of general formula (II-D0) are defined as specific groups or values to obtain compounds of formula (II-D1) and formula (II-D2):

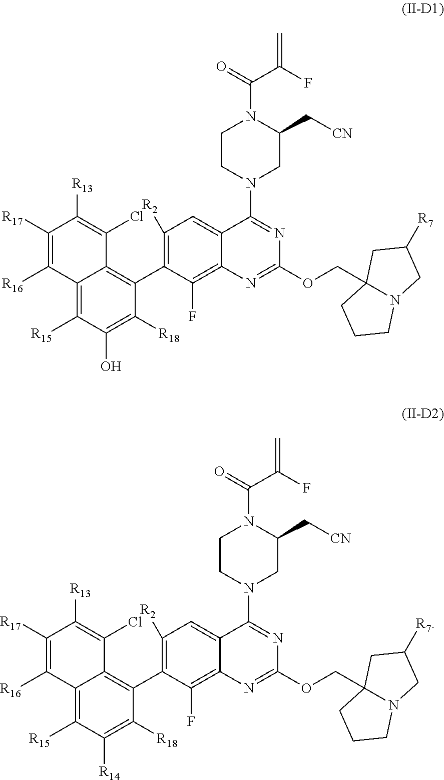

On the basis of the foregoing definitions of the groups, R₁₃ and R₁₅ to R₁₈ are each independently selected from hydrogen, halogen (especially fluorine and chlorine), cyano, hydroxyl, amino, C₁₋₆ alkyl (especially ethyl), and C₂₋₆ alkynyl (especially ethynyl), preferably hydrogen, fluorine, chlorine, cyano, methyl, ethyl, hydroxyl, and amino; and R₇ is preferably hydrogen or fluorine, R₁₄ is preferably hydroxyl, and the other groups also each have the definitions given above.

The compounds of formula (II-D1) and formula (II-D2) further have a better KRAS G12C mutation inhibitory activity, drug-resistant mutation inhibitory effect, and/or metabolic stability.

When the symbol “ [symbol] ” used in the description is not bonded with a bond, it represents the position where bond formation with another group occurs; or when “ [symbol] ” is connected to a double bond, it means that the bond may be located at any position in the plane of the double bond, for example, in formula (II), “ [symbol] R₄” means that R₄ and R₅ can form an E- or Z-configuration at the double bond.

In a preferred embodiment of the present invention, the compound according to formula (II) can be selected from the compounds of Examples 1-29 described below, more preferably the compounds of Examples 1-4, 7, 8, 10-16, and 18-24.

In another aspect, the present invention provides a pharmaceutical composition comprising a compound of formula (II) or a pharmaceutically acceptable salt or stereoisomer thereof as described above, and a pharmaceutically acceptable diluent or carrier.

In addition, the present invention further provides the use of the above compound or pharmaceutically acceptable salt or stereoisomer thereof in the preparation of a KRAS G12C mutation inhibitor.

As shown herein, the term “pharmaceutically acceptable salt” refers to a compound prepared in situ during the final separation and purification of the above compound, or a compound prepared by purifying a compound as a free acid or a free base, respectively and reacting the compound with an appropriate acid or base.

As shown herein, the term “aryl”, alone or in combination with another radical, means a carbocyclic aromatic monocyclic group containing 6-10 carbon atoms, which may be further fused with one or more 5- or 6-membered carbocyclic groups that may be aromatic and saturated or unsaturated.

The term “heteroaryl” refers to an aromatic monocyclic, bicyclic or tricyclic ring system which may contain 1-4 heteroatoms selected from N, O, and S. In the case of a bicyclic or tricyclic ring system, the term “heterocyclyl” may be in the form of a fused, bridged, or spiro ring. The term “heterocyclyl” may optionally be substituted with one or more substituents.

The term “heterocyclyl” refers to a saturated or unsaturated monocyclic, bicyclic or tricyclic ring system, in which at least one ring is a non-aromatic ring, which may contain 1-4 heteroatoms selected from N, O, and S. In the case of a bicyclic or tricyclic ring system, the term “heterocyclyl” may be in the form of a fused, bridged, or spiro ring. The term “heterocyclyl” may optionally be substituted with one or more substituents.

The term “halo” or “halogen” includes fluorine (F), chlorine (Cl), bromine (Br), or iodine (I).

In the presence of asymmetric atoms (such as carbon atoms) in the compound of formula (II), it can exist in the form of enantiomers, diastereomers, or a combination of enantiomers or diastereomers at any ratio (such as racemates).

For compounds provided with both chemical name and structural formula in the present description, when the chemical name and structural formula are inconsistent, the structural formula shall prevail.

In another embodiment, there is also provided a method for treating a disease with the compound or pharmaceutical composition of the present invention, wherein the disease includes, but is not limited to, diseases involving KRAS G12C mutation (e.g., cancer, such as lung cancer). The method comprises administering the compound of the present invention or a pharmaceutically acceptable salt or stereoisomer thereof to a subject in need thereof.

The metabolic stability described in the present description includes the metabolic stability in liver microsomes. The drug-resistant mutations described in the present description include Y96C, R68S, H95Q, Q99L, and other drug-resistant mutations.

### General Synthesis Method

The compounds in the present invention can be prepared by the synthesis methods and reactions shown in the following figures using commercially available reagents, or prepared by other reagents and conventional methods familiar to technicians.

The compounds of the present invention can be prepared by many methods, including standard chemical methods. Illustrative general synthesis methods are listed hereinafter, and the compound of formula (II) can be prepared by methods known in the field of organic synthesis. When referring to the methods of the examples described hereinafter, it should be understood that some substituents can be replaced by groups well known in the art, so that similar derivatives can be obtained without departing from the gist of the present invention. If necessary, protective groups are used for sensitive or reactive groups according to general principles or chemical methods. The protecting group is operated according to the standard method of organic synthesis (T. W. Greene and P. G. M. Wuts, Protective Groups in Organic Synthesis, 3rd edition, Wiley, New York 1999). These groups are removed at a convenient stage during compound synthesis using methods very clear to those skilled in the art. The selection method and reaction conditions and the treatment sequence thereof should conform to the preparation of the compound of formula (II).

Those skilled in the art will be able to identify whether there is a stereocenter in the compound of formula (II). Therefore, the present invention includes possible stereoisomers and includes both racemic compounds and single enantiomers. When the desired compound is a single enantiomer, it can be obtained by stereospecific synthesis or by resolution of the end product or any convenient intermediate. Resolution of the end products, intermediates or starting materials can be achieved by any suitable method known in the art. See, for example, E. L. Eliel, S. H. Wilen and L. N. Mander, “Stereochemistry of Organic Compounds” (Wiley-interscience, 1994).

### EXAMPLES

The following examples can further describe the present invention; however, these examples should not be taken as limitations to the scope of the present invention. The compound of formula (II) of the present invention includes, but is not limited to, the compounds described in Examples 1-29, wherein the compounds described in Examples 1 and 3-29 can also be prepared by referring to the synthesis route of Example 2.

### Preparation of Intermediates

Intermediate 1: The synthesis route of triisopropyl((8-potassium trifluoroboronate-2-yl)naphth-1-yl)ethynyl)silane was as follows.

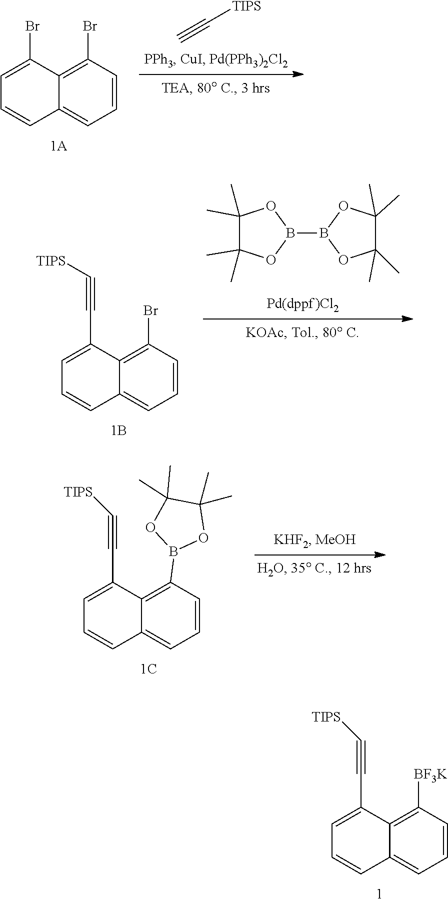

### Step I: Synthesis of ((8-bromonaphth-1-yl)ethynyl)triisopropylsilane

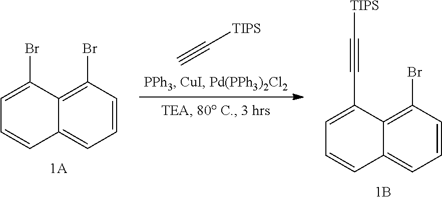

After 1,8-dichloronaphthalene (5.00 g, 17.4 mmol) and (triisopropylsilyl)acetylene (4.15 g, 22.7 mmol) were dissolved in triethylamine (100 mL), triphenylphosphine (458 mg, 1.75 mmol), bis(triphenylphosphino)dichloropalladium (613 mg, 874 umol), and cuprous iodide (333 mg, 1.75 mmol) were added and reacted at 80° C. for 3 hours. The reaction was complete as monitored by HPLC. After concentration and purification by column chromatography, ((8-bromonaphth-1-yl)ethynyl)triisopropylsilane was obtained as a yellow solid (5.80 g, yield 85.6%).

¹H NMR (400 MHz, CDCl₃): δ=7.92 (dd, J1=1.1, J2=7.3 Hz, 1H), 7.87-7.78 (m, 3H), 7.43 (t, J=7.7 Hz, 1H), 7.31-7.29 (m, 1H), 1.23-1.21 (m, 21H) ppm.

### Step II: Synthesis of triisopropyl((8-pinacol borate-2-yl)naphth-1-yl)ethynyl)silane

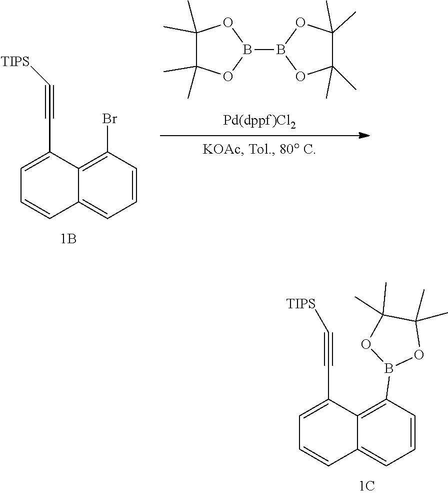

After ((8-bromonaphth-1-yl)ethynyl)triisopropylsilane (5.80 g, 14.9 mmol) and 4,4,5,5-tetramethyl-2-(4,4,5,5-tetramethyl-1,3,2-dioxaborolan-2-yl)-1,3,2-dioxaborolane (4.18 g, 16.4 mmol) were dissolved in toluene (60 mL) under nitrogen protection, potassium acetate (4.41 g, 44.9 mmol) and 1,1-bis(diphenylphosphino)ferrocene palladium chloride (1.10 g, 1.50 mmol) were then added. The reaction was carried out at 80° C. for 12 hours. The reaction was complete as monitored by HPLC. After concentration, column chromatography and prep-HPLC purification, triisopropyl((8-pinacol borate-2-yl)naphth-1-yl)ethynyl)silane as a yellow solid (2.30 g, yield 35.3%) was obtained.

¹H NMR (400 MHz, CDCl₃): δ=7.89-7.75 (m, 4H), 7.49-7.37 (m, 2H), 1.45 (s, 12H), 1.19 (s, 21H) ppm.

### Step III: Synthesis of triisopropyl((8-potassium trifluoroboronate-2-yl)naphth-1-yl)ethynyl)silane

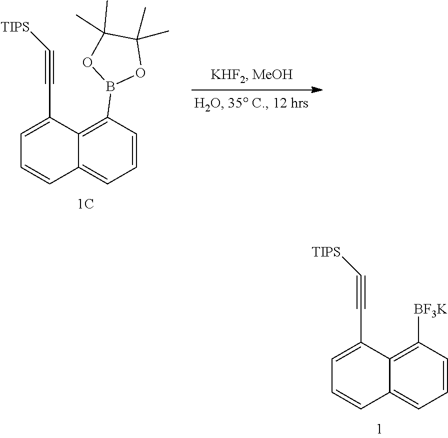

Under nitrogen protection, triisopropyl((8-pinacol borate-2-yl)naphth-1-yl)ethynyl)silane (1.50 g, 3.45 mmol) and potassium hydrogen fluoride (1.35 g, 17.2 mmol) were dissolved in methanol (24 mL) and water (4 mL). The reaction was carried out at 35° C. for 12 hours. The reaction was complete as monitored by TLC. The reaction liquid was filtered to obtain a filter cake, which was dried to obtain triisopropyl((8-potassium trifluoroboronate-2-yl)naphth-1-yl)ethynyl)silane as a white solid (1.50 g, crude).

¹H NMR (400 MHz, (CD₃)₂SO): δ=7.83 (d, J=6.9 Hz, 1H), 7.79-7.74 (m, 1H), 7.66-7.58 (m, 2H), 7.28 (dt, J1=3.1, J2=7.5 Hz, 2H), 1.17-1.10 (m, 21H) ppm.

Intermediate 2: The synthesis route of triisopropyl((6-(methoxymethoxy)-8-(4,4,5,5-tetramethyl-1,3,2-dioxaborolan-2-yl)naphth-1-yl)ethynyl)silane was as follows.

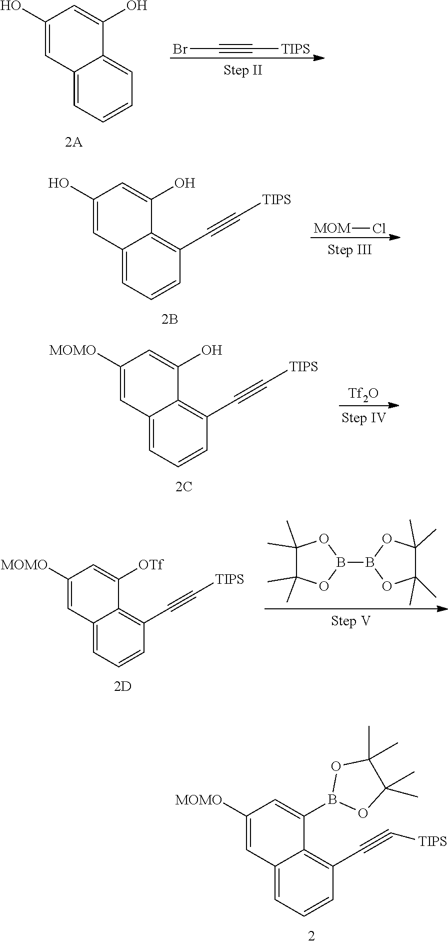

### Step I: Synthesis of (bromoethynyl)triisopropylsilane

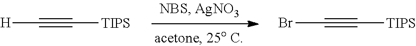

Ethynyltriisopropylsilane (24.0 g, 132 mmol), N-bromosuccinimide (26.9 g, 151 mmol), and silver nitrate (2.24 g, 13.2 mmol) were dissolved in acetone (240 mL). After nitrogen displacement three times, the mixture was stirred at 25° C. for 1 hour. The reaction was complete as monitored by TLC. The reaction liquid was directly concentrated and slurried with petroleum ether, and the filtrate was concentrated to obtain a product of (bromoethynyl)triisopropylsilane as a colorless oil (33.5 g, yield 97.4%).

¹H NMR (400 MHz, CDCl₃): δ=1.16-0.99 (m, 21H) ppm.

### Step II: Synthesis of 8-((triisopropylsilyl)ethynyl)naphthalene-1,3-diol

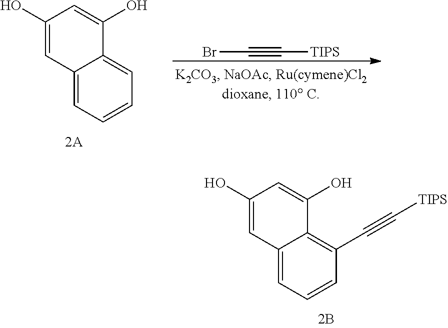

Naphthalene-1,3-diol (10.0 g, 62.4 mmol), (bromoethynyl)triisopropylsilane (17.9 g, 68.7 mmol), potassium acetate (12.2 g, 125 mmol), and bis(4-methylisopropylphenyl)ruthenium(II) dichloride (3.82 g, 6.24 mmol) were dissolved in dioxane (200 mL). After nitrogen displacement three times, the mixture was stirred at 110° C. for 12 hours. After the reaction was complete as monitored by TLC, the reaction liquid was directly concentrated and purified by a fast silica gel column to obtain 8-((triisopropylsilyl)ethynyl)naphthalene-1,3-diol (6.50 g, 30.6% yield).

¹H NMR (400 MHz, CDCl₃): δ=9.32 (s, 1H), 7.69-7.57 (m, 1H), 7.52-7.44 (m, 1H), 7.33-7.27 (m, 1H), 6.75 (d, J=2.5 Hz, 1H), 6.64 (d, J=2.5 Hz, 1H), 5.04 (br s, 1H), 1.23-1.16 (m, 21H) ppm.

### Step III: Synthesis of 3-(methoxymethoxy)-8-((triisopropylsilyl)ethynyl)naphth-1-ol

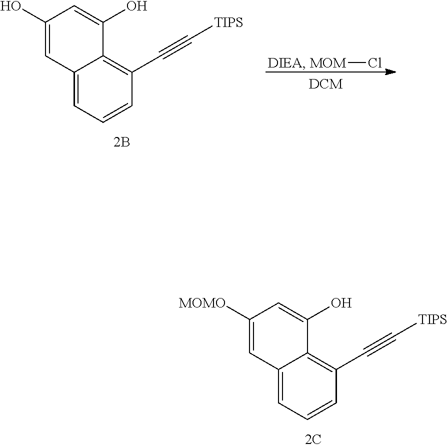

At 0° C., chloromethyl methyl ether (1.57 g, 19.5 mmol) was dropwise added to 8-((triisopropylsilyl)ethynyl)naphthalene-1,3-diol (6.50 g, 19.1 mmol) and diisopropylethylamine (4.93 g, 38.2 mmol) in dichloromethane (65.0 mL), and the mixture was then heated to 20° C. and further stirred for 2 hours. After the reaction was complete as monitored by TLC, the reaction liquid was added to water for quenching, adjusted to pH=6-7 with 1 M hydrochloric acid, and extracted with dichloromethane. The organic phase was dried and concentrated, and the crude product was purified by a fast silica gel column to obtain a product of 3-(methoxymethoxy)-8-((triisopropylsilyl)ethynyl)naphth-1-ol (3.50 g, yield 47.7%).

¹H NMR (400 MHz, CDCl₃): δ=9.27 (s, 1H), 7.74-7.64 (m, 1H), 7.53-7.47 (m, 1H), 7.36-7.28 (m, 1H), 6.98 (d, J=2.5 Hz, 1H), 6.78 (d, J=2.5 Hz, 1H), 5.27 (s, 2H), 3.52 (s, 3H), 1.23-1.15 (m, 21H) ppm.

### Step IV: Synthesis of 3-(methoxymethoxy)-8-((triisopropylsilyl)ethynyl)naphthalene-1-trifluoromethanesulfonate

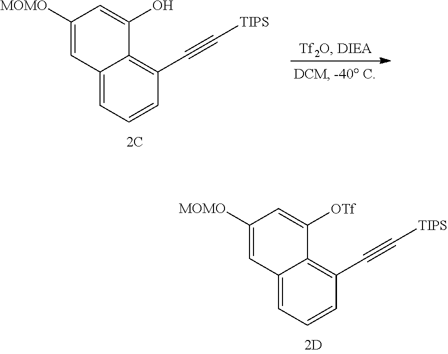

At −40° C., trifluoromethanesulfonic anhydride (4.11 g, 14.6 mmol) was dropwise added to 3-(methoxymethoxy)-8-((triisopropylsilyl)ethynyl)naphth-1-ol (3.50 g, 9.10 mmol) and diisopropylethylamine (4.70 g, 36.4 mmol) in dichloromethane (30.0 mL). Stirring was continued at this temperature for 0.5 hours, and the reaction was complete as monitored by TLC. The reaction liquid was added to water for quenching and extracted with dichloromethane, the organic phase was dried and concentrated, and the crude product was purified by a fast silica gel column to obtain (3-(methoxymethoxy)-8-((triisopropylsilyl)ethynyl)naphth-1-yl) trifluoromethanesulfonate as a yellow oil (4.60 g, yield 97.8%).

¹H NMR (400 MHz, CDCl₃): δ=7.82-7.67 (m, 2H), 7.47-7.42 (m, 2H), 7.32 (d, J=2.4 Hz, 1H), 5.30 (s, 2H), 3.53 (s, 3H), 1.26-1.15 (m, 21H) ppm.

### Step V: Synthesis of triisopropyl((6-(methoxymethoxy)-8-(4,4,5,5-tetramethyl-1,3,2-dioxaborolan-2-yl)naphth-1-yl)ethynyl)silane

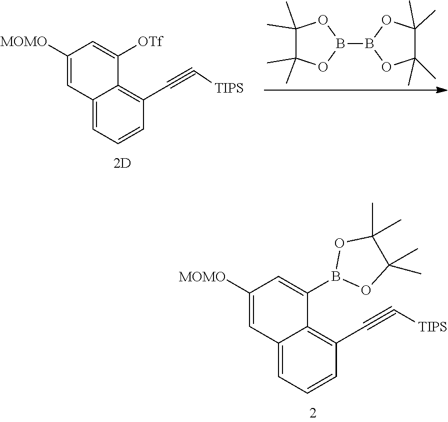

(3-(Methoxymethoxy)-8-((triisopropylsilyl)ethynyl)naphth-1-yl) trifluoromethanesulfonate (4.60 g, 8.90 mmol), bis(pinacolato)diboron (4.07 g, 16.0 mmol), 1,1-bis(diphenylphosphino)ferrocene palladium chloride (397 mg, 0.542 mmol), potassium acetate (2.18 g, 22.3 mmol), and dioxane (46.0 mL) were reacted at 120° C. for 12 hours. After the reaction was complete as monitored by LCMS, the reaction liquid was concentrated and purified by column chromatography to obtain a product of triisopropyl((6-(methoxymethoxy)-8-(4,4,5,5-tetramethyl-1,3,2-dioxaborolan-2-yl)naphth-1-yl)ethynyl)silane (3.60 g, yield 81.8%).

¹H NMR (400 MHz, CDCl₃): δ=7.67-7.55 (m, 2H), 7.39 (d, J=2.6 Hz, 1H), 7.33-7.23 (m, 2H), 5.21 (s, 2H), 3.48-3.36 (m, 3H), 1.35 (s, 12H), 1.11-1.07 (m, 21H) ppm.

Intermediate 3: The synthesis route of potassium (8-chloro-3-(methoxymethoxy)naphth-1-yl)trifluoroboronate was as follows.

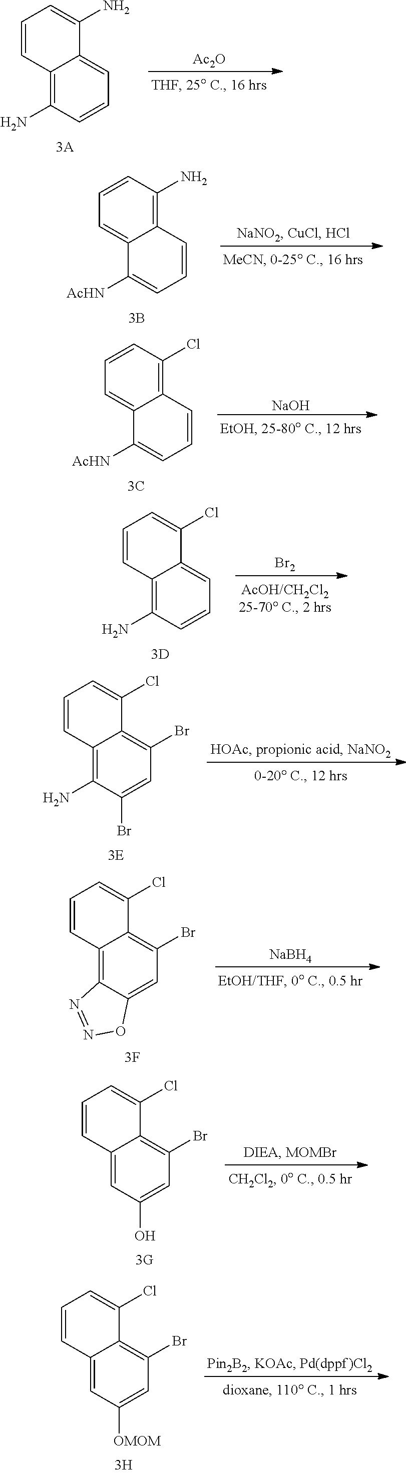

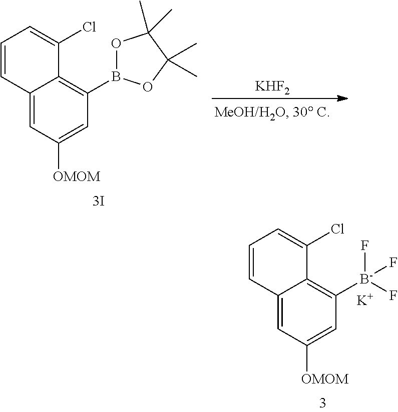

### Step I: Synthesis of N-(5-aminonaphth-1-yl)acetamide

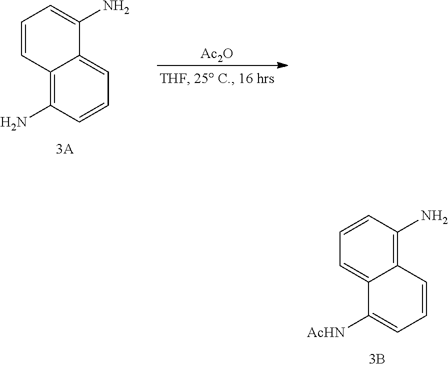 [SMILES: "Ac2O" ; Nc1cccc2c(N)cccc12 ; CC(=O)Nc1cccc2c(N)cccc12]

Naphthalene-1,5-diamine (10.0 g, 63.2 mmol) was dissolved in 300 mL of tetrahydrofuran, a solution of acetic anhydride (6.45 g, 63.2 mmol) dissolved in 60 mL of tetrahydrofuran was then added dropwise at 0° C., and the reaction liquid was stirred at 25° C. for 16 hours. After the reaction was complete as detected by LCMS, the reaction liquid was dried by rotary evaporation under reduced pressure, the crude product was slurried with methanol and filtered, and the filtrate was dried by rotary evaporation to obtain N-(5-aminonaphth-1-yl)acetamide (10.0 g, yield 78.2%).

¹H NMR (400 MHz, (CD₃)₂SO): δ=9.70 (s, 1H), 7.89 (d, J=8.4 Hz, 1H), 7.58 (d, J=7.3 Hz, 1H), 7.31 (t, J=7.9 Hz, 1H), 7.26-7.19 (m, 2H), 6.73-6.64 (m, 1H), 5.73 (s, 2H), 2.15 (s, 3H) ppm.

### Step II: Synthesis of N-(5-chloronaphth-1-yl)acetamide

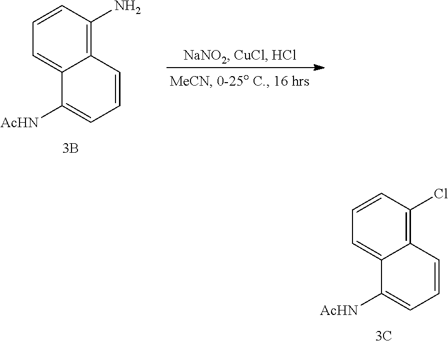

N-(5-aminonaphth-1-yl)acetamide (60.5 g, 302 mmol) was dissolved in 400 mL of hydrochloric acid (12 M) and 500 mL of acetonitrile and stirred at 0° C. for 2 hours, and sodium nitrite (25.1 g, 363 mmol) was then slowly added. Stirring was continued at 0° C. for 2 hours, cuprous chloride (45.0 g, 454 mmol) was then slowly added, and the reaction was then carried out at 25° C. for 12 hours. After the reaction was complete as detected by LCMS, the reaction liquid was concentrated, the crude product was dissolved with ethyl acetate and filtered, the filtrate was washed with a saturated sodium bicarbonate solution and a saturated sodium chloride aqueous solution, and the organic phase was concentrated to obtain N-(5-chloronaphth-1-yl)acetamide (42.6 g, yield 36.9%).

¹H NMR (400 MHz, (CD₃)₂SO): δ=10.04 (br s, 1H), 8.12-8.00 (m, 2H), 7.83-7.71 (m, 2H), 7.70-7.63 (m, 1H), 7.54 (br t, J=7.9 Hz, 1H), 2.19 (s, 3H) ppm.

### Step III: Synthesis of 5-chloronaphthalen-1-amine

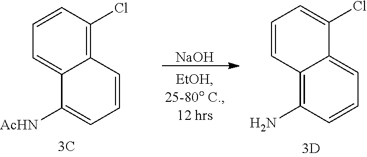 [SMILES: CC(=O)Nc1cccc2c(Cl)cccc12 ; "NaOH" ; Nc1cccc2c(Cl)cccc12]

At 25° C., N-(5-chloronaphth-1-yl)acetamide (41.2 g, 187 mmol) was dissolved in 300 mL of ethanol, sodium hydroxide (10 M, 937 mmol) was added and reacted at 80° C. for 12 hours. The reaction was complete as detected by LCMS. The reaction liquid was dried by rotary evaporation under reduced pressure, the crude product was extracted with methyl tert-butyl ether and washed with a saturated sodium chloride aqueous solution, and the organic phase was dried by rotary evaporation to obtain 5-chloronaphthalen-1-amine (33.0 g, yield 80.6%).

¹H NMR (400 MHz, (CD₃)₂SO): δ=8.09 (d, J=8.4 Hz, 1H), 7.58 (d, J=7.3 Hz, 1H), 7.40-7.26 (m, 3H), 6.80-6.71 (m, 1H), 5.94 (br s, 2H) ppm.

### Step IV: Synthesis of 2,4-dibromo-5-chloronaphthalen-1-amine

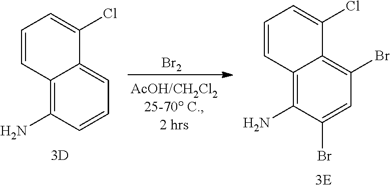

At 25° C., a solution of 5-chloronaphthalen-1-amine (31.6 g, 178 mmol) dissolved in 80 mL of dichloromethane was added to a solution of liquid bromine (62.4 g, 391 mmol) in acetic acid (100 mL), and after reaction at 70° C. for 2 hours, the reaction was complete as detected by LCMS. The reaction product was cooled to 25° C. and filtered, the filter cake was washed with acetic acid, a 15% sodium hydroxide solution was then added and stirred at room temperature for 30 minutes, and the filter cake was washed with water and dried by rotary evaporation to obtain 2,4-dibromo-5-chloronaphthalen-1-amine (44.4 g, yield 51.8%).

¹H NMR (400 MHz, (CD₃)₂SO): δ=8.31 (d, J=8.6 Hz, 1H), 7.90 (s, 1H), 7.73 (d, J=7.4 Hz, 1H), 7.46 (t, J=8.1 Hz, 1H), 6.61-5.89 (m, 2H) ppm.

### Step V: Synthesis of 5-bromo-6-chloronaphtho[1,2-d][1,2,3]oxadiazole

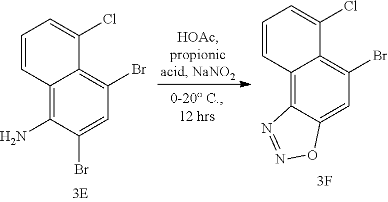 [SMILES: Nc1c(Br)cc(Br)c2c(Cl)cccc12 ; Clc1cccc2c1c(Br)cc1onnc12]

At 0° C., propionic acid (101 g, 1.37 mol) was slowly added to a solution of 2,4-dibromo-5-chloronaphthalen-1-amine (45.8 g, 136 mmol) in acetic acid (300 mL), sodium nitrite (14.1 g, 204 mmol) was then added, and the mixture was stirred at 0° C. for 0.5 hours, then heated to 20° C., and stirred for 11.5 hours. After the reaction was complete as detected by LCMS and TLC, the reaction liquid was poured into ice water and filtered, and the filter cake was dried by rotary evaporation to obtain 5-bromo-6-chloronaphtho[1,2-d][1,2,3]oxadiazole (28.2 g, yield 43.6%).

¹H NMR (400 MHz, (CD₃)₂SO): δ=7.66 (dd, J=1.2, 8.1 Hz, 1H), 7.60-7.56 (m, 1H), 7.46 (dd, J=1.3, 7.8 Hz, 1H), 7.27 (s, 1H) ppm.

### Step VI: Synthesis of 4-bromo-5-chloronaphthalen-2-ol

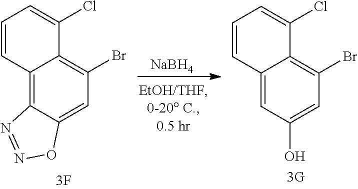 [SMILES: Clc1cccc2c1c(Br)cc1onnc12 ; "NaBH4" ; Oc1cc(Br)c2c(Cl)cccc2c1]

5-Bromo-6-chloronaphtho[1,2-d][1,2,3]oxadiazole (28.2 g, 99.5 mmol) was added to 100 mL of ethanol and 100 mL of tetrahydrofuran, and sodium borohydride (7.27 g, 192 mmol) was slowly added at 0° C. and reacted at 0° C. for 0.5 hours. After the reaction was complete as detected by TLC, the reaction liquid was quenched by adding 1 M hydrochloric acid and then adjusted to pH 7-8 with a 20% sodium hydroxide solution, and after extraction with ethyl acetate, the organic phase was washed with water, dried, concentrated, and separated by column chromatography to obtain 4-bromo-5-chloronaphthalen-2-ol (6.48 g, yield 25.3%).

¹H NMR (400 MHz, (CD₃)₂SO): δ=10.36 (s, 1H), 7.78 (dd, J=1.0, 8.3 Hz, 1H), 7.57 (d, J=2.5 Hz, 1H), 7.48 (dd, J=1.3, 7.4 Hz, 1H), 7.38 (d, J=8.0 Hz, 1H), 7.27 (d, J=2.5 Hz, 1H) ppm.

### Step VII: Synthesis of 1-bromo-8-chloro-3-(methoxymethoxy)naphthalene

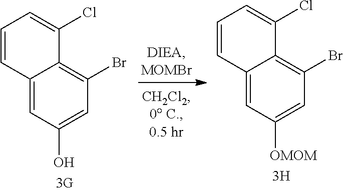

4-Bromo-5-chloronaphthalen-2-ol (5.89 g, 22.8 mmol) and N,N-diisopropylethylamine (5.91 g, 45.7 mmol) were dissolved in 60 mL of dichloromethane, and bromomethyl methyl ether (2.86 g, 22.8 mmol) was slowly dropwise added at 0° C. and reacted at 20° C. for 0.5 hours. After the reaction was complete as monitored by TLC, a saturated sodium chloride aqueous solution was added to the reaction liquid, the reaction liquid was extracted with dichloromethane, dried, and concentrated, and the crude product was separated by a silica gel column to obtain 1-bromo-8-chloro-3-(methoxymethoxy)naphthalene (5.00 g, yield 72.5%).

¹H NMR (400 MHz, CDCl₃): δ=7.63 (d, J=2.5 Hz, 1H), 7.61 (dd, J=0.9, 8.3 Hz, 1H), 7.45 (dd, J=1.3, 7.5 Hz, 1H), 7.32 (d, J=2.5 Hz, 1H), 7.26-7.21 (m, 1H), 5.22 (s, 2H), 3.48 (s, 1H), 3.46 (s, 3H) ppm.

### Step VIII: Synthesis of pinacol 2-(8-chloro-3-(methoxymethoxy)naphth-1-yl)boronate

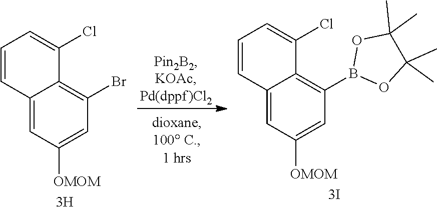

1-Bromo-8-chloro-3-(methoxymethoxy)naphthalene (5.00 g, 16.5 mmol), bis(pinacolato)diboron (4.63 g, 18.2 mmol), potassium acetate (4.88 g, 49.7 mol), and 1,1-bis(diphenylphosphino)ferrocene palladium chloride (1.21 mg, 1.66 mol) were dissolved in dioxane (4 M, 72.3 uL), and the mixture was then stirred at 110° C. for 1 hour. After the reaction was complete as monitored by TLC, the reaction liquid was extracted with water and ethyl acetate, the organic phase was dried and filtered, and the filtrate was concentrated and separated by a silica gel column to obtain pinacol 2-(8-chloro-3-(methoxymethoxy)naphth-1-yl)boronate (4.18 g, yield 72.3%).

¹H NMR (400 MHz, CDCl₃): δ=7.68-7.63 (m, 1H), 7.44 (dd, J=1.1, 7.4 Hz, 1H), 7.41 (d, J=2.5 Hz, 1H), 7.36 (d, J=2.5 Hz, 1H), 7.35-7.30 (m, 1H), 5.30 (s, 2H), 3.51 (s, 3H), 1.45 (s, 12H) ppm.

### Step IX: Synthesis of potassium (8-chloro-3-(methoxymethoxy)naphth-1-yl)trifluoroboronate

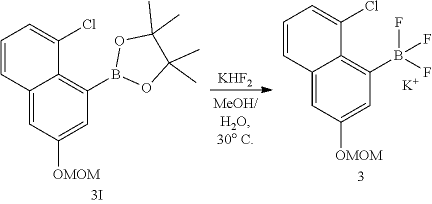

Pinanol 2-(8-chloro-3-(methoxymethoxy)naphth-1-yl)boronate (1.00 g, 2.87 mmol) was dissolved in 10 mL of methanol, potassium hydrogen fluoride (1.34 g, 17.2 mmol) dissolved in 1 mL of water was then added, and the reaction liquid was stirred at 30° C. for 7 hours. After the reaction was complete as monitored by TLC, the reaction liquid was dried by rotary evaporation to remove methanol and filtered, and the filter cake was dried by rotary evaporation to obtain potassium (8-chloro-3-(methoxymethoxy)naphth-1-yl)trifluoroboronate (835 mg, yield 88.6%).

¹H NMR (400 MHz, (CD₃)₂SO): δ=7.66-7.58 (m, 2H), 7.31 (d, J=7.0 Hz, 1H), 7.24-7.17 (m, 2H), 5.26 (s, 2H), 3.40 (s, 3H) ppm.

Intermediate 4: The synthesis route of tert-butyl (S)-4-(7-bromo-2-chloro-8-fluoroquinazolin-4-yl)-2-(cyanomethyl)piperazine-1-carboxylate was as follows.

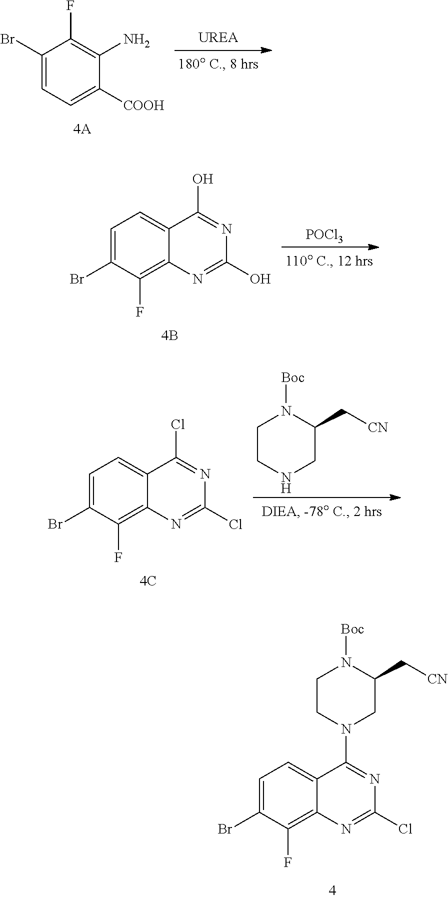 [SMILES: Nc1c(C(=O)O)ccc(Br)c1F ; Oc1nc(O)c2ccc(Br)c(F)c2n1 ; "POCl3" ; Fc1c(Br)ccc2c(Cl)nc(Cl)nc12 ; CC(C)(C)OC(=O)N1CCNC[C@@H]1CC#N ; CC(C)(C)OC(=O)N1CCN(c2nc(Cl)nc3c(F)c(Br)ccc23)C[C@@H]1CC#N]

### Step I: Synthesis of 7-bromo-8-fluoroquinazoline-2,4-diol

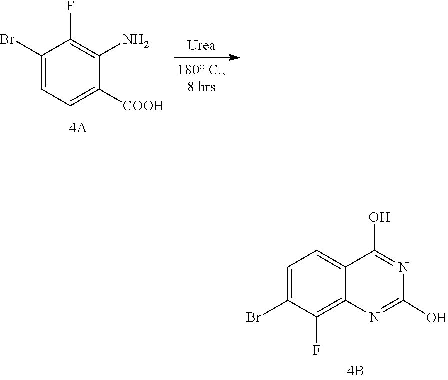 [SMILES: Nc1c(C(=O)O)ccc(Br)c1F ; Oc1nc(O)c2ccc(Br)c(F)c2n1]

2-Amino-4-bromo-3-fluorobenzoic acid (2.50 g, 10.7 mmol, 1.00 eq) and urea (5.13 g, 85.5 mmol, 8.00 eq) were reacted at 180° C. for 4 hours. The reaction was complete as monitored by LCMS. After adding water, the reaction liquid was boiled at 100° C. for 30 minutes and filtered, and the filter cake was washed with boiling water three times and then dried by rotary evaporation to obtain 7-bromo-8-fluoroquinazoline-2,4-diol (8.92 g, crude).

LCMS: m/z 259.9 [M+3]⁺.

¹H NMR (400 MHz, (CD₃)₂SO): δ=11.51 (br s, 2H), 7.63 (dd, J=0.7, 8.6 Hz, 1H), 7.43 (dd, J=5.9, 8.5 Hz, 1H).

### Step II: Synthesis of 7-bromo-2,4-dichloro-8-fluoroquinazoline

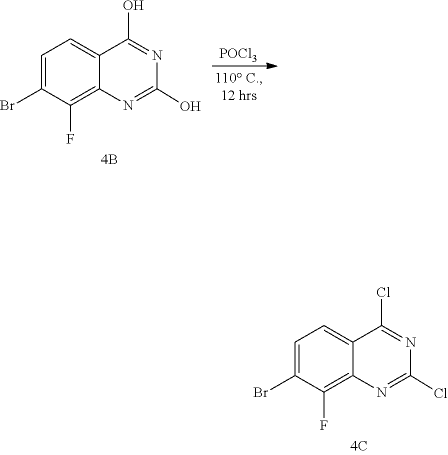 [SMILES: Oc1nc(O)c2ccc(Br)c(F)c2n1 ; "POCl3" ; Fc1c(Br)ccc2c(Cl)nc(Cl)nc12]

7-Bromo-8-fluoroquinazoline-2,4-diol (8.92 g, 34.4 mmol, 1.00 eq) was dissolved in 40.0 mL of phosphorus oxychloride, N,N-diisopropylethylamine (13.4 g, 103 mmol, 18.0 mL, 3.00 eq) was added, and the reaction liquid was reacted at 110° C. for 12 hours. After the reaction was complete as detected by LCMS, the reaction liquid was concentrated, then dissolved with dichloromethane, then filtered through a silica gel pad, and dried by rotary evaporation to obtain 7-bromo-2,4-dichloro-8-fluoroquinazoline (28.9 g, crude).

LCMS: m/z 296.9 [M+3]⁺.

¹H NMR (400 MHz, CDCl₃): δ=8.00-7.93 (m, 1H), 7.87 (dd, J=5.9, 9.0 Hz, 1H).

### Step III: Synthesis of tert-butyl (S)-4-(7-bromo-2-chloro-8-fluoroquinazolin-4-yl)-2-(cyanomethyl)piperazine-1-carboxylate

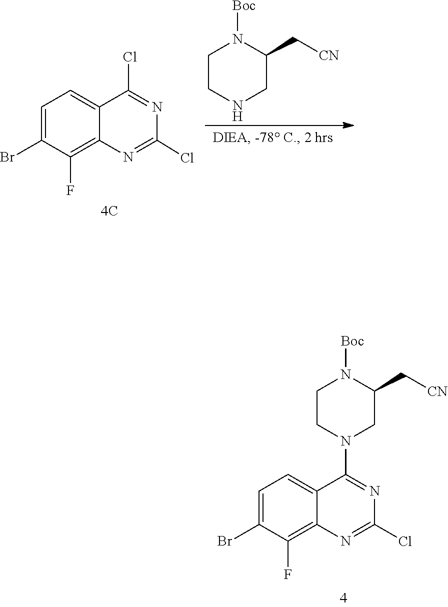 [SMILES: Fc1c(Br)ccc2c(Cl)nc(Cl)nc12 ; CC(C)(C)OC(=O)N1CCNC[C@@H]1CC#N ; CC(C)(C)OC(=O)N1CCN(c2nc(Cl)nc3c(F)c(Br)ccc23)C[C@@H]1CC#N]

7-Bromo-2,4-dichloro-8-fluoroquinazoline (25.8 g, 28.8 mmol, 33.0% purity, 1.00 eq) was dissolved in 100 mL of dichloromethane, N,N-diisopropylethylamine (11.2 g, 86.3 mmol, 3.00 eq) was added, the mixture was cooled to −78° C., (S)-tert-butyl 2-(cyanomethyl)piperazine-1-carboxylate (6.48 g, 28.8 umol, 1.00 eq) was added, and the reaction liquid was stirred at −78° C. for 2 hours. After the reaction was complete as monitored by TLC, water and dichloromethane were added to the reaction liquid at −78° C., and after extraction with dichloromethane, the organic phase was concentrated and purified by a fast silica gel column to obtain tert-butyl (S)-4-(7-bromo-2-chloro-8-fluoroquinazolin-4-yl)-2-(cyanomethyl)piperazine-1-carboxylate (7.62 g, 15.1 mmol, 52.6% yield).

LCMS: m/z 486.1 [M+3]⁺.

¹H NMR (400 MHz, CDCl₃): δ=7.63-7.53 (m, 2H), 4.65 (br s, 1H), 4.46-4.22 (m, 2H), 4.18-4.02 (m, 1H), 3.74 (br dd, J=3.9, 13.9 Hz, 1H), 3.62-3.27 (m, 2H), 2.93-2.78 (m, 1H), 2.76-2.66 (m, 1H), 1.52 (s, 9H).

Intermediate 5: The synthesis route of tert-butyl (S)-4-(7-bromo-2,8-difluoroquinazolin-4-yl)-2-(cyanomethyl)piperazine-1-carboxylate was as follows.

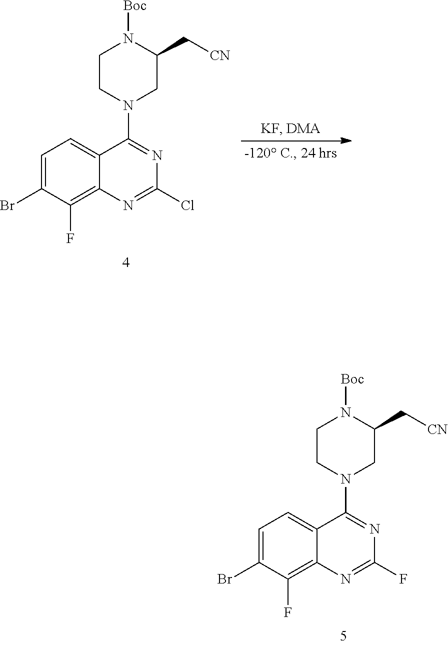 [SMILES: CC(C)(C)OC(=O)N1CCN(c2nc(Cl)nc3c(F)c(Br)ccc23)C[C@@H]1CC#N ; CC(C)(C)OC(=O)N1CCN(c2nc(F)nc3c(F)c(Br)ccc23)C[C@@H]1CC#N]

Intermediate 4 was used as a raw material. Tert-butyl (S)-4-(7-bromo-2-chloro-8-fluoroquinazolin-4-yl)-2-(cyanomethyl)piperazine-1-carboxylate (1.00 g, 2.06 mmol, 1.00 eq) was dissolved in 20.0 mL of N,N-dimethylacetamide, potassium fluoride (7.19 g, 124 mmol, 2.90 mL, 60.0 eq) was added, and the reaction liquid was stirred at 120° C. for 24 hours. After the reaction was complete as monitored by LCMS, the reaction liquid was diluted with ethyl acetate and washed with brine, and the organic phase was concentrated and purified by a fast silica gel column to obtain tert-butyl (S)-4-(7-bromo-2,8-difluoroquinazolin-4-yl)-2-(cyanomethyl)piperazine-1-carboxylate (0.71 g, 1.40 mmol, 67.9% yield).

LCMS: m/z 470.1 [M+3]⁺.

¹H NMR (400 MHz, CDCl₃): δ=7.71-7.45 (m, 2H), 4.63 (br d, J=3.3 Hz, 1H), 4.41-4.25 (m, 2H), 4.09 (br s, 1H), 3.80 (br dd, J=4.1, 13.9 Hz, 1H), 3.66-3.34 (m, 2H), 2.85 (br dd, J=6.5, 17.1 Hz, 1H), 2.78-2.65 (m, 1H), 1.50 (s, 9H).

Intermediate 6: The synthesis route of tert-butyl (S)-4-(7-bromo-2,6,8-difluoroquinazolin-4-yl)-2-(cyanomethyl)piperazine-1-carboxylate was as follows:

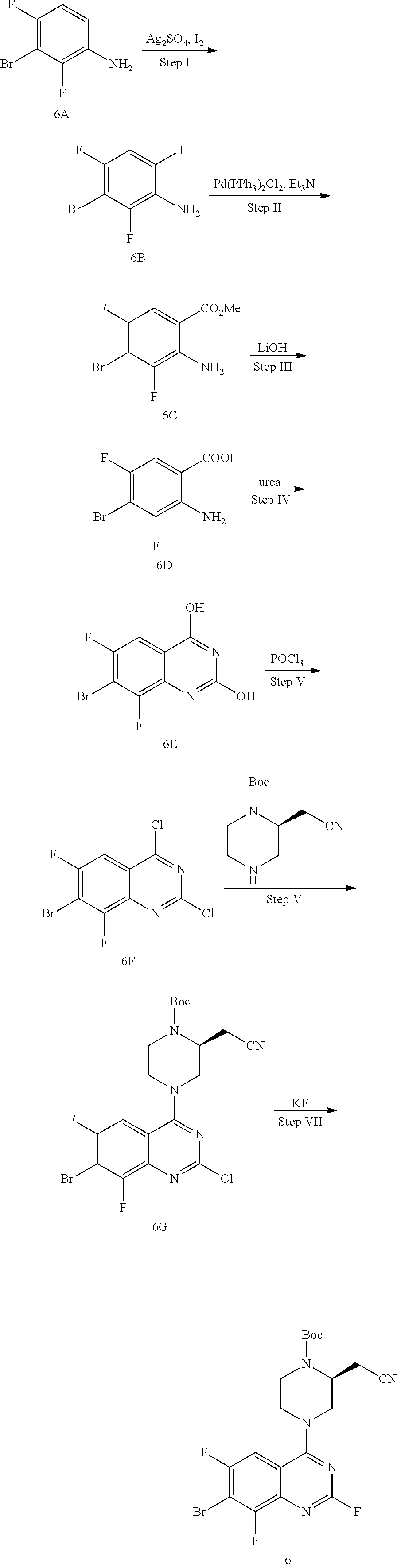

### Step I: Synthesis of 3-bromo-2,4-difluoro-6-iodoaniline

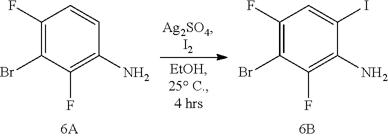 [SMILES: Nc1ccc(F)c(Br)c1F ; Nc1c(I)cc(F)c(Br)c1F]

3-Bromo-2,4-difluoroaniline (50.0 g, 240 mmol), silver sulfate (74.9 g, 240 mmol), and iodine (67.5 g, 265 mmol) were dissolved in ethanol (1000 mL) and reacted at 25° C. under nitrogen protection for 4 hours. The reaction was complete as monitored by LCMS. The reaction liquid was filtered, concentrated, and purified by column chromatography to obtain 3-bromo-2,4-difluoro-6-iodoaniline as a purple solid (70.5 g, yield 87.8%).

LCMS: m/z 335.9 [M+H]⁺.

¹H NMR (400 MHz, (CD₃)₂SO): δ=8.47-8.10 (m, 2H), 7.54 (dd, J=2.1, 8.0 Hz, 1H) ppm.

### Step II Synthesis of methyl 2-amino-4-bromo-3,5-difluorobenzoate

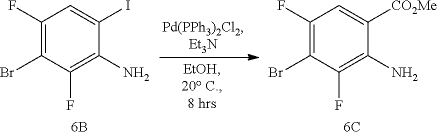 [SMILES: Nc1c(I)cc(F)c(Br)c1F ; COC(=O)c1cc(F)c(Br)c(F)c1N]

At 25° C., 3-bromo-2,4-difluoro-6-iodoaniline (34.0 g, 101 mmol) was dissolved in methanol (800 mL), [1,1-bis(diphenylphosphino)ferrocene]dichloropalladium dichloromethane (4.99 g, 6.11 mmol) was then added and then reacted for 5 minutes, triethylamine (72.1 g, 712 mmol) was then added, and finally, the reaction was carried out under carbon monoxide protection (15 psi) at 60° C. for 4 hours. The reaction was complete as monitored by LCMS and TLC. The reaction liquid was concentrated and passed through a column to obtain methyl 2-amino-4-bromo-3,5-difluorobenzoate as a yellow solid (18.0 g, yield 66.4%).

LCMS: m/z 265.9 [M+H]⁺.

¹H NMR (400 MHz, (CD₃)₂SO): δ=7.43 (dd, J=1.7, 9.4 Hz, 1H), 6.63 (br s, 2H), 3.82 (s, 3H) ppm.

### Step III: Synthesis of 2-amino-4-bromo-3,5-difluorobenzoic acid

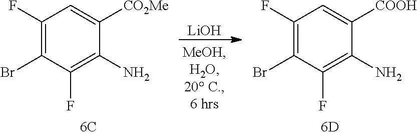 [SMILES: COC(=O)c1cc(F)c(Br)c(F)c1N ; "LiOH" ; Nc1c(C(=O)O)cc(F)c(Br)c1F]

After methyl 2-amino-4-bromo-3,5-difluorobenzoate (30.0 g, 112 mmol) was dissolved in methanol (30.0 mL), water (60.0 mL), and tetrahydrofuran (90.0 mL), lithium hydroxide monohydrate (4.73 g, 112 mmol) was added and then reacted at 25° C. for 12 hours. The reaction was complete as monitored by LCMS. After being concentrated, the reaction liquid was adjusted to pH 4 with 2M hydrochloric acid and then filtered to obtain a filter cake, which was dried to obtain 2-amino-4-bromo-3,5-difluorobenzoic acid as a brown solid (29.0 g, crude).

LCMS: m/z 253.9 [M+H]⁺.

¹H NMR (400 MHz, CD₃CN): 6=7.47 (dd, J=2.1, 9.5 Hz, 1H), 7.21-5.25 (m, 2H) ppm.

### Step IV: Synthesis of 7-bromo-6,8-difluoroquinazoline-2,4-diol

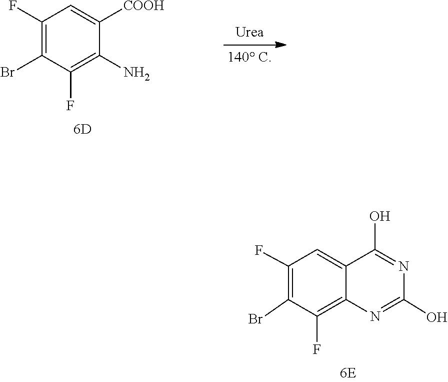 [SMILES: Nc1c(C(=O)O)cc(F)c(Br)c1F ; Oc1nc(O)c2cc(F)c(Br)c(F)c2n1]

Under nitrogen protection, 2-amino-4-bromo-3,5-difluorobenzoic acid (10.0 g, 39.6 mmol) was dissolved in urea (23.8 g, 396 mmol) and then reacted at 140° C. for 4 hours. The reaction was complete as monitored by LCMS. The reaction liquid was cooled to 100° C., water (10 mL) was added, the reaction liquid was then filtered, and the filter cake was dried to obtain 7-bromo-6,8-difluoroquinazoline-2,4-diol as a gray solid (8.98 g, crude).

LCMS: m/z 277.9 [M+H]⁺.

¹H NMR (400 MHz, (CD₃)₂SO): δ=11.67-11.44 (m, 2H), 7.63-7.56 (m, 1H) ppm.

### Step V: Synthesis of 7-bromo-2,4-dichloro-6,8-difluoroquinazoline

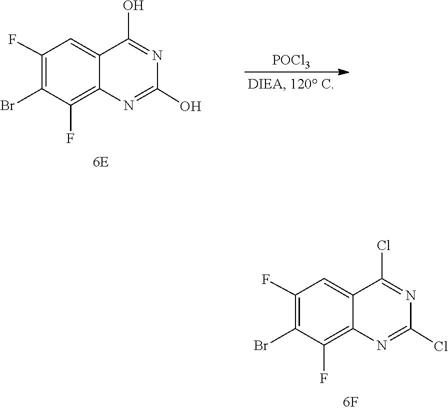 [SMILES: Oc1nc(O)c2cc(F)c(Br)c(F)c2n1 ; "POCl3" ; Fc1cc2c(Cl)nc(Cl)nc2c(F)c1Br]

At 25° C., 7-bromo-6,8-difluoroquinazoline-2,4-diol (10.0 g, 36.1 mmol) was dissolved in phosphorus oxychloride (300 mL) and then reacted at 120° C. for 5 minutes. Then, N,N-diisopropylethylamine (93.3 g, 721 mmol) was added and then reacted at 120° C. for 4 hours. The reaction was complete as monitored by TLC. The reaction liquid was concentrated to obtain 7-bromo-2,4-dichloro-6,8-difluoroquinazoline as a yellow solid (40.0 g, crude), which was directly used for the next step of reaction.

### Step VI: Synthesis of tert-butyl (S)-4-(7-bromo-2-chloro-6,8-difluoroquinazolin-4-yl)-2-(cyanomethyl)piperazine-1-carboxylate

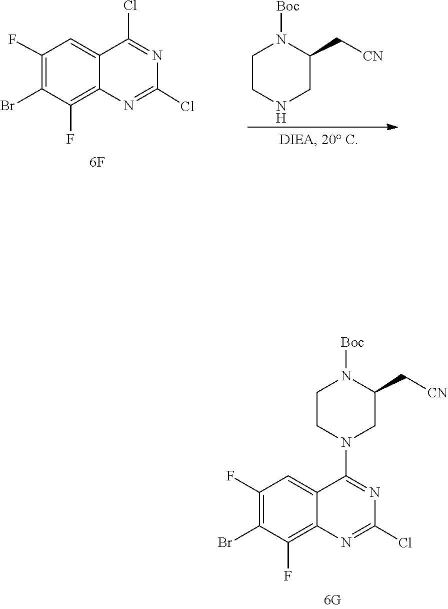 [SMILES: Fc1cc2c(Cl)nc(Cl)nc2c(F)c1Br ; CC(C)(C)OC(=O)N1CCNC[C@@H]1CC#N ; CC(C)(C)OC(=O)N1CCN(c2nc(Cl)nc3c(F)c(Br)c(F)cc23)C[C@@H]1CC#N]

At 25° C., tert-butyl (S)-2-(cyanomethyl)piperazine-1-carboxylate (3.95 g, 17.5 mmol) was dissolved in dichloromethane (100 mL), N,N-diisopropylethylamine (13.5 g, 105 mmol) was then added, 7-bromo-2,4-dichloro-6,8-difluoroquinazoline (11.0 g, 35.04 mmol) was then added at −60° C., and the mixture was then reacted for 30 minutes. The reaction was complete as monitored by LCMS.

The reaction liquid was quenched with water at −60° C., extracted with dichloromethane, washed with a saturated sodium chloride aqueous solution, and dried over sodium sulfate, and after filtration, concentration and column chromatography, tert-butyl (S)-4-(7-bromo-2-chloro-6,8-difluoroquinazolin-4-yl)-2-(cyanomethyl)piperazine-1-carboxylate as a yellow solid (2.30 g, yield 13.1%) was obtained.

LCMS: m/z 504.1 [M+H]⁺.

¹H NMR (400 MHz, CDCl₃): δ=7.50-7.42 (m, 1H), 4.65 (br s, 1H), 4.37-4.22 (m, 2H), 4.18-4.03 (m, 1H), 3.76 (br dd, J=3.6, 13.7 Hz, 1H), 3.64-3.34 (m, 2H), 2.93-2.80 (m, 1H), 2.76-2.66 (m, 1H), 1.52 (s, 9H) ppm.

### Step VII: Synthesis of tert-butyl (S)-4-(7-bromo-2,6,8-trifluoroquinazolin-4-yl)-2-(cyanomethyl)piperazine-1-carboxylate

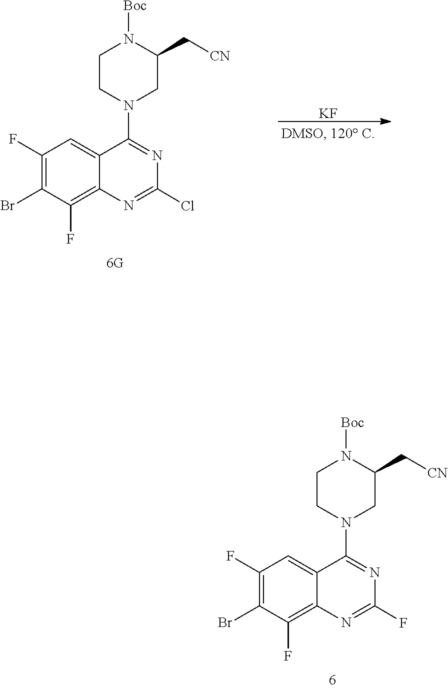 [SMILES: CC(C)(C)OC(=O)N1CCN(c2nc(Cl)nc3c(F)c(Br)c(F)cc23)C[C@@H]1CC#N ; "KF" ; CC(C)(C)OC(=O)N1CCN(c2nc(F)nc3c(F)c(Br)c(F)cc23)C[C@@H]1CC#N]

Tert-butyl (S)-4-(7-bromo-2-chloro-6,8-difluoroquinazolin-4-yl)-2-(cyanomethyl)piperazine-1-carboxylate (2.70 g, 5.37 mmol) and potassium fluoride (12.4 g, 214 mmol) were dissolved in dimethyl sulfoxide (8 mL) and then reacted at 120° C. under nitrogen protection for 4 hours. The reaction was complete as monitored by LCMS. The reaction liquid was quenched by adding water, extracted with dichloromethane, washed with a saturated sodium chloride aqueous solution, and dried over sodium sulfate, and after filtration, concentration and column chromatography, tert-butyl (S)-4-(7-bromo-2,6,8-trifluoroquinazolin-4-yl)-2-(cyanomethyl)piperazine-1-carboxylate as a yellow solid (1.01 g, yield 38.6%) was obtained.

LCMS: m/z 488.0 [M+H]⁺.

¹H NMR (400 MHz, CDCl₃): δ=7.54-7.47 (m, 1H), 4.65 (br s, 1H), 4.37-4.25 (m, 2H), 4.20-4.05 (m, 1H), 3.85 (br dd, J=4.0, 13.9 Hz, 1H), 3.63 (dt, J=3.8, 11.6 Hz, 1H), 3.49 (br s, 1H), 2.84 (br d, J=5.5 Hz, 1H), 2.75-2.65 (m, 1H), 1.52 (s, 9H) ppm.

### Example 1: 2-((S)-4-(7-(8-ethynyl-3-hydroxynaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 1)

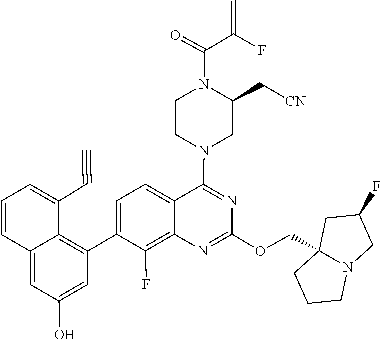 [SMILES: C#Cc1cccc2cc(O)cc(-c3ccc4c(N5CCN(C(=O)C(=C)F)[C@@H](CC#N)C5)nc(OC[C@@]56CCCN5C[C@H](F)C6)nc4c3F)c12]

### Specific Synthesis Route of Compound 1

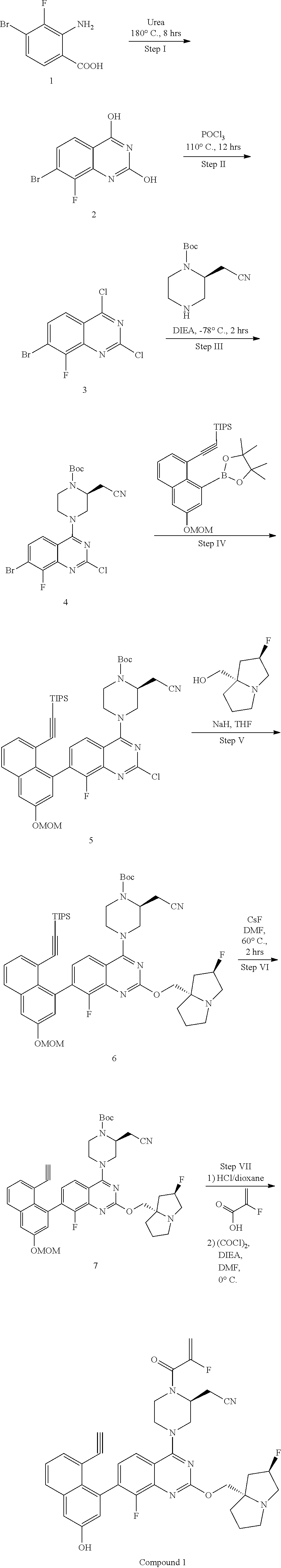

Starting from the intermediate No. 4 compound, the specific synthesis process of Compound 1 was as follows:

### Step IV: Synthesis of tert-butyl (S′)-4-(2-chloro-8-fluoro-7-(3-((methoxymethoxy)-8-((tri isopropylsilyl)ethynyl)naphth-1-yl)quinazolin-4-yl)-2-(cyanomethyl)piperazine-1-carboxylate

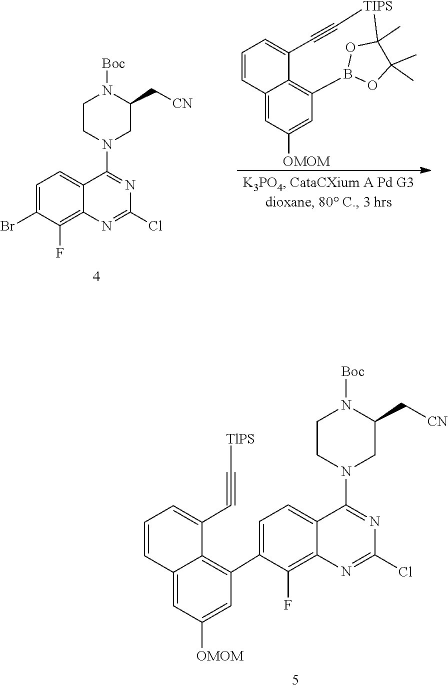

Tert-butyl (S)-4-(7-bromo-2-chloro-8-fluoroquinazolin-4-yl)-2-(cyanomethyl)piperazine-1-carboxylate (500 mg, 1.03 mmol), triisopropyl((6-(methoxymethoxy)-8-(4,4,5,5-tetramethyl-1,3,2-dioxaboran-2-yl)naphth-1-yl)ethynyl)silane (510 mg, 1.03 mmol) were dissolved in water (1.25 mL) and glycol dimethyl ether (12.5 mL). Methanesulfonato(diadamantyl-N-butylphosphino)-2′-amino-1,1′-biphenyl-2-yl)palladium(II) (93.3 mg, 128 umol) and potassium carbonate (656 mg, 3.09 mmol) were added, and the reaction liquid was stirred at 80° C. for 2 hours. After the reaction was complete as monitored by LCMS, the reaction liquid was concentrated and purified by a fast silica gel column to obtain tert-butyl (S)-4-(2-chloro-8-fluoro-7-(3-((methoxymethoxy)-8-((triisopropylsilyl)ethynyl)naphth-1-yl)quinazolin-4-yl)-2-(cyanomethyl)piperazine-1-carboxylate (1.40 g, yield 55.6%).

LCMS: m/z 667.3 [M+H]⁺.

¹H NMR (400 MHz, CDCl₃): δ=7.75 (d, J=8.1 Hz, 1H), 7.65-7.56 (m, 2H), 7.47-7.32 (m, 3H), 7.07 (d, J=2.0 Hz, 1H), 5.24 (s, 2H), 4.65 (br s, 1H), 4.46-4.08 (m, 3H), 3.77-3.64 (m, 1H), 3.57-3.34 (m, 5H), 3.01-2.65 (m, 2H), 1.46 (d, J=3.6 Hz, 9H), 0.83-0.72 (m, 18H), 0.61-0.32 (m, 3H) ppm.

### Step V: Synthesis of tert-butyl (S)-2-(cyanomethyl)-4-(8-fluoro-2-((2R,7aS)-2-fluoro-tetrahydrofuran-1H-pyrrolizin-7a(5H)-yl)methoxy)-7-(3-(methoxymethyl)oxy)-8-(triisopropylsilyl)ethynyl)naphth-1-yl)quinazolin-4-yl)piperazine-1-carboxylate

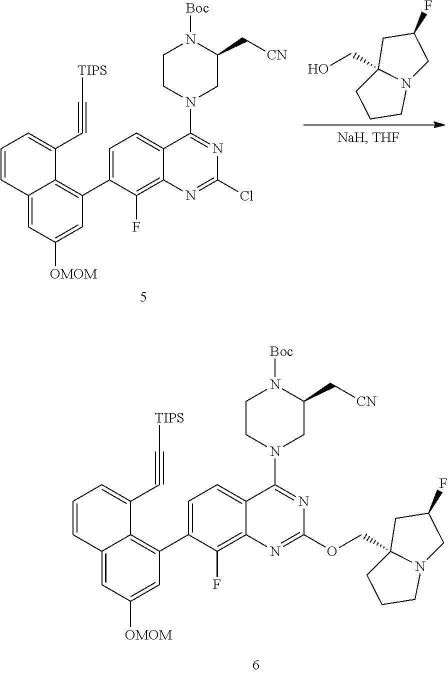

((2R,7aS)-2-fluorotetrahydro-1H-pyrrolizin-7a(5H)-yl)methanol (185 mg, 1.17 mmol) was dissolved in tetrahydrofuran (6.00 mL), and sodium hydride (62.1 mg, 1.55 mmol, 60% purity) was added at 0° C. Under nitrogen protection, the reaction liquid was stirred at 20° C. for 1 hour. A solution of tert-butyl (S)-4-(2-chloro-8-fluoro-7-(3-((methoxymethoxy)-8-((triisopropylsilyl)ethynyl)naphth-1-yl)quinazolin-4-yl)-2-(cyanomethyl)piperazine-1-carboxylate (62.1 mg, 1.55 mmol, 60% purity) in tetrahydrofuran (3.00 mL) was added to the reaction liquid, and the reaction liquid was stirred for 1 hour. After the reaction was complete as monitored by LCMS, the reaction liquid was quenched with water and extracted with ethyl acetate, and the organic phase was washed with brine and then concentrated to obtain tert-butyl (S)-2-(cyanomethyl)-4-(8-fluoro-2-((2R,7aS)-2-fluoro-tetrahydrofuran-1H-pyrrolizin-7a(5H)-yl)methoxy)-7-(3-(methoxymethyl)oxy)-8-(triisopropylsilyl)ethynyl)naphth-1-yl)quinazolin-4-yl)piperazine-1-carboxylate (400 mg, crude).

LCMS: m/z 895.7 [M+H]⁺.

### Step VI: Synthesis of tert-butyl (S)-2-(cyanomethyl)-4-(7-(8-ethynyl-3-(methoxymethoxy)naphth-1-yl)-8-fluoro-2-(2R,7aS)-2-fluorotetrahydro-1H-pyrrolizin-7a(5H)-yl)methoxy)quinazolin-4-yl)piperazine-1-carboxylate

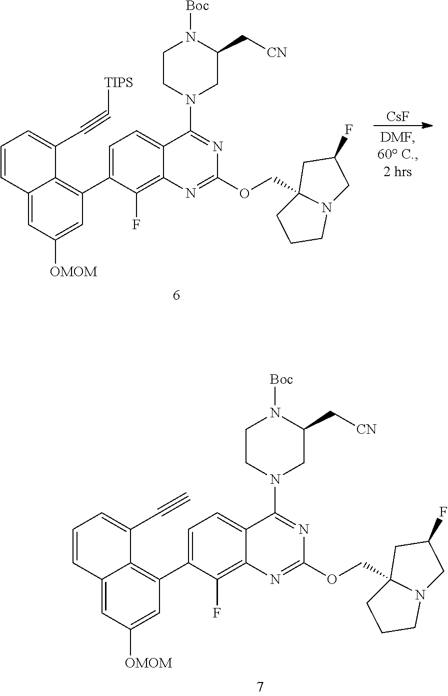

Tert-butyl (S)-2-(cyanomethyl)-4-(8-fluoro-2-((2R,7aS)-2-fluoro-tetrahydrofuran-1H-pyrrolizin-7a(5H)-yl)methoxy)-7-(3-(methoxymethyl)oxy)-8-(triisopropylsilyl)ethynyl)naphth-1-yl)quinazolin-4-yl)piperazine-1-carboxylate (400 mg, 447 umol) was dissolved in N,N-dimethylformamide (5.0 mL), and cesium fluoride (203 mg, 1.34 mmol) was added to the reaction liquid. The reaction liquid was stirred at 60° C. for 1 hour. After the reaction was complete as monitored by LCMS, the reaction liquid was extracted with ethyl acetate and washed with brine, and the organic phase was concentrated and passed through a fast silica gel column to obtain tert-butyl (S)-2-(cyanomethyl)-4-(7-(8-ethynyl-3-(methoxymethoxy)naphth-1-yl)-8-fluoro-2-(2R,7aS)-2-fluorotetrahydro-1H-pyrrolizin-7a(5H)-yl)methoxy)quinazolin-4-yl)piperazine-1-carboxylate (240 mg, yield 72.7%).

LCMS: m/z 739.4 [M+H]⁺.

### Step VII: Synthesis of 2-((S)-4-(7-(8-ethynyl-3-hydroxynaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

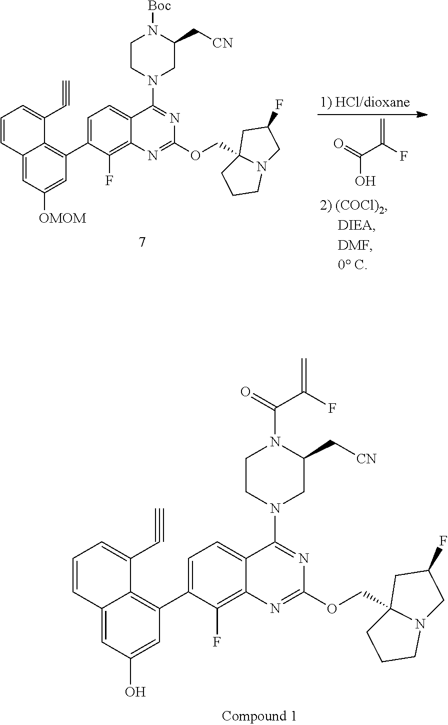

Solution A: At 0° C., tert-butyl (S)-2-(cyanomethyl)-4-(7-(8-ethynyl-3-(methoxymethoxy)naphth-1-yl)-8-fluoro-2-(2R,7aS)-2-fluorotetrahydro-1H-pyrrolizin-7a(5H)-yl)methoxy)quinazolin-4-yl)piperazine-1-carboxylate (240 mg, 325 umol) was dissolved in dioxane hydrochloride (4.00 M, 5.85 mL) and stirred for one hour. After the reaction liquid was concentrated, N,N-dimethylformamide (2.00 mL) and N,N-diisopropylethylamine (126 mg, 975 umol) were added.

Solution B: At 0° C., 2-fluoroacrylic acid (32.2 mg, 357 umol) was dissolved in dichloromethane (1.00 mL) and N,N-dimethylformamide (0.1 mL), and oxalyl chloride (53.6 mg, 422 umol) was added and reacted at this temperature for 10 minutes. At 0° C., solution B was added to solution A and stirred for 20 minutes. The reaction was complete as monitored by LCMS. The reaction liquid was filtered, and after separation and purification by prep-HPLC, 2-((S)-4-(7-(8-ethynyl-3-hydroxynaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (16.0 mg, yield 7.24%) was obtained.

LCMS: m/z 667.5 [M+H]⁺.

¹H NMR (400 MHz, (CD₃)₂SO): δ=10.13 (d, J=2.0 Hz, 1H), 7.94-7.76 (m, 2H), 7.49-7.38 (m, 2H), 7.35-7.21 (m, 2H), 7.03 (d, J=2.4 Hz, 1H), 5.52-5.17 (m, 3H), 5.07-4.74 (m, 1H), 4.45-3.93 (m, 5H), 3.59-3.39 (m, 2H), 3.19-2.98 (m, 5H), 2.88-2.78 (m, 1H), 2.20-1.99 (m, 4H), 1.92-1.72 (m, 4H) ppm.

### Example 2: 2-((S)-4-(7-(8-chloro-3-hydroxynaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 2)

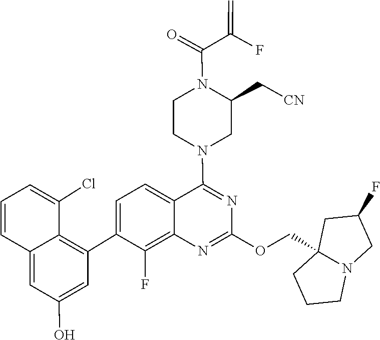 [SMILES: C=C(F)C(=O)N1CCN(c2nc(OC[C@@]34CCCN3C[C@H](F)C4)nc3c(F)c(-c4cc(O)cc5cccc(Cl)c45)ccc23)C[C@@H]1CC#N]

The specific synthesis route of Compound 2 was as follows:

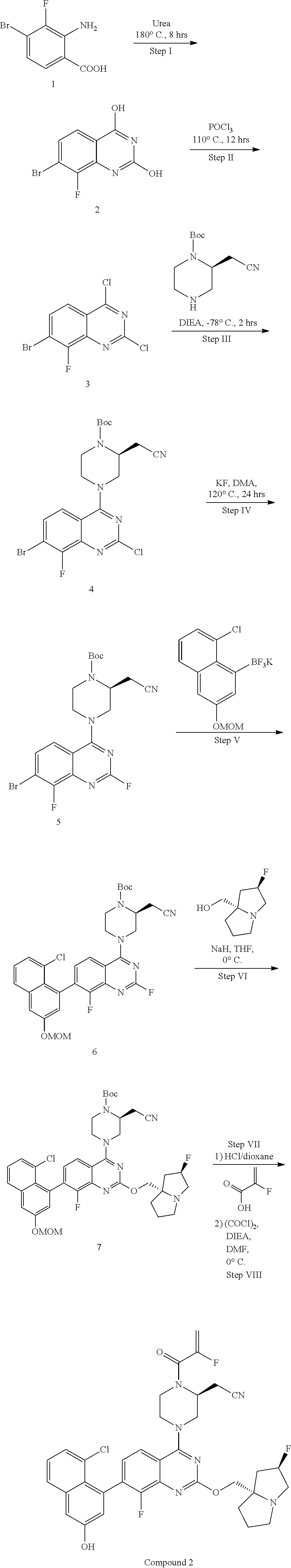

Starting from the intermediate No. 5 compound, the specific synthesis process of Compound 2 was as follows:

### Step V: Synthesis of tert-butyl (S)-4-(7-(8-chloro-3-(methoxymethoxy)naphth-1-yl)-2,8-difluoroquinazolin-4-yl)-2-(cyanomethyl)piperazine-1-carboxylate

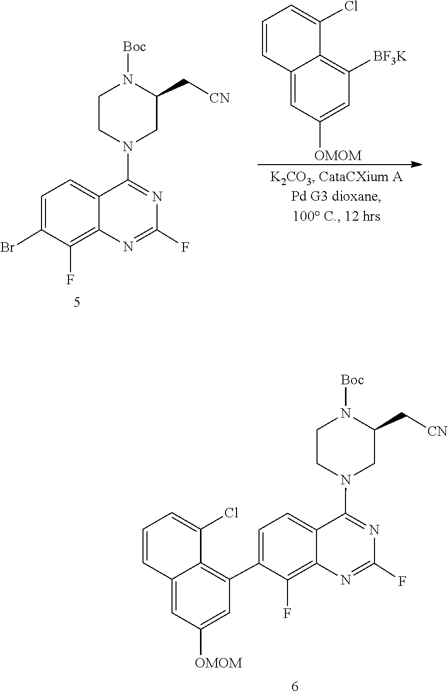

Tert-butyl (S)-4-(7-bromo-2,8-difluoroquinazolin-4-yl)-2-(cyanomethyl)piperazine-1-carboxylate (2.00 g, 4.27 mmol, 1.00 eq) and potassium (8-chloro-3-(methoxymethoxy)-1-naphthol)fluoroboronate (2.81 g, 8.54 mmol, 2.00 eq) were dissolved in 30.0 mL of dioxane, methanesulfonato(diadamantyl-N-butylphosphino)-2′-amino-1,1′-biphenyl-2-yl)palladium(II) (622 mg, 854 umol, 0.20 eq) and potassium carbonate (1.18 g, 8.54 mmol, 2.00 eq) were added, and the reaction liquid was stirred at 100° C. for 12 hours. After the reaction was complete as monitored by LCMS, the reaction liquid was concentrated and purified by a fast silica gel column to obtain tert-butyl (S)-4-(7-(8-chloro-3-(methoxymethoxy)naphth-1-yl)-2,8-difluoroquinazolin-4-yl)-2-(cyanomethyl)piperazine-1-carboxylate (2.00 g, 1.23 mmol, 76.8% yield).

LCMS: m/z 610.1 [M+H]⁺.

¹H NMR (400 MHz, CDCl₃): δ=7.79-7.72 (m, 2H), 7.54 (d, J=2.4 Hz, 1H), 7.42-7.34 (m, 3H), 7.18 (d, J=2.4 Hz, 1H), 5.33 (s, 2H), 4.73-4.67 (m, 1H), 4.48-4.39 (m, 3H), 3.90-3.81 (m, 1H), 3.71-3.60 (m, 2H), 3.54 (s, 3H), 2.90-2.85 (m, 1H), 2.83-2.76 (m, 1H), 1.54 (s, 9H).

### Step VI: Synthesis of tert-butyl (S)-4-(7-(8-chloro-3-(methoxymethoxy)naphth-1-yl)-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate

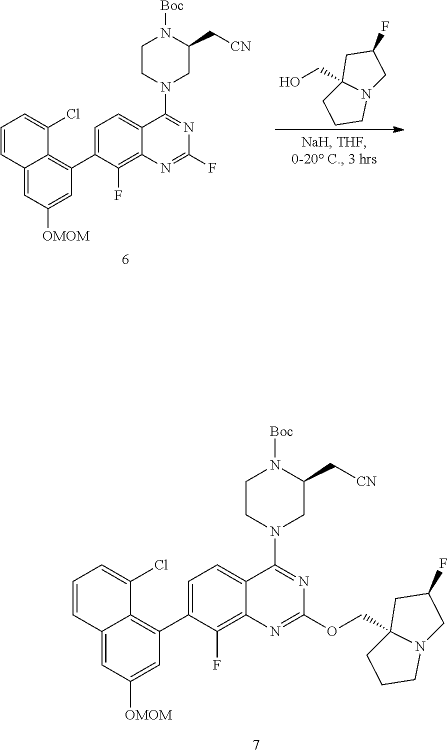

((2R,7aS)-2-fluorotetrahydro-1H-pyrrolizin-7a(5H)-yl)methanol (78.3 mg, 492 umol, 3.00 eq) was dissolved in 4.00 mL of tetrahydrofuran, sodium hydride (29.5 mg, 738 umol, 60.0% purity, 4.50 eq) was added at 0° C., and the reaction liquid was stirred at 20° C. for 1 hour. Tert-butyl (S)-4-(7-(8-chloro-3-(methoxymethoxy)naphth-1-yl)-2,8-difluoroquinazolin-4-yl)-2-(cyanomethyl)piperazine-1-carboxylate (100 mg, 164 umol, 1.00 eq) was added to the reaction liquid, and the reaction liquid was further stirred at 20° C. for 2 hours. After the reaction was complete as monitored by LCMS, the reaction liquid was quenched with water and extracted with ethyl acetate and washed with brine, and the organic phase was concentrated and purified by a fast silica gel column to obtain tert-butyl (S)-4-(7-(8-chloro-3-(methoxymethoxy)naphth-1-yl)-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate (90 mg, 98.9 umol, 60.3% yield).

LCMS: m/z 749.3 [M+H]⁺.

¹H NMR (400 MHz, (CD₃)₂SO): δ=8.00-7.84 (m, 2H), 7.72-7.65 (m, 1H), 7.52-7.42 (m, 2H), 7.35-7.28 (m, 1H), 7.25-7.14 (m, 1H), 5.41-5.35 (m, 2H), 5.28-5.18 (m, 1H), 4.67-4.50 (m, 2H), 4.33-4.08 (m, 4H), 3.95-3.88 (m, 1H), 3.55-3.47 (m, 2H), 3.46-3.43 (m, 3H), 2.99-2.91 (m, 2H), 2.84-2.72 (m, 2H), 1.87-1.73 (m, 6H), 1.48-1.42 (m, 9H), 0.81-0.70 (m, 2H).

### Step VII: Synthesis of 2-((S)-4-(7-(8-chloro-3-hydroxynaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile

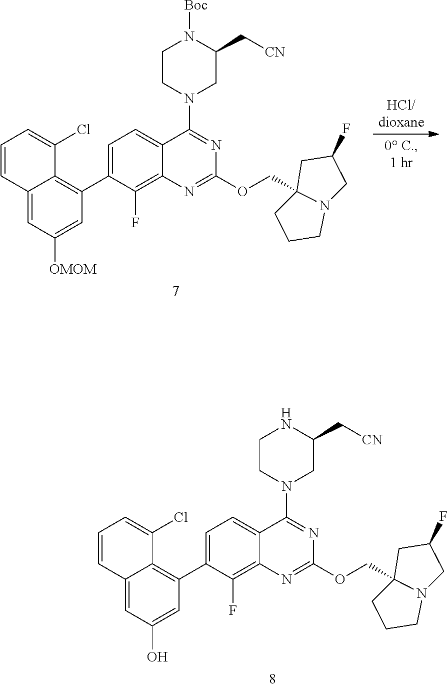

At 0° C., 2.00 mL of dioxane hydrochloride was added to tert-butyl (S)-4-(7-(8-chloro-3-(methoxymethoxy)naphth-1-yl)-8-fluoro-2-(((2R,7aS)-2-fluorotetrahydro-1H-pyrrolizin-7a(5H)-yl)methoxy)quinazolin-4-yl)-2-(cyanomethyl)piperazine-1-carboxylate (80.0 mg, 107 umol, 1.00 eq), and the reaction liquid was stirred at 0° C. for 1 hour. After the reaction was complete as monitored by LCMS, the reaction was concentrated to obtain 2-((S)-4-(7-(8-chloro-3-hydroxynaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile (100 mg, crude).

LCMS: m/z 605.3 [M+H]⁺.

### Step VIII: Synthesis of 2-((S)-4-(7-(8-chloro-3-hydroxynaphth-1-yl)-8-fluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

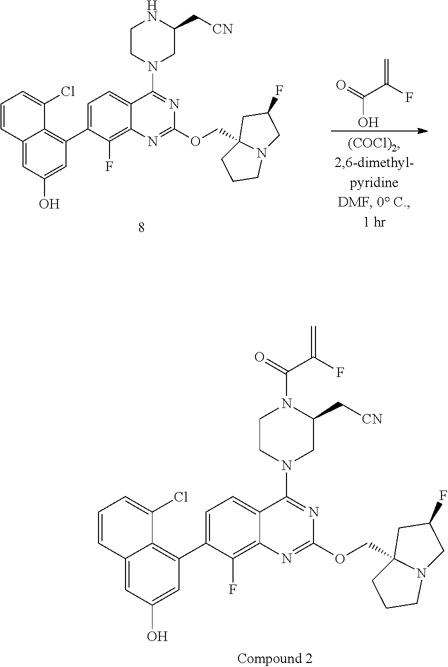

2-Fluoroprop-2-enoic acid (16.4 mg, 182 umol, 1.10 eq) was dissolved in 1.00 mL of dichloromethane, oxalyl chloride (23.1 mg, 182 umol, 15.9 uL, 1.10 eq) was added to the reaction liquid at 0° C., and N,N-dimethylformamide (1.21 mg, 16.5 umol, 1.27 uL, 0.10 eq) was then added and stirred for 30 minutes; subsequently, the reaction liquid was dropwise added to a solution of 2-((S)-4-(7-(8-chloro-3-hydroxynaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile (100 mg, 165 umol, 1.00 eq) and 2,6-dimethylpyridine (53.1 mg, 496 umol, 57.7 uL, 3.00 eq) in N,N-dimethylformamide (3.00 mL) and stirred for 1 hour. After the reaction was complete as monitored by LCMS, the reaction liquid was concentrated and then purified by prep-HPLC to obtain 2-((S)-4-(7-(8-chloro-3-hydroxynaphth-1-yl)-8-fluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (19.6 mg, 28.4 umol, 17.2% yield).

LCMS: m/z 677.3 [M+H]⁺.

¹H NMR (400 MHz, (CD₃)₂SO): δ=10.25 (br s, 1H), 7.91-7.80 (m, 2H), 7.45-7.27 (m, 4H), 7.03 (d, J=2.6 Hz, 1H), 5.47-5.33 (m, 2H), 5.28-5.17 (m, 1H), 5.04-4.72 (m, 1H), 4.34-4.24 (m, 2H), 4.16-3.99 (m, 3H), 3.62-3.47 (m, 3H), 3.13-2.99 (m, 4H), 2.89-2.77 (m, 1H), 2.23-1.66 (m, 7H).

### Example 3: 2-((2S)-4-(7-(8-chloro-3-hydroxynaphth-1-yl)-6,8-difluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 3)

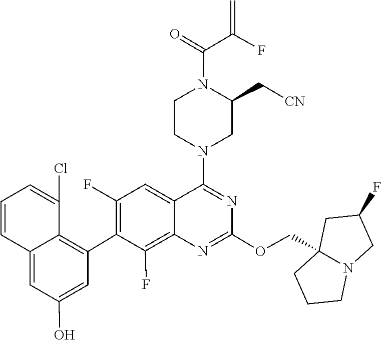 [SMILES: C=C(F)C(=O)N1CCN(c2nc(OC[C@@]34CCCN3C[C@H](F)C4)nc3c(F)c(-c4cc(O)cc5cccc(Cl)c45)c(F)cc23)C[C@@H]1CC#N]

LCMS: m/z 695.2 [M+H]⁺.

### Example 4: 2-((S)-4-(7-(8-ethynyl-7-fluoro-3-hydroxynaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 4)

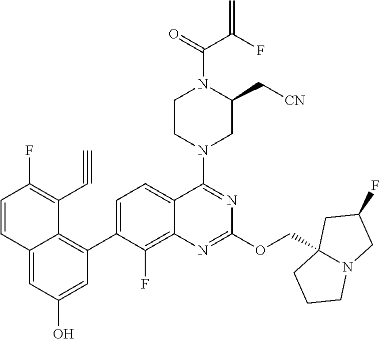 [SMILES: C#Cc1c(F)ccc2cc(O)cc(-c3ccc4c(N5CCN(C(=O)C(=C)F)[C@@H](CC#N)C5)nc(OC[C@@]56CCCN5C[C@H](F)C6)nc4c3F)c12]

The specific synthesis route of Compound 4 was as follows:

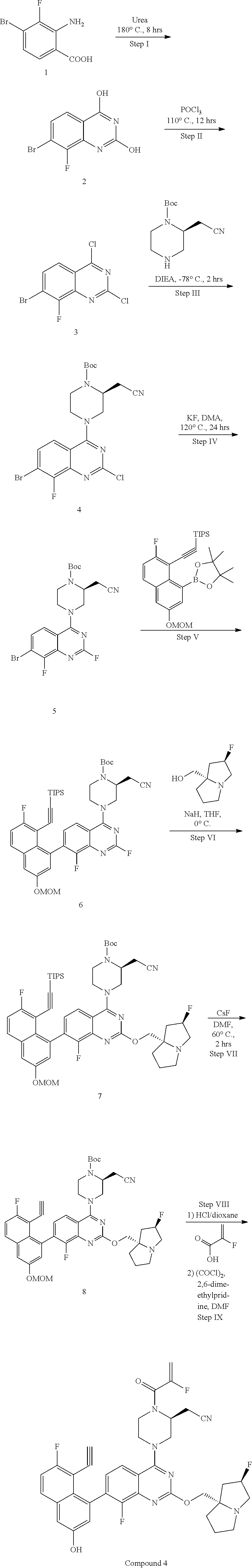

Starting from the intermediate No. 5 compound, the specific synthesis process of Compound 4 was as follows:

### Step V: Synthesis of tert-butyl (S)-2-(cyanomethyl)-4-(2,8-difluoro-7-(7-fluoro-3-(methoxymethoxy)-8-(triisopropylsilyl)ethynyl)naphth-1-yl)quinazolin-4-yl)piperazine-1-carboxylate

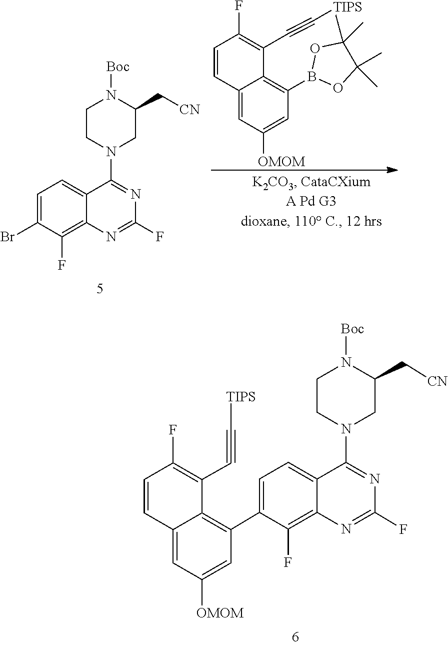

Tert-butyl (S)-4-(7-bromo-2,8-difluoroquinazolin-4-yl)-2-(cyanomethyl)piperazine-1-carboxylate (2.00 g, 4.27 mmol) and ((2-fluoro-6-(methoxymethoxy)-8-(4,4,5,5-tetramethyl-1,3,2-dioxaboran-2-yl)naphth-1-yl)ethynyl)triisopropylsilane (2.41 g, 4.70 mmol) were dissolved in 30.0 mL of dioxane, methane sulfonato(diadamantyl-N-butylphosphino)-2-amino-1,1-biphenyl-2-yl)palladium(11)(622 mg, 854 umol) and potassium carbonate (1.18 g, 8.54 mmol) were added, and the reaction liquid was stirred at 110° C. for 12 hours. After the reaction was complete as monitored by LCMS, the reaction liquid was concentrated and purified by a fast silica gel column to obtain tert-butyl (S)-2-(cyanomethyl)-4-(2,8-difluoro-7-(7-fluoro-3-(methoxymethoxy)-8-(triisopropylsilyl)ethynyl)naphth-1-yl)quinazolin-4-yl)piperazine-1-carboxylate (700 mg, 802 umol, 18.8% yield).

LCMS: m/z 774.5 [M+H]⁺.

¹H NMR (400 MHz, CDCl₃): δ=7.84-7.71 (m, 2H), 7.51 (d, J=2.4 Hz, 1H), 7.48-7.43 (m, 1H), 7.32 (t, J=8.8 Hz, 1H), 7.18 (d, J=2.4 Hz, 1H), 5.31 (s, 2H), 4.71 (br s, 1H), 4.53-4.06 (m, 4H), 3.53 (s, 3H), 2.88-2.64 (m, 2H), 1.94-1.84 (m, 2H), 1.25 (s, 9H), 0.85 (d, J=7.6 Hz, 18H), 0.58-0.49 (m, 3H).

### Step VI: Synthesis of tert-butyl (S)-2-(cyanomethyl)-4-(8-fluoro-7-(7-fluoro-3-(methoxymethoxy)-8-(triisopropylsilyl)ethynyl)naphth-1-yl)-2-(2R,7aS)-2-fluorotetrahydro-1H-pyrrolizin-7a(5H)-yl)methoxy)quinazolin-4-yl)piperazine-1-carboxylate

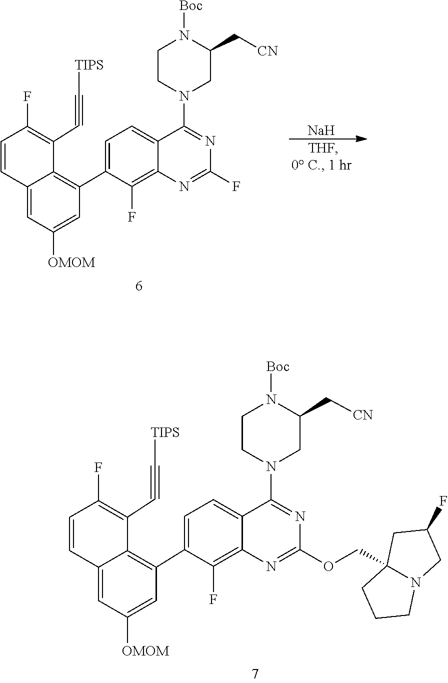

((2R,7aS)-2-fluorotetrahydro-1H-pyrrolizin-7a(5H)-yl)methanol (370 mg, 2.33 mmol) was dissolved in 20.0 mL of tetrahydrofuran, sodium hydride (140 mg, 3.49 mmol, 60.0% purity) was added at 0° C., and the reaction liquid was stirred at 20° C. for 1 hour. Tert-butyl (S)-2-(cyanomethyl)-4-(2,8-difluoro-7-(7-fluoro-3-(methoxymethoxy)-8-(triisopropylsilyl)ethynyl)naphth-1-yl)quinazolin-4-yl)piperazine-1-carboxylate (600 mg, 775 umol) was added to the reaction liquid, and the reaction liquid was further stirred at 20° C. for 2 hours. After the reaction was complete as monitored by LCMS, the reaction liquid was quenched with water, extracted with ethyl acetate and washed with brine, and the organic phase was concentrated and purified by a fast silica gel column to obtain tert-butyl (S)-2-(cyanomethyl)-4-(8-fluoro-7-(7-fluoro-3-(methoxymethoxy)-8-(triisopropylsilyl)ethynyl)naphth-1-yl)-2-(2R,7aS)-2-fluorotetrahydro-1H-pyrrolizin-7a(5H)-yl)methoxy)quinazolin-4-yl)piperazine-1-carboxylate (420 mg, 460 umol, 59.3% yield).

LCMS: m/z 913.3 [M+H]⁺.

### Step VII: Synthesis of tert-butyl (S)-2-(cyanomethyl)-4-(7-(8-ethynyl-7-fluoro-3-(methoxymethoxy)naphth-1-yl)-8-fluoro-2-(2R,7aS)-2-fluorotetrahydro-1H-pyrrolizin-7a(5H)-yl)methoxy)quinazolin-4-yl)piperazine-1-carboxylate

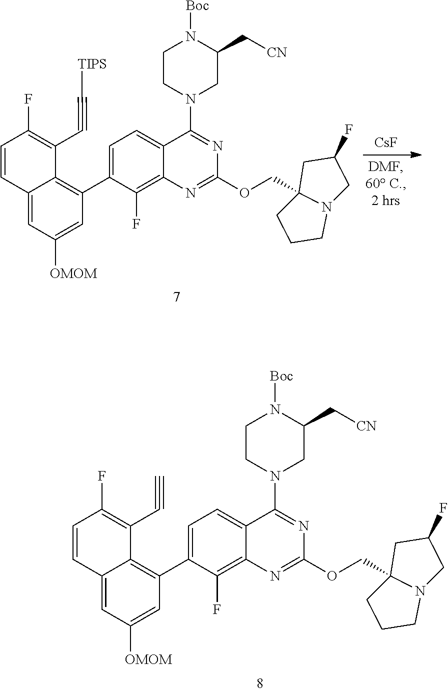

Tert-butyl (S)-2-(cyanomethyl)-4-(8-fluoro-7-(7-fluoro-3-(methoxymethoxy)-8-(triisopropylsilyl)ethynyl)naphth-1-yl)-2-(2R,7aS)-2-fluorotetrahydro-1H-pyrrolizin-7a(5H)-yl)methoxy)quinazolin-4-yl)piperazine-1-carboxylate (470 mg, 514 umol) was dissolved in 5.00 mL of N,N-dimethylformamide, and cesium fluoride (235 mg, 1.54 mmol) was added to the reaction liquid.

The reaction liquid was stirred at 60° C. for 1 hour. After the reaction was complete as monitored by LCMS, the reaction liquid was poured into ice water, extracted with ethyl acetate, and washed with brine, and the organic phase was concentrated to obtain tert-butyl (S)-2-(cyanomethyl)-4-(7-(8-ethynyl-7-fluoro-3-(methoxymethoxy)naphth-1-yl)-8-fluoro-2-(2R,7aS)-2-fluorotetrahydro-1H-pyrrolizin-7a(5H)-yl)methoxy)quinazolin-4-yl)piperazine-1-carboxylate (400 mg, crude).

LCMS: m/z 757.4 [M+H]⁺.

¹H NMR (400 MHz, CDCl₃): δ=7.78-7.71 (m, 1H), 7.53 (d, J=8.4 Hz, 1H), 7.43 (d, J=2.4 Hz, 1H), 7.26-7.20 (m, 1H), 7.19-7.10 (m, 2H), 5.37-5.19 (m, 3H), 4.59 (br s, 1H), 4.43-4.17 (m, 4H), 4.06-3.94 (m, 1H), 3.54 (br dd, J=3.2, 13.8 Hz, 1H), 3.46 (s, 3H), 3.45-3.09 (m, 5H), 2.98 (br d, J=4.4 Hz, 1H), 2.86 (br s, 1H), 2.81-2.63 (m, 2H), 2.37-2.11 (m, 3H), 1.97-1.87 (m, 3H), 1.45 (s, 9H).

### Step VIII: Synthesis of 2-((S)-4-(7-(8-ethynyl-7-fluoro-3-hydroxynaphth-1-yl)-8-fluoro-2-((2R,7aS)-2-fluorotetrahydro-1H-pyrrolizin-7a(5H)-yl)methoxy)quinazolin-4-yl)piperazin-2-yl)acetonitrile

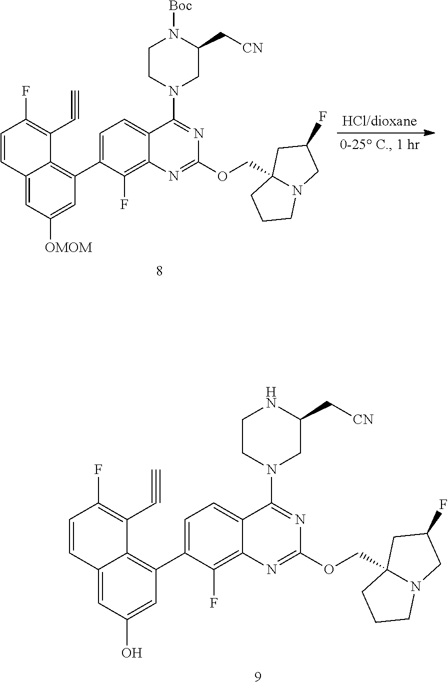

At 0° C., a dioxane hydrochloride solution (4.00 M, 6.00 mL) was added to tert-butyl (S)-2-(cyanomethyl)-4-(7-(8-ethynyl-7-fluoro-3-(methoxymethoxy)naphth-1-yl)-8-fluoro-2-(2R,7aS)-2-fluorotetrahydro-1H-pyrrolizin-7a(5H)-yl)methoxy)quinazolin-4-yl)piperazine-1-carboxylate (100 mg, 132 umol), and the reaction liquid was stirred at 25° C. for 1 hour. After the reaction was complete as monitored by LCMS, the reaction liquid was concentrated to obtain 2-((S)-4-(7-(8-ethynyl-7-fluoro-3-hydroxynaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile (100 mg, crude, hydrochloride).

LCMS: m/z 613.4 [M+H]⁺.

### Step IX: Synthesis of 2-((S)-4-(7-(8-ethynyl-7-fluoro-3-hydroxynaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

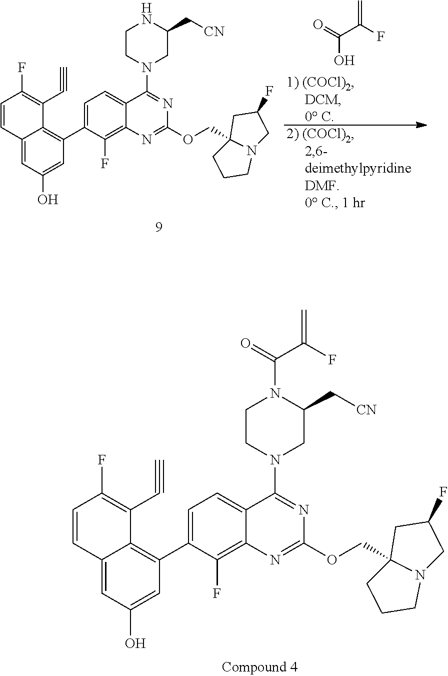

2-Fluoroprop-2-enoic acid (12.9 mg, 144 umol) was dissolved in 1.00 mL of dichloromethane, oxalyl chloride (18.2 mg, 144 umol, 12.6 uL) was added to the reaction liquid at 0° C., and a drop of N,N-dimethylformamide was then added and stirred for 30 minutes; subsequently, the reaction liquid was dropwise added to a solution of 2-((S)-4-(7-(8-ethynyl-7-fluoro-3-hydroxynaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile (80.0 mg, 131 umol) and 2,6-dimethylpyridine (42.0 mg, 392 umol, 45.6 uL) in N,N-dimethylformamide (2.00 mL) and stirred for 1 hour. After the reaction was complete as monitored by LCMS, the reaction liquid was concentrated and purified by prep-HPLC to obtain 2-((S)-4-(7-(8-ethynyl-7-fluoro-3-hydroxynaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (9.00 mg, 12.9 umol, 9.89% yield).

LCMS: m/z 685.6 [M+H]⁺.

¹H NMR (400 MHz, (CD₃)₂SO): δ=10.17 (br s, 1H), 7.97 (dd, J=6.0, 9.1 Hz, 1H), 7.82 (dd, J=8.8, 13.2 Hz, 1H), 7.46 (t, J=8.8 Hz, 1H), 7.37 (d, J=2.4 Hz, 1H), 7.30-7.23 (m, 1H), 7.08 (s, 1H), 5.48-5.33 (m, 2H), 5.28-5.18 (m, 1H), 5.04-4.67 (m, 1H), 4.34-4.21 (m, 2H), 4.14-3.93 (m, 3H), 3.83 (d, J=6.8 Hz, 1H), 3.52 (br d, J=12.0 Hz, 1H), 3.33 (br s, 1H), 3.22-2.91 (m, 5H), 2.87-2.76 (m, 1H), 2.18-1.70 (m, 7H).

### Example 5: 2-((S)-4-(7-(8-chloronaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 5)

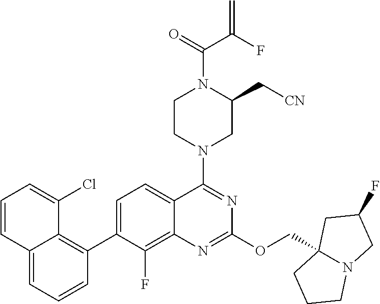 [SMILES: C=C(F)C(=O)N1CCN(c2nc(OC[C@@]34CCCN3C[C@H](F)C4)nc3c(F)c(-c4cccc5cccc(Cl)c45)ccc23)C[C@@H]1CC#N]

LCMS: m/z 661.2 [M+H]⁺.

### Example 6: 2-((2S)-4-(7-(8-chloronaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile (Compound 6)

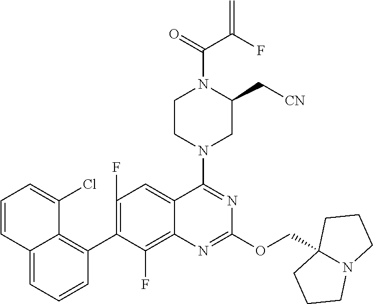

The specific synthesis route of Compound 6 was as follows:

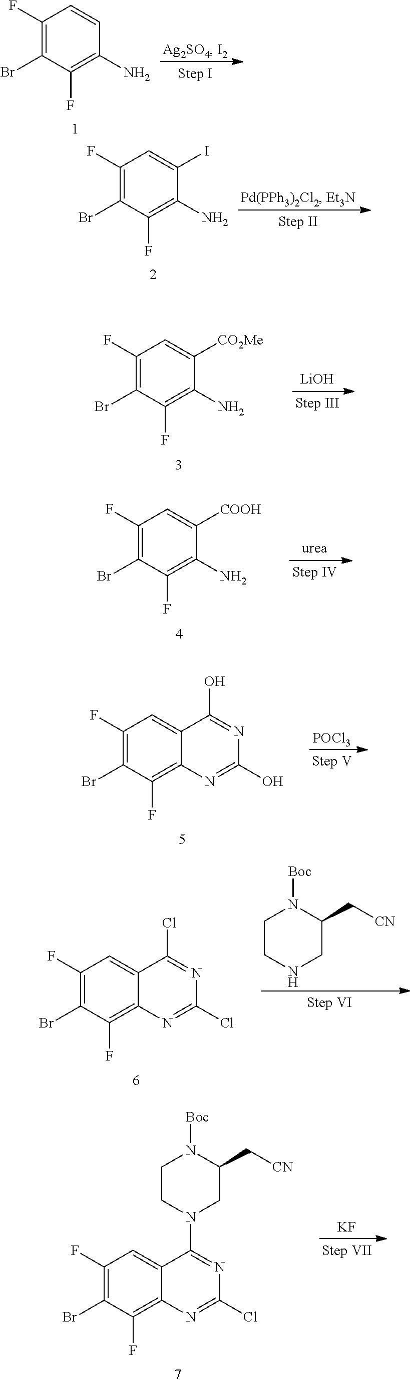

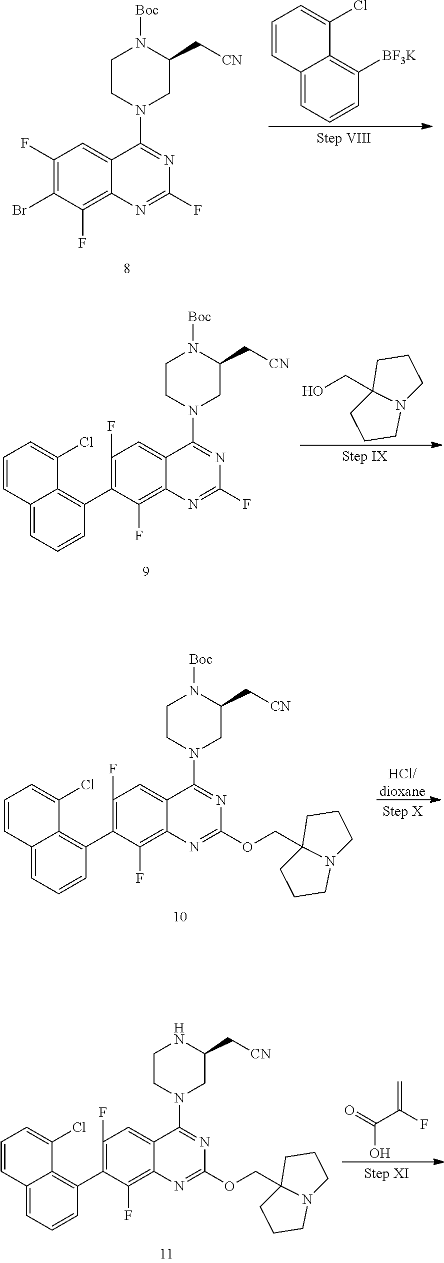

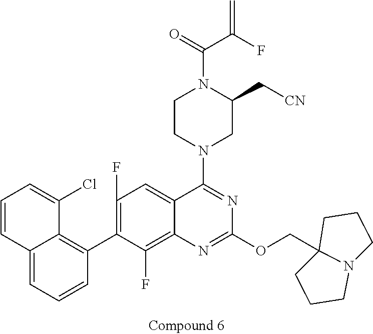 [SMILES: C=C(F)C(=O)N1CCN(c2nc(OCC34CCCN3CCC4)nc3c(F)c(-c4cccc5cccc(Cl)c45)c(F)cc23)C[C@@H]1CC#N]

Starting from the intermediate No. 6 compound, the specific synthesis of Compound 6 was as follows:

### Step VIII: Synthesis of tert-butyl (2S)-4-(7-(8-chloronaphth-1-yl)-2,6,8-trifluoroquinazolin-4-yl)-2-(cyanomethyl)piperazine-1-carboxylate

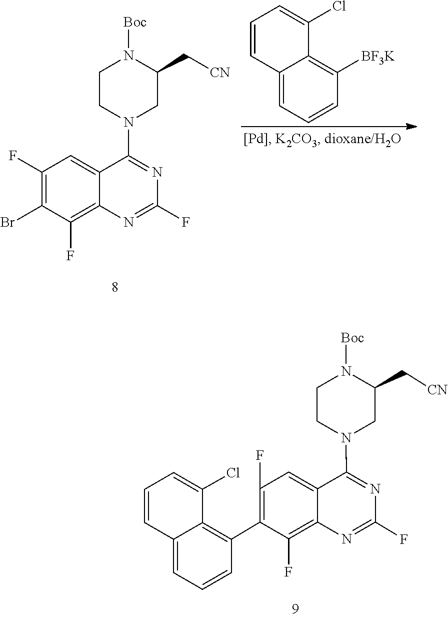

Tert-butyl (S)-4-(7-bromo-2,6,8-trifluoroquinazolin-4-yl)-2-(cyanomethyl)piperazine-1-carboxylate (100 mg, 205 umol), potassium (8-chloro-1-naphthalene)-fluoroboronate (150 mg, 558 umol), cesium fluoride (99.9 mg, 658 umol), and 1,1-bis(tert-butylphosphino)ferrocene palladium chloride (26.8 mg, 41.1 umol) were dissolved in water (0.25 mL) and N,N-dimethylformamide (0.75 mL), then aerated, and finally reacted at 100° C. under nitrogen protection for 15 minutes. Six reactions were carried out in parallel, and the reaction was complete as monitored by LCMS. The reaction liquid was quenched by adding water, extracted with dichloromethane, washed with a saturated sodium chloride aqueous solution, and dried over sodium sulfate, and after filtration, concentration and purification by column chromatography, tert-butyl (2S)-4-(7-(8-chloronaphth-1-yl)-2,6,8-trifluoroquinazolin-4-yl)-2-(cyanomethyl)piperazine-1-carboxylate as a yellow solid (540 mg, yield 77.1%) was obtained.

LCMS: m/z 568.2 [M+H]⁺.

¹H NMR (400 MHz, CDCl₃): δ=8.02 (d, J=6.4 Hz, 1H), 7.94-7.87 (m, 1H), 7.65-7.54 (m, 2H), 7.50-7.40 (m, 3H), 4.69 (br dd, J=3.1, 6.0 Hz, 1H), 4.46-4.31 (m, 2H), 4.24-4.07 (m, 1H), 3.88-3.76 (m, 1H), 3.71-3.38 (m, 2H), 2.95 (s, 2H), 1.53 (s, 9H) ppm.

### Step IX: Synthesis of tert-butyl (2S)-4-(7-(8-chloronaphth-1-yl)-6,8-difluoro-2-(tetrahydro-1H-pyrrolizin-7a(5H)-yl)methoxy)quinazolin-4-yl)-2-(cyanomethyl)piperazine-1-carboxylate

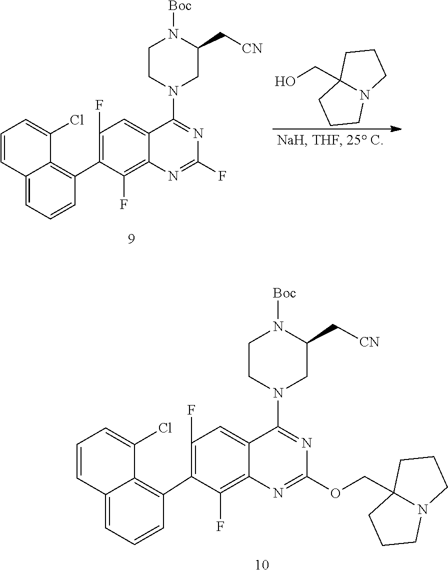 [SMILES: CC(C)(C)OC(=O)N1CCN(c2nc(F)nc3c(F)c(-c4cccc5cccc(Cl)c45)c(F)cc23)C[C@@H]1CC#N ; OCC12CCCN1CCC2 ; CC(C)(C)OC(=O)N1CCN(c2nc(OCC34CCCN3CCC4)nc3c(F)c(-c4cccc5cccc(Cl)c45)c(F)cc23)C[C@@H]1CC#N]

After (tetrahydro-1H-pyrrolizin-7a(5H)-yl)methanol (402 mg, 2.85 mmol) was dissolved in tetrahydrofuran (6 mL) under nitrogen protection at 0° C., sodium hydride (117 mg, 2.95 mmol) was added and then reacted at 20° C. for 1 hour. Then, tert-butyl (2S)-4-(7-(8-chloronaphth-1-yl)-2,6,8-trifluoroquinazolin-4-yl)-2-(cyanomethyl)piperazine-1-carboxylate (540 mg, 950 umol) was added and then reacted for 20 minutes. The reaction was complete as monitored by LCMS. The reaction liquid was quenched with water at 0° C. and extracted with ethyl acetate, washed with a saturated sodium chloride aqueous solution, and dried over sodium sulfate, and after filtration, concentration and purification by column chromatography, tert-butyl (2S)-4-(7-(8-chloronaphth-1-yl)-6,8-difluoro-2-(tetrahydro-1H-pyrrolizin-7a(5H)-yl)methoxy)quinazolin-4-yl)-2-(cyanomethyl)piperazine-1-carboxylate (380 mg, yield 57.9%) was obtained.

LCMS: m/z 689.4 [M+H]⁺.

¹H NMR (400 MHz, CDCl₃): δ=8.01 (d, J=8.0 Hz, 1H), 7.89 (d, J=8.1 Hz, 1H), 7.64-7.51 (m, 2H), 7.50-7.39 (m, 2H), 7.36-7.29 (m, 1H), 4.69 (br d, J=2.9 Hz, 1H), 4.36-4.21 (m, 4H), 3.68-3.54 (m, 1H), 3.52-3.31 (m, 2H), 3.20 (br d, J=1.1 Hz, 2H), 2.86 (br d, J=6.3 Hz, 1H), 2.70-2.63 (m, 2H), 2.18-2.09 (m, 2H), 1.96-1.87 (m, 4H), 1.83-1.77 (m, 2H), 1.75-1.66 (m, 2H), 1.53 (s, 9H) ppm.

### Step X: Synthesis of 2-((2S)-4-(7-(8-chloronaphth-1-yl)-6,8-difluoro-2-((tetrahydro-1H-pyrrolizin-7a(5H)-yl)methoxy)quinazolin-4-yl)piperazin-2-yl)acetonitrile

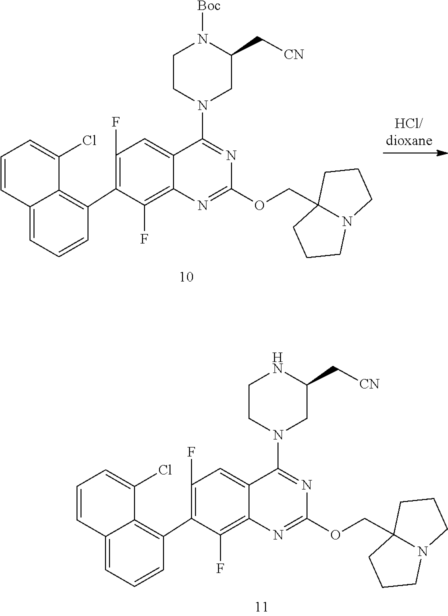 [SMILES: CC(C)(C)OC(=O)N1CCN(c2nc(OCC34CCCN3CCC4)nc3c(F)c(-c4cccc5cccc(Cl)c45)c(F)cc23)C[C@@H]1CC#N ; N#CC[C@H]1CN(c2nc(OCC34CCCN3CCC4)nc3c(F)c(-c4cccc5cccc(Cl)c45)c(F)cc23)CCN1]

Tert-butyl (2S)-4-(7-(8-chloronaphth-1-yl)-6,8-difluoro-2-(tetrahydro-1H-pyrrolizin-7a(5H)-yl)methoxy)quinazolin-4-yl)-2-(cyanomethyl)piperazine-1-carboxylate (153 mg, 222 umol) was dissolved in hydrochloric acid/dioxane (4 mL), then aerated, and finally reacted at 0° C. for 30 minutes. The reaction was complete as monitored by LCMS. The reaction liquid was concentrated to obtain 2-((2S)-4-(7-(8-chloronaphth-1-yl)-6,8-difluoro-2-((tetrahydro-1H-pyrrolizin-7a(5H)-yl)methoxy)quinazolin-4-yl)piperazin-2-yl)acetonitrile as a yellow solid (130 mg, crude).

LCMS: m/z 589.3 [M+H]⁺.

### Step XI: Synthesis of 2-((2S)-4-(7-(8-chloronaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile

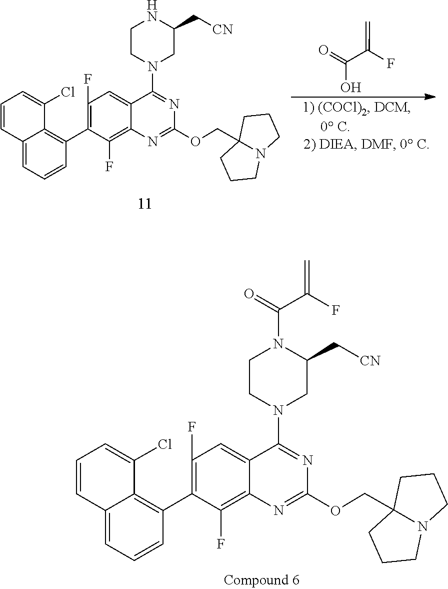

After 2-fluoroacrylic acid (59.6 mg, 662 umol) was dissolved in dichloromethane (6 mL) at 0° C., oxalyl chloride (86.8 mg, 684 umol) was added and reacted at this temperature for 30 minutes, and then, after 2-((2S)-4-(7-(8-chloronaphth-1-yl)-6,8-difluoro-2-((tetrahydro-1H-pyrrolizin-7a(5H)-yl)methoxy)quinazolin-4-yl)piperazin-2-yl)acetonitrile (130 mg, 220 umol) dissolved in N,N-dimethylformamide (2 mL) was added, a solution of N,N-diisopropylethylamine (28.5 mg, 220 umol) was added. Finally, the reaction was carried out at 0° C. for 30 minutes. The reaction was complete as monitored by LCMS. The reaction liquid was filtered and subjected to prep-HPLC and SFC for separation and purification to obtain 2-((2S)-4-(7-(8-chloronaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile as a yellow solid. After SFC resolution, two target compound peaks 6A and 6B were obtained. Compounds 6A and 6B were axially chiral isomers of each other.

6A: (17.3 mg, yield 11.6%), LCMS: m/z 661.5 [M+H]⁺.

¹H NMR (400 MHz, CD₃CN): 6=8.18-8.11 (m, 1H), 8.03 (d, J=8.3 Hz, 1H), 7.70 (t, J=7.8 Hz, 1H), 7.65-7.61 (m, 1H), 7.60-7.49 (m, 3H), 5.34-5.19 (m, 2H), 5.05-4.81 (m, 1H), 4.43-4.08 (m, 6H), 3.74-3.52 (m, 2H), 3.42-3.32 (m, 1H), 3.16-2.86 (m, 5H), 2.65-2.58 (m, 2H), 1.87-1.77 (m, 4H), 1.72-1.56 (m, 2H) ppm.

6B: (16.1 mg, yield 10.9%), LCMS: m/z 661.5 [M+H]⁺.

¹H NMR (400 MHz, CD₃CN): 6=8.15 (d, J=8.4 Hz, 1H), 8.03 (d, J=8.3 Hz, 1H), 7.70 (t, J=7.6 Hz, 1H), 7.63 (d, J=7.5 Hz, 1H), 7.59-7.51 (m, 3H), 5.31 (br t, J=3.8 Hz, 1H), 5.24 (br d, J=3.8 Hz, 1H), 5.06-4.83 (m, 1H), 4.35-4.04 (m, 6H), 3.71-3.49 (m, 2H), 3.41-3.32 (m, 1H), 3.12 (br dd, J=8.7, 16.9 Hz, 1H), 2.97 (br d, J=5.6 Hz, 4H), 2.65-2.58 (m, 2H), 1.86-1.77 (m, 4H), 1.68-1.60 (m, 2H) ppm.

### Example 7: 2-((2S)-4-(7-(8-ethynyl-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile

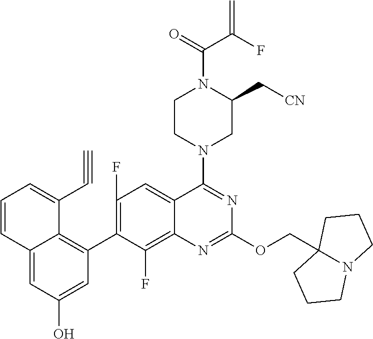 [SMILES: C#Cc1cccc2cc(O)cc(-c3c(F)cc4c(N5CCN(C(=O)C(=C)F)[C@@H](CC#N)C5)nc(OCC56CCCN5CCC6)nc4c3F)c12]

The specific synthesis route of Compound 7 was as follows:

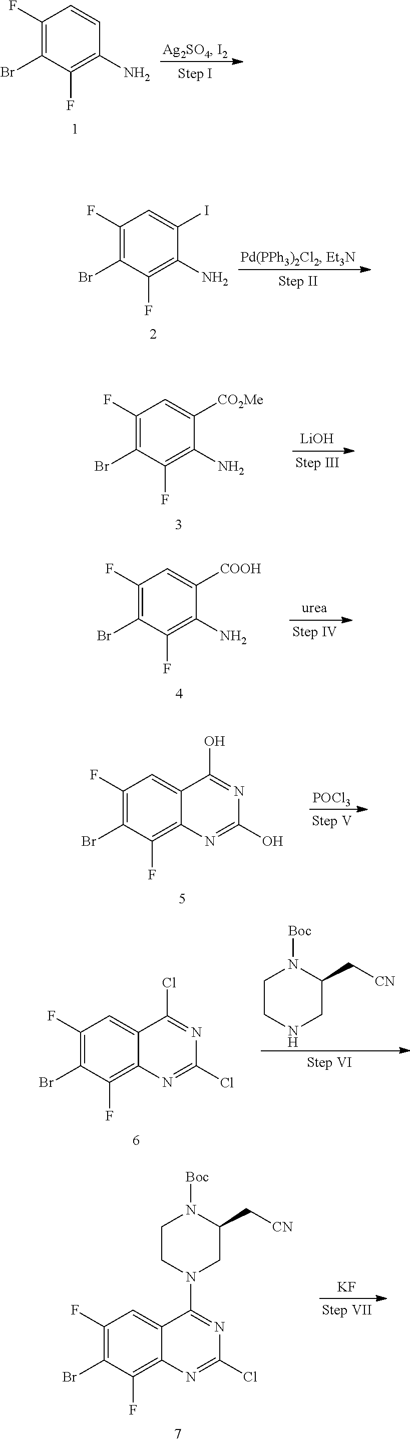

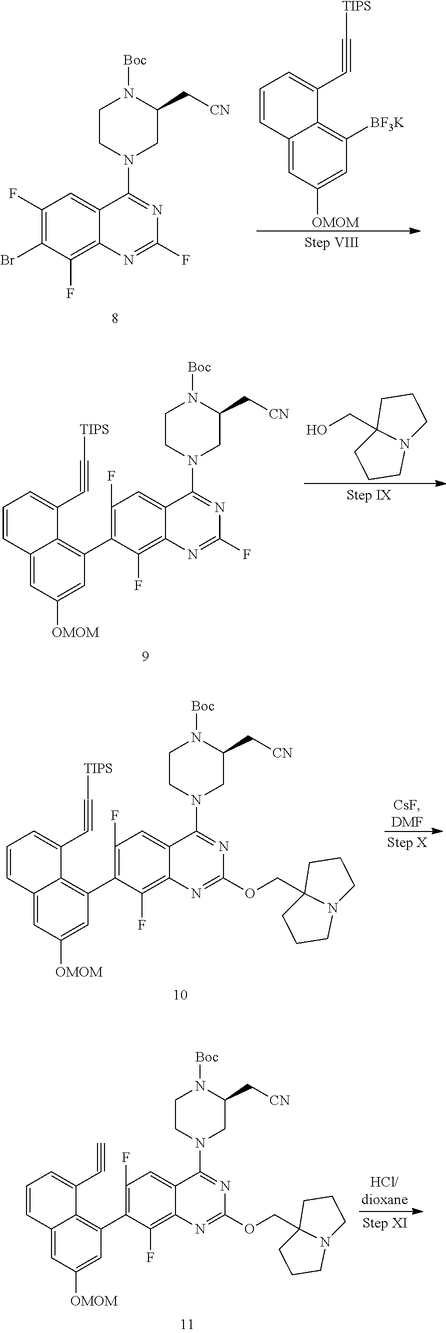

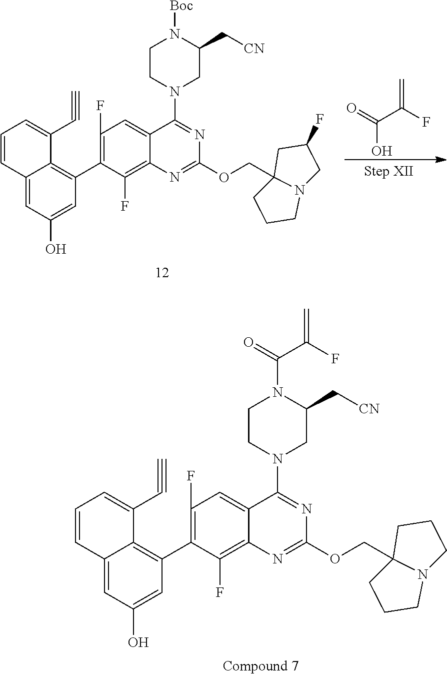

Starting from the intermediate No. 6 compound, the specific synthesis process of Compound 7 was as follows.

### Step VIII Synthesis of tert-butyl (2S)-2-(cyanomethyl)-4-(2,6,8-trifluoro-7-(3-(methoxymethoxy)-8-((triisopropylsilyl)ethynyl)naphth-1-yl)quinazolin-4-yl)piperazine-1-carboxylate

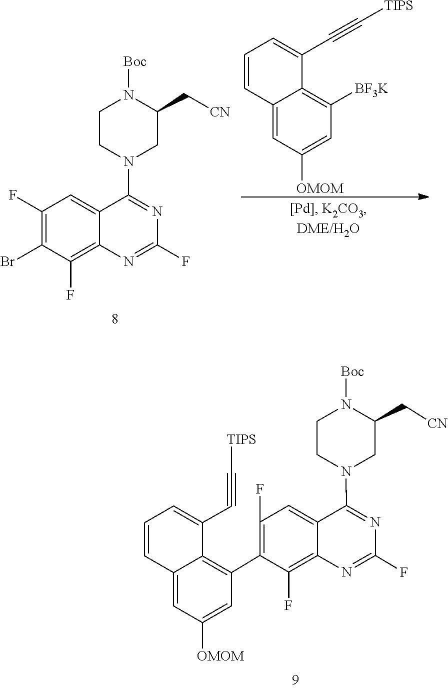

Potassium tert-butyl (S)-4-(7-bromo-2,6,8-trifluoroquinazolin-4-yl)-2-(cyanomethyl)piperazine-1-carboxylate (100 mg, 205 umol), 3-methoxymethoxy-8-((triisopropylsilyl)ethynyl)naphthalene-1-fluoroboronate (195 mg, 411 umol), [(bis(1-adamantyl)butylphosphino)-2-(2′-amino-1,1′-biphenyl)]palladium(II) methanesulfonate (14.9 mg, 20.5 umol), and potassium carbonate (71.0 mg, 514 umol) were dissolved in glycol dimethyl ether (4 mL) and water (0.4 mL) and then reacted at 80° C. under nitrogen protection for 1 hour. Nine reactions were carried out in parallel, and the reaction was complete as monitored by LCMS. The reaction liquid was concentrated and purified by column chromatography to obtain tert-butyl (2S)-2-(cyanomethyl)-4-(2,6,8-trifluoro-7-(3-(methoxymethoxy)-8-((triisopropylsilyl)ethynyl)naphth-1-yl)quinazolin-4-yl)piperazine-1-carboxylate as a yellow solid (1.00 g, yield 69.8%).

LCMS: m/z 774.3 [M+H]⁺.

¹H NMR (400 MHz, CDCl₃): δ=7.84 (d, J=8.1 Hz, 1H), 7.68 (d, J=7.1 Hz, 1H), 7.56 (d, J=2.5 Hz, 1H), 7.47-7.40 (m, 2H), 7.18 (t, J=2.9 Hz, 1H), 5.32 (s, 2H), 4.71 (br s, 1H), 4.52-4.19 (m, 3H), 3.94-3.63 (m, 2H), 3.54 (s, 2H), 3.61-3.48 (m, 1H), 3.47-3.16 (m, 1H), 3.06-2.75 (m, 2H), 1.55-1.52 (m, 9H), 0.90-0.82 (m, 18H), 0.61-0.52 (m, 3H) ppm.

### Step IX: Synthesis of tert-butyl (2S)-2-(cyanomethyl)-4-(6,8-difluoro-7-(3-(methoxymethoxy)-8-((triisopropylsilyl)ethynyl-)naphth-1-yl)-2-((tetrahydro-1H-pyrrolizin-7a(5H)-yl)methoxy)quinazolin-4-yl)piperazine-1-carboxylate

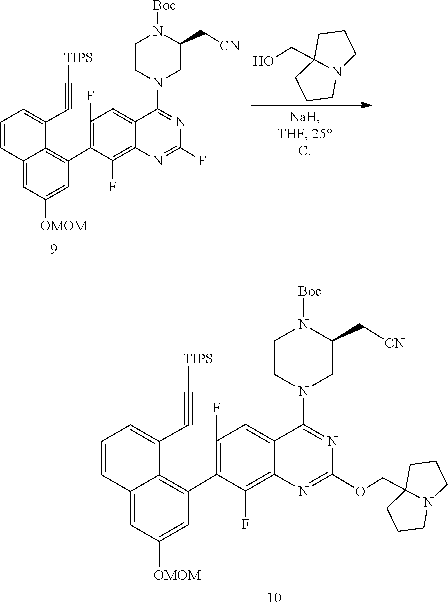

After (tetrahydro-1H-pyrrolizin-7a(5H)-yl)methanol (301 mg, 2.13 mmol) was dissolved in tetrahydrofuran (6 mL) at 0° C. under nitrogen protection, sodium hydride (90.9 mg, 2.27 mmol) was added, and then, after the reaction was carried out at 20° C. for 1 hours, tert-butyl (2S)-2-(cyanomethyl)-4-(2,6,8-trifluoro-7-(3-(methoxymethoxy)-8-((triisopropylsilyl)ethynyl)naphth-1-yl)quinazolin-4-yl)piperazine-1-carboxylate (550 mg, 710 umol) was added and reacted at this temperature for 30 minutes. The reaction was complete as monitored by LCMS. The reaction liquid was quenched by adding water at 0° C., extracted with ethyl acetate, washed with a saturated sodium chloride aqueous solution, and dried over sodium sulfate, and after filtration, concentration and column chromatography, tert-butyl (2S)-2-(cyanomethyl)-4-(6,8-difluoro-7-(3-(methoxymethoxy)-8-((triisopropylsilyl)ethynyl-)naphth-1-yl)-2-((tetrahydro-1H-pyrrolizin-7a(5H)-yl)methoxy)quinazolin-4-yl)piperazine-1-carboxylate was obtained as a yellow solid (400 mg, yield 62.8%).

LCMS: m/z 895.5 [M+H]⁺.

¹H NMR (400 MHz, CDCl₃): δ=7.82 (d, J=8.4 Hz, 1H), 7.71-7.64 (m, 1H), 7.54 (d, J=2.1 Hz, 1H), 7.41 (dt, J=3.1, 7.7 Hz, 1H), 7.35-7.28 (m, 1H), 7.19 (t, J=2.6 Hz, 1H), 5.32 (d, J=1.0 Hz, 2H), 4.79-4.46 (m, 3H), 4.32-4.11 (m, 3H), 3.73-3.41 (m, 7H), 3.37-3.05 (m, 2H), 3.02-2.67 (m, 4H), 2.42-2.14 (m, 3H), 2.02-1.74 (m, 4H), 1.53 (s, 9H), 0.89-0.81 (m, 18H), 0.62-0.50 (m, 3H) ppm.

### Step X: Synthesis of tert-butyl (2S)-2-(cyanomethyl)-4-(7-(8-ethynyl3-(methoxymethoxy)naphth-1-yl)-6,8-difluoro-2-((tetrahydro-1H-pyrrolizin-7a(5H)-yl)methoxy)quinazolin-4-yl)piperazine-1-carboxylate

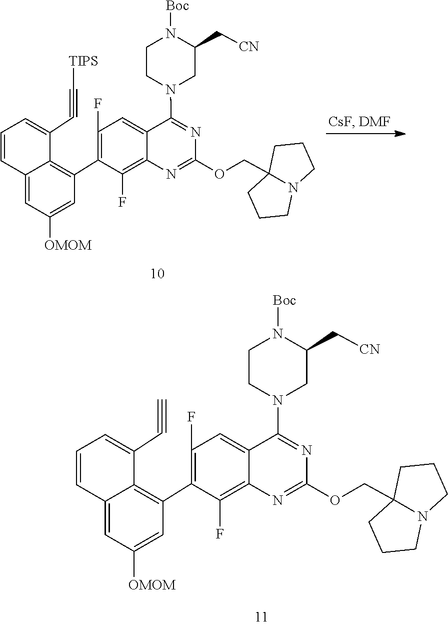

Under nitrogen protection, tert-butyl (2S)-2-(cyanomethyl)-4-(6,8-difluoro-7-(3-(methoxymethoxy)-8-((triisopropylsilyl)ethynyl-)naphth-1-yl)-2-((tetrahydro-1H-pyrrolizin-7a(5H)-yl)methoxy)quinazolin-4-yl)piperazine-1-carboxylate (400 mg, 446 umol) and cesium fluoride (271 mg, 1.79 mmol) were dissolved in N,N-dimethylformamide (5 mL) and reacted at 60° C. for 1 hour. The reaction was complete as monitored by LCMS. The reaction liquid was quenched by adding water, extracted with ethyl acetate, washed with a saturated sodium chloride aqueous solution, and dried over sodium sulfate, and after filtration, concentration and column chromatography, tert-butyl (2S)-2-(cyanomethyl)-4-(7-(8-ethynyl3-(methoxymethoxy)naphth-1-yl)-6,8-difluoro-2-((tetrahydro-1H-pyrrolizin-7a(5H)-yl)methoxy)quinazolin-4-yl)piperazine-1-carboxylate as a yellow solid (200 mg, yield 60.5%) was obtained.

LCMS: m/z 739.4 [M+H]⁺.

¹H NMR (400 MHz, CDCl₃): δ=7.85 (d, J=7.5 Hz, 1H), 7.62 (dd, J=1.0, 7.1 Hz, 1H), 7.55 (d, J=2.6 Hz, 1H), 7.41 (t, J=7.8 Hz, 1H), 7.30 (br d, J=9.3 Hz, 1H), 7.22 (t, J=2.8 Hz, 1H), 5.33 (s, 2H), 4.67 (br s, 1H), 4.25 (br d, J=10.9 Hz, 4H), 4.16-4.03 (m, 1H), 3.73-3.63 (m, 1H), 3.54 (s, 3H), 3.43 (br s, 4H), 3.05-2.90 (m, 1H), 2.89-2.70 (m, 3H), 2.69-2.58 (m, 1H), 2.21 (br d, J=1.9 Hz, 2H), 1.97 (br d, J=5.0 Hz, 4H), 1.78 (br s, 2H), 1.52 (s, 9H) ppm.

### Step XI: Synthesis of 2-((2S)-4-(7-(8-ethynyl-3-hydroxynaphth-1-yl)-6,8-difluoro-2-((tetrahydro-1H-pyrrolizin-7a(5H)-yl)methoxy)quinazolin-4-yl)piperazin-2-yl)acetonitrile

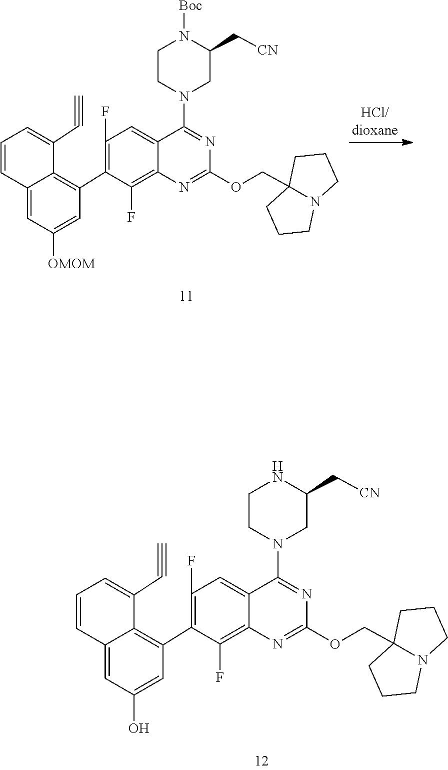

Under nitrogen protection, tert-butyl (2S)-2-(cyanomethyl)-4-(7-(8-ethynyl3-(methoxymethoxy)naphth-1-yl)-6,8-difluoro-2-((tetrahydro-1H-pyrrolizin-7a(5H)-yl)methoxy)quinazolin-4-yl)piperazine-1-carboxylate (200 mg, 270 umol) was dissolved in hydrochloric acid/dioxane (4 mL, 4M) and then reacted at 25° C. for 1 hour. The reaction was complete as monitored by LCMS. The reaction liquid was concentrated to obtain 2-((2S)-4-(7-(8-ethynyl-3-hydroxynaphth-1-yl)-6,8-difluoro-2-((tetrahydro-1H-pyrrolizin-7a(5H)-yl)methoxy)quinazolin-4-yl)piperazin-2-yl)acetonitrile as a yellow solid (160 mg, crude).

LCMS: m/z 595.3 [M+H]⁺.

### Step XII: Synthesis of 2-((2S)-4-(7-(8-ethynyl-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile

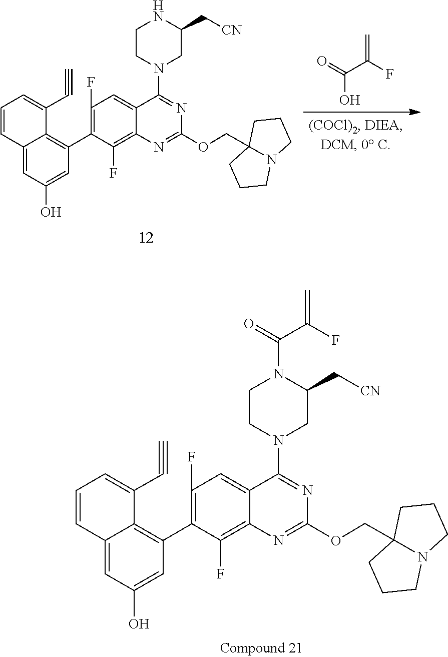

After 2-fluoroacrylic acid (96.9 mg, 1.08 mmol) was dissolved in dichloromethane (4 mL) at 0° C., oxalyl chloride (140 mg, 1.10 mmol) was added and reacted at this temperature for 10 minutes, and then, after 2-((2S)-4-(7-(8-ethynyl-3-hydroxynaphth-1-yl)-6,8-difluoro-2-((tetrahydro-1H-pyrrolizin-7a(5H)-yl)methoxy)quinazolin-4-yl)piperazin-2-yl)acetonitrile (160 mg, 269 umol) dissolved in N,N-dimethylformamide (2 mL) was added, a solution of N,N-diisopropylethylamine (104 mg, 807 umol) was added. Finally, the reaction was carried out at 0° C. for 30 minutes. The reaction was complete as monitored by LCMS. The reaction liquid was filtered and subjected to prep-HPLC and SFC for separation and purification to obtain 2-((2S)-4-(7-(8-ethynyl-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile as a white solid, and after SFC resolution, two target compound peaks 7A and 7B were obtained. Compounds 7A and 7B were axially chiral isomers of each other.

7A: (12.8 mg, yield 8.57%), LCMS: m/z 667.3 [M+H]⁺.

¹H NMR (400 MHz, CD₃CN): 6=7.91-7.79 (m, 1H), 7.57-7.47 (m, 2H), 7.45-7.35 (m, 2H), 7.15 (br d, J=2.1 Hz, 1H), 5.34-5.17 (m, 2H), 5.07-4.75 (m, 1H), 4.30-4.07 (m, 5H), 3.92-3.67 (m, 1H), 3.61-3.52 (m, 1H), 3.34 (br t, J=11.5 Hz, 1H), 3.17-3.07 (m, 1H), 3.01-2.94 (m, 2H), 2.87 (br s, 2H), 2.63-2.56 (m, 2H), 1.83-1.75 (m, 5H), 1.68-1.58 (m, 3H) ppm.

7B: (6.00 mg, yield 3.18%), LCMS: m/z 667.3 [M+H]⁺.

¹H NMR (400 MHz, CD₃CN): 6=7.87 (d, J=8.3 Hz, 1H), 7.54 (d, J=7.0 Hz, 1H), 7.50-7.36 (m, 3H), 7.15 (br d, J=1.6 Hz, 1H), 5.35-5.16 (m, 2H), 5.08-4.73 (m, 1H), 4.28-4.05 (m, 4H), 3.88-3.21 (m, 4H), 3.18-2.81 (m, 6H), 2.65 (td, J=6.8, 9.9 Hz, 3H), 1.87-1.80 (m, 4H), 1.67 (br dd, J=7.3, 12.3 Hz, 2H) ppm.

### Example 8: 2-((2S)-4-(7-(8-ethynyl-3-hydroxynaphth-1-yl)-6,8-difluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 8)

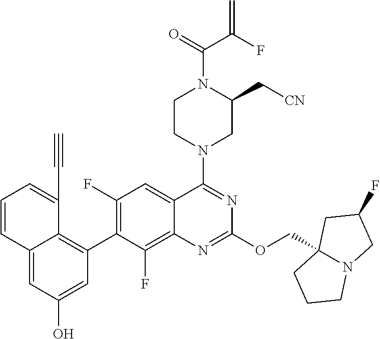 [SMILES: C#Cc1cccc2cc(O)cc(-c3c(F)cc4c(N5CCN(C(=O)C(=C)F)[C@@H](CC#N)C5)nc(OC[C@@]56CCCN5C[C@H](F)C6)nc4c3F)c12]

The specific synthesis route of Compound 8 was as follows:

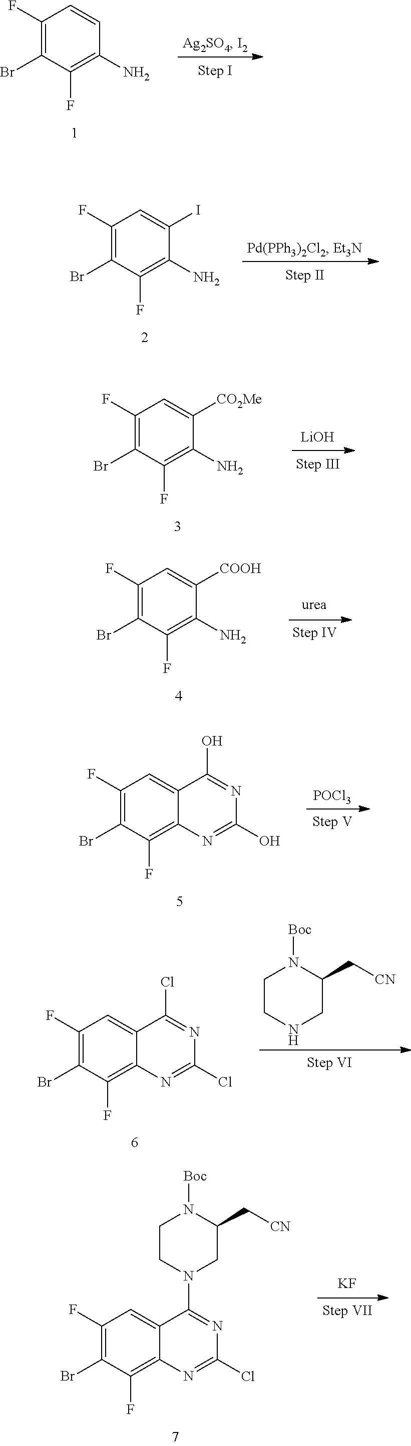

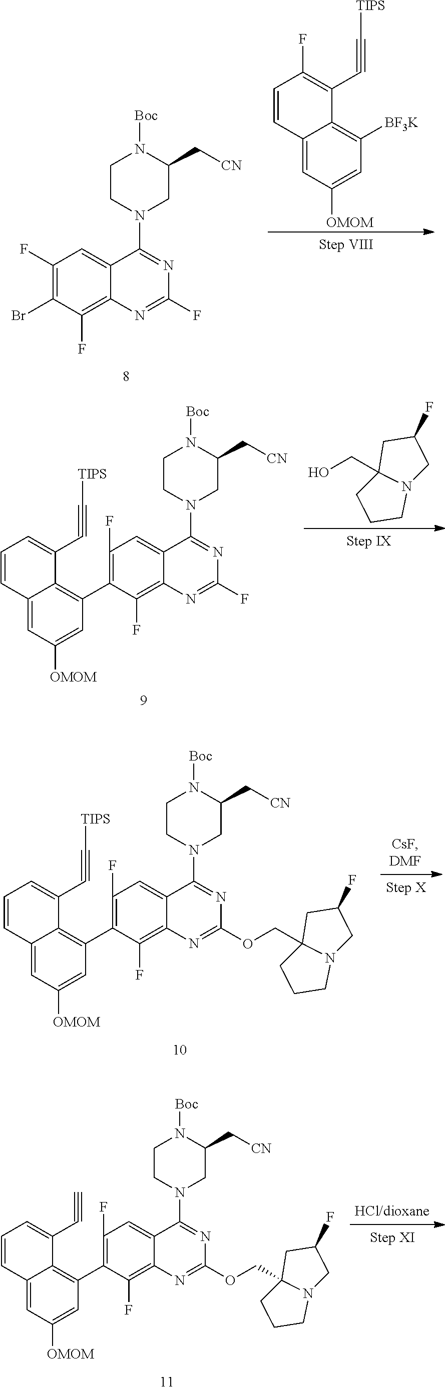

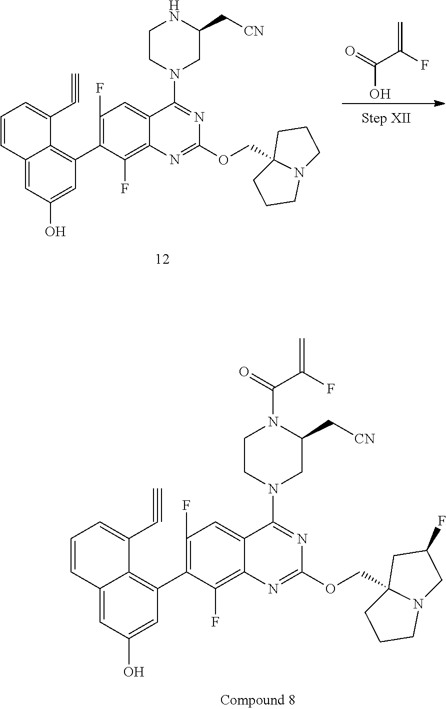

Starting from Intermediate 9 tert-butyl (2S′)-2-(cyanomethyl)-4-(2,6,8-trifluoro-7-(3-(methoxymethoxy)-8-((triisopropylsilyl)ethynyl)naphth-1-yl)quinazolin-4-yl)piperazine-1-carboxylate in the synthesis route of Compound 7, the specific synthesis process of Compound 8 was as follows:

### Step IX: Synthesis of tert-butyl (2S)-2-(cyanomethyl)-4-(6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphth-1-yl)quinazolin-4-yl)piperazine-1-carboxylate

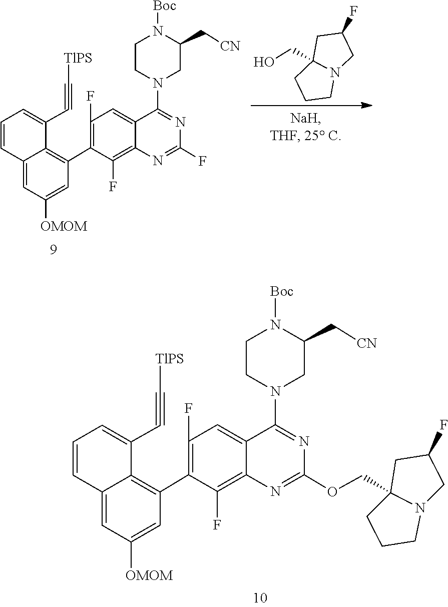

After ((2R,7aS)-2-fluorotetrahydro-1H-pyrrolizin-7a(5H)-yl)methanol (185 mg, 1.16 mmol) was dissolved in tetrahydrofuran (5 mL) under nitrogen protection at 0° C., sodium hydride (51.1 mg, 1.28 mmol) was added and reacted at 20° C. for 1 hour. Then, tert-butyl (2S)-2-(cyanomethyl)-4-(2,6,8-trifluoro-7-(3-(methoxymethoxy)-8-((triisopropylsilyl)ethynyl)naphth-1-yl)quinazolin-4-yl)piperazine-1-carboxylate (450 mg, 581 umol) was added and then reacted at 20° C. for 30 minutes.

The reaction was complete as monitored by LCMS. The reaction liquid was quenched by adding water at 0° C., extracted with ethyl acetate, washed with a saturated sodium chloride aqueous solution, and dried over sodium sulfate, and after filtration, concentration and column chromatography, tert-butyl (2S)-2-(cyanomethyl)-4-(6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphth-1-yl)quinazolin-4-yl)piperazine-1-carboxylate as a yellow solid (480 mg, yield 90.4%) was obtained.

LCMS: m/z 913.6 [M+H]⁺.

¹H NMR (400 MHz, CDCl₃): δ=7.82 (d, J=8.4 Hz, 1H), 7.67 (ddd, J=1.1, 2.6, 7.2 Hz, 1H), 7.54 (d, J=2.0 Hz, 1H), 7.45-7.38 (m, 1H), 7.30 (s, 1H), 7.20-7.12 (m, 1H), 5.32 (s, 3H), 4.79-4.64 (m, 1H), 4.22 (br d, J=12.1 Hz, 4H), 3.69-3.56 (m, 1H), 3.54 (s, 3H), 3.51-3.16 (m, 5H), 3.13-2.99 (m, 1H), 2.95-2.73 (m, 2H), 2.53-2.10 (m, 4H), 2.04-1.83 (m, 3H), 1.53 (s, 9H), 0.90-0.80 (m, 18H), 0.61-0.45 (m, 3H) ppm.

### Step X: Synthesis of tert-butyl (2S)-2-(cyanomethyl)-4-(7-(8-ethynyl-3-(methoxymethoxy)naphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate

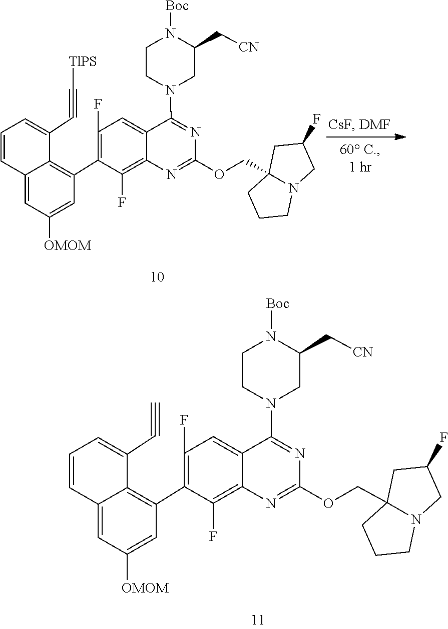

Under nitrogen protection, tert-butyl (2S)-2-(cyanomethyl)-4-(6,8-difluoro-2-(((2R,7aS)-2-fluorotetrahydro-1H-pyrrolizin-7a(5H)-yl)methoxy)-7-(3-(methoxymethoxy)-8-((triisopropylsilyl)ethynyl)naphth-1-yl)quinazolin-4-yl)piperazine-1-carboxylate (480 mg, 525 umol) and cesium fluoride (319 mg, 2.10 mmol) were dissolved in N,N-dimethylformamide (5 mL) and then reacted at 60° C. for 1 hour. The reaction was complete as monitored by LCMS. The reaction liquid was quenched by adding water at 0° C., extracted with ethyl acetate, washed with a saturated sodium chloride aqueous solution, and dried over sodium sulfate, and after filtration, concentration and column chromatography, tert-butyl (2S)-2-(cyanomethyl)-4-(7-(8-ethynyl-3-(methoxymethoxy)naphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate as a yellow oil (350 mg, yield 87.9%) was obtained.

LCMS: m/z 757.4 [M+H]⁺.

¹H NMR (400 MHz, CDCl₃): δ=8.02 (s, 1H), 7.89-7.82 (m, 1H), 7.62 (d, J=7.1 Hz, 1H), 7.56 (d, J=2.4 Hz, 1H), 7.46-7.37 (m, 1H), 7.31 (br d, J=9.3 Hz, 1H), 7.22 (dd, J=2.5, 5.4 Hz, 1H), 5.52-5.31 (m, 3H), 4.66 (br s, 1H), 4.55-4.35 (m, 2H), 4.31-4.21 (m, 1H), 4.14-4.05 (m, 1H), 3.65 (br d, J=13.9 Hz, 1H), 3.55 (s, 3H), 3.50-3.28 (m, 3H), 3.22-3.00 (m, 2H), 2.61 (br s, 3H), 2.51-2.19 (m, 3H), 2.02 (br s, 4H), 1.52 (s, 9H) ppm.

### Step XI: Synthesis of 2-((2S)-4-(7-(8-ethynyl-3-hydroxynaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile

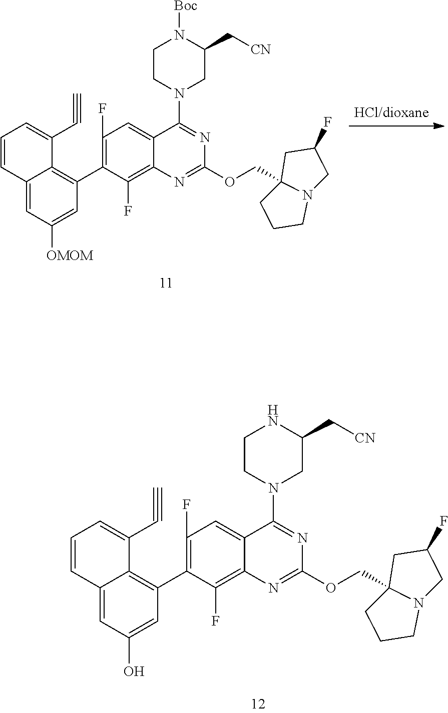

Under nitrogen protection, tert-butyl (2S)-2-(cyanomethyl)-4-(7-(8-ethynyl-3-(methoxymethoxy)naphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate (170 mg, 224 umol) was dissolved in hydrochloric acid/dioxane (3 mL) and then reacted at 0° C. for 1 hour. The reaction was complete as monitored by LCMS. The reaction liquid was concentrated to obtain 2-((2S)-4-(7-(8-ethynyl-3-hydroxynaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile as a yellow solid (130 mg, crude).

LCMS: m/z 613.2 [M+H]⁺.

### Step XII: Synthesis of 2-((2S)-4-(7-(8-ethynyl-3-hydroxynaphth-1-yl)-6,8-difluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

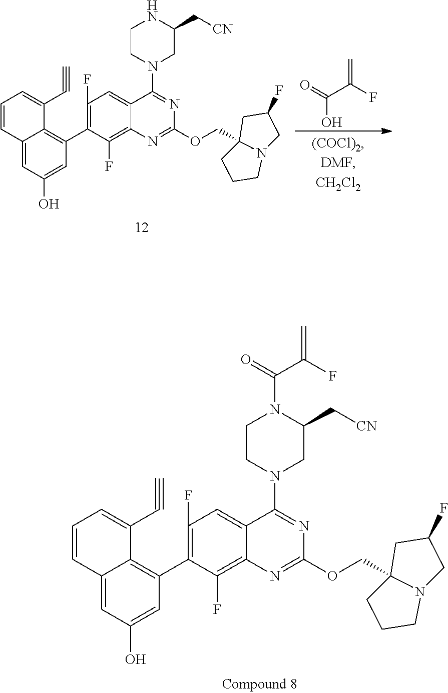

After 2-fluoroacrylic acid (76.4 mg, 848 umol) was dissolved in dichloromethane (4 mL) at 0° C., oxalyl chloride (110 mg, 870.0 umol) was added and reacted at this temperature for 10 minutes, and then, after 2-((2S)-4-(7-(8-ethynyl-3-hydroxynaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile (130 mg, 212 umol) dissolved in N,N-dimethylformamide (2 mL) was added, a solution of N,N-diisopropylethylamine (82.2 mg, 636 umol) was added. Finally, the reaction was carried out at 0° C. for 30 minutes. The reaction was complete as monitored by LCMS. The reaction liquid was filtered and subjected to prep-HPLC and SFC for separation and purification to obtain 2-((2S)-4-(7-(8-ethynyl-3-hydroxynaphth-1-yl)-6,8-difluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile as a yellow solid, and after SFC resolution, two target compound peaks 8A and 8B were obtained. Compounds 8A and 8B were axially chiral isomers of each other.

8A: (8.1 mg, yield 5.54%), LCMS: m/z 685.5 [M+H]⁺.

¹H NMR (400 MHz, CD₃CN): 6=7.88 (d, J=8.4 Hz, 1H), 7.58-7.48 (m, 2H), 7.46-7.37 (m, 2H), 7.16 (d, J=2.5 Hz, 1H), 5.38-5.17 (m, 3H), 4.92 (dt, J=4.6, 7.2 Hz, 1H), 4.30-4.03 (m, 5H), 3.88-3.50 (m, 2H), 3.37 (br t, J=11.3 Hz, 1H), 3.18-3.03 (m, 4H), 2.94-2.82 (m, 3H), 2.07-2.01 (m, 2H), 1.91-1.70 (m, 4H) ppm.

8B: (7.70 mg, yield 5.20%), LCMS: m/z 685.4 [M+H]⁺.

¹H NMR (400 MHz, CD₃CN): 6=7.88 (d, J=8.3 Hz, 1H), 7.57-7.41 (m, 3H), 7.39 (d, J=2.4 Hz, 1H), 7.16 (d, J=2.5 Hz, 1H), 5.37-5.13 (m, 3H), 5.10-4.69 (m, 1H), 4.33-3.98 (m, 5H), 3.79-3.26 (m, 3H), 3.22-3.05 (m, 4H), 3.01-2.81 (m, 3H), 2.08-2.02 (m, 2H), 1.92-1.78 (m, 4H) ppm.

### Example 9: (S)-2-(4-(7-(8-chloronaphth-1-yl)-8-fluoro-2-((tetrahydro-1H-pyrrolizin-7a(5H)-yl)methyl)amino)quinazolin-4-yl)-1-(2-fluoroacryloyl)piperazin-2-yl)acetonitrile (Compound 9)

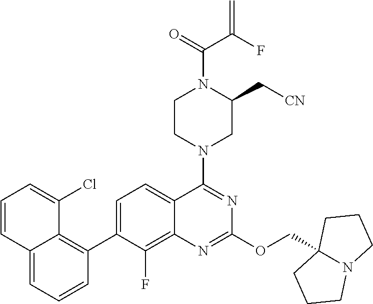

The specific synthesis route of Compound 9 was as follows:

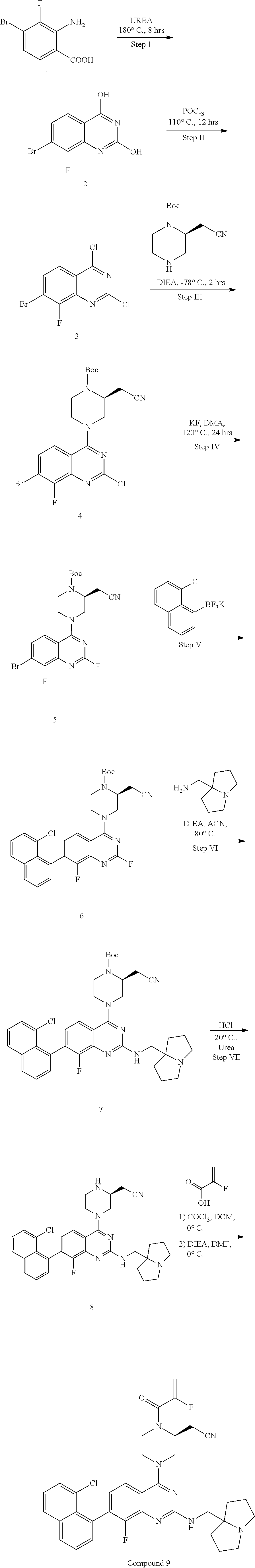

Starting from the intermediate No. 5 compound, the specific synthesis process of Compound 9 was as follows:

### Step V: Synthesis of tert-butyl (S)-4-(7-(8-chloronaphth-1-yl)-2,8-difluoroquinazolin-4-yl)-2-(cyanomethyl)piperazine-1-carboxylate

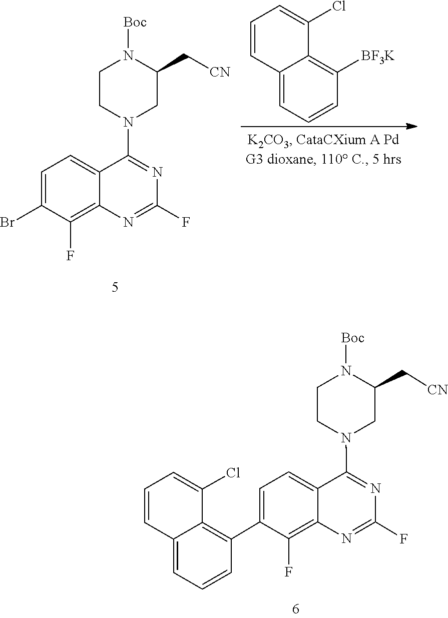

Tert-butyl (S)-4-(7-bromo-2,8-difluoroquinazolin-4-yl)-2-(cyanomethyl)piperazine-1-carboxylate (400 mg, 854 umol), potassium (8-chloro-1-naphthyl)trifluoroboronate (321 mg, 1.20 mmol), methanesulfonato(diadamantyl-N-butylphosphino)-2′-amino-1,1′-biphenyl-2-yl)palladium(11)(124 mg, 171 umol), and potassium carbonate (236 mg, 1.71 mmol) were dissolved in 2.50 mL of dioxane, and after nitrogen displacement three times, the mixture was stirred at 110° C. for 5 hours. After the reaction was complete as monitored by LCMS, the reaction liquid was diluted with ethyl acetate and washed with brine, and the organic phase was concentrated under reduced pressure and purified by a fast silica gel column to obtain tert-butyl (S)-4-(7-(8-chloronaphth-1-yl)-2,8-difluoroquinazolin-4-yl)-2-(cyanomethyl)piperazine-1-carboxylate (420 mg, 764 umol, 89.4% yield).

¹H NMR (400 MHz, CDCl₃): δ=8.06-7.94 (m, 2H), 7.90 (d, J=8.0 Hz, 1H), 7.78-7.72 (m, 1H), 7.63-7.53 (m, 2H), 7.46-7.41 (m, 2H), 4.75-4.63 (m, 1H), 4.49-4.41 (m, 2H), 4.28-4.16 (m, 1H), 3.67-3.59 (m, 1H), 3.54-3.42 (m, 1H), 2.91-2.74 (m, 2H), 2.51-2.29 (m, 1H), 1.54 (s, 9H) ppm.

### Step VI: Synthesis of tert-butyl (S)-4-(7-(8-chloronaphth-1-yl)-8-fluoro-2-(((tetrahydro-1H-pyrrolizin-7a(5H)-yl)methyl)amino)quinazolin-4-yl)-2-(cyanomethyl)piperazine-1-carboxylate

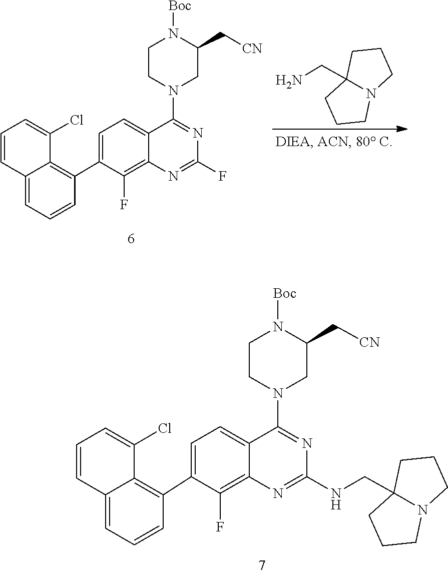 [SMILES: CC(C)(C)OC(=O)N1CCN(c2nc(F)nc3c(F)c(-c4cccc5cccc(Cl)c45)ccc23)C[C@@H]1CC#N ; NCC12CCCN1CCC2 ; CC(C)(C)OC(=O)N1CCN(c2nc(NCC34CCCN3CCC4)nc3c(F)c(-c4cccc5cccc(Cl)c45)ccc23)C[C@@H]1CC#N]

Tert-butyl (S)-4-(7-(8-chloronaphth-1-yl)-2,8-difluoroquinazolin-4-yl)-2-(cyanomethyl)piperazine-1-carboxylate (200 mg, 363 umol), (tetrahydro-1H-pyrrolizin-7a(5H)-yl)methanamine (81.5 mg, 581 umol), and DIEA (117 mg, 909 umol) were dissolved in acetonitrile (3.00 mL) and stirred at 80° C. for 2 hours. After the reaction was complete as monitored by LCMS and TLC, the reaction liquid was directly concentrated under reduced pressure and purified by a fast silica gel column to obtain tert-butyl (S)-4-(7-(8-chloronaphth-1-yl)-8-fluoro-2-(((tetrahydro-1H-pyrrolizin-7a(5H)-yl)methyl)amino)quinazolin-4-yl)-2-(cyanomethyl)piperazine-1-carboxylate (110 mg, 164 umol, 45.1% yield).

¹H NMR (400 MHz, CDCl₃): δ=8.05-7.91 (m, 2H), 7.90-7.79 (m, 1H), 7.71 (td, J=7.6, 15.6 Hz, 1H), 7.62-7.51 (m, 2H), 7.50-7.36 (m, 2H), 4.70 (br d, J=2.1 Hz, 1H), 4.41-4.19 (m, 2H), 3.68-3.40 (m, 3H), 3.30 (td, J=6.8, 13.5 Hz, 2H), 3.02-2.64 (m, 5H), 2.13-2.02 (m, 2H), 1.96-1.80 (m, 4H), 1.67-1.60 (m, 4H), 1.55-1.51 (m, 9H) ppm.

### Step VII: Synthesis of (S)-2-(4-(7-(8-chloronaphth-1-yl)-8-fluoro-2-(((tetrahydro-1H-pyrrolizin-7a(5H)-yl)methyl)amino)quinazolin-4-yl)piperazin-2-yl)acetonitrile

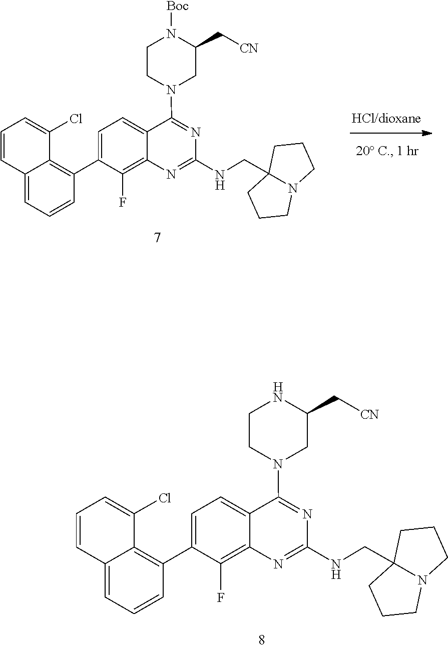 [SMILES: CC(C)(C)OC(=O)N1CCN(c2nc(NCC34CCCN3CCC4)nc3c(F)c(-c4cccc5cccc(Cl)c45)ccc23)C[C@@H]1CC#N ; N#CC[C@H]1CN(c2nc(NCC34CCCN3CCC4)nc3c(F)c(-c4cccc5cccc(Cl)c45)ccc23)CCN1]

Tert-butyl (S)-4-(7-(8-chloronaphth-1-yl)-8-fluoro-2-(((tetrahydro-1H-pyrrolizin-7a(5H)-yl)methyl)amino)quinazolin-4-yl)-2-(cyanomethyl)piperazine-1-carboxylate (100 mg, 149 umol) was dissolved in hydrochloric acid/1,4-dioxane (4M, 0.2 mL), and after nitrogen displacement three times, the mixture was stirred at 20° C. for 1 hour. After the reaction was complete as monitored by LCMS, the reaction liquid was concentrated under reduced pressure to obtain (S)-2-(4-(7-(8-chloronaphth-1-yl)-8-fluoro-2-(((tetrahydro-1H-pyrrolizin-7a(5H)-yl)methyl)amino)quinazolin-4-yl)piperazin-2-yl)acetonitrile (85.0 mg, crude).

### Step VIII: Synthesis of (S)-2-(4-(7-(8-chloronaphth-1-yl)-8-fluoro-2-((tetrahydro-1H-pyrrolizin-7a(5H)-yl)methyl)amino)quinazolin-4-yl)-1-(2-fluoroacryloyl)piperazin-2-yl)acetonitrile

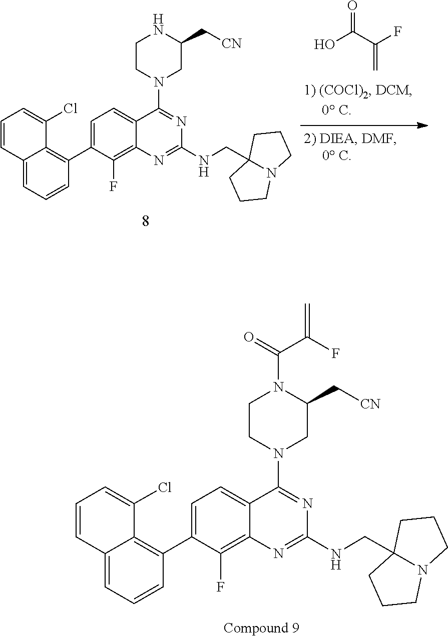

2-Fluoroprop-2-enoic acid (13.3 mg, 149 umol) was dissolved in 2.00 mL of dichloromethane, oxalyl chloride (3.03 mg, 23.8 umol, 33.7 uL) was added to the reaction liquid at 0° C., N,N-dimethylformamide (108 ug, 1.49 umol) was then added, and the mixture was heated to 20° C. and stirred for 30 minutes. Subsequently, at 0° C., the above reaction liquid was dropwise added to a solution of (S)-2-(4-(7-(8-chloronaphth-1-yl)-8-fluoro-2-(((tetrahydro-1H-pyrrolizin-7a(5H)-yl)methyl)amino)quinazolin-4-yl)piperazin-2-yl)acetonitrile (85.0 mg, 149 umol) and DIEA(192 mg, 1.49 mmol) in N,N-dimethylformamide (2.00 mL), and the mixture was heated to 20° C. and stirred for 1 hour. After the reaction was complete as monitored by LCMS, the reaction liquid was concentrated and then purified by prep-HPLC and SFC to obtain (S)-2-(4-(7-(8-chloronaphth-1-yl)-8-fluoro-2-((tetrahydro-1H-pyrrolizin-7a(5H)-yl)methyl)amino)quinazolin-4-yl)-1-(2-fluoroacryloyl)piperazin-2-yl)acetonitrile (12.5 mg, 19.4 umol, 13.8% yield).

LCMS: m/z 642.5 [M+H]⁺.

¹H NMR (400 MHz, CD₃CN): 6=8.08 (d, J=7.9 Hz, 1H), 8.01 (d, J=8.1 Hz, 1H), 7.72-7.62 (m, 2H), 7.62-7.57 (m, 1H), 7.54-7.47 (m, 2H), 7.20-7.05 (m, 1H), 6.52 (br s, 1H), 5.35-5.18 (m, 2H), 5.06-4.79 (m, 1H), 4.29 (br d, J=13.4 Hz, 2H), 4.20-3.93 (m, 1H), 3.80-3.55 (m, 3H), 3.54-3.35 (m, 3H), 3.33-3.20 (m, 2H), 3.14-3.04 (m, 1H), 3.03-2.95 (m, 1H), 2.94-2.81 (m, 2H), 2.07-1.99 (m, 3H), 1.90-1.84 (m, 2H), 1.84-1.74 (m, 2H) ppm.

### Example 10: 2-((2S)-4-(6-chloro-7-(8-ethynyl-7-fluoro-3-hydroxynaphth-1-yl)-8-fluoro-2-((2R,7aS)-2-fluorotetrahydro-1H-pyrrolizin-7a(5H)-ylmethoxy)quinazolin-4-yl)-1-(2-fluoroacryloyl)piperazin-2-yl)acetonitrile (Compound 10)

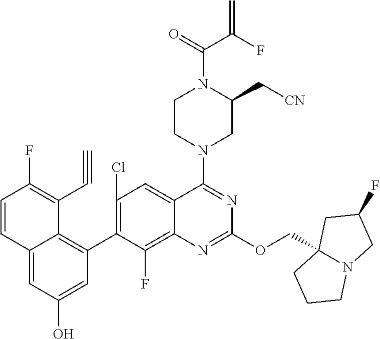 [SMILES: C#Cc1c(F)ccc2cc(O)cc(-c3c(Cl)cc4c(N5CCN(C(=O)C(=C)F)[C@@H](CC#N)C5)nc(OC[C@@]56CCCN5C[C@H](F)C6)nc4c3F)c12]

LCMS: m/z 719.2 [M+H]⁺.

### Example 11: (S)-2-(4-(7-(8-ethynylnaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (Compound 11)

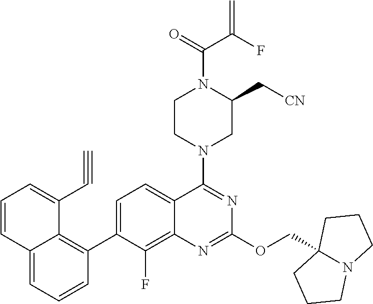

The specific synthesis route of Compound 11 was as follows:

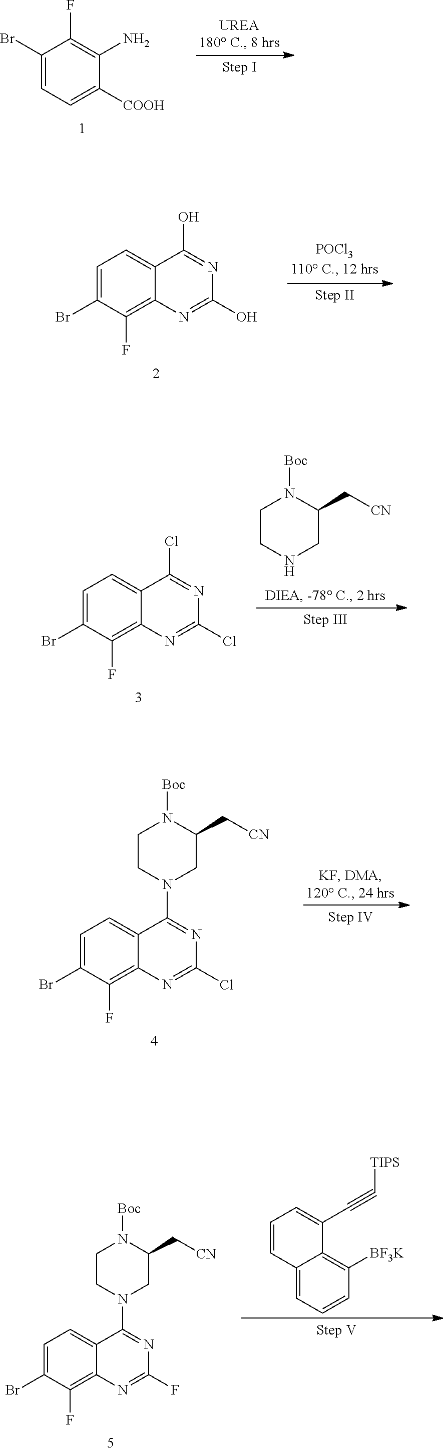

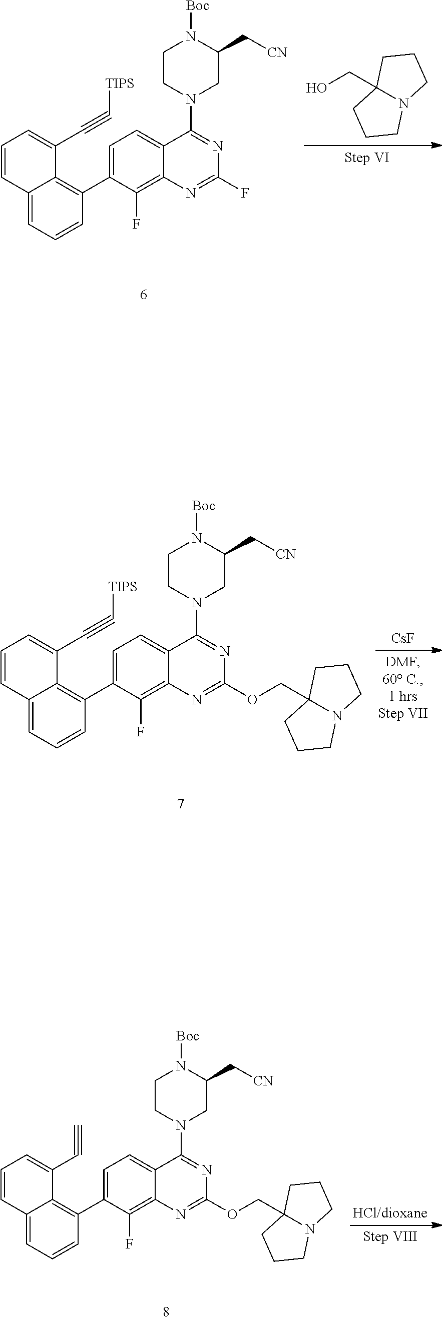

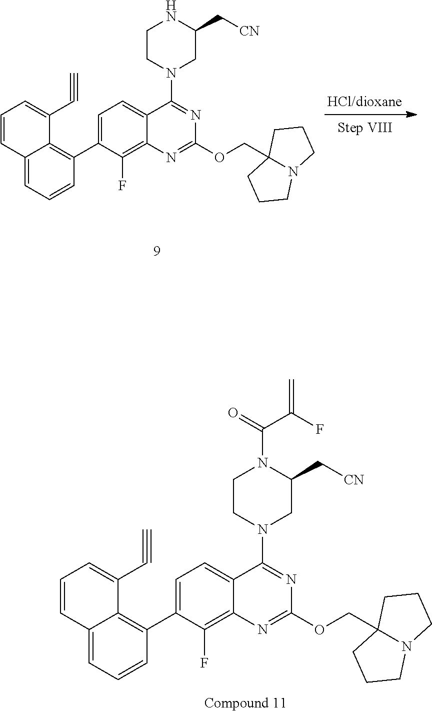 [SMILES: C#Cc1cccc2cccc(-c3ccc4c(N5CCN[C@@H](CC#N)C5)nc(OCC56CCCN5CCC6)nc4c3F)c12 ; C#Cc1cccc2cccc(-c3ccc4c(N5CCN(C(=O)C(=C)F)[C@@H](CC#N)C5)nc(OCC56CCCN5CCC6)nc4c3F)c12]

Starting from the intermediate No. 5 compound, the specific synthesis process of Compound 11 was as follows:

### Step V: Synthesis of tert-butyl (S)-2-(cyanomethyl)-4-(2,8-difluoro-7-(8-((triisopropylsilyl)ethynyl)naphth-1-yl)quinazolin-4-yl)piperazine-1-carboxylate

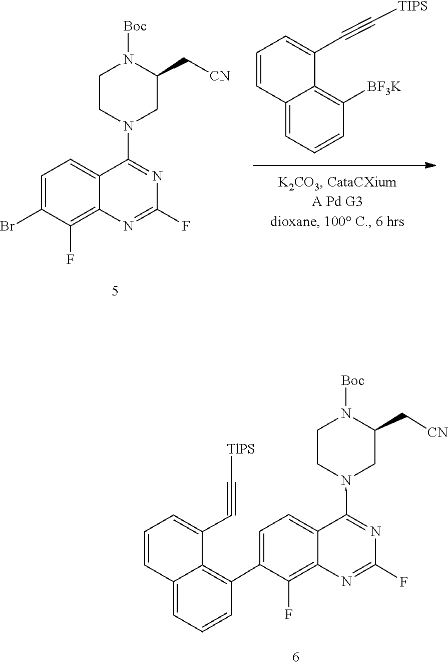

Tert-butyl (S)-4-(7-bromo-2,8-difluoroquinazolin-4-yl)-2-(cyanomethyl)piperazine-1-carboxylate (300 mg, 641 umol), potassium (8-(2-triisopropylsilyl)-1-naphthyl)trifluoroboronate (531 mg, 1.28 mmol), methanesulfonato(diadamantyl-N-butylphosphino)-2′-amino-1,1′-biphenyl-2-yl)palladium(II)(93.0 mg, 128 umol), and potassium carbonate (177 mg, 1.28 mmol) were dissolved in 3.00 mL of dioxane, and the reaction liquid was stirred at 100° C. for 6 hours under nitrogen atmosphere protection. After the reaction was complete as monitored by LCMS, the reaction liquid was concentrated under reduced pressure and purified by a fast silica gel column to obtain tert-butyl (S)-2-(cyanomethyl)-4-(2,8-difluoro-7-(8-((triisopropylsilyl)ethynyl)naphth-1-yl)quinazolin-4-yl)piperazine-1-carboxylate (330 mg, 474 umol, 74.0% yield).

¹H NMR (400 MHz, CDCl₃): δ=7.95 (t, J=7.6 Hz, 2H), 7.82 (br d, J=7.2 Hz, 1H), 7.74 (br dd, J=9.2, 12.0 Hz, 1H), 7.57 (t, J=7.6 Hz, 1H), 7.53-7.44 (m, 2H), 7.41 (br d, J=6.8 Hz, 1H), 4.71 (br s, 1H), 4.53-4.26 (m, 2H), 4.16 (br s, 1H), 3.91-3.29 (m, 3H), 3.10-2.67 (m, 2H), 1.54 (br d, J=4.4 Hz, 9H), 1.02-0.76 (m, 18H), 0.55 (quin, J=7.2 Hz, 3H) ppm.

### Step VI. Synthesis of tert-butyl (S)-2-(cyanomethyl)-4-(8-fluoro-2-((tetrahydro-1H-pyrrolizin-7a(5H)-yl)methoxy)-7-(8-((triisopropylsilyl)ethynyl)naphth-1-yl)quinazolin-4-yl)piperazine-1-carboxylate

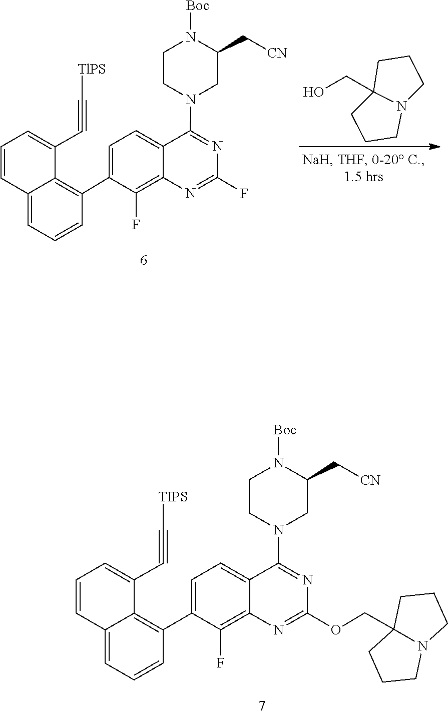

(Tetrahydro-1H-pyrrolizin-7a(5H)-yl)methanol (244 mg, 1.72 mmol) was dissolved in 10.0 mL of tetrahydrofuran, sodium hydride (77.6 mg, 1.94 mmol, 60.0% purity) was added at 0° C., the reaction liquid was stirred at 0° C. for 0.5 hours, tert-butyl (S)-2-(cyanomethyl)-4-(2,8-difluoro-7-(8-((triisopropylsilyl)ethynyl)naphth-1-yl)quinazolin-4-yl)piperazine-1-carboxylate (300 mg, 431 umol) was dissolved in 5.00 mL of tetrahydrofuran, which was dropwise added to the above reaction liquid, and the reaction liquid was further stirred at 20° C. for 1 hour. After the reaction was complete as monitored by LCMS, the reaction liquid was quenched with a saturated ammonium chloride aqueous solution, extracted with ethyl acetate, and washed with brine, and the organic phase was concentrated and purified by a fast silica gel column to obtain tert-butyl (S)-2-(cyanomethyl)-4-(8-fluoro-2-((tetrahydro-1H-pyrrolizin-7a(5H)-yl)methoxy)-7-(8-((triisopropylsilyl)ethynyl)naphth-1-yl)quinazolin-4-yl)piperazine-1-carboxylate (310 mg, 379 umol, 88.0% yield).

LCMS: m/z 817.3 [M+H]⁺.

### Step VII: Synthesis of tert-butyl (S)-2-(cyanomethyl)-4-(7-(8-ethynylnaphth-1-yl)-8-fluoro-2-((tetrahydro-1H-pyrrolizin-7a(5H)-yl)methoxy)quinazolin-4-yl)piperazine-1-carboxylate

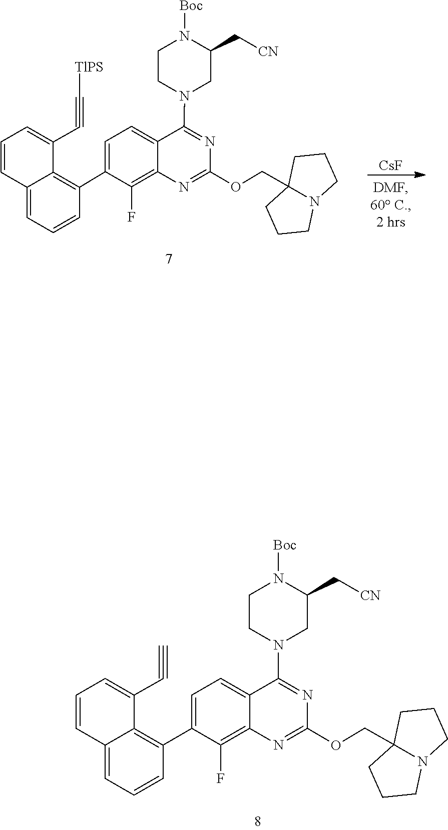

Tert-butyl (S)-2-(cyanomethyl)-4-(8-fluoro-2-((tetrahydro-1H-pyrrolizin-7a(5H)-yl)methoxy)-7-(8-((triisopropylsilyl)ethynyl)naphth-1-yl)quinazolin-4-yl)piperazine-1-carboxylate (260 mg, 318 umol) was dissolved in N,N-dimethylformamide (10.0 mL), and cesium fluoride (145 mg, 955 umol) was added. The reaction liquid was reacted at 60° C. for 2 hours. The reaction was complete as monitored by LCMS. The reaction liquid was diluted by adding water, extracted with ethyl acetate, washed with brine, and dried, and the organic phase was concentrated and purified by a fast silica gel column to obtain tert-butyl (S)-2-(cyanomethyl)-4-(7-(8-ethynylnaphth-1-yl)-8-fluoro-2-((tetrahydro-1H-pyrrolizin-7a(5H)-yl)methoxy)quinazolin-4-yl)piperazine-1-carboxylate (220 mg, 288 umol, 90.4% yield).

LCMS: m/z 661.5 [M+H]⁺.

¹H NMR (400 MHz, CDCl₃): δ=7.96 (d, J=8.4 Hz, 2H), 7.74 (d, J=7.2 Hz, 1H), 7.63-7.54 (m, 2H), 7.49-7.41 (m, 2H), 7.27-7.21 (m, 1H), 4.68 (br s, 1H), 4.46-4.22 (m, 4H), 4.15-4.01 (m, 1H), 3.69-3.55 (m, 1H), 3.50-3.16 (m, 4H), 2.88-2.54 (m, 5H), 2.24-2.11 (m, 2H), 2.01-1.85 (m, 4H), 1.79-1.66 (m, 2H), 1.53 (s, 9H) ppm.

### Step VIII: Synthesis of (S)-2-(4-(7-(8-ethynylnaphth-1-yl)-8-fluoro-2-((tetrahydro-1H-pyrrolizin-7a(5H)-yl)methoxy)quinazolin-4-yl)piperazin-2-yl)acetonitrile

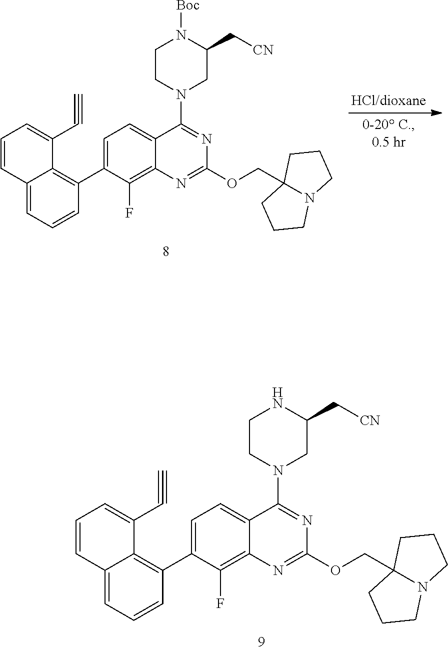 [SMILES: C#Cc1cccc2cccc(-c3ccc4c(N5CCN(C(=O)OC(C)(C)C)[C@@H](CC#N)C5)nc(OCC56CCCN5CCC6)nc4c3F)c12 ; C#Cc1cccc2cccc(-c3ccc4c(N5CCN[C@@H](CC#N)C5)nc(OCC56CCCN5CCC6)nc4c3F)c12]

At 0° C., 2.00 mL of a hydrochloric acid/1,4-dioxane solution was added to tert-butyl (S)-2-(cyanomethyl)-4-(7-(8-ethynylnaphth-1-yl)-8-fluoro-2-((tetrahydro-1H-pyrrolizin-7a(5H)-yl)methoxy)quinazolin-4-yl)piperazine-1-carboxylate (200 mg, 303 umol), and the reaction liquid was stirred at 20° C. for 0.5 hours. After the reaction was complete as monitored by LCMS, the reaction liquid was concentrated under reduced pressure to obtain (S)-2-(4-(7-(8-ethynylnaphth-1-yl)-8-fluoro-2-((tetrahydro-1H-pyrrolizin-7a(5H)-yl)methoxy)quinazolin-4-yl)piperazin-2-yl)acetonitrile (180 mg, crude).

LCMS: m/z 561.3 [M+H]⁺.

### Step IX: Synthesis of (S)-2-(4-(7-(8-ethynylnaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile

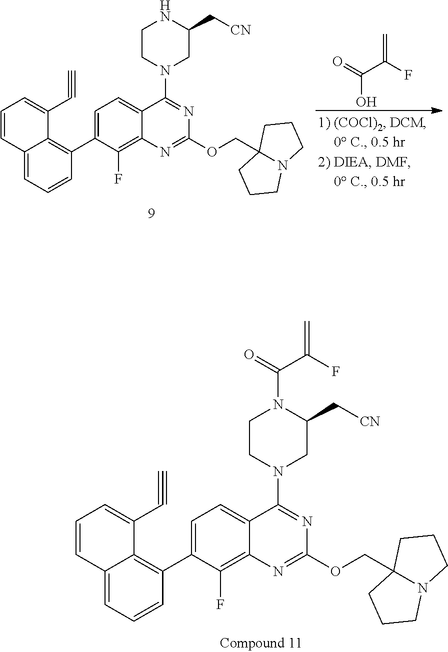

2-Fluoroprop-2-enoic acid (34.7 mg, 385 umol) was dissolved in 1.00 mL of dichloromethane, oxalyl chloride (48.9 mg, 385 umol, 33.7 uL) was added to the reaction liquid at 0° C., and N,N-dimethylformamide (2.35 mg, 32.1 umol, 2.47 uL) was then added and stirred for 0.5 hours; subsequently, the reaction liquid was dropwise added to a solution of (S)-2-(4-(7-(8-ethynylnaphth-1-yl)-8-fluoro-2-((tetrahydro-1H-pyrrolizin-7a(5H)-yl)methoxy)quinazolin-4-yl)piperazin-2-yl)acetonitrile (180 mg, 321 umol) and N,N-diisopropylethylamine (83.0 mg, 642 umol, 112 uL) in N,N-dimethylformamide (1.00 mL) and stirred for 0.5 hours. After the reaction was complete as monitored by LCMS, the reaction liquid was concentrated and purified by prep-HPLC to obtain (S)-2-(4-(7-(8-ethynylnaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (22.9 mg, 36.0 umol, 11.2% yield).

LCMS: m/z 633.6 [M+H]⁺.

¹H NMR (400 MHz, CD₃CN): 6=8.07 (d, J=8.4 Hz, 2H), 7.79-7.70 (m, 2H), 7.69-7.61 (m, 1H), 7.56-7.47 (m, 2H), 7.28 (ddd, J=4.4, 6.8, 8.5 Hz, 1H), 5.41-5.15 (m, 2H), 4.93 (br d, J=3.2 Hz, 1H), 4.36-4.21 (m, 2H), 4.12 (s, 2H), 3.95-3.28 (m, 4H), 3.12 (br dd, J=7.2, 18.1 Hz, 1H), 3.04-2.81 (m, 4H), 2.66-2.56 (m, 2H), 2.03-1.96 (m, 2H), 1.89-1.74 (m, 4H), 1.71-1.58 (m, 2H) ppm.

### Example 12: (S)-2-(4-(7-(8-ethynyl-7-fluoro-3-hydroxynaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (Compound 12)

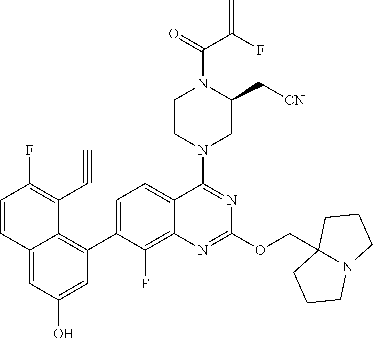 [SMILES: C#Cc1c(F)ccc2cc(O)cc(-c3ccc4c(N5CCN(C(=O)C(=C)F)[C@@H](CC#N)C5)nc(OCC56CCCN5CCC6)nc4c3F)c12]

The specific synthesis route of Compound 12 was as follows:

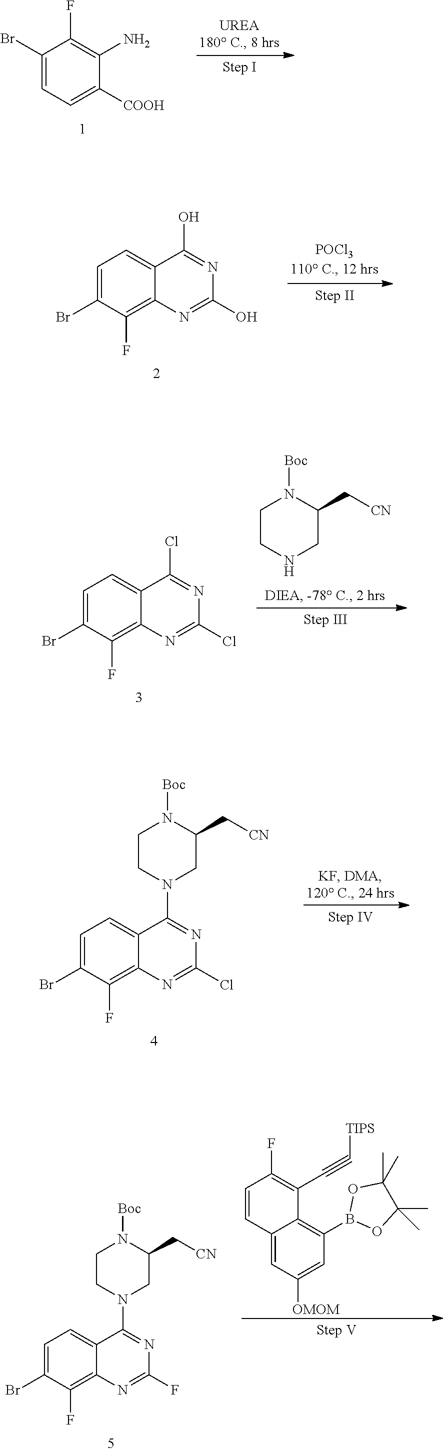

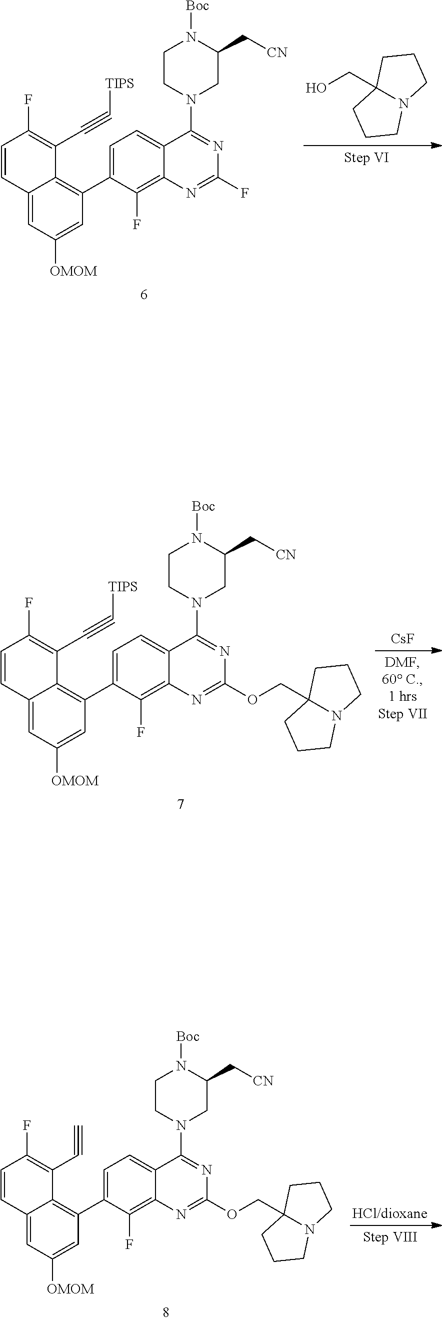

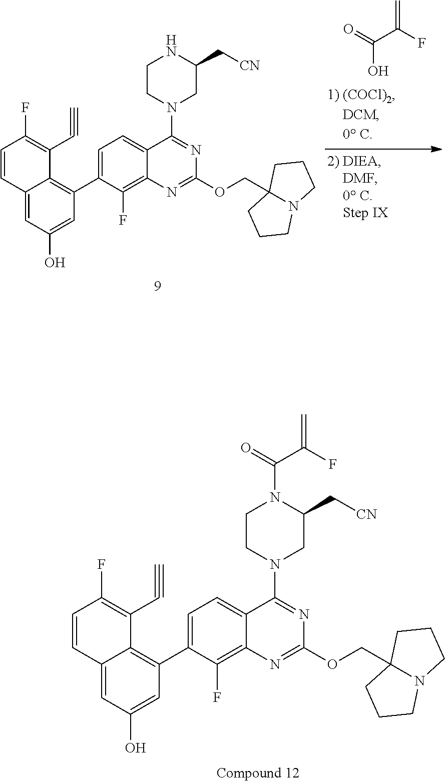

Starting from the intermediate No. 5 compound, the specific synthesis process of Compound 12 was as follows:

### Step V: Synthesis of tert-butyl (S)-2-(cyanomethyl)-4-(2,8-difluoro-7-(7-fluoro-3-(methoxymethoxy)-8-((triisopropylsilyl)ethynyl)naphth-1-yl)quinazolin-4-yl)piperazine-1-carboxylate

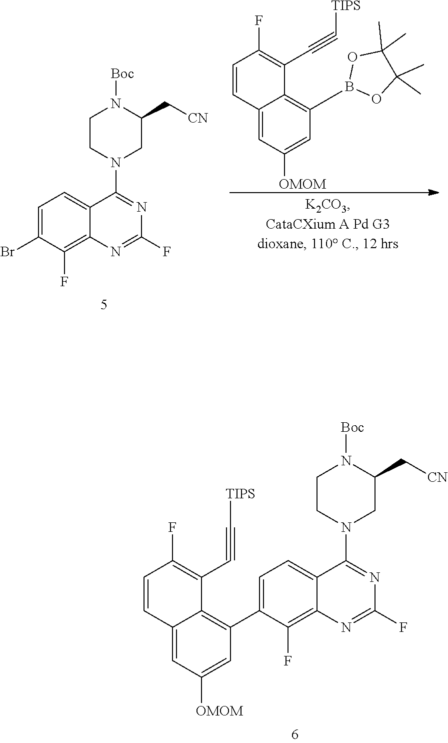

Tert-butyl (S)-4-(7-bromo-2,8-difluoroquinazolin-4-yl)-2-(cyanomethyl)piperazine-1-carboxylate (300 mg, 641 umol) and ((2-fluoro-6-(methoxymethoxy)-8-(4,4,5,5-tetramethyl-1,3,2-dioxaboran-2-yl)naphth-1-yl)ethynyl)triisopropylsilane (394 mg, 769 umol) were dissolved in 5.00 mL of dioxane, methanesulfonato(diadamantyl-N-butylphosphino)-2′-amino-1,1′-biphenyl-2-yl)palladium(II) (93.3 mg, 128 umol) and potassium carbonate (177 mg, 1.28 mmol) were added, and the reaction liquid was stirred at 110° C. for 12 hours. Five reactions were carried out in parallel. After the reaction was complete as monitored by LCMS, the reaction liquid was concentrated and purified by a fast silica gel column to obtain tert-butyl (S)-2-(cyanomethyl)-4-(2,8-difluoro-7-(7-fluoro-3-(methoxymethoxy)-8-((triisopropylsilyl)ethynyl)naphth-1-yl)quinazolin-4-yl)piperazine-1-carboxylate (1.05 g, 1.36 mmol, 42.4% yield).

LCMS: m/z 774.5 [M+H]⁺.

¹H NMR (400 MHz, CDCl₃): δ=7.80 (dd, J=5.6, 9.1 Hz, 1H), 7.76-7.70 (m, 1H), 7.51 (d, J=2.4 Hz, 1H), 7.49-7.42 (m, 1H), 7.32 (t, J=8.8 Hz, 1H), 7.18 (d, J=2.4 Hz, 1H), 5.31 (s, 2H), 4.71 (br s, 1H), 3.53 (s, 3H), 2.91-2.70 (m, 2H), 1.54 (d, J=4.4 Hz, 9H), 1.25 (s, 6H), 0.87-0.83 (m, 18H), 0.54 (quin, J=7.6 Hz, 3H) ppm.

### Step VI: Synthesis of tert-butyl (S)-2-(cyanomethyl)-4-(8-fluoro-7-(7-fluoro-3-(methoxymethoxy)-8-((triisopropylsilyl)ethynyl)naphth-1-yl)-2-((tetrahydro-1H-pyrrolizin-7a(5H)-yl)methoxy)quinazolin-4-yl)piperazine-1-carboxylate

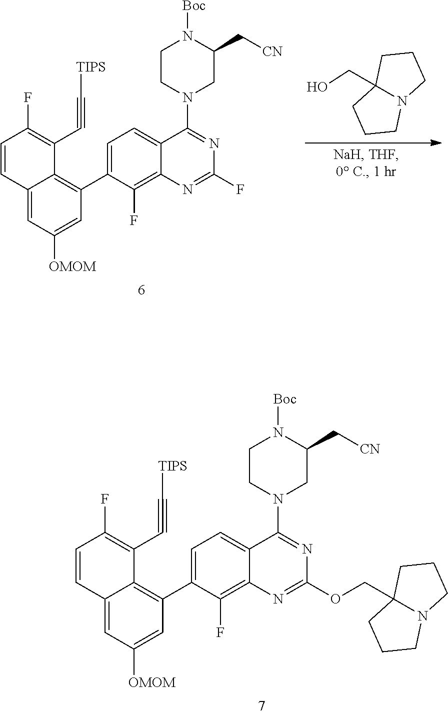

(Tetrahydro-1H-pyrrolizin-7a(5H)-yl)methanol (493 mg, 3.49 mmol) was dissolved in 20.0 mL of tetrahydrofuran, sodium hydride (209 mg, 5.23 mmol, 60.0% purity) was added at 0° C., and the reaction liquid was stirred at 20° C. for 1 hour. Tert-butyl (S)-2-(cyanomethyl)-4-(2,8-difluoro-7-(7-fluoro-3-(methoxymethoxy)-8-((triisopropylsilyl)ethynyl)naphth-1-yl)quinazolin-4-yl)piperazine-1-carboxylate (900 mg, 1.16 mmol) was added to the reaction liquid, and the reaction liquid was further stirred at 20° C. for 2 hours. After the reaction was complete as monitored by LCMS, the reaction liquid was quenched with water and extracted with ethyl acetate, and washed with brine, and the organic phase was concentrated and purified by a fast silica gel column to obtain tert-butyl (S)-2-(cyanomethyl)-4-(8-fluoro-7-(7-fluoro-3-(methoxymethoxy)-8-((triisopropylsilyl)ethynyl)naphth-1-yl)-2-((tetrahydro-1H-pyrrolizin-7a(5H)-yl)methoxy)quinazolin-4-yl)piperazine-1-carboxylate (0.69 g, 765 umol, 65.8% yield).

LCMS: m/z 895.6 [M+H]⁺.

¹H NMR (400 MHz, CDCl₃): δ=7.79 (dd, J=5.6, 8.8 Hz, 1H), 7.68-7.59 (m, 1H), 7.49 (d, J=2.4 Hz, 1H), 7.36-7.28 (m, 2H), 7.19 (t, J=2.4 Hz, 1H), 5.30 (s, 2H), 4.71 (br dd, J=4.4, 5.3 Hz, 1H), 4.33-4.21 (m, 2H), 3.53 (d, J=0.8 Hz, 3H), 3.36-3.11 (m, 2H), 2.97-2.62 (m, 4H), 2.39-2.18 (m, 2H), 2.10-1.69 (m, 8H), 1.69-1.58 (m, 4H), 1.53 (s, 9H), 0.84 (td, J=4.0, 7.6 Hz, 18H), 0.53 (quin, J=7.2 Hz, 3H) ppm.

### Step VII: Synthesis of tert-butyl (S)-2-(cyanomethyl)-4-(7-(8-ethynyl-7-fluoro-3-(methoxymethoxy)naphth-1-yl)-8-fluoro-2-((tetrahydro-1H-pyrrolizin-7a(5H)-yl)methoxy)quinazolin-4-yl)piperazine-1-carboxylate

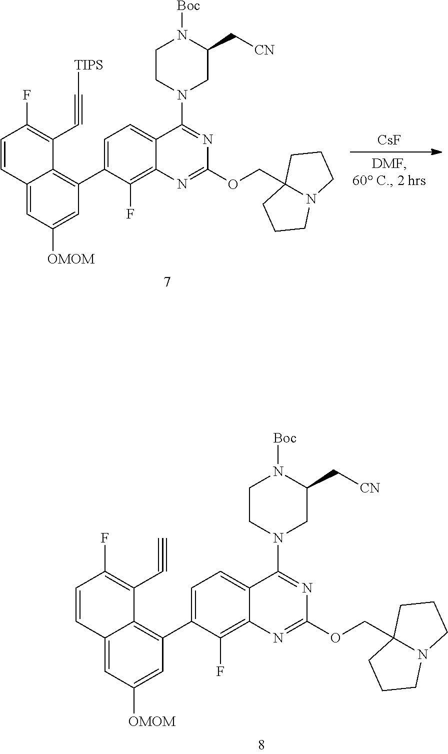

Tert-butyl (S)-2-(cyanomethyl)-4-(8-fluoro-7-(7-fluoro-3-(methoxymethoxy)-8-((triisopropylsilyl)ethynyl)naphth-1-yl)-2-((tetrahydro-1H-pyrrolizin-7a(5H)-yl)methoxy)quinazolin-4-yl)piperazine-1-carboxylate (580 mg, 648 umol) was dissolved in 20.0 mL of N,N-dimethylformamide, and cesium fluoride (295 mg, 1.94 mmol) was added to the reaction liquid. The reaction liquid was stirred at 60° C. for 2 hours. After the reaction was complete as monitored by LCMS, the reaction liquid was extracted with ethyl acetate and washed with brine, and the organic phase was concentrated and passed through a fast silica gel column to obtain tert-butyl (S)-2-(cyanomethyl)-4-(7-(8-ethynyl-7-fluoro-3-(methoxymethoxy)naphth-1-yl)-8-fluoro-2-((tetrahydro-1H-pyrrolizin-7a(5H)-yl)methoxy)quinazolin-4-yl)piperazine-1-carboxylate (0.56 g, crude)

LCMS: m/z 739.2 [M+H]⁺.

¹H NMR (400 MHz, CDCl₃): δ=7.87-7.77 (m, 1H), 7.61 (br d, J=8.4 Hz, 1H), 7.51 (br s, 1H), 7.34-7.28 (m, 1H), 7.26-7.18 (m, 2H), 5.31 (s, 2H), 4.68 (br d, J=1.2 Hz, 1H), 4.51-4.26 (m, 4H), 3.73-3.64 (m, 1H), 3.54 (s, 3H), 3.45 (br d, J=2.8 Hz, 3H), 3.06-2.71 (m, 6H), 2.23 (br s, 2H), 2.03-1.91 (m, 4H), 1.80 (br d, J=0.8 Hz, 3H), 1.52 (br s, 9H) ppm.

### Step VIII: Synthesis of (S)-2-(4-(7-(8-ethynyl-7-fluoro-3-hydroxynaphth-1-yl)-8-fluoro-2-((tetrahydro-1H-pyrrolizin-7a(5H)-yl)methoxy)quinazolin-4-yl)piperazin-2-yl)acetonitrile

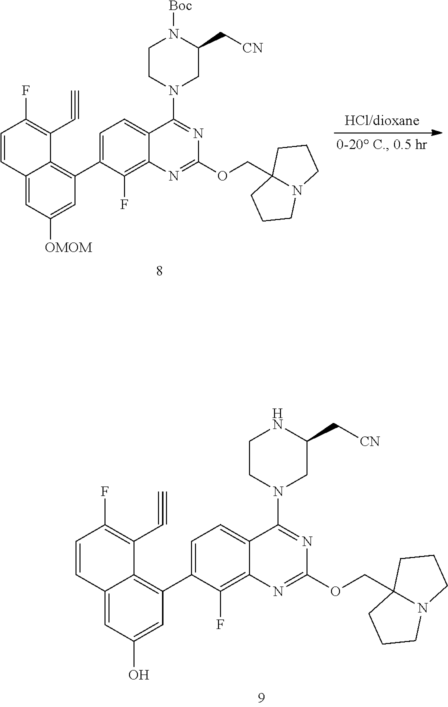

At 0° C., a hydrochloric acid/1,4-dioxane solution (4.00 M, 3.85 mL) was added to tert-butyl (S)-2-(cyanomethyl)-4-(7-(8-ethynyl-7-fluoro-3-(methoxymethoxy)naphth-1-yl)-8-fluoro-2-((tetrahydro-1H-pyrrolizin-7a(5H)-yl)methoxy)quinazolin-4-yl)piperazine-1-carboxylate (250 mg, 338 umol), and the reaction liquid was stirred at 20° C. for 0.5 hours. After the reaction was complete as monitored by LCMS, the reaction liquid was concentrated to obtain (S)-2-(4-(7-(8-ethynyl-7-fluoro-3-hydroxynaphth-1-yl)-8-fluoro-2-((tetrahydro-1H-pyrrolizin-7a(5H)-yl)methoxy)quinazolin-4-yl)piperazin-2-yl)acetonitrile (200 mg, crude, hydrochloride).

LCMS: m/z 595.3 [M+H]⁺.

### Step IX: Synthesis of (S)-2-(4-(7-(8-ethynyl-7-fluoro-3-hydroxynaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile

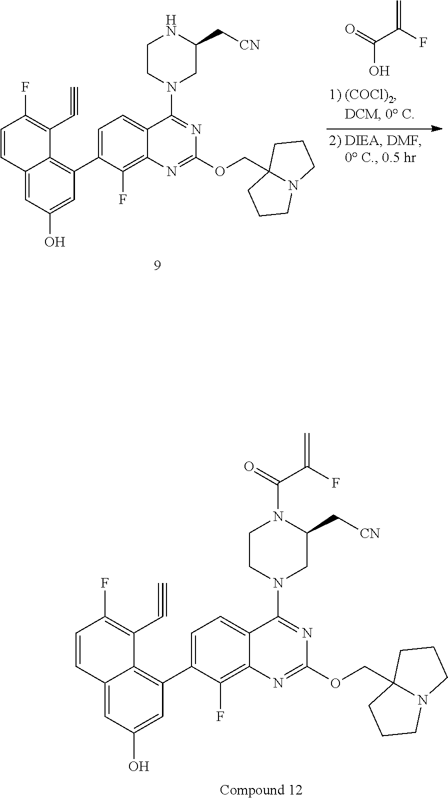

2-Fluoroprop-2-enoic acid (30.3 mg, 336 umol) was dissolved in 1.00 mL of dichloromethane, oxalyl chloride (51.2 mg, 404 umol, 35.3 uL) was added to the reaction liquid at 0° C., and N,N-dimethylformamide (1.23 mg, 16.8 umol, 1.29 uL) was then added and stirred for 30 minutes; subsequently, the reaction liquid was dropwise added to a solution of (S)-2-(4-(7-(8-ethynyl-7-fluoro-3-hydroxynaphth-1-yl)-8-fluoro-2-((tetrahydro-1H-pyrrolizin-7a(5H)-yl)methoxy)quinazolin-4-yl)piperazin-2-yl)acetonitrile (100 mg, 168 umol) and N,N-diisopropylethylamine (109 mg, 841 umol, 146 uL) in N,N-dimethylformamide (2.00 mL) and stirred for 0.5 hours. After the reaction was complete as monitored by LCMS, the reaction liquid was concentrated and then subjected to prep-HPLC and SFC for resolution and purification to obtain (S)-2-(4-(7-(8-ethynyl-7-fluoro-3-hydroxynaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (35.0 mg, 52.5 umol, 31.2% yield).

LCMS: m/z 667.5 [M+H]⁺.

¹H NMR (400 MHz, (CD₃)₂SO): δ=10.67 (br s, 1H), 10.27 (s, 1H), 9.13-8.40 (m, 1H), 7.97 (dd, J=6.0, 9.1 Hz, 1H), 7.91-7.82 (m, 1H), 7.47 (t, J=9.2 Hz, 1H), 7.41-7.29 (m, 2H), 7.13 (s, 1H), 5.50-5.20 (m, 2H), 5.01-4.69 (m, 1H), 4.64-4.50 (m, 2H), 4.36 (br t, J=10.8 Hz, 2H), 4.20-3.87 (m, 1H), 3.81 (s, 1H), 3.51 (br d, J=5.2 Hz, 4H), 3.24-3.13 (m, 3H), 2.23-2.08 (m, 4H), 2.06-1.95 (m, 4H) ppm.

### Example 13: (S)-2-(4-(7-(8-ethynyl-3-hydroxynaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (Compound 13)

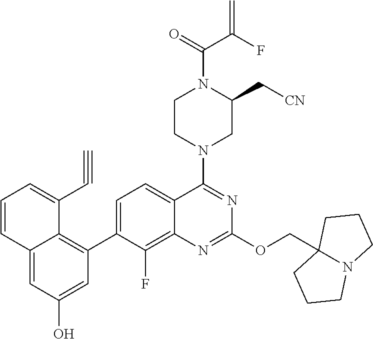 [SMILES: C#Cc1cccc2cc(O)cc(-c3ccc4c(N5CCN(C(=O)C(=C)F)[C@@H](CC#N)C5)nc(OCC56CCCN5CCC6)nc4c3F)c12]

The specific synthesis route of Compound 13 was as follows:

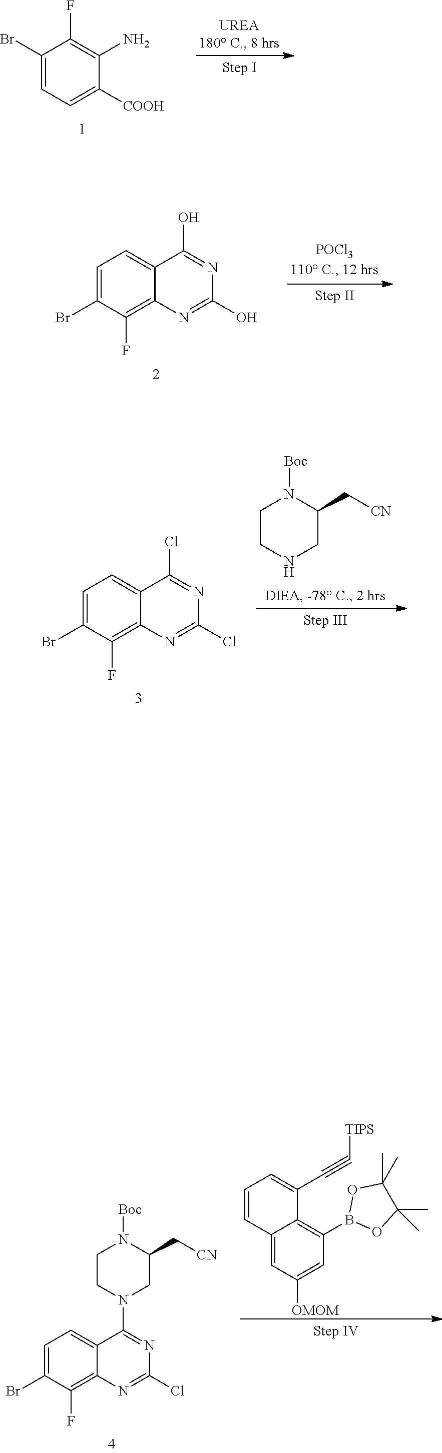

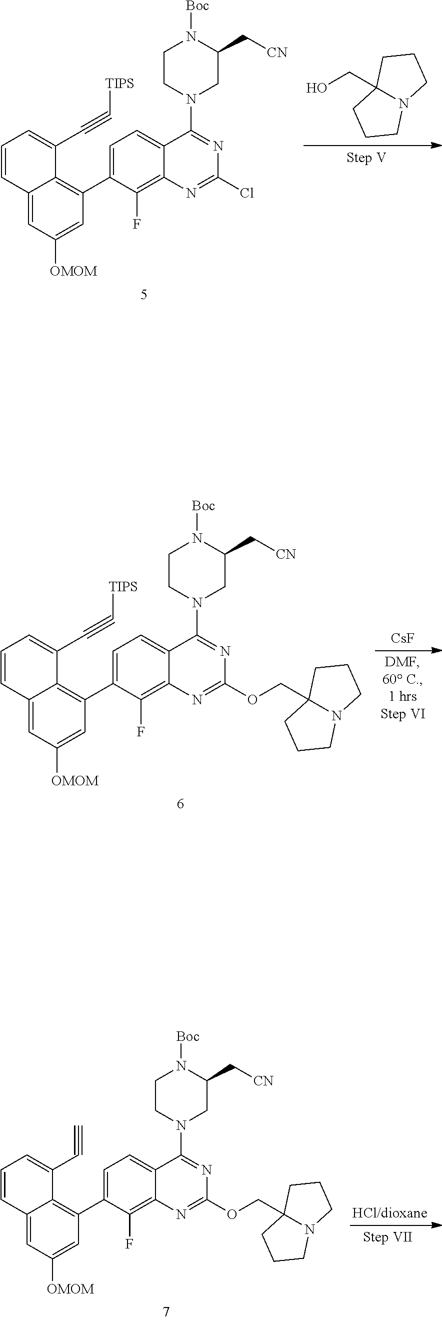

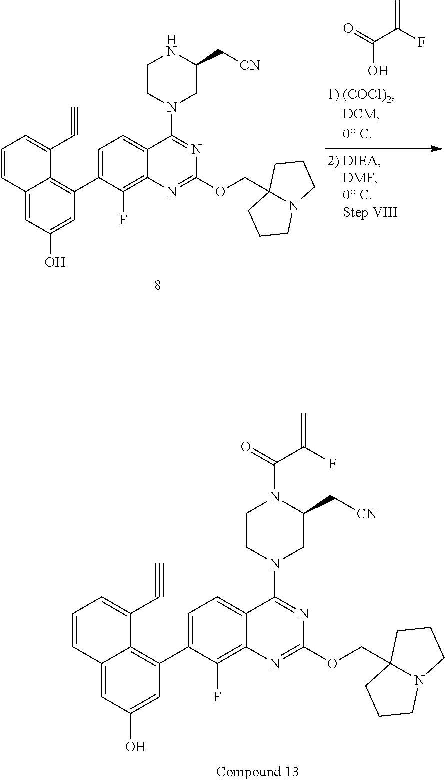

Starting from the intermediate No. 4 compound, the specific synthesis process of Compound 13 was as follows:

### Step IV. Synthesis of tert-butyl (S)-4-(2-chloro-8-fluoro-7-(3-(methoxymethyl)oxy)-8-((triisopropylsilyl)-ethynyl)naphth-1-yl)quinazolin-4-yl)-2-(cyanomethyl)piperazine-1-carboxylate

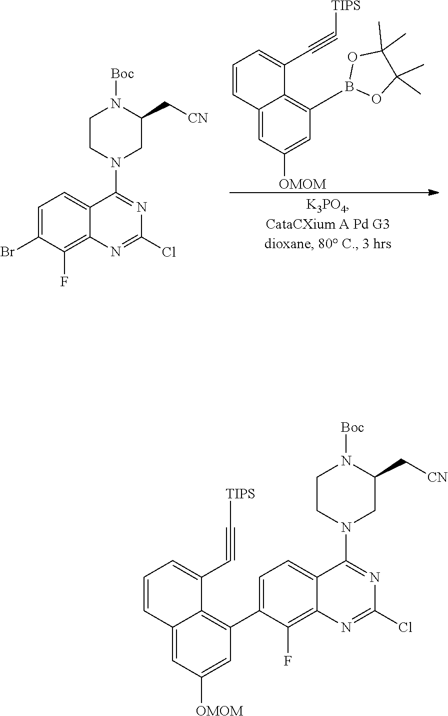

Tert-butyl (S)-4-(7-bromo-2-chloro-8-fluoroquinazolin-4-yl)-2-(cyanomethyl)piperazine-1-carboxylate (500 mg, 1.03 mmol) and triisopropyl((6-(methoxymethyl)oxy)-8-(4,4,5,5-tetramethyl-1,3,2-dioxaborolan-2-yl)naphth-1-yl)ethynyl)silane (510 mg, 1.03 mmol) were dissolved in water (1.25 mL) and glycol dimethyl ether (12.5 mL). Methanesulfonato(diadamantyl-N-butylphosphino)-2′-amino-1,1′-biphenyl-2-yl)palladium(II) (93.3 mg, 128 umol) and potassium carbonate (656 mg, 3.09 mmol) were added, and the reaction liquid was stirred at 80° C. for 2 hours. Three reactions were carried out in parallel. After the reaction was complete as monitored by LCMS, the reaction liquid was concentrated and purified by a fast silica gel column to obtain tert-butyl (S)-4-(2-chloro-8-fluoro-7-(3-(methoxymethyl)oxy)-8-((triisopropylsilyl)-ethynyl)naphth-1-yl)quinazolin-4-yl)-2-(cyanomethyl)piperazine-1-carboxylate (1.40 g, yield 55.60%).

¹H NMR (400 MHz, CDCl₃): δ=7.75 (d, J=8.1 Hz, 1H), 7.65-7.56 (m, 2H), 7.47-7.32 (m, 3H), 7.07 (d, J=2.0 Hz, 1H), 5.24 (s, 2H), 4.65 (br s, 1H), 4.46-4.08 (m, 3H), 3.77-3.64 (m, 1H), 3.57-3.34 (m, 5H), 3.01-2.65 (m, 2H), 1.46 (d, J=3.6 Hz, 9H), 0.83-0.72 (m, 18H), 0.61-0.32 (m, 3H) ppm.

### Step V: Synthesis of tert-butyl (S)-2-(cyanomethyl)-4-(8-fluoro-7-(3-(methoxymethoxy)-8-(triisopropylsilyl)ethynyl)naphth-1-yl)-2-((tetrahydro-1H-pyrrolizin-7a(5H)-yl)methoxy)quinazolin-4-yl)piperazine-1-carboxylate

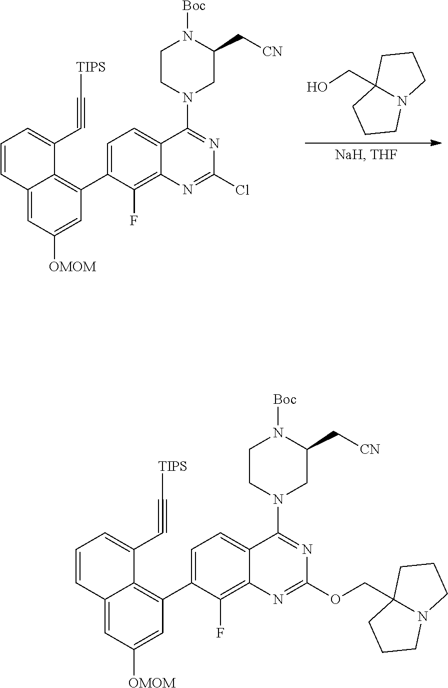

(Tetrahydro-1H-pyrrolizin-7a(5H)-yl)methanol (164.5 mg, 1.17 mmol, 3 eq) was dissolved in 6.00 mL of tetrahydrofuran, sodium hydride (62.1 mg, 1.55 mmol) was added at 0° C., and the reaction liquid was stirred under nitrogen protection at 20° C. for 1 hour. A solution of tert-butyl (S)-4-(2-chloro-8-fluoro-7-(3-(methoxymethyl)oxy)-8-((triisopropylsilyl)-ethynyl)naphth-1-yl)quinazolin-4-yl)-2-(cyanomethyl)piperazine-1-carboxylate (300 mg, 388 umol) in tetrahydrofuran (3 mL) was added to the reaction liquid, and the reaction liquid was stirred for 1 hour. After the reaction was complete as monitored by LCMS, the reaction liquid was quenched with water, extracted with ethyl acetate, and washed with brine, and the organic phase was concentrated to obtain tert-butyl (S)-2-(cyanomethyl)-4-(8-fluoro-7-(3-(methoxymethoxy)-8-(triisopropylsilyl)ethynyl)naphth-1-yl)-2-((tetrahydro-1H-pyrrolizin-7a(5H)-yl)methoxy)quinazolin-4-yl)piperazine-1-carboxylate (400 mg, crude).

LCMS: m/z 877.8 [M+H]⁺.

### Step VI: Synthesis of tert-butyl (S)-2-(cyanomethyl)-4-(7-(8-ethynyl-3-(methoxymethoxy)naphth-1-yl)-8-fluoro-2-((tetrahydro-1H-pyrrolizin-7a(5H)-yl)methoxy)quinazolin-4-yl)piperazine-1-carboxylate

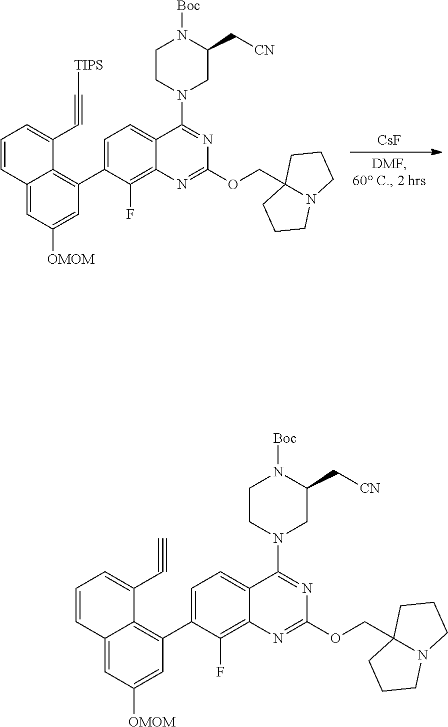

Tert-butyl (S)-2-(cyanomethyl)-4-(8-fluoro-7-(3-(methoxymethoxy)-8-(triisopropylsilyl)ethynyl)naphth-1-yl)-2-((tetrahydro-1H-pyrrolizin-7a(5H)-yl)methoxy)quinazolin-4-yl)piperazine-1-carboxylate (270 mg, 331 umol) was dissolved in N,N-dimethylformamide (5.0 mL), and cesium fluoride (295 mg, 1.94 mmol) was added to the reaction liquid. The reaction liquid was stirred at 60° C. for 1 hour. After the reaction was complete as monitored by LCMS, the reaction liquid was extracted with ethyl acetate and washed with brine, and the organic phase was concentrated and passed through a fast silica gel column to obtain tert-butyl (S)-2-(cyanomethyl)-4-(7-(8-ethynyl-3-(methoxymethoxy)naphth-1-yl)-8-fluoro-2-((tetrahydro-1H-pyrrolizin-7a(5H)-yl)methoxy)quinazolin-4-yl)piperazine-1-carboxylate (200 mg, yield 60.8%).

LCMS: m/z 721.4 [M+H]⁺.

### Step VII: Synthesis of (S)-2-(4-(7-(8-ethynyl-3-hydroxynaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile

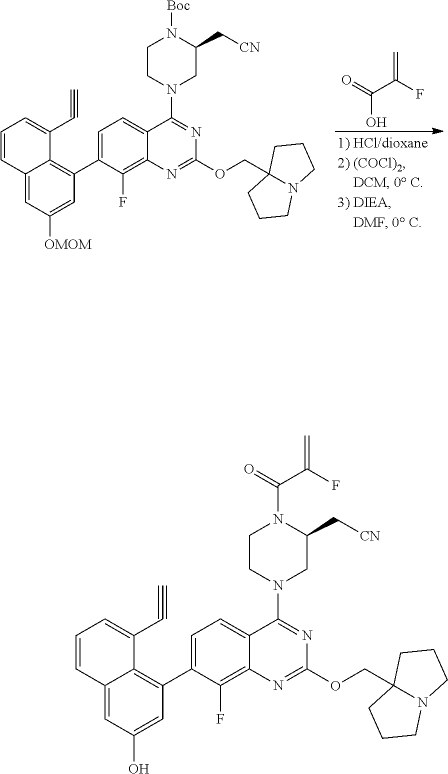

Solution A: At 0° C., tert-butyl (S)-2-(cyanomethyl)-4-(7-(8-ethynyl-3-(methoxymethoxy)naphth-1-yl)-8-fluoro-2-((tetrahydro-1H-pyrrolizin-7a(5H)-yl)methoxy)quinazolin-4-yl)piperazine-1-carboxylate (200 mg, 277 umol) was dissolved in dioxane hydrochloride (4.00 M, 5.00 mL) and stirred
for one hour. After the reaction liquid was concentrated, N,N-dimethylformamide (2.00 mL) and N,N-diisopropylethylamine (107 mg, 832 umol, 144 uL) were added.

Solution B: At 0° C., 2-fluoroacrylic acid (30.0 mg, 332 umol) was dissolved in dichloromethane (1.00 mL) and N,N-dimethylformamide (0.1 mL), oxalyl chloride (52.8 mg, 416 umol) was added and reacted at this temperature for 20 minutes, and the reaction was then carried out at 0° C. for 10 minutes.

At 0° C., solution B was added to solution A and stirred for 20 minutes. The reaction was complete as monitored by LCMS. The reaction liquid was filtered and subjected to prep-HPLC for separation and purification to obtain (S)-2-(4-(7-(8-ethynyl-3-hydroxynaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (16 mg, yield 8.71%).

LCMS: m/z 649.5 [M+H]⁺.

¹H NMR (400 MHz, (CD₃)₂SO): δ=10.53-9.54 (m, 1H), 7.92 (s, 1H), 7.82 (dd, J₁=8.5, J₂=13.6 Hz, 1H), 7.50-7.39 (m, 2H), 7.32 (d, J=2.5 Hz, 1H), 7.29-7.23 (m, 1H), 7.06-7.02 (m, 1H), 5.51-5.22 (m, 2H), 5.09-4.63 (m, 1H), 4.36-4.21 (m, 2H), 4.03 (s, 2H), 3.59-3.46 (m, 3H), 3.45-3.39 (m, 2H), 3.17-3.04 (m, 1H), 2.99-2.88 (m, 2H), 2.58-2.53 (m, 2H), 1.96-1.86 (m, 2H), 1.85-1.70 (m, 5H), 1.58 (td, J₁=7.5, J₂=11.9 Hz, 2H) ppm.

### Example 14: (S)-2-(4-(7-(8-ethynyl-7-fluoronaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (Compound 14)

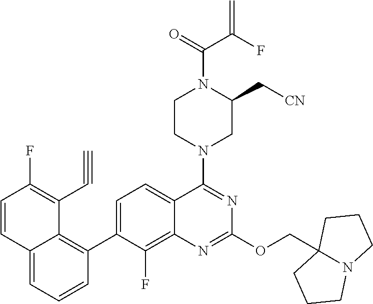 [SMILES: C#Cc1c(F)ccc2cccc(-c3ccc4c(N5CCN(C(=O)C(=C)F)[C@@H](CC#N)C5)nc(OCC56CCCN5CCC6)nc4c3F)c12]

The specific synthesis route of Compound 14 was as follows:

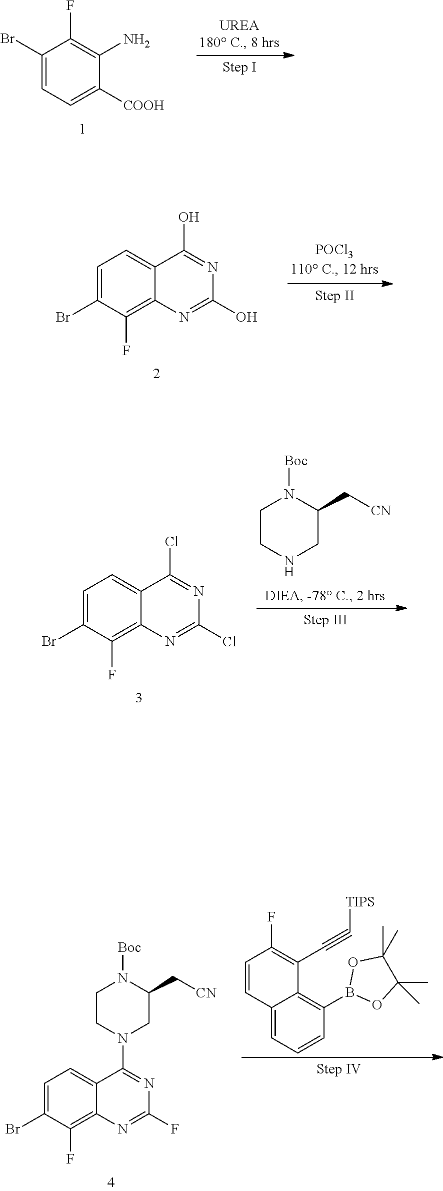

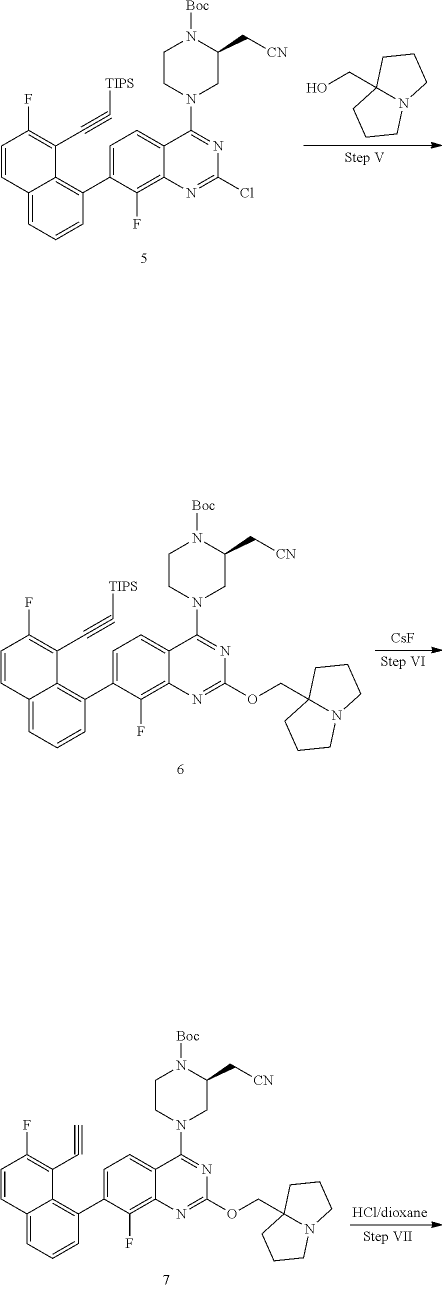

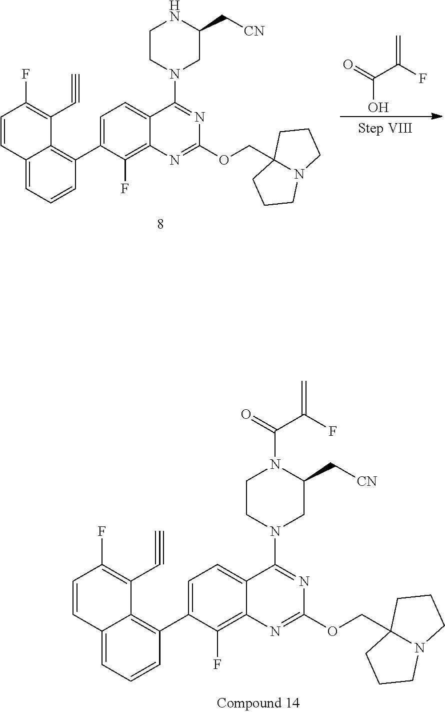

Starting from the intermediate No. 4 compound, the specific synthesis process of Compound 14 was as follows:

### Step IV: Synthesis of tert-butyl (S)-4-(2-chloro-8-fluoro-7-(7-fluoro-8-((triisopropyl silyl)ethynyl)naphth-1-yl)quinazolin-4-yl)-2-(cyanomethyl)piperazine-1-carboxylate

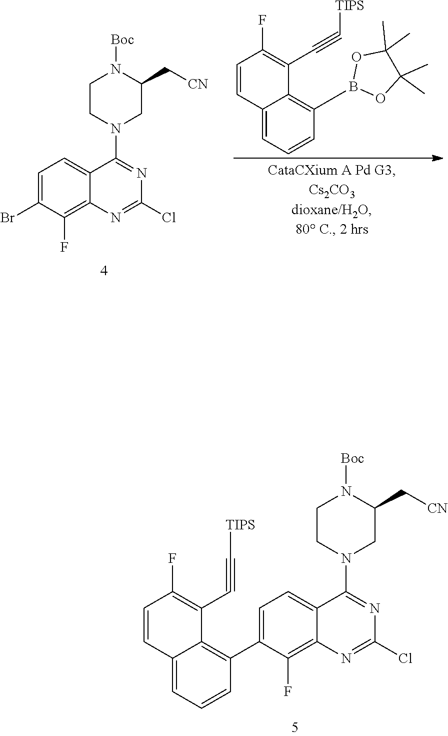

Tert-butyl (S′)-4-(7-bromo-2-chloro-8-fluoroquinazolin-4-yl)-2-(cyanomethyl)piperazine-1-carboxylate (100 mg, 206 umol) and 2-(2-fluoro-8-(4,4,5,5-tetramethyl-1,3,2-pinacol borate-2-yl)-1-naphthalene)ethynyl-triisopropylsilane (93.3 mg, 206 umol) were dissolved in 1.00 mL of dioxane and 0.2 mL of water, and methanesulfonato(diadamantyl-N-butylphosphino)-2′-amino-1,1′-biphenyl-2-yl)palladium(II)(30.1 mg, 41.3 umol) and potassium carbonate (57.0 mg, 413 umol) were added to the reaction liquid. The reaction liquid was reacted at 80° C. for 2 hours in a nitrogen atmosphere. Ten reactions were carried out in parallel. After the reaction was complete as monitored by LCMS and TLC, the reaction liquid was concentrated under reduced pressure and purified by a fast silica gel column to obtain tert-butyl (S)-4-(2-chloro-8-fluoro-7-(7-fluoro-8-((triisopropylsilyl)ethynyl)naphth-1-yl)quinazolin-4-yl)-2-(cyanomethyl)piperazine-1-carboxylate (1.12 g, 1.53 mmol, 74.3% yield).

LCMS: m/z 730.1 [M+H]⁺.

¹H NMR (400 MHz, CDCl₃): δ=7.98-7.86 (m, 2H), 7.75-7.65 (m, 1H), 7.57-7.46 (m, 2H), 7.43 (br d, J=7.1 Hz, 1H), 7.39-7.33 (m, 1H), 4.72 (br s, 1H), 4.55-4.02 (m, 3H), 3.65-3.41 (m, 2H), 3.07-2.72 (m, 2H), 1.88-1.85 (m, 1H), 1.54 (d, J=4.3 Hz, 9H), 0.90-0.78 (m, 18H), 0.53 (quin, J=7.5 Hz, 3H) ppm.

### Step V: Synthesis of tert-butyl (S)-2-(cyanomethyl)-4-(8-fluoro-7-(7-fluoro-8-((triisopropylsilyl)ethynyl)naphth-1-yl)-2-((tetrahydro-1H-pyrrolizin-7a(5H)-yl)methoxy)quinazolin-4-yl)piperazine-1-carboxylate

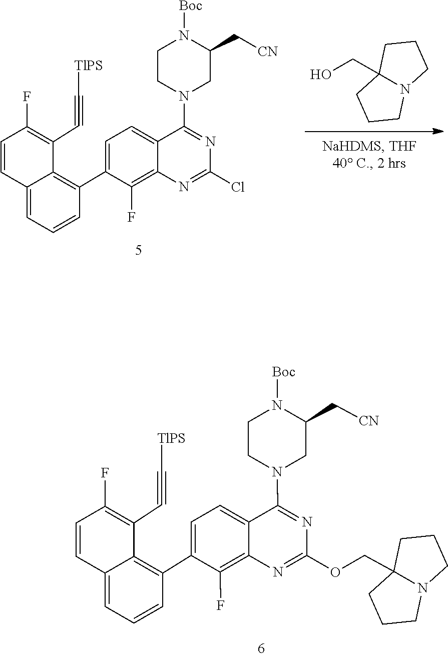

Tert-butyl (S)-4-(2-chloro-8-fluoro-7-(7-fluoro-8-((triisopropylsilyl)ethynyl)naphth-1-yl)quinazolin-4-yl)-2-(cyanomethyl)piperazine-1-carboxylate (500 mg, 685 umol) and (tetrahydro-1H-pyrrolizin-7a(5H)-yl)methanol (193 mg, 1.37 mmol) were dissolved in 2.00 mL of tetrahydrofuran, and sodium bis(trimethylsilyl)amide (1.00 M, 1.37 mL) was added. The reaction liquid was reacted at 40° C. for 2 hours. The reaction was complete as monitored by LCMS and TLC. The reaction liquid was quenched with a saturated ammonium chloride aqueous solution at 0° C. and extracted with ethyl acetate, and the organic phase was concentrated under reduced pressure and purified by a fast silica gel column to obtain tert-butyl (S)-2-(cyanomethyl)-4-(8-fluoro-7-(7-fluoro-8-((triisopropylsilyl)ethynyl)naphth-1-yl)-2-((tetrahydro-1H-pyrrolizin-7a(5H)-yl)methoxy)quinazolin-4-yl)piperazine-1-carboxylate (310 mg, 371 umol, 54.2% yield).

LCMS: m/z 835.5 [M+H]⁺.

¹H NMR (400 MHz, CDCl₃): δ=7.98-7.84 (m, 2H), 7.64 (br d, J=8.6 Hz, 1H), 7.57-7.50 (m, 1H), 7.47-7.41 (m, 1H), 7.38-7.30 (m, 2H), 4.76-4.51 (m, 3H), 4.29 (br d, J=10.0 Hz, 2H), 3.96-3.83 (m, 1H), 3.69-3.17 (m, 4H), 2.99-2.84 (m, 3H), 2.50-2.30 (m, 2H), 2.24-2.10 (m, 2H), 2.01-1.90 (m, 2H), 1.68 (br s, 4H), 1.52 (br d, J=1.5 Hz, 9H), 0.89-0.78 (m, 18H), 0.64-0.45 (m, 3H) ppm.

### Step VI: Synthesis of tert-butyl (S)-2-(cyanomethyl)-4-(7-(8-ethynyl-7-fluoronaphth-1-yl)-8-fluoro-2-((tetrahydro-1H-pyrrolizin-7a(5H)-yl)methoxy)quinazolin-4-yl)piperazine-1-carboxylate

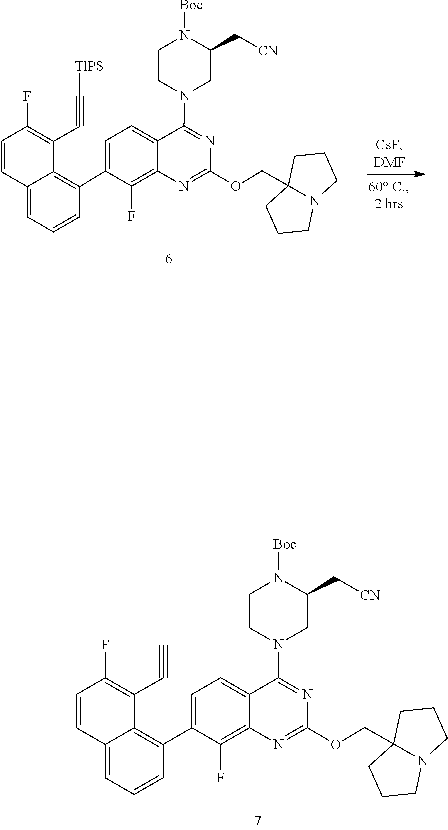

Tert-butyl (S)-2-(cyanomethyl)-4-(8-fluoro-7-(7-fluoro-8-((triisopropylsilyl)ethynyl)naphth-1-yl)-2-((tetrahydro-1H-pyrrolizin-7a(5H)-yl)methoxy)quinazolin-4-yl)piperazine-1-carboxylate (300 mg, 359 umol) was dissolved in 2.00 mL of an N,N-dimethylformamide solution, and cesium fluoride (164 mg, 1.08 mmol) was added. The reaction liquid was reacted at 60° C. for 2 hours. The reaction was complete as monitored by LCMS and TLC. The reaction liquid was extracted with ethyl acetate, washed with brine, and dried, and the organic phases were combined, concentrated under reduced pressure and purified by a fast silica gel column to obtain tert-butyl (S)-2-(cyanomethyl)-4-(7-(8-ethynyl-7-fluoronaphth-1-yl)-8-fluoro-2-((tetrahydro-1H-pyrrolizin-7a(5H)-yl)methoxy)quinazolin-4-yl)piperazine-1-carboxylate (240 mg, 354 umol, 98.4% yield).

LCMS: m/z 679.4 [M+H]⁺.

¹H NMR (400 MHz, CDCl₃): δ=7.98-7.90 (m, 2H), 7.60 (d, J=8.4 Hz, 1H), 7.56-7.51 (m, 1H), 7.48 (br d, J=7.3 Hz, 1H), 7.35 (t, J=8.8 Hz, 1H), 7.23 (br dd, J=1.0, 6.5 Hz, 1H), 4.75-4.63 (m, 1H), 4.33-4.22 (m, 3H), 3.80-3.71 (m, 2H), 3.20-3.07 (m, 2H), 2.98-2.79 (m, 4H), 2.70-2.59 (m, 2H), 2.11 (td, J=6.0, 12.3 Hz, 2H), 1.93-1.81 (m, 6H), 1.71-1.65 (m, 2H), 1.53 (s, 9H) ppm.

### Step VII: Synthesis of (S)-2-(4-(7-(8-ethynyl-7-fluoronaphth-1-yl)-8-fluoro-2-((tetrahydro-1H-pyrrolizin-7a(5H)-yl)methoxy)quinazolin-4-yl)piperazin-2-yl)acetonitrile

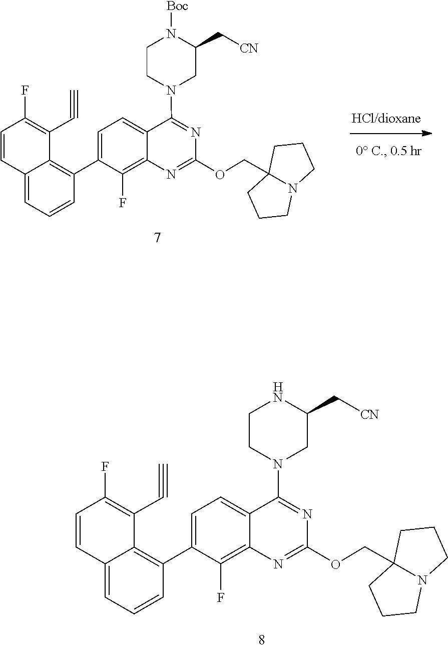 [SMILES: C#Cc1c(F)ccc2cccc(-c3ccc4c(N5CCN(C(=O)OC(C)(C)C)[C@@H](CC#N)C5)nc(OCC56CCCN5CCC6)nc4c3F)c12 ; C#Cc1c(F)ccc2cccc(-c3ccc4c(N5CCN[C@@H](CC#N)C5)nc(OCC56CCCN5CCC6)nc4c3F)c12]

At 0° C., dioxane hydrochloride (4.00 M, 4.00 mL) was dropwise added to tert-butyl (S)-2-(cyanomethyl)-4-(7-(8-ethynyl-7-fluoronaphth-1-yl)-8-fluoro-2-((tetrahydro-1H-pyrrolizin-7a(5H)-yl)methoxy)quinazolin-4-yl)piperazine-1-carboxylate (200 mg, 295 umol). The reaction liquid was reacted at 0° C. for 0.5 hours. The reaction was complete as monitored by LCMS. The reaction liquid was concentrated under reduced pressure to obtain (S)-2-(4-(7-(8-ethynyl-7-fluoronaphth-1-yl)-8-fluoro-2-((tetrahydro-1H-pyrrolizin-7a(5H)-yl)methoxy)quinazolin-4-yl)piperazin-2-yl)acetonitrile (200 mg, crude).

LCMS: m/z 579.3 [M+H]⁺.

### Step VIII: Synthesis of (S)-2-(4-(7-(8-ethynyl-7-fluoronaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile

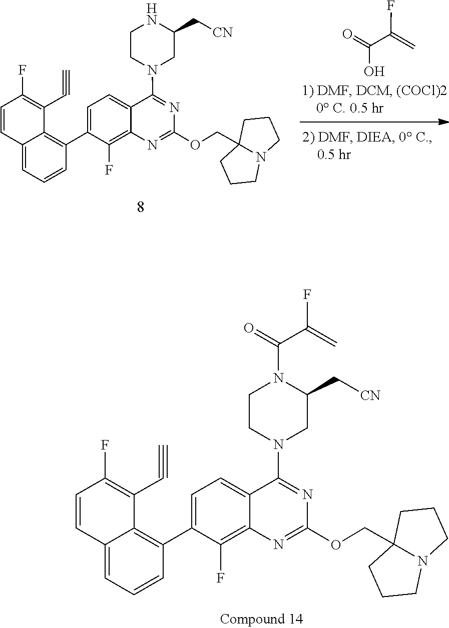

2-Fluoroacrylic acid (31.1 mg, 346 umol) was dissolved in 4.00 mL of dichloromethane, and oxalyl chloride (52.7 mg, 415 umol) was dropwise added at 0° C.; subsequently, N,N-dimethylformamide (2.53 mg, 34.6 umol) was dropwise added, and the reaction liquid was stirred at 0° C. for 0.5 hours. Thereafter, the above reaction liquid was dropwise added to a solution of (S)-2-(4-(7-(8-ethynyl-7-fluoronaphth-1-yl)-8-fluoro-2-((tetrahydro-1H-pyrrolizin-7a(5H)-yl)methoxy)quinazolin-4-yl)piperazin-2-yl)acetonitrile (200 mg, 346 umol) and diisopropylethylamine (134 mg, 1.04 mmol) in 2.00 mL of N,N-dimethylformamide. The reaction liquid was stirred at 0° C. for 0.5 hours. The reaction was complete as monitored by LCMS. The reaction liquid was concentrated under reduced pressure and purified by prep-HPLC to obtain (S)-2-(4-(7-(8-ethynyl-7-fluoronaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (100 mg, 152 umol, 44.1% yield, 99.2% purity).

LCMS: m/z 651.5 [M+H]⁺.

¹H NMR (400 MHz, (CD₃)₂SO): δ=8.27-8.14 (m, 2H), 7.86 (t, J=9.5 Hz, 1H), 7.72-7.53 (m, 3H), 7.32 (t, J=7.5 Hz, 1H), 5.49-5.21 (m, 2H), 5.02-4.70 (m, 1H), 4.32 (br t, J=12.1 Hz, 4H), 3.91 (s, 1H), 3.67-3.36 (m, 3H), 3.30-2.70 (m, 6H), 2.27-1.52 (m, 9H) ppm.

### Example 15: 2-((S)-4-(7-(8-ethynylnaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 15)

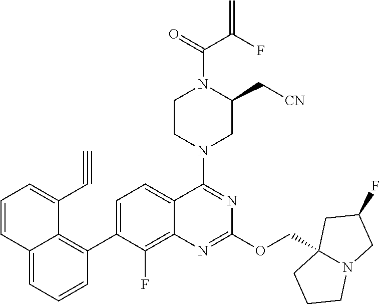 [SMILES: C#Cc1cccc2cccc(-c3ccc4c(N5CCN(C(=O)C(=C)F)[C@@H](CC#N)C5)nc(OC[C@@]56CCCN5C[C@H](F)C6)nc4c3F)c12]

The specific synthesis route of Compound 15 was as follows:

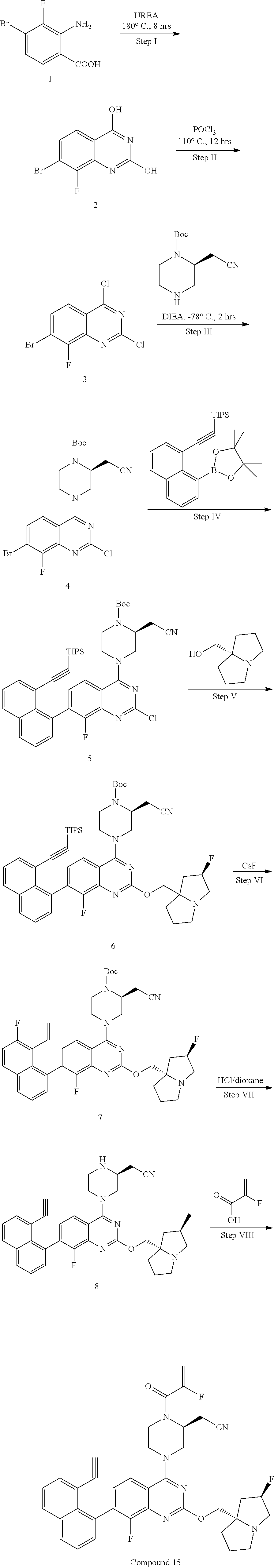

Starting from the intermediate No. 4 compound, the specific synthesis process of Compound 15 was as follows:

### Step IV: Synthesis of tert-butyl (S)-4-(2-chloro-8-fluoro-7-(8-((triisopropylsilyl)ethynyl)naphth-1-yl)quinazolin-4-yl)-2-(cyanomethyl)piperazine-1-carboxylate

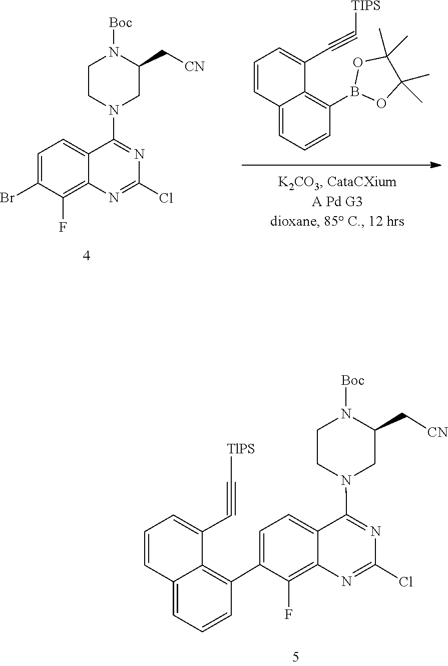

Tert-butyl (S)-4-(7-bromo-2-chloro-8-fluoroquinazolin-4-yl)-2-(cyanomethyl)piperazine-1-carboxylate (400 mg, 825 umol), triisopropyl((8-(4,4,5,5-tetramethyl-1,3,2-pinacol borate-2-yl)naphth-1-yl)ethynyl)silane (537 mg, 1.24 mmol), methanesulfonato(diadamantyl-N-butylphosphino)-2′-amino-1,1′-biphenyl-2-yl)palladium(II) (120 mg, 165 umol), and potassium carbonate (228 mg, 1.65 mmol) were dissolved in 1,4-dioxane (3 mL) and water (0.1 mL). After nitrogen displacement three times, the reaction liquid was stirred at 80° C. for 5 hours. After the reaction was complete as monitored by LCMS and TLC, water was added to the reaction liquid, which was extracted with ethyl acetate, and the organic phase was concentrated and purified by a fast silica gel column to obtain tert-butyl (S)-4-(2-chloro-8-fluoro-7-(8-((triisopropylsilyl)ethynyl)naphth-1-yl)quinazolin-4-yl)-2-(cyanomethyl)piperazine-1-carboxylate (800 mg, 1.12 mmol, 68.0% yield).

¹H NMR (400 MHz, CDCl₃): δ=7.95 (t, J=8.2 Hz, 2H), 7.85-7.79 (m, 1H), 7.73-7.66 (m, 1H), 7.57 (t, J=7.7 Hz, 1H), 7.53-7.46 (m, 2H), 7.41 (br d, J=7.1 Hz, 1H), 4.72 (br s, 1H), 4.52-4.26 (m, 2H), 3.68-3.42 (m, 2H), 3.07-2.70 (m, 2H), 1.93 (s, 2H), 1.25 (s, 9H), 0.88-0.84 (m, 18H), 0.53 (quin, J=7.4 Hz, 3H) ppm.

### Step V: Synthesis of tert-butyl (S)-2-(cyanomethyl)-4-(8-fluoro-2-(((2R,7aS)-2-fluorotetrahydro-1H-pyrrolizin-7a(5H)-yl)methoxy)-7-(8-((triisopropylsilyl)ethynyl)naphth-1-yl)quinazolin-4-yl)piperazine-1-carboxylate

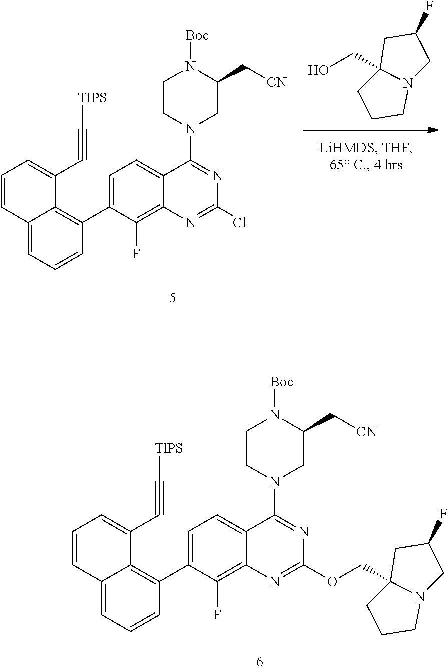

LiHMDS in tetrahydrofuran (1 M, 2.29 mL) was dropwise added to tert-butyl (S)-4-(2-chloro-8-fluoro-7-(8-((triisopropylsilyl)ethynyl)naphth-1-yl)quinazolin-4-yl)-2-(cyanomethyl)piperazine-1-carboxylate (800 mg, 1.12 mmol) and ((2R,7aS)-2-fluorotetrahydro-1H-pyrrolizin-7a(5H)-yl)methanol (250 mg, 1.57 mmol) in tetrahydrofuran (8 mL). After nitrogen displacement, the reaction liquid was stirred at 65° C. for 4 hours. After the reaction was complete as monitored by LCMS and TLC, the reaction product was quenched with a saturated ammonium chloride aqueous solution and extracted with ethyl acetate, and the organic phase was concentrated and purified by column chromatography to obtain tert-butyl (S)-2-(cyanomethyl)-4-(8-fluoro-2-(((2R,7aS)-2-fluorotetrahydro-1H-pyrrolizin-7a(5H)-yl)methoxy)-7-(8-((triisopropylsilyl)ethynyl)naphth-1-yl)quinazolin-4-yl)piperazine-1-carboxylate (700 mg, 838 umol, 74.6% yield).

¹H NMR (400 MHz, CDCl₃): δ=7.94 (dd, J=2.9, 8.1 Hz, 2H), 7.81 (d, J=7.1 Hz, 1H), 7.65-7.52 (m, 2H), 7.50-7.44 (m, 1H), 7.40 (d, J=6.9 Hz, 1H), 7.36-7.29 (m, 1H), 5.45-5.17 (m, 1H), 4.81-4.61 (m, 1H), 4.40-4.31 (m, 1H), 4.30-4.04 (m, 4H), 3.57 (dd, J=3.9, 13.8 Hz, 1H), 3.44 (br dd, J=3.8, 13.4 Hz, 1H), 3.33-3.14 (m, 4H), 3.05-2.84 (m, 2H), 2.80 (br d, J=7.0 Hz, 1H), 2.38-2.12 (m, 3H), 2.03-1.88 (m, 3H), 1.54 (d, J=2.4 Hz, 9H), 0.84 (dt, J=3.0, 7.2 Hz, 18H), 0.54 (qd, J=7.4, 14.9 Hz, 3H) ppm.

### Step VI: Synthesis of tert-butyl (S)-2-(cyanomethyl)-4-(7-(8-ethynylnaphth-1-yl)-8-fluoro-2-(((2R,7aS)-2-fluorotetrahydro-1H-pyrrolizin-7a(5H)-yl)methoxy)quinazolin-4-yl)piperazine-1-carboxylate

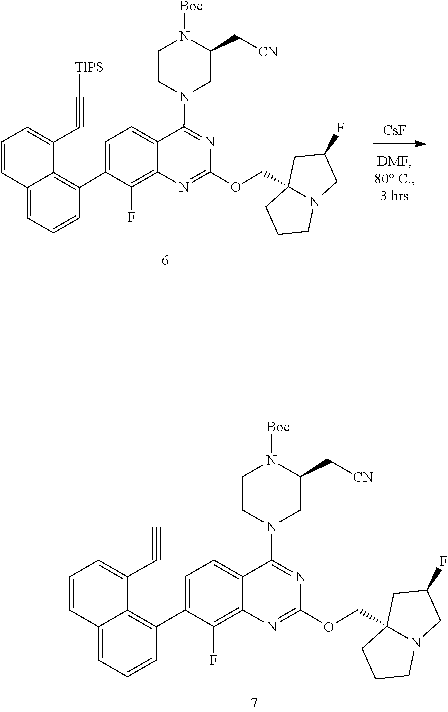

Tert-butyl (S)-2-(cyanomethyl)-4-(8-fluoro-2-(((2R,7aS)-2-fluorotetrahydro-1H-pyrrolizin-7a(5H)-yl)methoxy)-7-(8-((triisopropylsilyl)ethynyl)naphth-1-yl)quinazolin-4-yl)piperazine-1-carboxylate (600 mg, 718 umol) and cesium fluoride (327 mg, 2.16 mmol) were added to DMF (10 mL). After nitrogen displacement three times, the reaction liquid was stirred at 80° C. for 3 hours. After the reaction was complete as monitored by LCMS and TLC, the reaction liquid was diluted with ethyl acetate and washed with brine, and the organic phase was concentrated and then subjected to column chromatography to obtain tert-butyl (S)-2-(cyanomethyl)-4-(7-(8-ethynylnaphth-1-yl)-8-fluoro-2-(((2R,7aS)-2-fluorotetrahydro-1H-pyrrolizin-7a(5H)-yl)methoxy)quinazolin-4-yl)piperazine-1-carboxylate (400 mg, 589 umol, 82.0% yield).

¹H NMR (400 MHz, CDCl₃): δ=7.97 (d, J=8.1 Hz, 2H), 7.75 (td, J=1.3, 7.1 Hz, 1H), 7.62-7.52 (m, 2H), 7.50-7.42 (m, 2H), 7.29 (d, J=1.9 Hz, 1H), 5.46-5.16 (m, 1H), 4.82-4.57 (m, 1H), 4.49-4.25 (m, 3H), 4.25-4.17 (m, 1H), 3.70-3.54 (m, 1H), 3.51-3.33 (m, 2H), 3.32-3.11 (m, 3H), 3.05-2.94 (m, 1H), 2.93-2.72 (m, 2H), 2.64-2.47 (m, 1H), 2.36-2.11 (m, 3H), 1.99-1.79 (m, 4H), 1.53 (s, 9H) ppm.

### Step VII: Synthesis of 2-((S)-4-(7-(8-ethynylnaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile

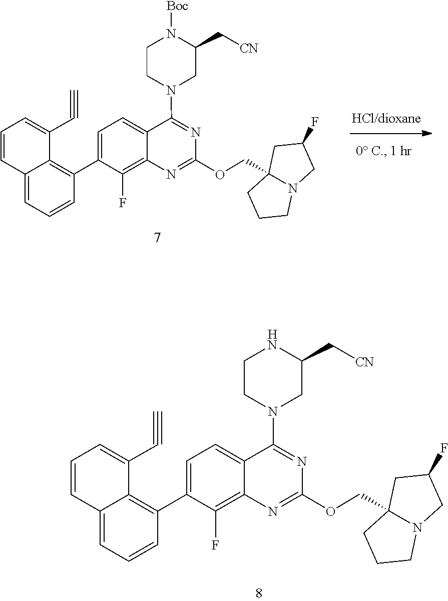 [SMILES: C#Cc1cccc2cccc(-c3ccc4c(N5CCN(C(=O)OC(C)(C)C)[C@@H](CC#N)C5)nc(OC[C@@]56CCCN5C[C@H](F)C6)nc4c3F)c12 ; C#Cc1cccc2cccc(-c3ccc4c(N5CCN[C@@H](CC#N)C5)nc(OC[C@@]56CCCN5C[C@H](F)C6)nc4c3F)c12]

At 0° C., tert-butyl (S)-2-(cyanomethyl)-4-(7-(8-ethynylnaphth-1-yl)-8-fluoro-2-(((2R,7aS)-2-fluorotetrahydro-1H-pyrrolizin-7a(5H)-yl)methoxy)quinazolin-4-yl)piperazine-1-carboxylate (200 mg, 294 umol) was dissolved in hydrochloric acid/1,4-dioxane (4 M, 3 mL), and the reaction liquid was further reacted at this temperature for 1 hour. After the reaction was complete as monitored by LCMS, the reaction liquid was quenched to pH=8 by triethylamine and directly used in the next step.

### Step VIII. Synthesis of 2-((S)-4-(7-(8-ethynylnaphth-1-yl)-8-fluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

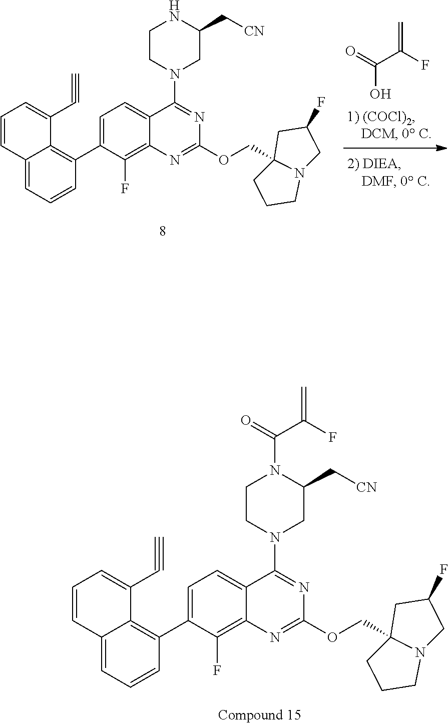

At 0° C., oxalyl chloride (372 mg, 2.94 mmol) was dropwise added to 2-fluoroprop-2-enoic acid (264 mg, 2.94 mmol) in dichloromethane (2.00 mL); subsequently, N,N-dimethylformamide (214 ug, 2.94 umol) was added, and the mixture was heated to 20° C. and stirred for 1 hour. Subsequently, at 0° C., the above reaction liquid was dropwise added to a solution of 2-((S′)-4-(7-(8-ethynylnaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile (170 mg, 293 umol) and DIEA (379 mg, 2.94 mmol) in N,N-dimethylformamide (2.00 mL), heated to 20° C., and stirred for 1 hour. After the reaction was complete as monitored by LCMS, the reaction liquid was concentrated, 2 mL of DMF was then added, and after filtration, the filtrate was purified by prep-HPLC and SFC to obtain 2-((S)-4-(7-(8-ethynylnaphth-1-yl)-8-fluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (60.0 mg, 91.6 umol, 31.2% yield).

LCMS: m/z 651.5 [M+H]⁺.

¹H NMR (400 MHz, CD₃₀D): 6=8.03 (ddd, J=1.0, 3.3, 8.2 Hz, 2H), 7.83 (dd, J=1.5, 8.6 Hz, 1H), 7.71 (d, J=7.1 Hz, 1H), 7.66-7.57 (m, 1H), 7.53-7.43 (m, 2H), 7.32 (ddd, J=2.4, 6.5, 8.7 Hz, 1H), 5.48-5.18 (m, 3H), 5.06-4.92 (m, 1H), 4.62-4.40 (m, 2H), 4.36-3.95 (m, 3H), 3.90-3.52 (m, 3H), 3.29-3.17 (m, 3H), 3.17-3.13 (m, 1H), 3.12-2.94 (m, 3H), 2.45-2.27 (m, 1H), 2.26-2.07 (m, 2H), 2.05-1.86 (m, 3H) ppm.

### Example 16: 2-((S)-4-(7-(8-ethynyl-7-fluoronaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 16)

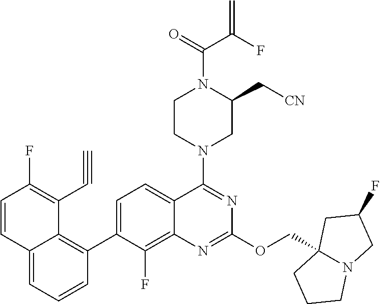 [SMILES: C#Cc1c(F)ccc2cccc(-c3ccc4c(N5CCN(C(=O)C(=C)F)[C@@H](CC#N)C5)nc(OC[C@@]56CCCN5C[C@H](F)C6)nc4c3F)c12]

The specific synthesis route of Compound 16 was as follows:

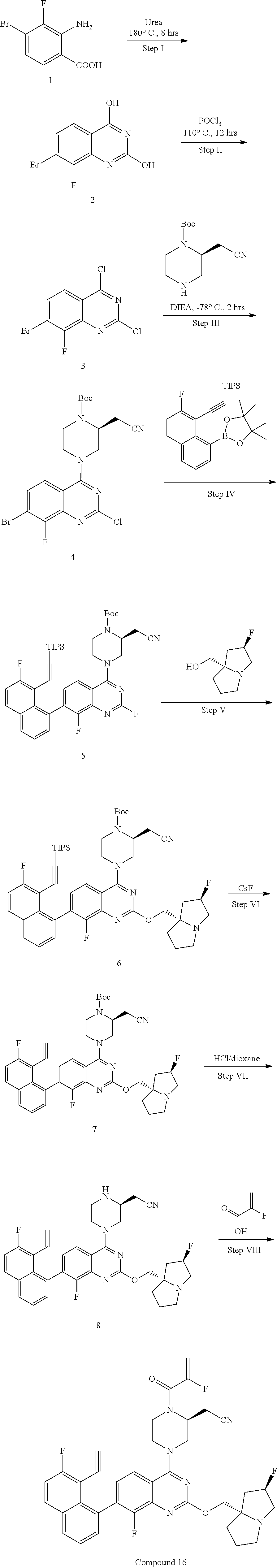

Starting from the intermediate No. 4 compound, the specific synthesis process of Compound 16 was as follows:

### Step IV: Synthesis of tert-butyl (S)-4-(2-chloro-8-fluoro-7-(7-fluoro-8-((triisopropylsilyl)ethynyl)naphth-1-yl)quinazolin-4-yl)-2-(cyanomethyl)piperazine-1-carboxylate

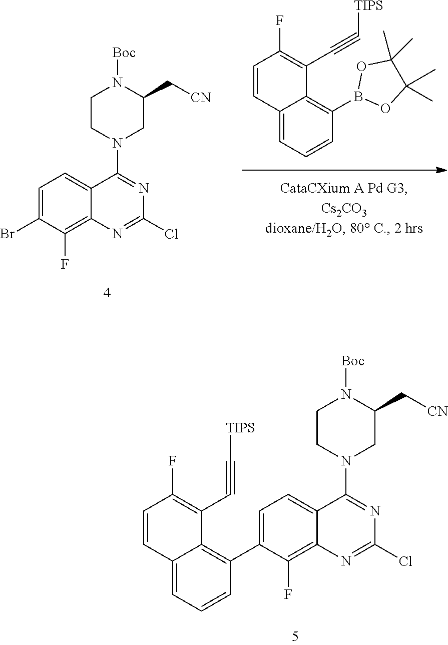

Tert-butyl (S′)-4-(7-bromo-2-chloro-8-fluoroquinazolin-4-yl)-2-(cyanomethyl)piperazine-1-carboxylate (100 mg, 206 umol) and 2-(2-fluoro-8-(4,4,5,5-tetramethyl-1,3,2-pinacol borate-2-yl)-1-naphthalene)ethynyl-triisopropylsilane (93.3 mg, 206 umol) were dissolved in 1.00 mL of dioxane and 0.2 mL of water, and methanesulfonato(diadamantyl-N-butylphosphino)-2′-amino-1,1′-biphenyl-2-yl)palladium(II) (30.1 mg, 41.3 umol) and potassium carbonate (57.0 mg, 413 umol) were added to the reaction liquid. The reaction liquid was reacted for 2 hours at 80° C. in a nitrogen atmosphere. Ten reactions were carried out in parallel. After the reaction was complete as monitored by LCMS and TLC, the reaction liquid was concentrated under reduced pressure and purified by a fast silica gel column to obtain tert-butyl (S)-4-(2-chloro-8-fluoro-7-(7-fluoro-8-((triisopropylsilyl)ethynyl)naphth-1-yl)quinazolin-4-yl)-2-(cyanomethyl)piperazine-1-carboxylate (1.12 g, 1.53 mmol, 74.3% yield).

LCMS: m/z 730.1 [M+H]⁺.

¹H NMR (400 MHz, CDCl₃): δ=7.98-7.86 (m, 2H), 7.75-7.65 (m, 1H), 7.57-7.46 (m, 2H), 7.43 (br d, J=7.1 Hz, 1H), 7.39-7.33 (m, 1H), 4.72 (br s, 1H), 4.55-4.02 (m, 3H), 3.65-3.41 (m, 2H), 3.07-2.72 (m, 2H), 1.88-1.85 (m, 1H), 1.54 (d, J=4.3 Hz, 9H), 0.90-0.78 (m, 18H), 0.53 (quin, J=7.5 Hz, 3H) ppm.

### Step V: Synthesis of tert-butyl (S)-2-(cyanomethyl)-4-(8-fluoro-7-(7-fluoro-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate

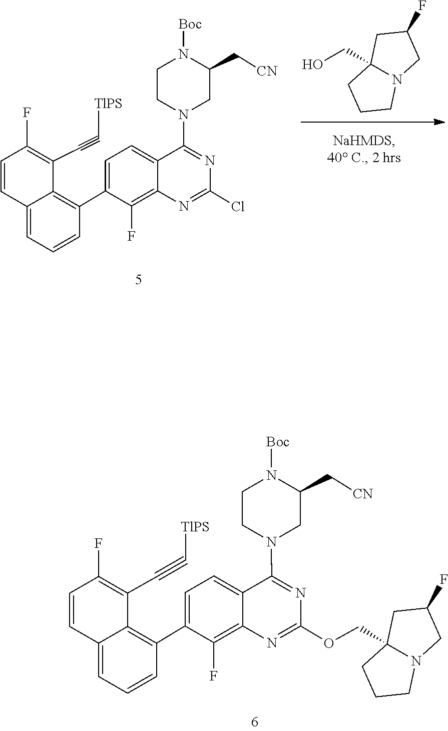

Tert-butyl (S)-4-(2-chloro-8-fluoro-7-(7-fluoro-8-((triisopropylsilyl)ethynyl)naphth-1-yl)quinazolin-4-yl)-2-(cyanomethyl)piperazine-1-carboxylate (400 mg, 548 umol) and ((2R,7aS)-2-fluorotetrahydro-1H-pyrrolizin-7a(5H)-yl)methanol (174 mg, 1.10 mmol) were dissolved in 10.0 mL of tetrahydrofuran, and sodium bis(trimethylsilyl)amide (1.00 M, 1.10 mL) was added. The reaction liquid was reacted at 40° C. for 2 hours. The reaction was complete as monitored by LCMS and TLC.

The reaction liquid was quenched with a saturated ammonium chloride aqueous solution at 0° C. and extracted with ethyl acetate, and the organic phase was concentrated under reduced pressure and purified by a fast silica gel column to obtain tert-butyl (S)-2-(cyanomethyl)-4-(8-fluoro-7-(7-fluoro-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate (320 mg, 375 umol, 68.5% yield).

¹H NMR (400 MHz, CDCl₃): δ=7.98-7.87 (m, 2H), 7.66-7.57 (m, 1H), 7.56-7.50 (m, 1H), 7.43 (d, J=7.0 Hz, 1H), 7.38-7.32 (m, 2H), 5.44-5.17 (m, 1H), 4.71 (br s, 1H), 4.43-4.16 (m, 4H), 3.64-3.39 (m, 2H), 3.37-3.12 (m, 3H), 2.99 (br d, J=3.9 Hz, 1H), 2.90-2.73 (m, 2H), 2.31-2.10 (m, 3H), 2.00-1.82 (m, 5H), 1.54 (d, J=2.4 Hz, 9H), 0.85 (dt, J=3.0, 7.6 Hz, 18H), 0.54 (qd, J=7.4, 14.8 Hz, 3H) ppm.

### Step VI: Synthesis of tert-butyl (S)-2-(cyanomethyl)-4-(7-(8-ethynyl-7-fluoronaphth-1-yl)-8-fluoro-2-(((2R,7aS)-2-fluorotetrahydro-1H-pyrrolizin-7a(5H)-yl)methoxy)quinazolin-4-yl)piperazine-1-carboxylate

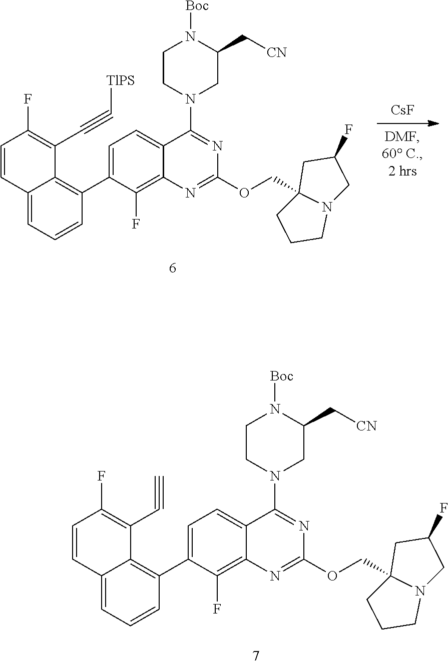

Tert-butyl (S)-2-(cyanomethyl)-4-(8-fluoro-7-(7-fluoro-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate (310 mg, 363 umol) was dissolved in 6.00 mL of an N,N-dimethylformamide solution, and cesium fluoride (166 mg, 1.09 mmol) was added. The reaction liquid was reacted at 60° C. for 2 hours. The reaction was complete as monitored by LCMS and TLC. The reaction liquid was extracted with ethyl acetate, washed with brine, and dried, and the organic phases were combined, concentrated under reduced pressure and purified by a fast silica gel column to obtain tert-butyl (S)-2-(cyanomethyl)-4-(7-(8-ethynyl-7-fluoronaphth-1-yl)-8-fluoro-2-(((2R,7aS)-2-fluorotetrahydro-1H-pyrrolizin-7a(5H)-yl)methoxy)quinazolin-4-yl)piperazine-1-carboxylate (0.24 g, 344 umol, 94.8% yield).

¹H NMR (400 MHz, CDCl₃): δ=7.90-7.84 (m, 2H), 7.53 (d, J=8.6 Hz, 1H), 7.50-7.44 (m, 1H), 7.42-7.37 (m, 1H), 7.30-7.24 (m, 1H), 7.16 (s, 1H), 5.32-5.10 (m, 1H), 4.67-4.53 (m, 1H), 4.34-3.95 (m, 5H), 3.72-3.63 (m, 1H), 3.56-3.47 (m, 1H), 3.40-3.07 (m, 5H), 2.91 (br s, 1H), 2.74-2.66 (m, 1H), 2.27-2.05 (m, 3H), 1.91-1.77 (m, 4H), 1.45 (s, 9H) ppm.

### Step VII: Synthesis of 2-((S)-4-(7-(8-ethynyl-7-fluoronaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile

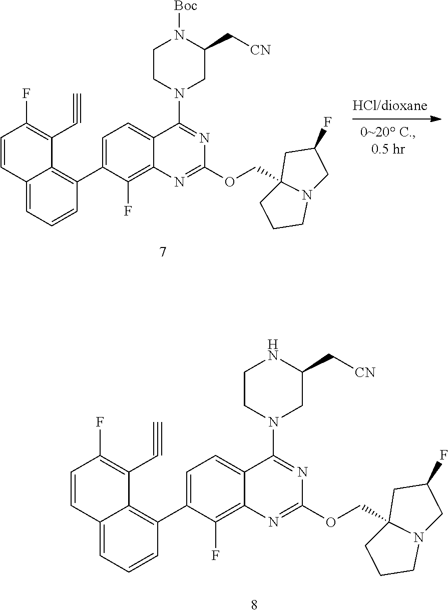 [SMILES: C#Cc1c(F)ccc2cccc(-c3ccc4c(N5CCN(C(=O)OC(C)(C)C)[C@@H](CC#N)C5)nc(OC[C@@]56CCCN5C[C@H](F)C6)nc4c3F)c12 ; C#Cc1c(F)ccc2cccc(-c3ccc4c(N5CCN[C@@H](CC#N)C5)nc(OC[C@@]56CCCN5C[C@H](F)C6)nc4c3F)c12]

At 0° C., hydrochloric acid/dioxane (4.00 M, 4.00 mL) was dropwise added to tert-butyl (S)-2-(cyanomethyl)-4-(7-(8-ethynyl-7-fluoronaphth-1-yl)-8-fluoro-2-(((2R,7aS)-2-fluorotetrahydro-1H-pyrrolizin-7a(5H)-yl)methoxy)quinazolin-4-yl)piperazine-1-carboxylate (200 mg, 287 umol). The reaction liquid was reacted at 20° C. for 0.5 hours. The reaction was complete as monitored by LCMS. The reaction liquid was concentrated under reduced pressure to obtain 2-((S)-4-(7-(8-ethynyl-7-fluoronaphth-1-yl)-8-fluoro-2-(((2R,7aS)-2-fluorotetrahydro-1H-pyrrolizin-7a(5H)-yl)methoxy)quinazolin-4-yl)piperazin-2-yl)acetonitrile (200 mg, crude).

LCMS: m/z 597.3 [M+H]⁺.

### Step VIII: Synthesis of 2-((S)-4-(7-(8-ethynyl-7-fluoronaphth-1-yl)-8-fluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

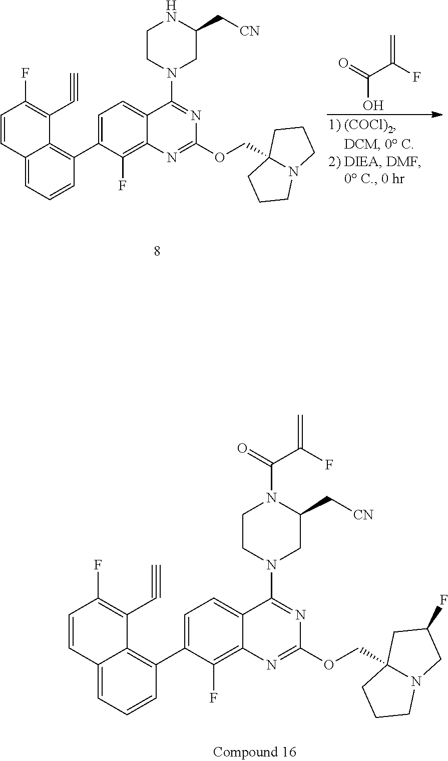

2-Fluoroacrylic acid (30.2 mg, 335 umol) was dissolved in 4.00 mL of dichloromethane, and oxalyl chloride (51.1 mg, 402 umol) was dropwise added at 0° C.; subsequently, N,N-dimethylformamide (2.45 mg, 33.5 umol) was dropwise added, and the reaction liquid was stirred at 0° C. for 0.5 hours. Thereafter, the above reaction liquid was dropwise added to a solution of 2-((S)-4-(7-(8-ethynyl-7-fluoronaphth-1-yl)-8-fluoro-2-(((2R,7aS)-2-fluorotetrahydro-11H-pyrrolizin-7a(5H)-yl)methoxy)quinazolin-4-yl)piperazin-2-yl)acetonitrile (200 mg, 335 umol) and diisopropyl ethyl amine (130 mg, 1.01 mmol) in 2.00 mL N,N-dimethylformamide. The reaction liquid was stirred at 0° C. for 0.5 hours. The reaction was complete as monitored by LCMS. The reaction liquid was concentrated under reduced pressure and purified by prep-HPLC to obtain 2-((S)-4-(7-(8-ethynyl-7-fluoronaphth-1l-yl)-8-fluoro-2-((2R,7aS)-2-fluorotetrahydro-11H-pyrrolizin-7a(5H)-yl)methoxy)quinazolin-4-yl)-1-(2-fluoroacryloyl)piperazin-2-yl)acetonitrile (110 mg, 163 umol, 48.7% yield).

LCMS: m/z 669.5 [M+H]⁺.

¹H NMR (400 MHz, (CD₃)₂SO): δ=8.26-8.14 (m, 2H), 7.84 (t, J=9.4 Hz, 1H), 7.71-7.53 (m, 3H), 7.33-7.25 (m, 1H), 5.49-5.31 (m, 2H), 5.28-5.17 (m, 1H), 5.03-4.72 (m, 1H), 4.36-4.22 (m, 2H), 4.13-4.08 (m, 1H), 4.05-3.99 (m, 1H), 3.91 (d, J=5.9 Hz, 1H), 3.60-3.48 (m, 1H), 3.46-3.35 (m, 1H), 3.30-3.23 (m, 1H), 3.17-3.00 (m, 4H), 2.87-2.78 (m, 1H), 2.49-2.45 (m, 2H), 2.17-1.96 (m, 3H), 1.89-1.71 (m, 3H) ppm.

### Example 17: (S)-2-(4-(7-(8-chloronaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (Compound 17)

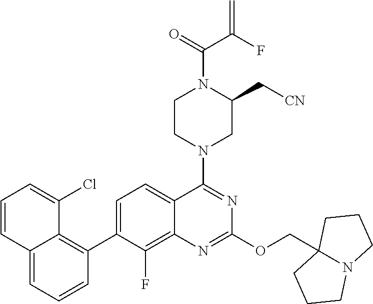 [SMILES: C=C(F)C(=O)N1CCN(c2nc(OCC34CCCN3CCC4)nc3c(F)c(-c4cccc5cccc(Cl)c45)ccc23)C[C@@H]1CC#N]

The specific synthesis route of Compound 17 was as follows:

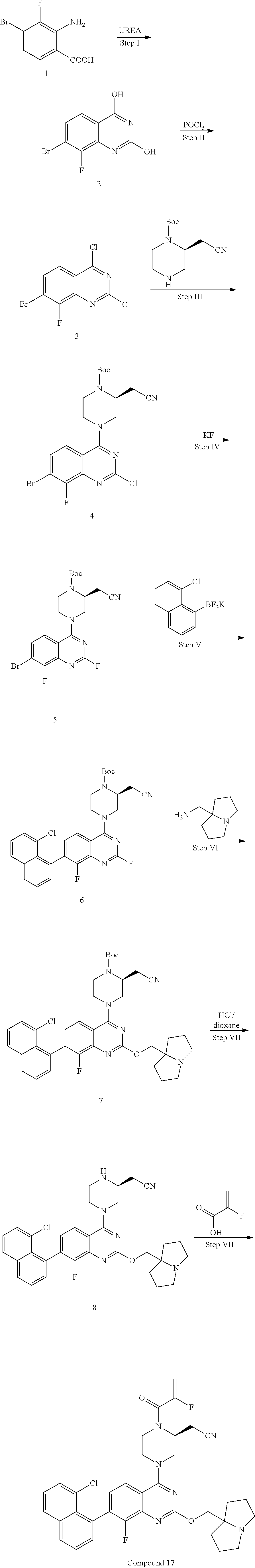

Starting from the intermediate No. 5 compound, the specific synthesis process of Compound 17 was as follows:

### Step V: Synthesis of tert-butyl(S)-4-(7-(8-chloronaphth-1-yl)-2,8-difluoroquinazolin-4-yl)-2-(cyanomethyl) piperazine-1-carboxylate

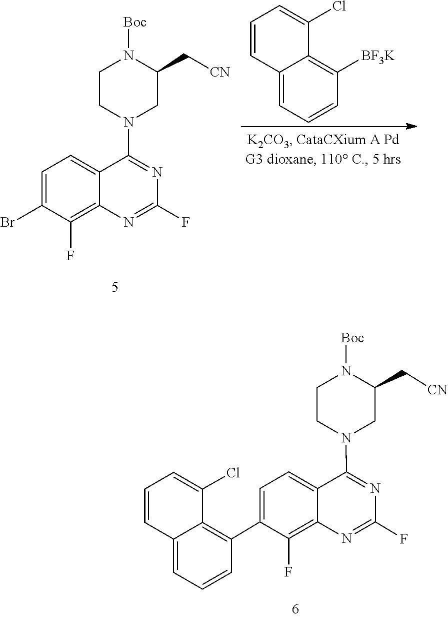

Tert-butyl(S)-4-(7-bromo-2,8-difluoroquinazolin-4-yl)-2-(cyanomethyl) piperazine-1-carboxylate (400 mg, 854 umol), potassium (8-chloro-1-naphthyl) trifluoroboronate (321 mg, 1.20 mmol), methanesulfonato (diadamantyl-N-butylphosphino)-2′-amino-1,1′-biphenyl-2-yl) palladium (II) (124 mg, 171 umol), and potassium carbonate (236 mg, 1.71 mmol) were dissolved in 2.50 mL of dioxane, and after nitrogen displacement three times, the mixture was stirred at 110° C. for 5 hours. After the reaction was complete as monitored by LCMS, the reaction liquid was diluted with ethyl acetate and washed with brine, and the organic phase was concentrated under reduced pressure and purified by a fast silica gel column to obtain tert-butyl(S)-4-(7-(8-chloronaphth-1-yl)-2,8-difluoroquinazolin-4-yl)-2-(cyanomethyl) piperazine-1-carboxylate (420 mg, 764 umol, 89.4% yield).

¹H NMR (400 MHZ, CDCl₃): δ=8.06-7.94 (m, 2H), 7.90 (d, J=8.0 Hz, 1H), 7.78-7.72 (m, 1H), 7.63-7.53 (m, 2H), 7.46-7.41 (m, 2H), 4.75-4.63 (m, 1H), 4.49-4.41 (m, 2H), 4.28-4.16 (m, 1H), 3.67-3.59 (m, 1H), 3.54-3.42 (m, 1H), 2.91-2.74 (m, 2H), 2.51-2.29 (m, 1H), 1.54 (s, 9H) ppm.

### Step VI: Synthesis of tert-butyl (S)-4-(7-(8-chloronaphth-1-yl)-8-fluoro-2-((tetrahydro-1H-pyrrolizin-7a(5H)-yl)methoxy)quinazolin-4-yl)-2-(cyanomethyl)piperazine-1-carboxylate

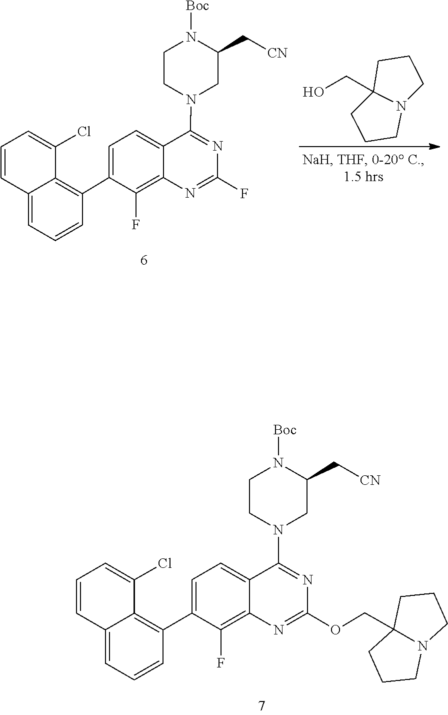 [SMILES: CC(C)(C)OC(=O)N1CCN(c2nc(F)nc3c(F)c(-c4cccc5cccc(Cl)c45)ccc23)C[C@@H]1CC#N ; OCC12CCCN1CCC2 ; CC(C)(C)OC(=O)N1CCN(c2nc(OCC34CCCN3CCC4)nc3c(F)c(-c4cccc5cccc(Cl)c45)ccc23)C[C@@H]1CC#N]

(Tetrahydro-1H-pyrrolizin-7a(5H)-yl)methanol (205 mg, 1.45 mmol) was dissolved in 10.0 mL of tetrahydrofuran, sodium hydride (65.5 mg, 1.64 mmol, 60.0% purity) was added at 0° C., and the reaction liquid was stirred at 0° C. for 0.5 hours. Tert-butyl (S)-4-(7-(8-chloronaphth-1-yl)-2,8-difluoroquinazolin-4-yl)-2-(cyanomethyl)piperazine-1-carboxylate (200 mg, 364 umol) was dissolved in 2.00 mL of tetrahydrofuran and dropwise added to the reaction liquid, and the reaction liquid was further stirred at 20° C. for 1 hour. After the reaction was complete as monitored by LCMS, the reaction liquid was quenched with a saturated ammonium chloride aqueous solution, extracted with ethyl acetate, and washed with brine, and the organic phase was concentrated and purified by a fast silica gel column to obtain tert-butyl (S)-4-(7-(8-chloronaphth-1-yl)-8-fluoro-2-((tetrahydro-1H-pyrrolizin-7a(5H)-yl)methoxy)quinazolin-4-yl)-2-(cyanomethyl)piperazine-1-carboxylate (0.17 g, 185 umol, 50.8% yield).

LCMS: m/z 671.2 [M+H]⁺.

¹H NMR (400 MHz, CDCl₃): δ=8.31-8.05 (m, 1H), 7.97 (br d, J=7.2 Hz, 1H), 7.94-7.85 (m, 1H), 7.79-7.49 (m, 3H), 7.46-7.41 (m, 1H), 7.26-7.21 (m, 1H), 4.76-4.65 (m, 1H), 4.42-4.26 (m, 3H), 3.72-3.33 (m, 4H), 2.99-2.60 (m, 4H), 2.44-2.09 (m, 3H), 2.03-1.84 (m, 4H), 1.81-1.68 (m, 2H), 1.60 (br s, 2H), 1.57-1.51 (m, 9H) ppm.

### Step VII: Synthesis of (S)-2-(4-(7-(8-chloronaphth-1-yl)-8-fluoro-2-((tetrahydro-1H-pyrrolizin-7a(5H)-yl)methoxy)quinazolin-4-yl)piperazin-2-yl)acetonitrile

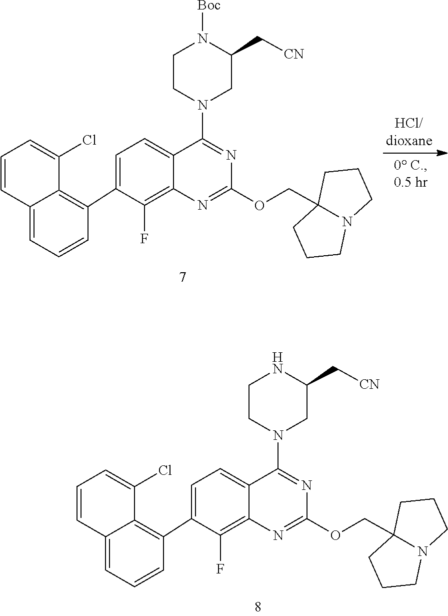 [SMILES: CC(C)(C)OC(=O)N1CCN(c2nc(OCC34CCCN3CCC4)nc3c(F)c(-c4cccc5cccc(Cl)c45)ccc23)C[C@@H]1CC#N ; N#CC[C@H]1CN(c2nc(OCC34CCCN3CCC4)nc3c(F)c(-c4cccc5cccc(Cl)c45)ccc23)CCN1]

At 0° C., 2.00 mL of hydrochloric acid/1,4-dioxane was added to tert-butyl (S)-4-(7-(8-chloronaphth-1-yl)-8-fluoro-2-((tetrahydro-1H-pyrrolizin-7a(5H)-yl)methoxy)quinazolin-4-yl)-2-(cyanomethyl)piperazine-1-carboxylate (150 mg, 223 umol), and the reaction liquid was stirred at 0° C. for 0.5 hours. After the reaction was complete as monitored by LCMS, the reaction liquid was concentrated under reduced pressure to obtain (S)-2-(4-(7-(8-chloronaphth-1-yl)-8-fluoro-2-((tetrahydro-1H-pyrrolizin-7a(5H)-yl)methoxy)quinazolin-4-yl)piperazin-2-yl)acetonitrile (150 mg, crude).

LCMS: m/z 571.3 [M+H]⁺.

### Step VIII: Synthesis of (S)-2-(4-(7-(8-chloronaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile

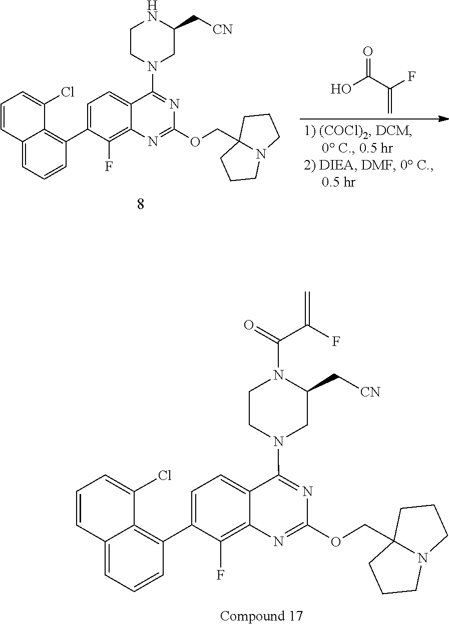

2-Fluoroprop-2-enoic acid (44.2 mg, 490 umol) was dissolved in 2.00 mL of dichloromethane, oxalyl chloride (74.7 mg, 588 umol, 51.5 uL) was added to the reaction liquid at 0° C., N,N-dimethylformamide (1.79 mg, 24.5 umol, 1.89 uL) was then added, and the mixture was stirred for 30 minutes; subsequently, the reaction liquid was dropwise added to a solution of (S)-2-(4-(7-(8-chloronaphth-1-yl)-8-fluoro-2-((tetrahydro-11H-pyrrolizin-7a(5H)-yl)methoxy)quinazolin-4-yl)piperazin-2-yl)acetonitrile (140 mg, 245 umol) and N,N-diisopropylethyl amine (95.1 mg, 735 umol, 128 uL) in N,N-dimethylformamide (2.00 mL) and stirred for 0.5 hours. After the reaction was complete as monitored by LCMS, the reaction liquid was concentrated and purified by prep-HPLC to obtain (S)-2-(4-(7-(8-chloronaphth-1l-yl)-8-fluoro-2-((tetrahydro-11H-pyrrolizin-7a(5H)-yl)methoxy)quinazolin-4-yl)-1-(2-fluoroacryloyl)piperazin-2-yl)acetonitrile (31.0 mg, 47.7 umol, 19.5% yield).

LCMS: m/z 643.5 [M+H]⁺.

¹H NMR (400 MHz, (CD₃)₂SO): δ=8.22-8.07 (m, 2H), 7.89 (dd, J=4.8, 8.4 Hz, 1H), 7.78-7.60 (m, 2H), 7.59-7.48 (m, 2H), 7.38-7.28 (m, 1H), 5.49-5.20 (m, 2H), 5.06-4.74 (m, 1H), 4.38-4.25 (m, 2H), 4.08 (br s, 4H), 3.62-3.44 (m, 2H), 3.13-2.92 (m, 3H), 2.63-2.56 (m, 2H), 2.02-1.53 (m, 9H) ppm.

### Example 18: 2-((2S′)-4-(7-(8-ethynylnaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile (Compound 18)

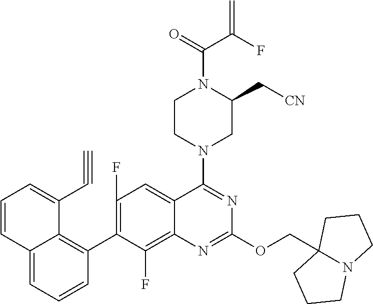 [SMILES: C#Cc1cccc2cccc(-c3c(F)cc4c(N5CCN(C(=O)C(=C)F)[C@@H](CC#N)C5)nc(OCC56CCCN5CCC6)nc4c3F)c12]

The specific synthesis route of Compound 18 was as follows:

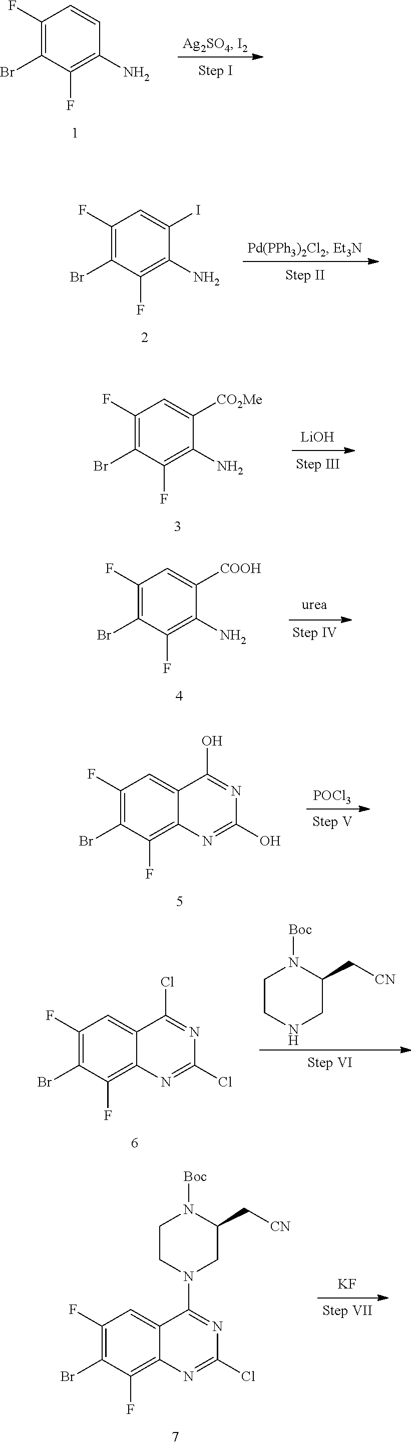

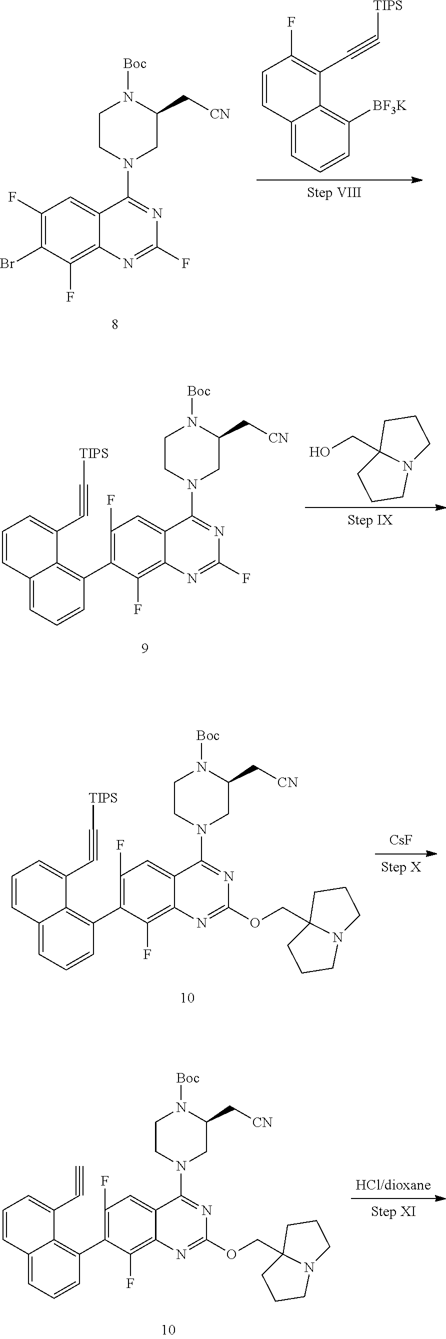

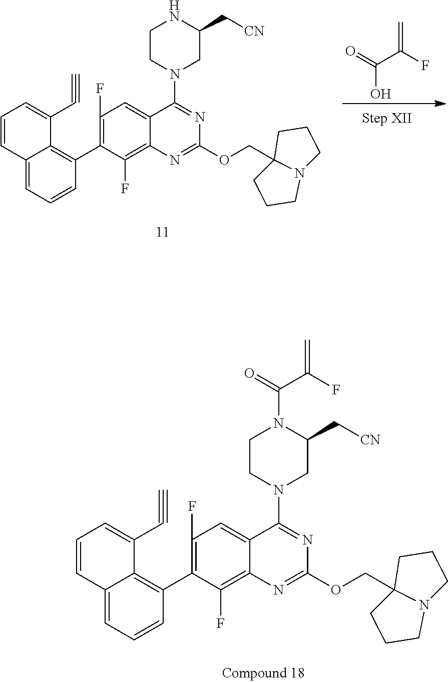

Starting from the intermediate No. 6 compound, the specific synthesis process of Compound 18 was as follows:

Step VIII: Synthesis of tert-butyl (2S)-2-(cyanomethyl)-4-(2,6,8-trifluoro-7-(8-((triisopropylsilyl) ethynyl) naphth-1-yl) quinazolin-4-yl) piperazine-1-carboxylate

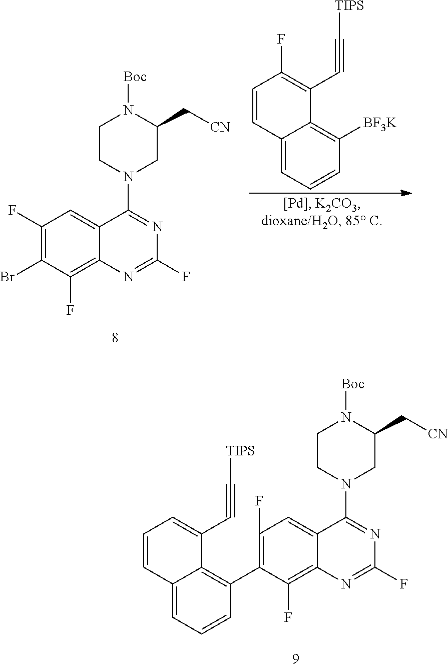

Under nitrogen protection, tert-butyl(S)-4-(7-bromo-2,6,8-trifluoroquinazolin-4-yl)-2-(cyanomethyl) piperazine-1-carboxylate (100 mg, 205 umol) and triisopropyl ((8-potassium trifluoroboronate-2-yl) naphth-1-yl) ethynyl) silane (200 mg, 482 umol), methylsulfonyloxy (diadamantyl-n-butylphosphino)-2-amino-1,1-biphenyl-2-yl) palladium (II) (29.9 mg, 41.1 umol), and potassium carbonate (100 mg, 723 umol) were dissolved in dioxane (2.5 mL) and water (0.25 mL) and reacted at 85° C. for 2 hours. The reaction was complete as monitored by LCMS. The reaction liquid was concentrated and subjected to column chromatography to obtain tert-butyl (2S)-2-(cyanomethyl)-4-(2,6,8-trifluoro-7-(8-((triisopropylsilyl)ethynyl)naphth-1-yl)quinazolin-4-yl)piperazine-1-carboxylate as a yellow solid (340 mg, yield 57.9%).

LCMS: m/z 713.3 [M+H]⁺.

¹H NMR (400 MHz, CDCl₃) δ=8.04-7.92 (m, 2H), 7.82 (d, J=7.1 Hz, 1H), 7.60 (t, J=7.7 Hz, 1H), 7.53-7.40 (m, 3H), 4.71 (br d, J=2.0 Hz, 1H), 4.39-4.05 (m, 3H), 3.76-3.51 (m, 2H), 3.49-3.16 (m, 1H), 3.06-2.73 (m, 2H), 1.56-1.51 (m, 9H), 0.91-0.82 (m, 18H), 0.63-0.50 (m, 3H) ppm.

### Step IX: Synthesis of tert-butyl (2S)-2-(cyanomethyl)-4-(6,8-difluoro-2-(tetrahydro-1H-pyrrolizin-7a(5H)-yl)methoxy)-7-(8-((triisopropylsilyl)ethynyl)naphth-1-yl)quinazolin-4-yl)piperazine-1-carboxylate

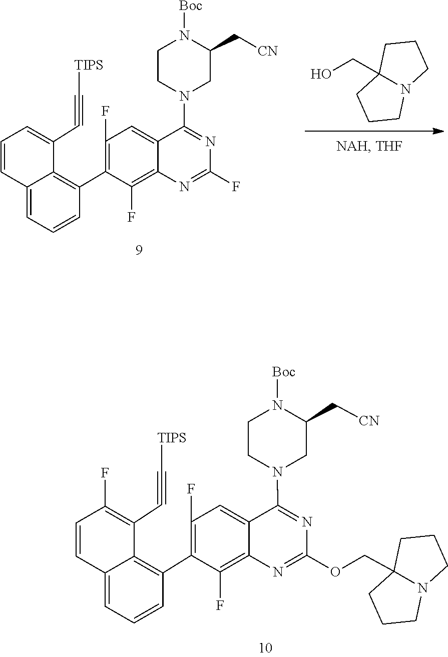

Under nitrogen protection at 0° C., (tetrahydro-1H-pyrrolizin-7a(5H)-yl)methanol (201 mg, 1.43 mmol) was dissolved in tetrahydrofuran (6 mL), sodium hydride (59.1 mg, 1.48 mmol) was then added, the reaction liquid was reacted at 20° C. for 1 hour, and tert-butyl (2S)-2-(cyanomethyl)-4-(2,6,8-trifluoro-7-(8-((triisopropylsilyl)ethynyl)naphth-1-yl)quinazolin-4-yl)piperazine-1-carboxylate (340 mg, 476 umol) was then added and reacted for 30 minutes. The reaction was complete as monitored by LCMS. Water was added to the reaction liquid at 0° C., which was extracted with ethyl acetate, washed with a sodium chloride aqueous solution, and dried, and after filtration, concentration and column chromatography, tert-butyl (2S)-2-(cyanomethyl)-4-(6,8-difluoro-2-(tetrahydro-1H-pyrrolizin-7a(5H)-yl)methoxy)-7-(8-((triisopropylsilyl)ethynyl)naphth-1-yl)quinazolin-4-yl)piperazine-1-carboxylate as a white solid (250 mg, yield 62.8%) was obtained.

LCMS: m/z 835.4 [M+H]⁺.

¹H NMR (400 MHz, CDCl₃) δ=7.96 (br dd, J₁=8.2, J₂=18.2 Hz, 2H), 7.81 (br d, J=7.1 Hz, 1H), 7.58 (t, J=7.6 Hz, 1H), 7.51-7.40 (m, 2H), 7.35-7.28 (m, 1H), 4.77-4.62 (m, 1H), 4.55-4.29 (m, 2H), 4.26-4.11 (m, 3H), 3.87-3.60 (m, 1H), 3.59-3.40 (m, 2H), 3.35-3.14 (m, 2H), 2.94-2.52 (m, 4H), 2.29-2.13 (m, 2H), 1.96 (br s, 3H), 1.85-1.67 (m, 3H), 1.53 (s, 9H), 0.91-0.82 (m, 18H), 0.61-0.48 (m, 3H) ppm.

### Step X: Synthesis of tert-butyl (2S)-2-(cyanomethyl)-4-(7-(8-ethynylnaphth-1-yl)-6,8-difluoro-2-((tetrahydro-1H-pyrrolizin-7a(5H)-yl)methoxy)quinazolin-4-yl)piperazine-1-carboxylate

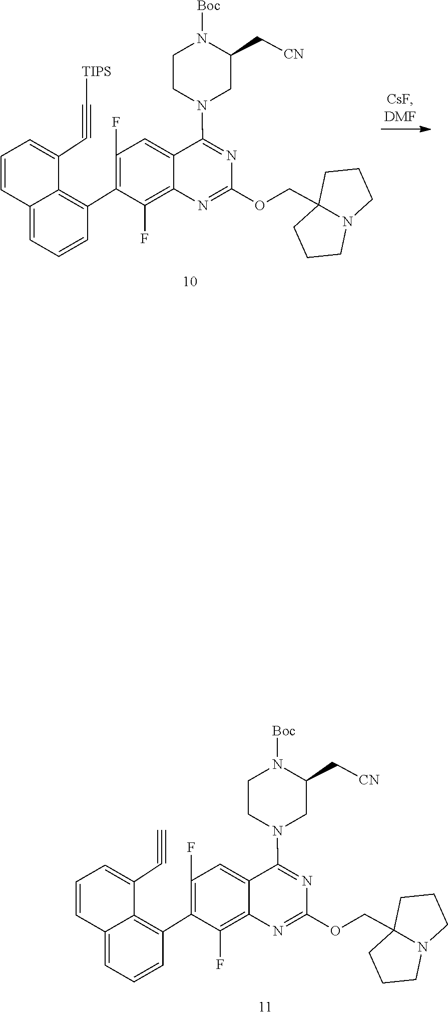

Under nitrogen protection, tert-butyl (2S)-2-(cyanomethyl)-4-(6,8-difluoro-2-(tetrahydro-1H-pyrrolizin-7a(5H)-yl)methoxy)-7-(8-((triisopropylsilyl)ethynyl)naphth-1-yl)quinazolin-4-yl)piperazine-1-carboxylate (250 mg, 299 umol) and cesium fluoride (181 mg, 1.20 mmol) were dissolved in N,N-dimethylformamide (3 mL) and then reacted at 60° C. for 1 hour. The reaction was complete as monitored by LCMS. The reaction liquid was quenched by adding water, extracted with ethyl acetate, washed with a sodium chloride aqueous solution, and dried, and after filtration, concentration and column chromatography, tert-butyl (2S)-2-(cyanomethyl)-4-(7-(8-ethynylnaphth-1-yl)-6,8-difluoro-2-((tetrahydro-1H-pyrrolizin-7a(5H)-yl)methoxy)quinazolin-4-yl)piperazine-1-carboxylate as a yellow solid (130 mg, yield 63.9%) was obtained.

LCMS: m/z 679.4 [M+H]⁺.

¹H NMR (400 MHz, CDCl₃) δ=7.99 (dd, J₁=8.0, J₂=14.4 Hz, 2H), 7.76 (dd, J₁=1.0, J₂=7.1 Hz, 1H), 7.61 (t, J=7.6 Hz, 1H), 7.50-7.44 (m, 2H), 7.33-7.29 (m, 1H), 4.68 (br d, J=1.8 Hz, 1H), 4.47-4.22 (m, 4H), 4.12-4.01 (m, 1H), 3.72-3.62 (m, 1H), 3.55-3.23 (m, 4H), 3.02-2.90 (m, 1H), 2.84-2.59 (m, 4H), 2.27-2.13 (m, 2H), 1.96 (br s, 4H), 1.80-1.73 (m, 2H), 1.53 (s, 9H) ppm.

### Step XI: Synthesis of 2-((2S)-4-(7-(8-ethynylnaphth-1-yl)-6,8-difluoro-2-((tetrahydro-1H-pyrrolizin-7a(5H)-yl)methoxy)quinazolin-4-yl)piperazin-2-yl)acetonitrile

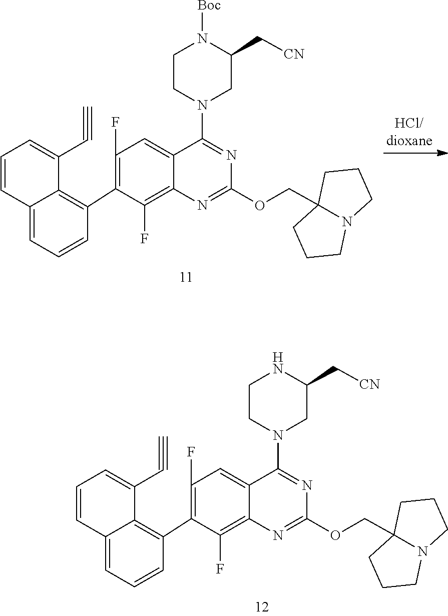 [SMILES: C#Cc1cccc2cccc(-c3c(F)cc4c(N5CCN(C(=O)OC(C)(C)C)[C@@H](CC#N)C5)nc(OCC56CCCN5CCC6)nc4c3F)c12 ; C#Cc1cccc2cccc(-c3c(F)cc4c(N5CCN[C@@H](CC#N)C5)nc(OCC56CCCN5CCC6)nc4c3F)c12]

Tert-butyl (2S)-2-(cyanomethyl)-4-(7-(8-ethynylnaphth-1-yl)-6,8-difluoro-2-((tetrahydro-1H-pyrrolizin-7a(5H)-yl)methoxy)quinazolin-4-yl)piperazine-1-carboxylate (130 mg, 191 umol) was dissolved in hydrochloric acid/dioxane (10 mL) and then reacted at 0° C. for 30 minutes. The reaction was complete as monitored by LCMS. The reaction liquid was concentrated to obtain 2-((2S)-4-(7-(8-ethynylnaphth-1-yl)-6,8-difluoro-2-((tetrahydro-1H-pyrrolizin-7a(5H)-yl)methoxy)quinazolin-4-yl)piperazin-2-yl)acetonitrile as a yellow solid (110 mg, crude).

LCMS: m/z 579.3 [M+H]⁺.

### Step XII: Synthesis of 2-((2S)-4-(7-(8-ethynylnaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile

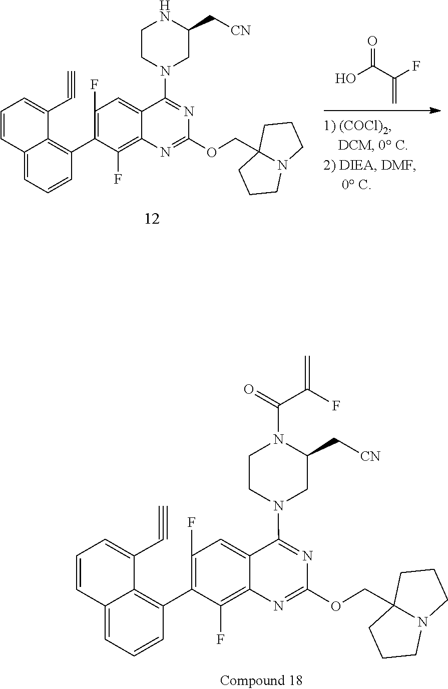

After 2-fluoroacrylic acid (58.3 mg, 648 umol) was dissolved in dichloromethane (4 mL) at 0° C., oxalyl chloride (84.3 mg, 664 umol) was added and reacted at this temperature for 10 minutes, and then, after 2-((2S)-4-(7-(8-ethynylnaphth-1-yl)-6,8-difluoro-2-((tetrahydro-1H-pyrrolizin-7a(5H)-yl)methoxy)quinazolin-4-yl)piperazin-2-yl)acetonitrile (11.0 mg, 162 umol) dissolved in N,N-dimethylformamide (2 mL) was added, a solution of N,N-diisopropylethylamine (41.8 mg, 324 umol) was added, and the resulting mixture was stirred at 0° C. for 30 minutes. The reaction was complete as monitored by LCMS. The reaction liquid was filtered and subjected to prep-HPLC and SFC for separation and purification to obtain 2-((2S)-4-(7-(8-ethynylnaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile as a white solid. After SFC resolution, two target compound peaks 18A and 18B were obtained. Compounds 18A and 18B were axially chiral isomers of each other.

18A: (7.80 mg, yield 7.12%), LCMS: m/z 651.5 [M+H]⁺.

¹H NMR (400 MHz, CD₃CN): 6=8.11 (ddd, J₁=0.9, J₂=8.3, J₃=12.2 Hz, 2H), 7.78 (dd, J₁=1.1, J₂=7.1 Hz, 1H), 7.72-7.65 (m, 1H), 7.59-7.48 (m, 3H), 5.36-5.18 (m, 2H), 5.12-4.71 (m, 1H), 4.30-4.04 (m, 5H), 3.79-3.47 (m, 2H), 3.44-3.32 (m, 1H), 3.14 (br dd, J₁=8.1, J₂=17.3 Hz, 1H), 3.01-2.92 (m, 3H), 2.90 (s, 1H), 2.59 (td, J₁=6.7, J₂=9.9 Hz, 2H), 1.90-1.72 (m, 6H), 1.62 (td, J₁=7.4, J₂=12.1 Hz, 2H) ppm.

18B: (6.70 mg, yield 6.24%), LCMS: m/z 651.5 [M+H]⁺.

¹H NMR (400 MHz, CD₃CN): 6=8.11 (ddd, J₁=1.1, J₂=8.3, J₃=12.1 Hz, 2H), 7.78 (dd, J₁=1.3, J₂=7.1 Hz, 1H), 7.69 (dd, J₁=7.2, J₂=8.2 Hz, 1H), 7.59-7.50 (m, 3H), 5.37-5.17 (m, 2H), 5.03-4.64 (m, 1H), 4.33-4.05 (m, 5H), 3.89-3.52 (m, 2H), 3.42-3.30 (m, 1H), 3.12 (br dd, J₁=7.4, J₂=17.4 Hz, 1H), 3.03-2.94 (m, 3H), 2.90 (dd, J₁=6.8, J₂=17.2 Hz, 1H), 2.61 (td, J=6.7, J₂=10.1 Hz, 2H), 1.92-1.74 (m, 6H), 1.69-1.59 (m, 2H) ppm.

### Example 19: 2-((2S)-4-(7-(8-ethynyl-7-fluoronaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile (Compound 19)

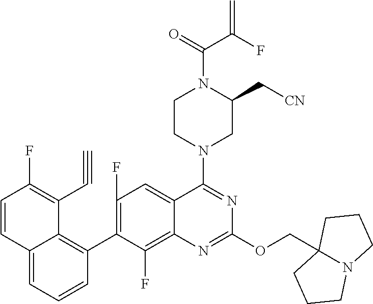 [SMILES: C#Cc1c(F)ccc2cccc(-c3c(F)cc4c(N5CCN(C(=O)C(=C)F)[C@@H](CC#N)C5)nc(OCC56CCCN5CCC6)nc4c3F)c12]

The specific synthesis route of Compound 19 was as shown below:

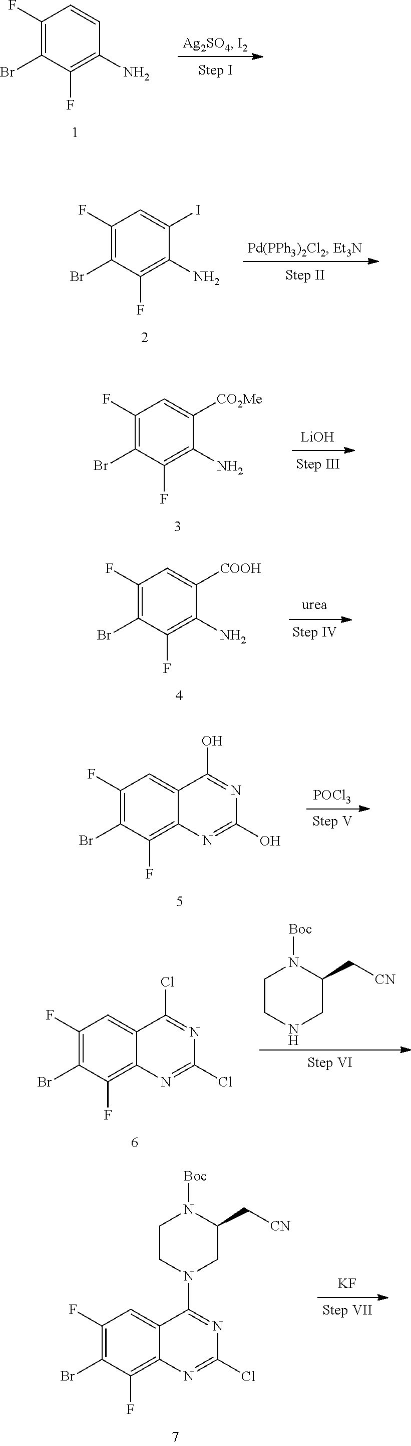

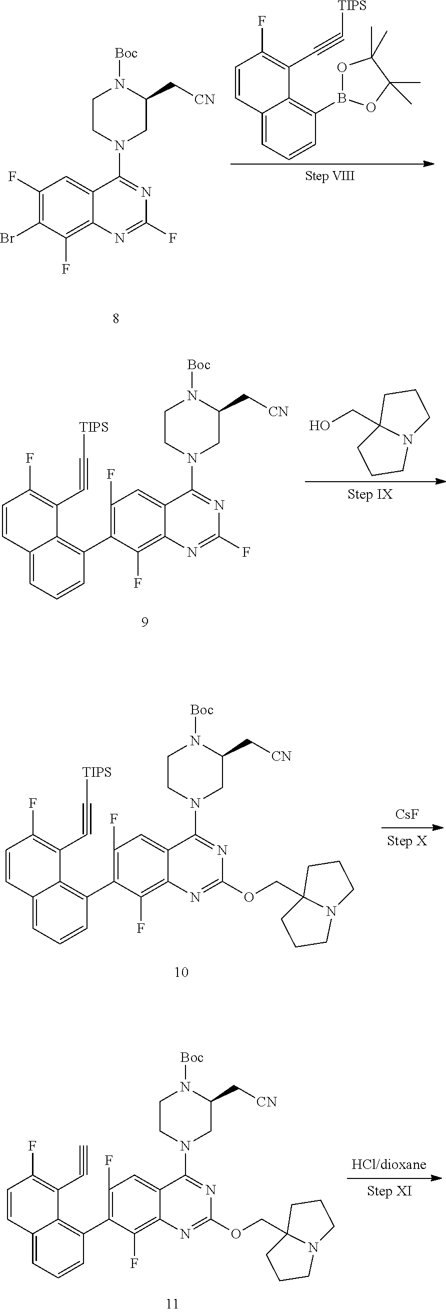

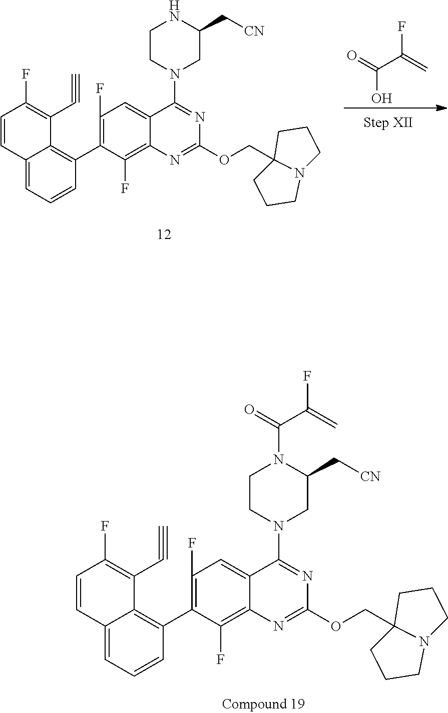

Starting from the intermediate No. 6 compound, the specific synthesis process of Compound 19 was as follows:

### Step VIII: Synthesis of tert-butyl (2S)-2-(cyanomethyl)-4-(2,6,8-trifluoro-7-(7-fluoro-8-((tri isopropylsilyl)ethynyl)naphth-1-yl)quinazolin-4-yl)piperazine-1-carboxylate

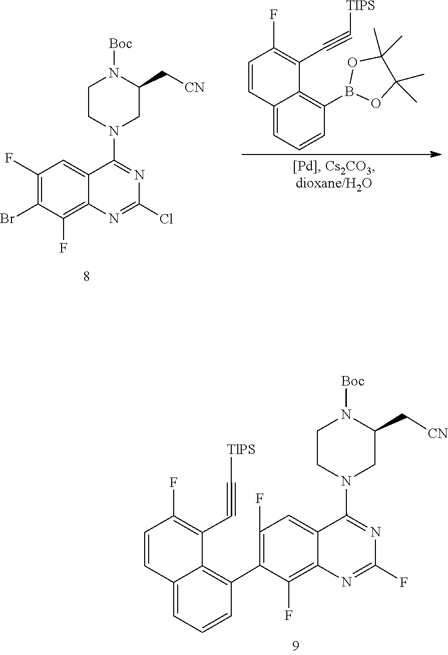

Tert-butyl (S)-4-(7-bromo-2,6,8-trifluoroquinazolin-4-yl)-2-(cyanomethyl)piperazine-1-carboxylate (220 mg, 452 umol) was dissolved in 3.00 mL of dioxane and 0.30 mL of water, and potassium carbonate (188 mg, 1.36 mmol) and methanesulfonato(diadamantyl-N-butylphosphino)-2′-amino-1,1′-biphenyl-2-yl)palladium(II)(65.9 mg, 90.4 umol) were added. The reaction liquid was reacted at 80° C. for 3 hours. The reaction was complete as monitored by LCMS. The reaction liquid was concentrated and purified by a fast silica gel column to obtain tert-butyl (2S)-2-(cyanomethyl)-4-(2,6,8-trifluoro-7-(7-fluoro-8-((triisopropylsilyl)ethynyl)naphth-1-yl)quinazolin-4-yl)piperazine-1-carboxylate (230 mg, 253 umol, 56.0% yield, 80.7% purity).

LCMS: m/z 732.5 [M+H]⁺.

### Step IX: Synthesis of tert-butyl (2S)-2-(cyanomethyl)-4-(6,8-difluoro-7-(7-fluoro-8-((triisopropylsilyl)ethynyl)naphth-1-yl)-2-((tetrahydro-1H-pyrrolizin-7a(5H)-yl)methoxy)quinazolin-4-yl)piperazine-1-carboxylate

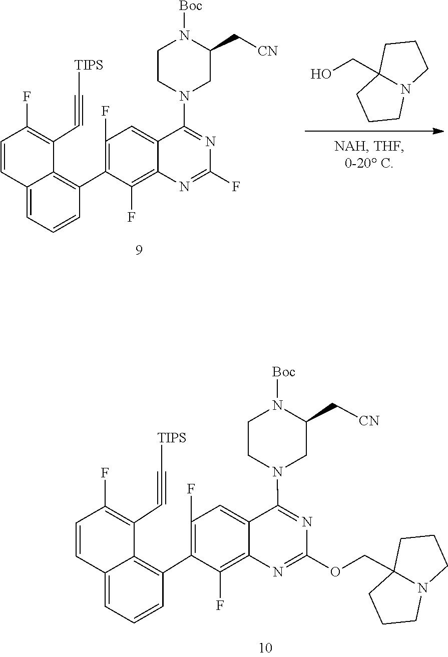

At 0° C. under nitrogen protection, (tetrahydro-1H-pyrrolizin-7a(5H)-yl)methanol (277 mg, 1.97 mmol) was dissolved in tetrahydrofuran (10.0 mL), sodium hydride (80.67 mg, 2.02 mmol, 60% purity) was then added, and the reaction was carried out at 20° C. for 1 hour. Then, tert-butyl (2S)-2-(cyanomethyl)-4-(2,6,8-trifluoro-7-(7-fluoro-8-((triisopropylsilyl)ethynyl)naphth-1-yl)quinazolin-4-yl)piperazine-1-carboxylate (360 mg, 491.87 umol) was added and then reacted for 2 hours. The reaction was complete as monitored by LCMS. The reaction liquid was quenched with water at 0° C. and extracted with ethyl acetate, washed with a saturated sodium chloride aqueous solution, and dried over sodium sulfate, and after filtration, concentration and purification by column chromatography, tert-butyl (2S)-2-(cyanomethyl)-4-(6,8-difluoro-7-(7-fluoro-8-((triisopropylsilyl)ethynyl)naphth-1-yl)-2-((tetrahydro-1H-pyrrolizin-7a(5H)-yl)methoxy)quinazolin-4-yl)piperazine-1-carboxylate (250 mg, 250.85 umol, 51.0% yield) was obtained.

LCMS: m/z 853.4 [M+H]⁺.

¹H NMR (400 MHz, CDCl₃): δ=7.99-7.90 (m, 2H), 7.58-7.53 (m, 1H), 7.46 (br d, J=7.0 Hz, 1H), 7.37-7.32 (m, 1H), 4.77-4.67 (m, 1H), 4.38-4.18 (m, 4H), 3.53-3.40 (m, 2H), 3.31-3.03 (m, 4H), 2.92-2.77 (m, 2H), 2.75-2.58 (m, 3H), 2.07-2.03 (m, 2H), 1.95-1.65 (m, 10H), 1.54 (d, J=3.2 Hz, 9H), 0.91-0.83 (m, 18H), 0.62-0.48 (m, 3H).

### Step X: Synthesis of tert-butyl (2S)-2-(cyanomethyl)-4-(7-(8-ethynyl-7-fluoronaphth-1-yl)-6,8-difluoro-2-((tetrahydro-1H-pyrrolin-7a(5H)-yl)methoxy)quinazolin-4-yl)piperazine-1-carboxylate

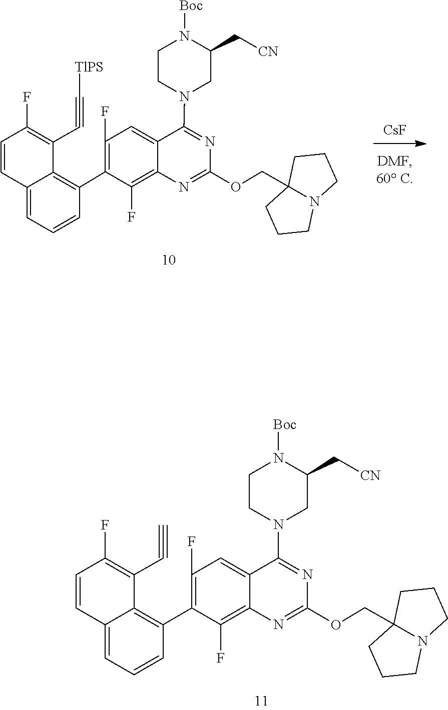

Tert-butyl (2S)-2-(cyanomethyl)-4-(6,8-difluoro-7-(7-fluoro-8-((triisopropylsilyl)ethynyl)naphth-1-yl)-2-((tetrahydro-1H-pyrrolizin-7a(5H)-yl)methoxy)quinazolin-4-yl)piperazine-1-carboxylate (250 mg, 293 umol) was dissolved in 5.00 mL of an N,N-dimethylformamide solution, and cesium fluoride (178 mg, 1.17 mmol) was added. The reaction liquid was reacted at 60° C. for 1 hours. The reaction was complete as monitored by LCMS. The reaction liquid was diluted by adding water, extracted with ethyl acetate, and washed with brine, and the organic phase was concentrated and purified by a fast silica gel column to obtain tert-butyl (2S)-2-(cyanomethyl)-4-(7-(8-ethynyl-7-fluoronaphth-1-yl)-6,8-difluoro-2-((tetrahydro-1H-pyrrolizin-7a(5H)-yl)methoxy)quinazolin-4-yl)piperazine-1-carboxylate (200 mg, 253 umol, 86.4% yield).

LCMS: m/z 697.4 [M+H]⁺.

### Step XI: Synthesis of 2-((2S)-4-(7-(8-ethynyl-7-fluoronaphth-1-yl)-6,8-difluoro-2-((tetrahydro-1H-pyrrolizin-7a(5H)-yl)methoxy)quinazolin-4-yl)piperazin-2-yl)acetonitrile

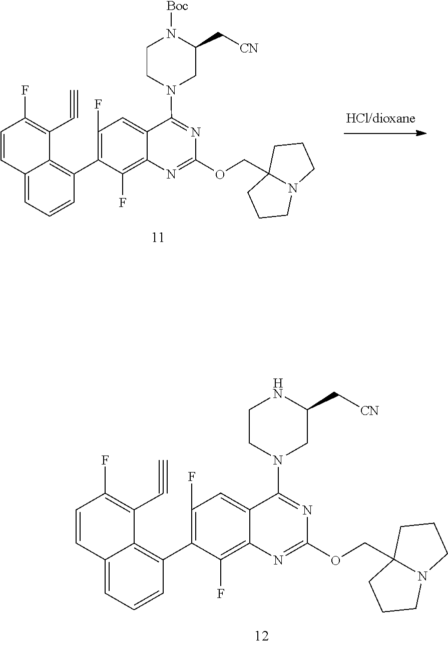 [SMILES: C#Cc1c(F)ccc2cccc(-c3c(F)cc4c(N5CCN(C(=O)OC(C)(C)C)[C@@H](CC#N)C5)nc(OCC56CCCN5CCC6)nc4c3F)c12 ; C#Cc1c(F)ccc2cccc(-c3c(F)cc4c(N5CCN[C@@H](CC#N)C5)nc(OCC56CCCN5CCC6)nc4c3F)c12]

At 0° C., tert-butyl (2S)-2-(cyanomethyl)-4-(7-(8-ethynyl-7-fluoronaphth-1-yl)-6,8-difluoro-2-((tetrahydro-1H-pyrrolizin-7a(5H)-yl)methoxy)quinazolin-4-yl)piperazine-1-carboxylate (300 mg, 322 umol) was dissolved in 3.00 mL of a hydrochloric acid/dioxane solution. The reaction liquid was reacted at 20° C. for 0.5 hours. The reaction was complete as monitored by LCMS. The reaction liquid was concentrated to obtain 2-((2S)-4-(7-(8-ethynyl-7-fluoronaphth-1-yl)-6,8-difluoro-2-((tetrahydro-1H-pyrrolizin-7a(5H)-yl)methoxy)quinazolin-4-yl)piperazin-2-yl)acetonitrile (171 mg, crude).

LCMS: m/z 597.2 [M+H]⁺.

### Step XII Synthesis of 2-((2S)-4-(7-(8-ethynyl-7-fluoronaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile

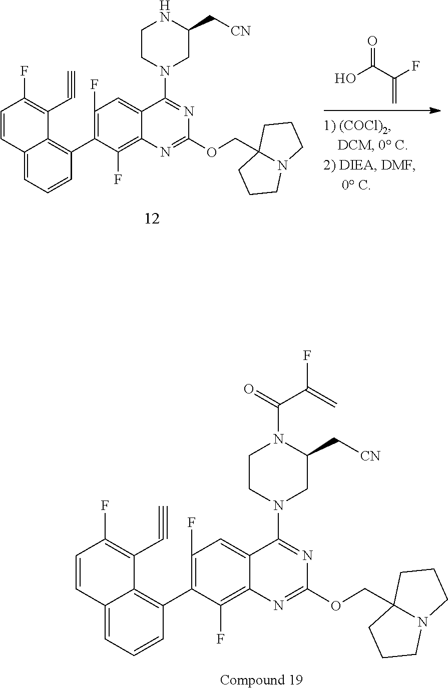

After 2-fluoroacrylic acid (103 mg, 1.15 mmol, 4.00 eq) was dissolved in dichloromethane (4.00 mL) at 0° C., oxalyl chloride (149 mg, 1.18 mmol, 102 μL, 4.10 eq) was added and reacted at this temperature for 10 minutes, 2-((2S)-4-(7-(8-ethynyl-7-fluoronaphth-1-yl)-6,8-difluoro-2-((tetrahydro-1H-pyrrolizin-7a(5H)-yl)methoxy)quinazolin-4-yl)piperazin-2-yl)acetonitrile (171 mg, 286 umol, 1.00 eq) dissolved in N,N-dimethylformamide (1.00 mL) was then added, a solution of N,N-diisopropylethylamine (111 mg, 859 umol, 149 μL, 3.00 eq) was then added, and the resulting mixture was stirred at 0° C. for 30 minutes. The reaction was complete as monitored by LCMS. The reaction liquid was filtered, and the filtrate was subjected to prep-HPLC and SFC for separation and purification to obtain 2-((2S)-4-(7-(8-ethynyl-7-fluoronaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile as a yellow solid.

After SFC resolution, two target compound peaks 19A and 19B were obtained. Compounds 19A and 19B were axially chiral isomers of each other.

19A: (15.7 mg, yield 8.39%), LCMS: m/z 669.5 [M+H]⁺, SFC: 100 ee %.

¹H NMR (400 MHz, CD₃CN): 6=8.16-8.11 (m, 2H), 7.71-7.65 (m, 1H), 7.64-7.60 (m, 1H), 7.54-7.43 (m, 2H), 5.37-5.17 (m, 2H), 5.05-4.79 (m, 1H), 4.31-4.21 (m, 2H), 4.11 (s, 2H), 3.57-3.48 (m, 1H), 3.46-3.34 (m, 1H), 3.23-3.08 (m, 2H), 3.03-2.91 (m, 3H), 2.60 (td, J=6.7, 9.9 Hz, 2H), 2.11 (br d, J=2.3 Hz, 4H), 1.87-1.77 (m, 4H), 1.69-1.57 (m, 2H) ppm.

19B: (16.6 mg, yield 8.86%), LCMS: m/z 669.5 [M+H]⁺, SFC: 97.4 ee %.

¹H NMR (400 MHz, CD₃CN): 6=8.16-8.10 (m, 2H), 7.70-7.65 (m, 1H), 7.64-7.60 (m, 1H), 7.54 (dd, J=1.6, 9.8 Hz, 1H), 7.47 (t, J=9.0 Hz, 1H), 5.37-5.17 (m, 2H), 5.04-4.82 (m, 1H), 4.30-4.15 (m, 2H), 4.11 (s, 2H), 3.59 (br d, J=13.2 Hz, 1H), 3.43-3.30 (m, 1H), 3.27 (d, J=0.6 Hz, 1H), 3.17-3.07 (m, 1H), 3.01-2.86 (m, 3H), 2.60 (td, J=6.8, 9.9 Hz, 2H), 2.07 (br s, 4H), 1.85-1.76 (m, 4H), 1.67-1.59 (m, 2H).

### Example 20: 2-((2S)-4-(7-(8-ethynyl-7-fluoro-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile (Compound 20)

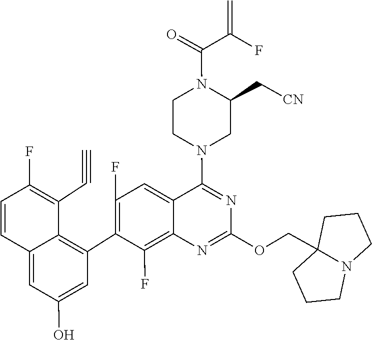 [SMILES: C#Cc1c(F)ccc2cc(O)cc(-c3c(F)cc4c(N5CCN(C(=O)C(=C)F)[C@@H](CC#N)C5)nc(OCC56CCCN5CCC6)nc4c3F)c12]

The specific synthesis route of Compound 20 was as follows:

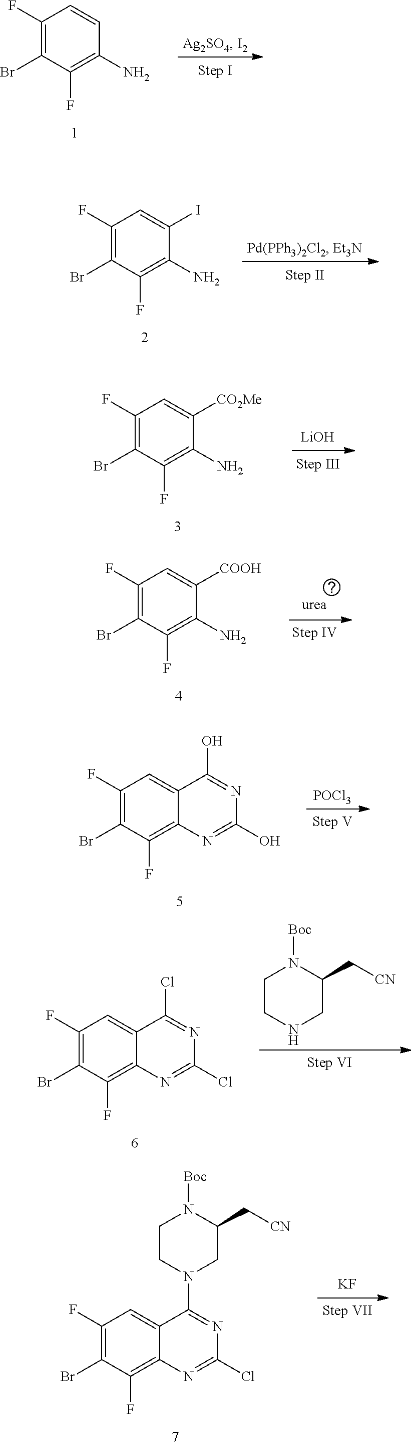

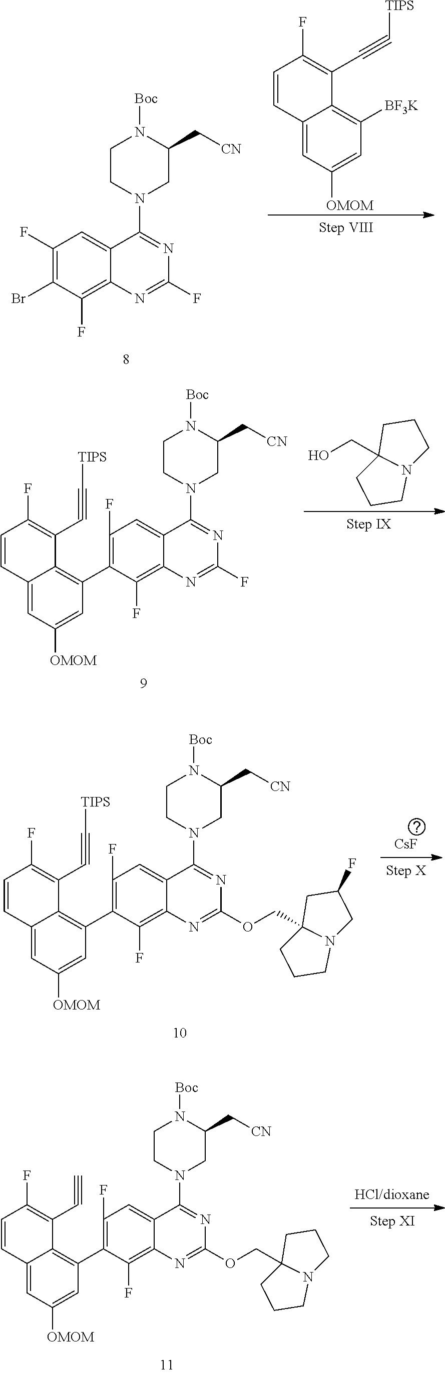

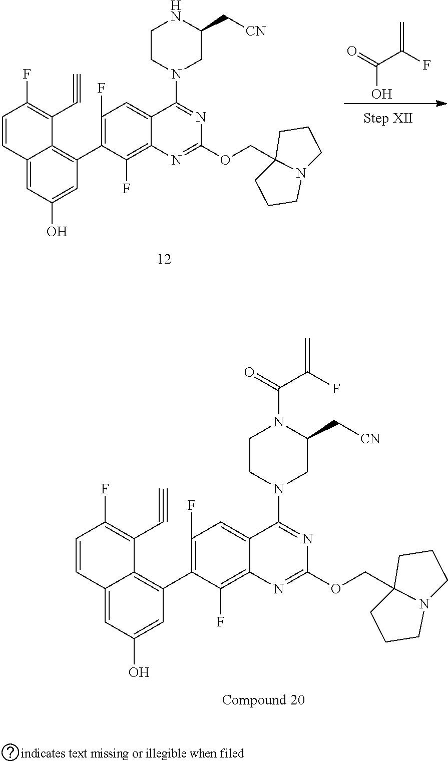

Starting from the intermediate No. 6 compound, the specific synthesis process of Compound 20 was as follows:

### Step VIII: Synthesis of tert-butyl (2S′)-2-(cyanomethyl)-4-(2,6,8-trifluoro-7-(7-fluoro-3-(methoxymethoxy)-8-((triisopropylsilyl)ethynyl)naphth-1-yl)quinazolin-4-yl)piperazine-1-carboxylate

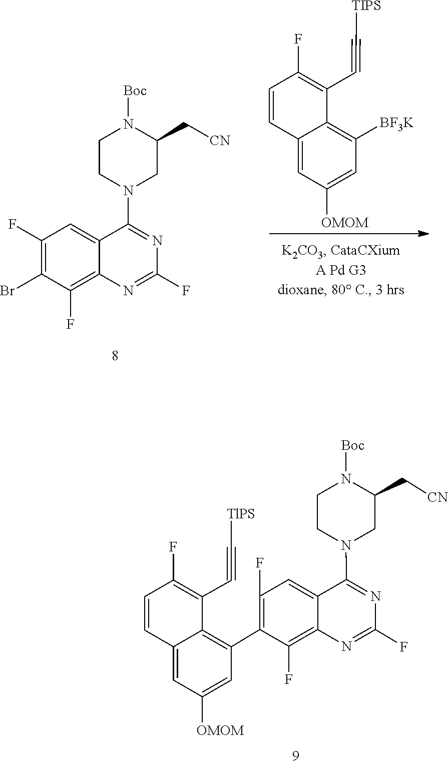

Tert-butyl (S)-4-(7-bromo-2,6,8-trifluoroquinazolin-4-yl)-2-(cyanomethyl)piperazine-1-carboxylate (1.00 g, 2.06 mmol, 1 eq) and potassium (7-fluoro-3-(methoxymethoxy)-8-((triisopropylsilyl)ethynyl)naphth-1-yl)trifluoroboronate were dissolved in 12.0 mL of dioxane and 1.20 mL of water, and potassium carbonate (852 mg, 6.17 mmol, 3 eq) and methanesulfonato(diadamantyl-N-butylphosphino)-2′-amino-1,1′-biphenyl-2-yl)palladium(II)(299 mg, 411 umol, 0.2 eq) were added. The reaction liquid was reacted at 80° C. for 3 hours. The reaction was complete as monitored by LCMS. The reaction liquid was concentrated and purified by a fast silica gel column to obtain tert-butyl (2S)-2-(cyanomethyl)-4-(2,6,8-trifluoro-7-(7-fluoro-3-(methoxymethoxy)-8-((triisopropylsilyl)ethynyl)naphth-1-yl)quinazolin-4-yl)piperazine-1-carboxylate (1.10 g, 1.39 mmol, 67.5% yield).

LCMS: m/z 792.8 [M+H]⁺.

¹H NMR: (400 MHz, CDCl₃): δ=7.81 (dd, J=5.7, 8.8 Hz, 1H), 7.59-7.50 (m, 1H), 7.49-7.38 (m, 1H), 7.32 (t, J=8.6 Hz, 1H), 7.21 (br s, 1H), 5.31 (s, 2H), 4.75-4.64 (m, 1H), 4.77-4.42 (m, 1H), 4.39-4.17 (m, 3H), 3.98-3.66 (m, 2H), 3.54 (s, 3H), 2.89-2.69 (m, 2H), 1.54 (br d, J=6.6 Hz, 9H), 0.91-0.83 (m, 18H), 0.65-0.44 (m, 3H).

### Step IX: Synthesis of tert-butyl (2S)-2-(cyanomethyl)-4-(6,8-difluoro-7-(7-fluoro-3-(methoxymethoxy)-8-(triisopropylsilyl)ethynyl)naphth-1-yl)-2-(tetrahydro-1H-pyrrolizin-7a(5H)-yl)methoxy)quinazolin-4-yl)piperazine-1-carboxylate

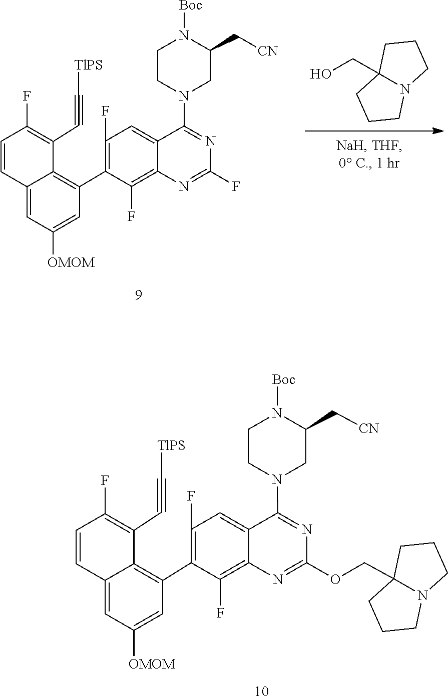

After (tetrahydro-1H-pyrrolizin-7a(5H)-yl)methanol (356 mg, 2.53 mmol, 4 eq) was dissolved in tetrahydrofuran (10.0 mL) at 0° C. under nitrogen protection, sodium hydride (103 mg, 2.59 mmol, 60.0% purity, 4.1 eq) was added and reacted at 20° C. for 1 hour. Tert-butyl (2S)-2-(cyanomethyl)-4-(2,6,8-trifluoro-7-(7-fluoro-3-(methoxymethoxy)-8-((triisopropylsilyl)ethynyl)naphth-1-yl)quinazolin-4-yl)piperazine-1-carboxylate (500 mg, 631 umol, 1 eq) was then added and reacted for 2 hours. The reaction was complete as monitored by LCMS. The reaction liquid was quenched with water at 0° C. and extracted with ethyl acetate, washed with a saturated sodium chloride aqueous solution, and dried over sodium sulfate, and after filtration, concentration and purification by column chromatography, tert-butyl (2S)-2-(cyanomethyl)-4-(6,8-difluoro-7-(7-fluoro-3-(methoxymethoxy)-8-(triisopropylsilyl)ethynyl)naphth-1-yl)-2-(tetrahydro-1H-pyrrolizin-7a(5H)-yl)methoxy)quinazolin-4-yl)piperazine-1-carboxylate (380 mg, 416 umol, 65.9% yield) was obtained.

LCMS: m/z 913.5 [M+H]⁺.

¹H NMR: (400 MHz, CDCl₃): δ=7.80 (dd, J=5.6, 8.6 Hz, 1H), 7.53 (d, J=2.4 Hz, 1H), 7.34-7.29 (m, 2H), 7.21 (d, J=2.4 Hz, 1H), 5.31 (s, 2H), 4.78-4.66 (m, 1H), 4.54-4.15 (m, 4H), 3.53 (s, 3H), 3.44-3.04 (m, 3H), 3.04-2.55 (m, 4H), 2.33-2.11 (m, 2H), 2.01-1.59 (m, 9H), 1.53 (d, J=1.6 Hz, 9H), 0.92-0.81 (m, 18H), 0.63-0.47 (m, 3H)ppm.

### Step X: Synthesis of tert-butyl (2S)-2-(cyanomethyl)-4-(7-(8-ethynyl-7-fluoro-3-(methoxymethoxy)naphth-1-yl)-6,8-difluoro-2-((tetrahydro-1H-pyrrolizin-7a(5H)-yl)methoxy)quinazolin-4-yl)piperazine-1-carboxylate

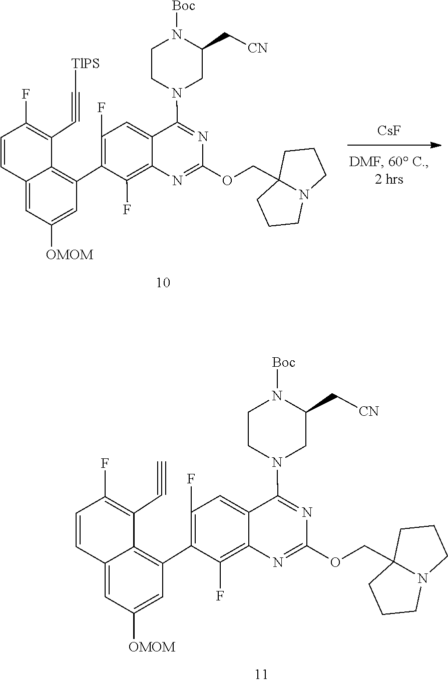

Tert-butyl (2S)-2-(cyanomethyl)-4-(6,8-difluoro-7-(7-fluoro-3-(methoxymethoxy)-8-(triisopropylsilyl)ethynyl)naphth-1-yl)-2-(tetrahydro-1H-pyrrolizin-7a(5H)-yl)methoxy)quinazolin-4-yl)piperazine-1-carboxylate (380 mg, 416 umol, 1 eq) was dissolved in 5.00 mL of an N,N-dimethylformamide solution, and cesium fluoride (252 mg, 1.66 mmol, 61.3 uL, 4 eq) was added. The reaction liquid was reacted at 60° C. for 1 hours. The reaction was complete as monitored by LCMS. The reaction liquid was diluted by adding water, extracted with ethyl acetate, and washed with brine, and the organic phase was concentrated and purified by a fast silica gel column to obtain tert-butyl (2S)-2-(cyanomethyl)-4-(7-(8-ethynyl-7-fluoro-3-(methoxymethoxy)naphth-1-yl)-6,8-difluoro-2-((tetrahydro-1H-pyrrolizin-7a(5H)-yl)methoxy)quinazolin-4-yl)piperazine-1-carboxylate (360 mg, 330 umol, 79.3% yield).

LCMS: m/z 757.3 [M+H]⁺.

¹H NMR: (400 MHz, CDCl₃): δ=7.83 (dd, J=5.8, 9.0 Hz, 1H), 7.55 (d, J=2.4 Hz, 1H), 7.36-7.28 (m, 2H), 7.26 (br s, 1H), 5.32 (s, 2H), 4.67 (br s, 1H), 4.59-4.20 (m, 4H), 3.86-3.60 (m, 2H), 3.54 (s, 3H), 3.51-3.41 (m, 2H), 3.12-2.95 (m, 2H), 2.90-2.76 (m, 3H), 2.53-2.21 (m, 4H), 2.03-1.79 (m, 6H), 1.52 (s, 9H) ppm.

### Step XI: Synthesis of 2-((2S)-4-(7-(8-ethynyl-7-fluoro-3-hydroxynaphth-1-yl)-6,8-difluoro-2-((tetrahydro-1H-pyrrolizin-7a(5H)-yl)methoxy)quinazolin-4-yl)piperazin-2-yl)acetonitrile

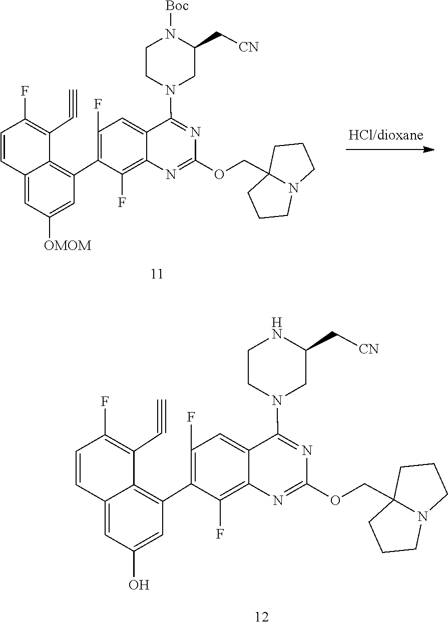

At 0° C., tert-butyl (2S)-2-(cyanomethyl)-4-(7-(8-ethynyl-7-fluoro-3-(methoxymethoxy)naphth-1-yl)-6,8-difluoro-2-((tetrahydro-1H-pyrrolizin-7a(5H)-yl)methoxy)quinazolin-4-yl)piperazine-1-carboxylate (260 mg, 343 umol, 1 eq) was dissolved in 5.00 mL of a hydrochloric acid/dioxane solution. The reaction liquid was reacted at 20° C. for 0.5 hours. The reaction was complete as monitored by LCMS. The reaction liquid was concentrated to obtain 2-((2S)-4-(7-(8-ethynyl-7-fluoro-3-hydroxynaphth-1-yl)-6,8-difluoro-2-((tetrahydro-1H-pyrrolizin-7a(5H)-yl)methoxy)quinazolin-4-yl)piperazin-2-yl)acetonitrile (210 mg, crude).

LCMS: m/z 613.1 [M+H]⁺.

### Step XII: Synthesis of 2-((2S)-4-(7-(8-ethynyl-7-fluoro-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile

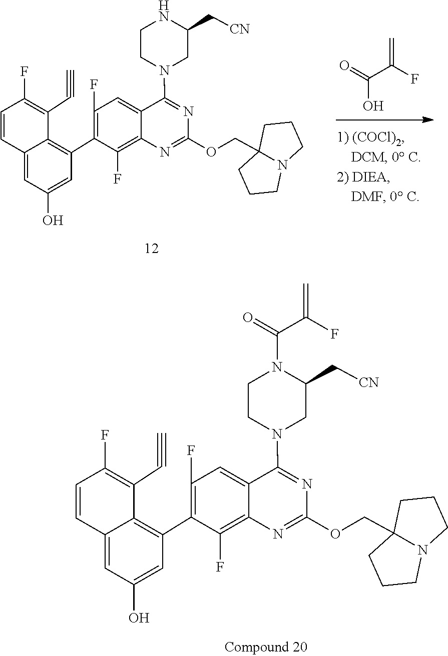

After 2-fluoroacrylic acid (41.1 mg, 457 umol 1, 4.00 eq) was dissolved in dichloromethane (2.00 mL) at 0° C., oxalyl chloride (59.4 mg, 468 umol, 41.0 uL, 4.10 eq) was added and reacted at this temperature for 10 minutes, 2-((2S)-4-(7-(8-ethynyl-7-fluoro-3-hydroxynaphth-1-yl)-6,8-difluoro-2-((tetrahydro-1H-pyrrolizin-7a(5H)-yl)methoxy)quinazolin-4-yl)piperazin-2-yl)acetonitrile (70.0 mg, 114 umol, 1.00 eq) dissolved in N,N-dimethylformamide (1.00 mL) was then added, a solution of N,N-diisopropylethylamine (44.3 mg, 342 umol, 59.7 uL, 3.00 eq) was then added, and the resulting mixture was stirred at 0° C. for 30 minutes. The reaction was complete as monitored by LCMS. The reaction liquid was filtered, and the filtrate was separated and purified by prep-HPLC and SFC to obtain 2-((2S)-4-(7-(8-ethynyl-7-fluoro-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile as a yellow solid.

After SFC resolution, two target compound peaks 20A and 20B were obtained. Compounds 20A and 20B were axially chiral isomers of each other.

20A: (5.3 mg, yield 6.80%), LCMS: m/z 685.6 [M+H]⁺, SFC: 97.1 ee %.

¹H NMR (400 MHz, CD₃CN): 6=7.91 (dd, J=5.8, 9.1 Hz, 1H), 7.50 (br d, J=10.0 Hz, 1H), 7.41 (d, J=2.1 Hz, 1H), 7.37 (t, J=9.0 Hz, 1H), 7.21 (d, J=1.8 Hz, 1H), 5.37-5.18 (m, 2H), 5.02-4.83 (m, 1H), 4.32-4.20 (m, 2H), 4.11 (s, 2H), 3.55-3.46 (m, 1H), 3.44-3.31 (m, 1H), 3.21-3.05 (m, 2H), 3.02-2.92 (m, 3H), 2.60 (td, J=6.8, 9.9 Hz, 2H), 2.11-2.10 (m, 4H), 1.85-1.81 (m, 4H), 1.66-1.61 (m, 2H) ppm.

20B: (5.7 mg, yield 7.30%), LCMS: m/z 685.6 [M+H]⁺, SFC: 98.7 ee %.

¹H NMR (400 MHz, CD₃CN): 6=7.91 (dd, J=5.8, 9.1 Hz, 1H), 7.53 (dd, J=1.5, 9.8 Hz, 1H), 7.41 (d, J=2.4 Hz, 1H), 7.36 (t, J=9.0 Hz, 1H), 7.20 (d, J=2.0 Hz, 1H), 5.37-5.18 (m, 2H), 5.02-4.79 (m, 1H), 4.30-4.18 (m, 2H), 4.16 (s, 2H), 3.64-3.51 (m, 1H), 3.45-3.32 (m, 1H), 3.24-3.00 (m, 4H), 2.89 (br dd, J=6.8, 17.1 Hz, 1H), 2.67-2.62 (m, 2H), 2.10 (br dd, J=3.2, 5.6 Hz, 4H), 1.86-1.81 (m, 4H), 1.70-1.64 (m, 2H) ppm.

### Example 21: 2-((2S)-4-(7-(8-ethynylnaphth-1-yl)-6,8-difluoro-2-((2R,7aS)-2-fluorotetrahydro-1H-pyrrolizin-7a(5H)-ylmethoxy)quinazolin-4-yl)-1-(2-fluoroacryloyl)piperazin-2-yl)acetonitrile (Compound 21)

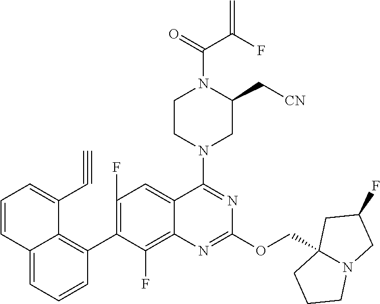 [SMILES: C#Cc1cccc2cccc(-c3c(F)cc4c(N5CCN(C(=O)C(=C)F)[C@@H](CC#N)C5)nc(OC[C@@]56CCCN5C[C@H](F)C6)nc4c3F)c12]

The specific synthesis route of Compound 21 was as follows:

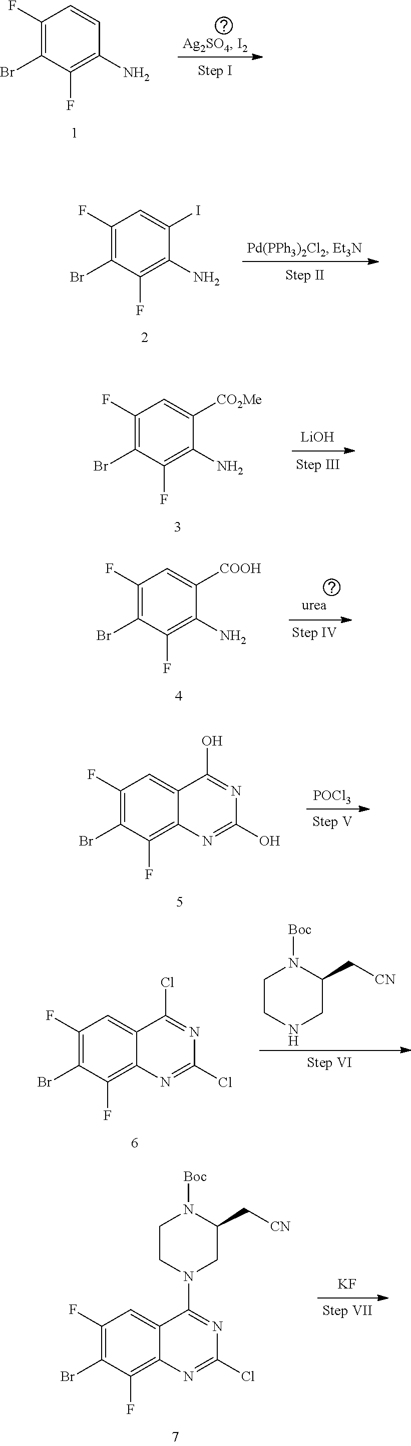

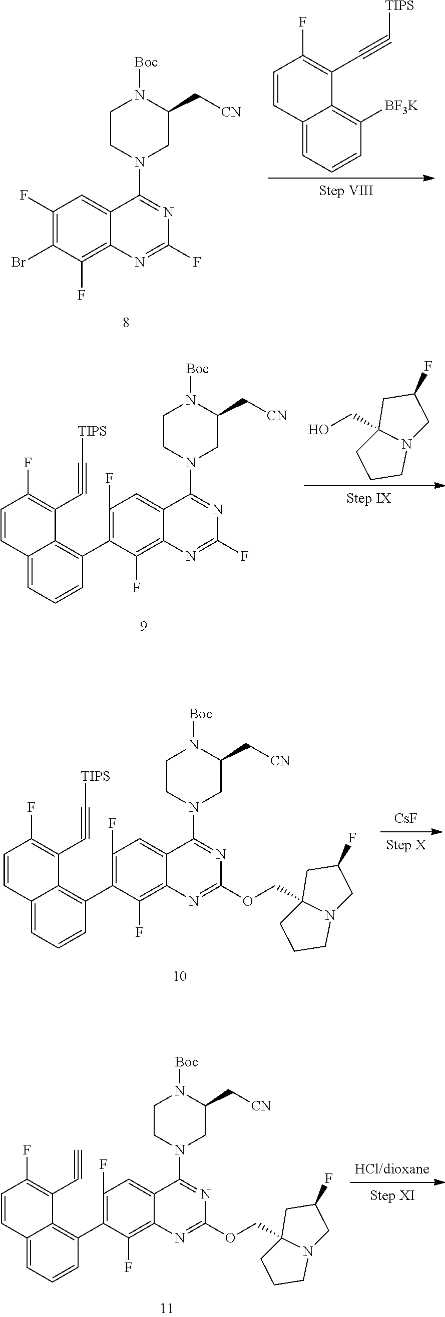

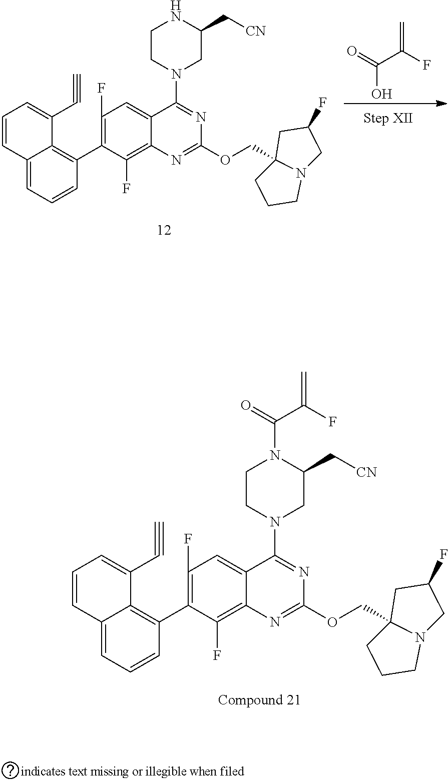

Starting from the intermediate No. 6 compound, the specific synthesis process of Compound 21 was as follows:

### Step VIII: Synthesis of tert-butyl (2S)-2-(cyanomethyl)-4-(2,6,8-trifluoro-7-(8-((triisopropylsilyl)ethynyl)naphth-1-yl)quinazolin-4-yl)piperazine-1-carboxylate

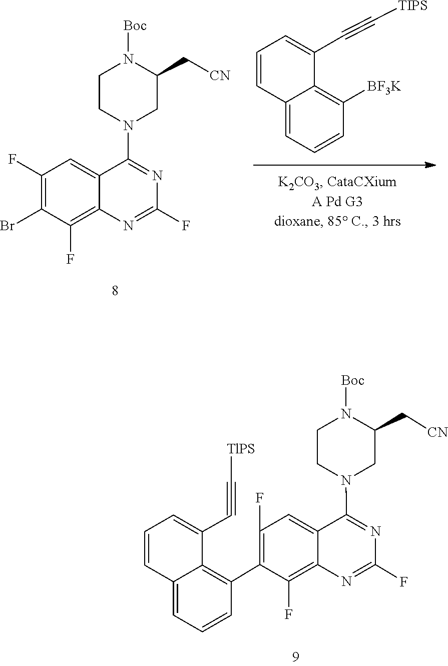

Under nitrogen protection, tert-butyl (S)-4-(7-bromo-2,6,8-trifluoroquinazolin-4-yl)-2-(cyanomethyl)piperazine-1-carb oxylate (100 mg, 205 umol), potassium (8-((triisopropylsilyl)ethynyl)naphth-1-yl)tetrafluoroboronate (136 mg, 329 umol), methylsulfonyloxy(diadamantyl-n-butylphosphino)-2′-amino-1,1′-biphenyl-2-yl)palladium(II) (29.9 mg, 41.1 umol), and potassium carbonate (100 mg, 723 umol) were dissolved in dioxane (2 mL) and water (2.5 mL) and then reacted at 85° C. for 3 hours. The reaction was complete as detected by LCMS. The reaction liquid was concentrated and purified by column chromatography to obtain tert-butyl (2S)-2-(cyanomethyl)-4-(2,6,8-trifluoro-7-(8-((triisopropylsilyl)ethynyl)naphth-1-yl)quinazolin-4-yl)piperazine-1-carboxylate as a yellow solid (300 mg, yield 51.1%).

LCMS: m/z 713.9 (M+H)+.

¹H NMR (400 MHz, CDCl₃): δ=8.04-7.93 (m, 2H), 7.83 (d, J=7.1 Hz, 1H), 7.60 (t, J=7.6 Hz, 1H), 7.52-7.40 (m, 3H), 4.78-4.63 (m, 1H), 4.38-4.07 (m, 3H), 3.75-3.45 (m, 3H), 2.89-2.64 (m, 2H), 1.54 (d, J=6.5 Hz, 9H), 0.91-0.83 (m, 18H), 0.65-0.52 (m, 3H) ppm.

### Step IX: Synthesis of tert-butyl (2S)-2-(cyanomethyl)-4-(6,8-difluoro-2-(((2R,7aS)-2-fluorotetrahydro-1H-pyrrolizin-7a(5H)-yl)methoxy)-7-(8-((triisopropylsilyl)ethynyl)naphth-1-yl)quinazolin-4-yl)piperazine-1-carboxylate

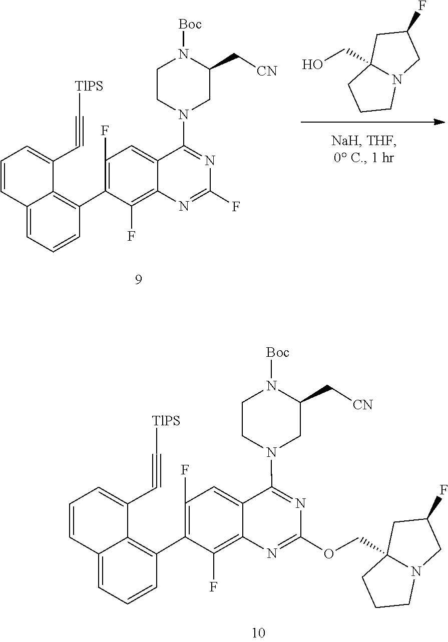

((2R,7aS)-2-fluorotetrahydro-1H-pyrrolizin-7a(5H)-yl)methanol (267 mg, 1.68 mmol) was dissolved in tetrahydrofuran (10 mL), sodium hydride (68.9 mg, 1.72 mmol) was added at 0° C. under nitrogen protection, and the mixture was heated to 20° C. and reacted for 1 hour. Tert-butyl (2S)-2-(cyanomethyl)-4-(2,6,8-trifluoro-7-(8-((triisopropylsilyl)ethynyl)naphth-1-yl)quinazolin-4-yl)piperazine-1-carboxylate (300 mg, 420 umol) was then added at 20° C. and reacted for 30 minutes. The reaction was complete as detected by LCMS. The reaction product was quenched by adding water at 0° C., extracted with ethyl acetate, washed with a saturated sodium chloride aqueous solution, dried, and concentrated. The crude product was purified by column chromatography to obtain tert-butyl (2S)-2-(cyanomethyl)-4-(6,8-difluoro-2-(((2R,7aS)-2-fluorotetrahydro-1H-pyrrolizin-7a(5H)-yl)methoxy)-7-(8-((triisopropylsilyl)ethynyl)naphth-1-yl)quinazolin-4-yl)piperazine-1-carboxylate as a yellow solid (300 mg, yield 83.6%).

LCMS: m/z 853.1 (M+H)+.

### Step X: Synthesis of tert-butyl (2S)-2-(cyanomethyl)-4-(7-(8-ethynylnaphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate

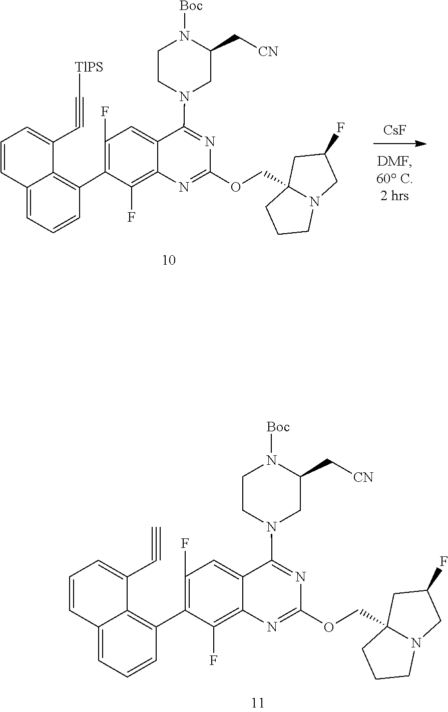

Under nitrogen protection, tert-butyl (2S)-2-(cyanomethyl)-4-(6,8-difluoro-2-(((2R,7aS)-2-fluorotetrahydro-1H-pyrrolizin-7a(5H)-yl)methoxy)-7-(8-((triisopropylsilyl)ethynyl)naphth-1-yl)quinazolin-4-yl)piperazine-1-carboxylate (300 mg, 351 umol) and cesium fluoride (213 mg, 1.41 mmol) were dissolved in N,N-dimethylformamide (10 mL) and reacted at 60° C. for 1 hour. The reaction was complete as detected by LCMS. Water was added to the reaction liquid, which was extracted with ethyl acetate, and the organic phase was washed with a saturated sodium chloride aqueous solution, dried, concentrated, and then purified by column chromatography to obtain tert-butyl (2S)-2-(cyanomethyl)-4-(7-(8-ethynylnaphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate (240 mg, yield 97.9%).

LCMS: m/z 696.8 (M+H)+.

¹H NMR (400 MHz, CDCl₃): δ=8.04-7.94 (m, 3H), 7.76 (d, J=7.0 Hz, 1H), 7.60 (t, J=7.7 Hz, 1H), 7.52-7.43 (m, 2H), 5.41-5.14 (m, 2H), 4.67 (br s, 1H), 4.33-4.15 (m, 4H), 4.14-3.99 (m, 1H), 3.68-3.56 (m, 1H), 3.33-3.12 (m, 6H), 3.09-2.97 (m, 2H), 1.93-1.85 (m, 6H), 1.53 (s, 9H) ppm.

### Step XI: Synthesis of 2-((2S)-4-(7-(8-ethynylnaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile

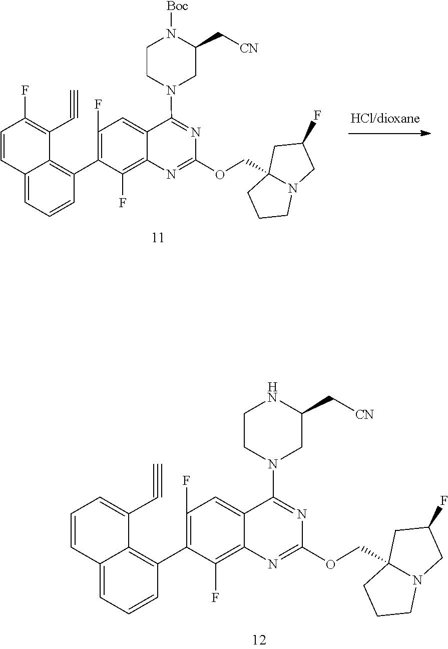 [SMILES: C#Cc1c(F)ccc2cccc(-c3c(F)cc4c(N5CCN(C(=O)OC(C)(C)C)[C@@H](CC#N)C5)nc(OC[C@@]56CCCN5C[C@H](F)C6)nc4c3F)c12 ; C#Cc1cccc2cccc(-c3c(F)cc4c(N5CCN[C@@H](CC#N)C5)nc(OC[C@@]56CCCN5C[C@H](F)C6)nc4c3F)c12]

Tert-butyl (2S)-2-(cyanomethyl)-4-(7-(8-ethynylnaphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate (240 mg, 344 umol) was dissolved in hydrochloric acid/dioxane (10 mL), and after nitrogen displacement three times, the mixture was reacted at 0° C. for 30 minutes. The reaction was complete as detected by LCMS. The reaction liquid was concentrated to obtain a crude product of 2-((2S)-4-(7-(8-ethynylnaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile (200 mg, crude).

LCMS: m/z 596.6 (M+H)+.

### Step XII: Synthesis of 2-((2S)-4-(7-(8-ethynylnaphth-1-yl)-6,8-difluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

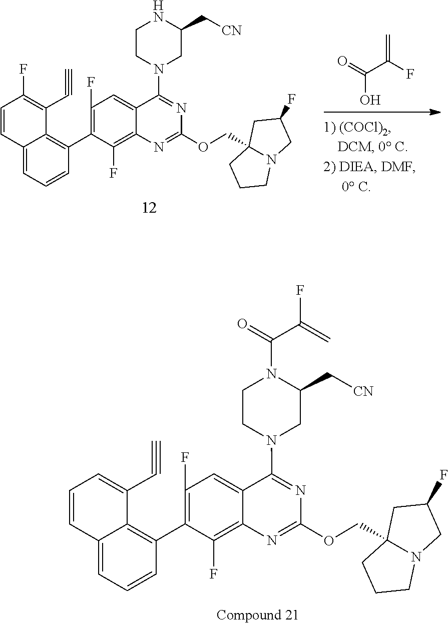

After 2-fluoroacrylic acid ((120 mg, 1.34 mmol) was dissolved in dichloromethane (10 mL) at 0° C., oxalyl chloride (174 mg, 1.37 mmol) was added and reacted at this temperature for 10 minutes, the reaction product was then added to a solution of 2-((2S)-4-(7-(8-ethynylnaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile (200 mg, 335 umol) and N,N-diisopropylethylamine (129 mg, 1.01 mmol) in N,N-dimethylformamide (2 mL), and the resulting mixture was stirred at 0° C. for 30 minutes. The reaction was complete as monitored by LCMS. The reaction liquid was filtered and subjected to prep-HPLC and SFC for separation and purification to obtain 2-((2S)-4-(7-(8-ethynylnaphth-1-yl)-6,8-difluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile as a yellow solid. The product was subjected to SFC chiral resolution to obtain two target compound peaks 21A and 21B. Compounds 21A and 21B were axially chiral isomers of each other.

21A: (22 mg, yield 9.81%), LCMS: m/z 669.5 (M+H)+.

¹H NMR (400 MHz, CD₃CN): 6=8.11 (ddd, J₁=1.1, J₂=8.3, J₃=12.1 Hz, 2H), 7.78 (dd, J₁=1.1, J₂=7.1 Hz, 1H), 7.71-7.66 (m, 1H), 7.60-7.51 (m, 3H), 5.35-5.24 (m, 2H), 5.22-5.17 (m, 1H), 5.08-4.77 (m, 1H), 4.27 (br d, J=13.6 Hz, 1H), 4.22-4.15 (m, 2H), 3.84-3.49 (m, 2H), 3.44-3.32 (m, 1H), 3.16-3.05 (m, 4H), 2.97-2.86 (m, 3H), 2.20-2.17 (m, 1H), 2.11-2.01 (m, 3H), 1.92 (br s, 4H) ppm.

21B: (23.3 mg, yield 10.3%), LCMS: m/z 669.5 (M+H)+.

¹H NMR (400 MHz, CD₃CN): 6=8.11 (ddd, J₁=0.9, J₂=8.3, J₃=12.0 Hz, 2H), 7.78 (dd, J₁=1.1, J₂=7.3 Hz, 1H), 7.72-7.66 (m, 1H), 7.59-7.53 (m, 2H), 7.50 (dd, J₁=1.8, J₂=9.9 Hz, 1H), 5.35-5.25 (m, 2H), 5.23-5.16 (m, 1H), 5.04-4.80 (m, 1H), 4.30-4.22 (m, 2H), 4.20-4.16 (m, 1H), 4.10-4.06 (m, 1H), 3.52 (br dd, J₁=2.9, J₂=14.1 Hz, 1H), 3.45-3.35 (m, 1H), 3.16-3.05 (m, 4H), 3.00-2.93 (m, 1H), 2.93-2.86 (m, 2H), 2.20-2.17 (m, 1H), 2.11-2.04 (m, 3H), 1.92-1.74 (m, 4H) ppm.

### Example 22: 2-((2S)-4-(7-(8-chloro-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile (Compound 22)

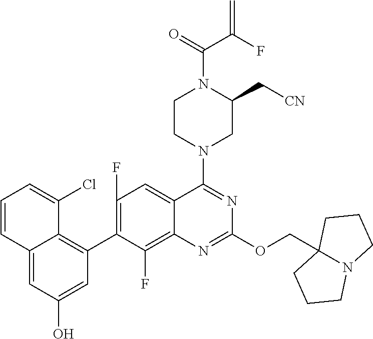 [SMILES: C=C(F)C(=O)N1CCN(c2nc(OCC34CCCN3CCC4)nc3c(F)c(-c4cc(O)cc5cccc(Cl)c45)c(F)cc23)C[C@@H]1CC#N]

The specific synthesis route of Compound 22 was as shown below:

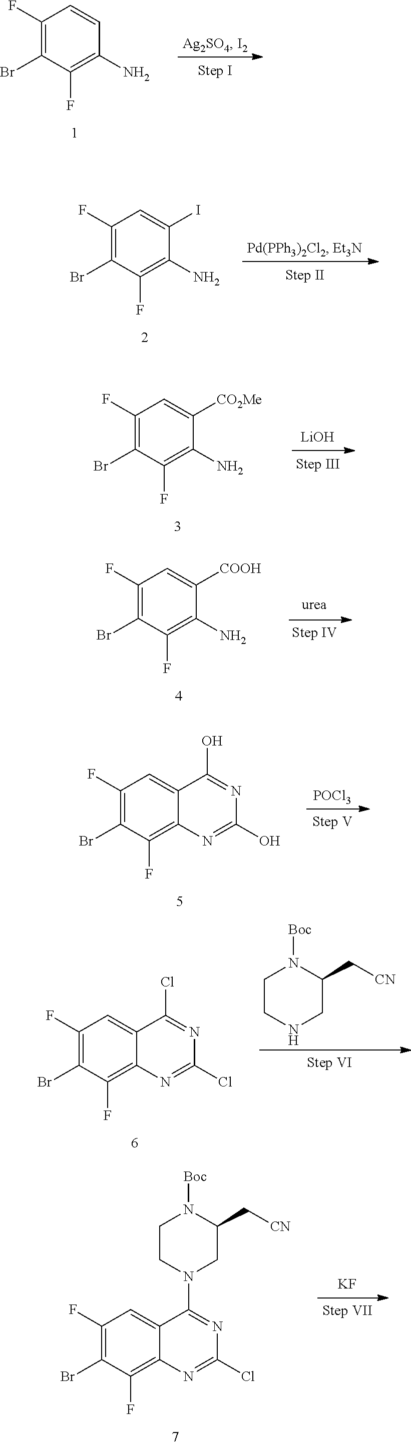

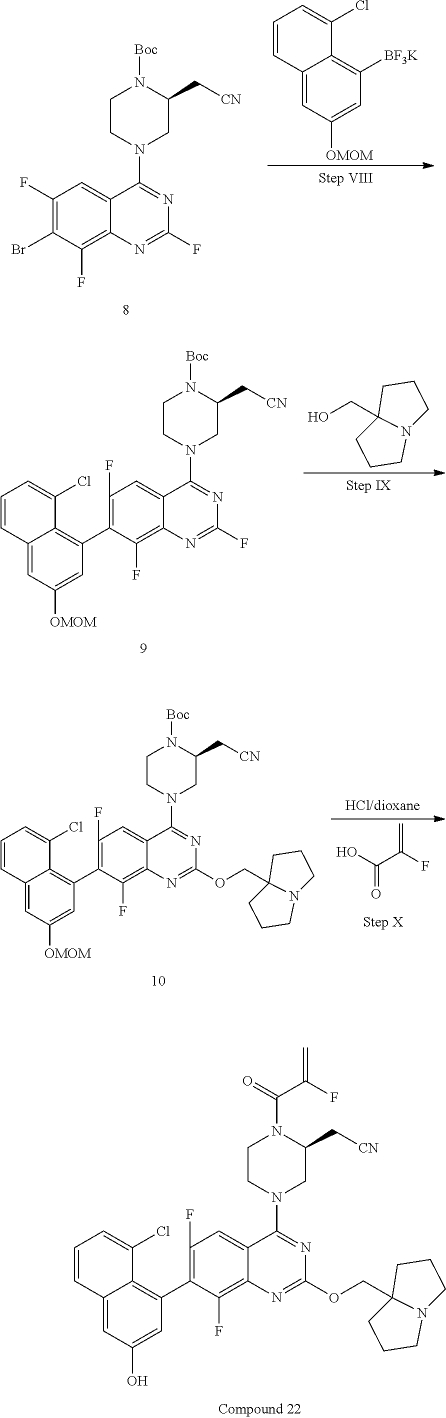

Starting from the intermediate No. 6 compound, the specific synthesis process of Compound 22 was as follows:

### Step VIII: Synthesis of tert-butyl (2S)-4-(7-(8-chloro-3-(methoxymethoxy)naphth-1-yl)-2,6,8-trifluoroquinazolin-4-yl)-2-(cyanomethyl)piperazine-1-carboxylate

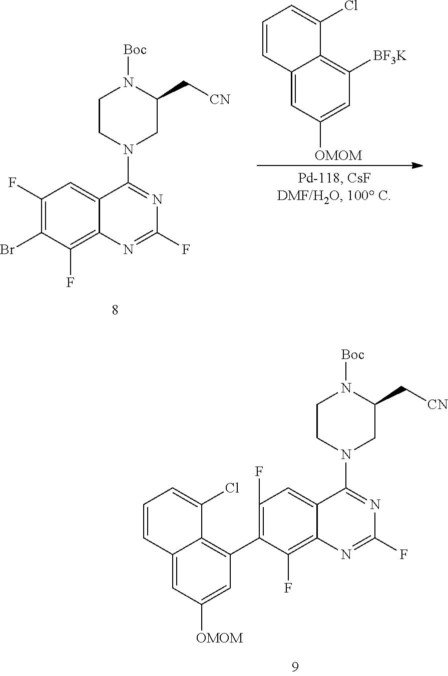

Tert-butyl (S)-4-(7-bromo-2,6,8-trifluoroquinazolin-4-yl)-2-(cyanomethyl)piperazine-1-carboxylate (700 mg, 1.44 mmol), potassium (8-chloro-3-(methoxymethoxy)-1-naphthalene)-fluoroboronate (700 mg, 2.13 mmol), cesium fluoride (99.9 mg, 658 umol), and 1,1-bis(tert-butylphosphino)ferrocene palladium chloride (187 mg, 287 umol) were dissolved in water (20.0 mL) and N,N-dimethylformamide (50.0 mL), then aerated, and finally reacted at 100° C. under nitrogen protection for 15 minutes. The reaction was complete as monitored by LCMS. The reaction liquid was extracted with water and ethyl acetate, the organic phase was washed with a saturated sodium chloride aqueous solution and dried over sodium sulfate, and after filtration, concentration and purification by column chromatography, tert-butyl (2S)-4-(7-(8-chloro-3-(methoxymethoxy)naphth-1-yl)-2,6,8-trifluoroquinazolin-4-yl)-2-(cyanomethyl)piperazine-1-carboxylate (500 mg, yield 55.3%) was obtained.

¹H NMR (400 MHz, CDCl₃): δ=7.71 (dd, J₁=1.6, J₂=7.8 Hz, 1H), 7.51 (d, J=2.5 Hz, 1H), 7.38 (br dd, J₁=1.8, J₂=9.3 Hz, 1H), 7.36-7.28 (m, 2H), 7.13 (t, J=2.6 Hz, 1H), 5.25 (s, 2H), 4.62 (br dd, J₁=3.6, J₂=5.9 Hz, 1H), 4.39-4.24 (m, 2H), 4.14-4.06 (m, 1H), 3.82-3.68 (m, 1H), 3.62-3.38 (m, 5H), 2.92-2.60 (m, 2H), 1.46 (s, 9H) ppm.

### Step IX: Synthesis of tert-butyl (2S)-4-(7-(8-chloro-3-(methoxymethoxy)naphth-1-yl)-6,8-difluoro-2-((tetrahydro-1H-pyrrolizin-7a(5H)-yl)methoxy)quinazolin-4-yl)-2-(cyanomethyl)piperazine-1-carboxylate

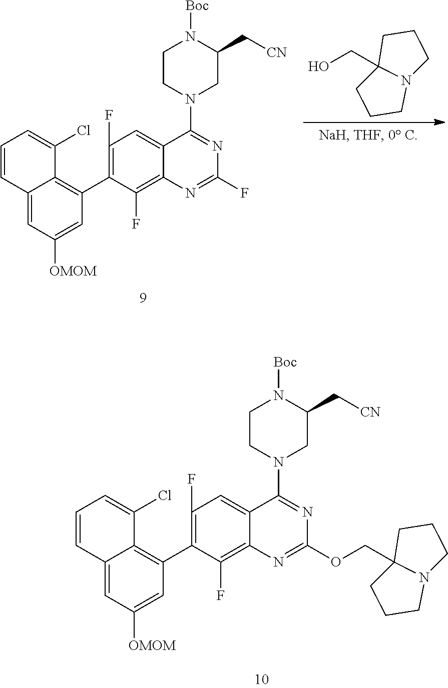

After (tetrahydro-1H-pyrrolizin-7a(5H)-yl)methanol (134 mg, 955 umol) was dissolved in tetrahydrofuran (6.00 mL) at 0° C. under nitrogen protection, sodium hydride (50.9 mg, 1.27 mmol, 60% purity) was added and then reacted at 20° C. for 1 hour. Tert-butyl (2S)-4-(7-(8-chloro-3-(methoxymethoxy)naphth-1-yl)-2,6,8-trifluoroquinazolin-4-yl)-2-(cyanomethyl)piperazine-1-carboxylate (200 mg, 318 umol) was then added and reacted for 2 hours. The reaction was complete as monitored by LCMS. The reaction liquid was quenched by adding water at 0° C., extracted with ethyl acetate, washed with a saturated sodium chloride aqueous solution, and dried over sodium sulfate, and after filtration, concentration and purification by column chromatography, tert-butyl (2S)-4-(7-(8-chloro-3-(methoxymethoxy)naphth-1-yl)-6,8-difluoro-2-((tetrahydro-1H-pyrrolizin-7a(5H)-yl)methoxy)quinazolin-4-yl)-2-(cyanomethyl)piperazine-1-carboxylate (200 mg, yield 83.8%) was obtained.

¹H NMR (400 MHz, CDCl₃): δ=7.69 (dd, J₁=1.5, J₂=7.9 Hz, 1H), 7.49 (d, J=2.4 Hz, 1H), 7.37-7.25 (m, 3H), 7.14 (dd, J₁=2.8, J₂=3.8 Hz, 1H), 4.80-4.66 (m, 2H), 4.64-4.52 (m, 1H), 4.50-4.35 (m, 1H), 4.28-4.18 (m, 1H), 4.11-4.04 (m, 1H), 3.98-3.85 (m, 2H), 3.82-3.66 (m, 1H), 3.62-3.51 (m, 1H), 3.50-3.44 (m, 3H), 3.37-3.22 (m, 1H), 3.22-3.07 (m, 1H), 2.99-2.76 (m, 3H), 2.49-2.28 (m, 2H), 2.28-2.16 (m, 2H), 2.13-2.01 (m, 3H), 1.98-1.89 (m, 3H), 1.44 (s, 9H) ppm.

### Step X: Synthesis of 2-((2S)-4-(7-(8-chloro-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile

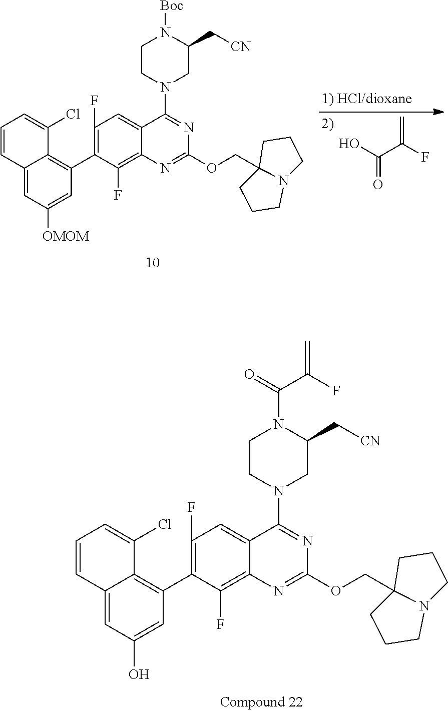

Solution A: At 0° C., tert-butyl (2S)-4-(7-(8-chloro-3-(methoxymethoxy)naphth-1-yl)-6,8-difluoro-2-((tetrahydro-1H-pyrrolizin-7a(5H)-yl)methoxy)quinazolin-4-yl)-2-(cyanomethyl)piperazine-1-carboxylate (100 mg, 133 umol) was dissolved in dioxane hydrochloride (4.00 M, 9.98 mL) and stirred for one hour. After the reaction liquid was concentrated, N,N-dimethylformamide (2.00 mL) and N,N-diisopropylethylamine (51.7 mg, 400 umol) were added.

Solution B: At 0° C., 2-fluoroacrylic acid (14.4 mg, 160 umol) was dissolved in dichloromethane (1.00 mL) and N,N-dimethylformamide, oxalyl chloride (25.4 mg, 200 umol) was added and reacted at this temperature for 20 minutes, and the reaction was then carried out at 0° C. for 30 minutes. At 0° C., solution B was added to solution A and stirred for 20 minutes. The reaction was complete as monitored by LCMS. The reaction liquid was filtered and subjected to prep-HPLC and SFC for separation and purification to obtain 2-((2S)-4-(7-(8-chloro-3-hydroxynaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile. The product was subjected to SFC chiral resolution to obtain two peaks 22A and 22B of the target compound. Compounds 22A and 22B were axially chiral isomers of each other.

22A: (25.0 mg, yield 27.4%, purity 99%), LCMS: m/z 677.5 (M+H)+.

¹H NMR (400 MHz, CD₃CN): 6=7.80 (dd, J=2.4, J₂=7.1 Hz, 1H), 7.54 (dd, J₁=1.6, J₂=9.9 Hz, 1H), 7.44-7.35 (m, 3H), 7.12 (d, J=2.6 Hz, 1H), 5.39-5.15 (m, 2H), 4.91 (br s, 1H), 4.33-3.98 (m, 5H), 3.93-3.45 (m, 2H), 3.33 (br s, 1H), 3.08 (br dd, J₁=7.8, J₂=16.3 Hz, 1H), 3.02-2.94 (m, 2H), 2.86 (dd, J₁=6.7, J₂=17.2 Hz, 1H), 2.66-2.55 (m, 2H), 2.11 (br dd, J₁=2.4, J₂=4.9 Hz, 2H), 1.87-1.79 (m, 4H), 1.68-1.59 (m, 2H) ppm.

22B: (23.0 mg, yield 25.2%, purity 99%), LCMS: m/z 677.5 (M+H)+.

¹H NMR (400 MHz, CD₃CN): 6=7.81 (dd, J=2.6, J₂=6.9 Hz, 1H), 7.55 (dd, J₁=1.6, J₂=9.9 Hz, 1H), 7.47-7.33 (m, 3H), 7.15 (d, J=2.5 Hz, 1H), 5.38-5.13 (m, 2H), 4.95 (br s, 1H), 4.35-3.91 (m, 5H), 3.87-3.43 (m, 2H), 3.34 (br s, 1H), 3.09 (br s, 1H), 3.02-2.89 (m, 3H), 2.61 (td, J₁=6.8, J₂=9.9 Hz, 2H), 1.87-1.80 (m, 4H), 1.64 (td, J₁=7.3, J₂=12.3 Hz, 2H) ppm.

### Example 23: 2-((2S)-4-(7-(8-ethynyl-7-fluoronaphth-1-yl)-6,8-difluoro-2-((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile (Compound 23)

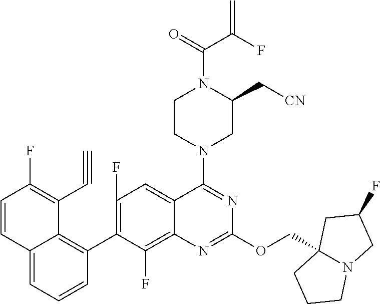 [SMILES: C#Cc1c(F)ccc2cccc(-c3c(F)cc4c(N5CCN(C(=O)C(=C)F)[C@@H](CC#N)C5)nc(OC[C@@]56CCCN5C[C@H](F)C6)nc4c3F)c12]

The specific synthesis route of Compound 23 was as shown below:

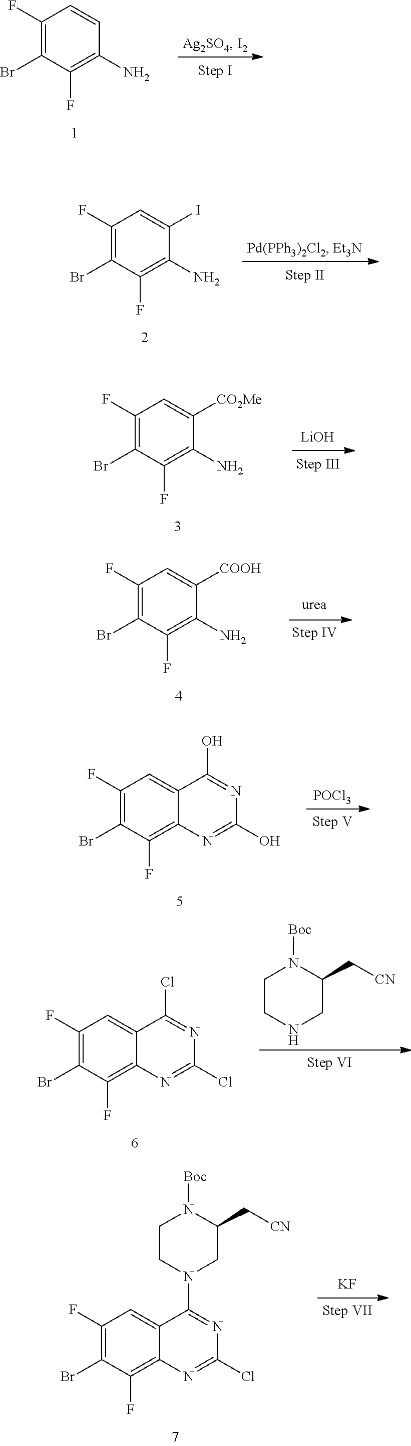

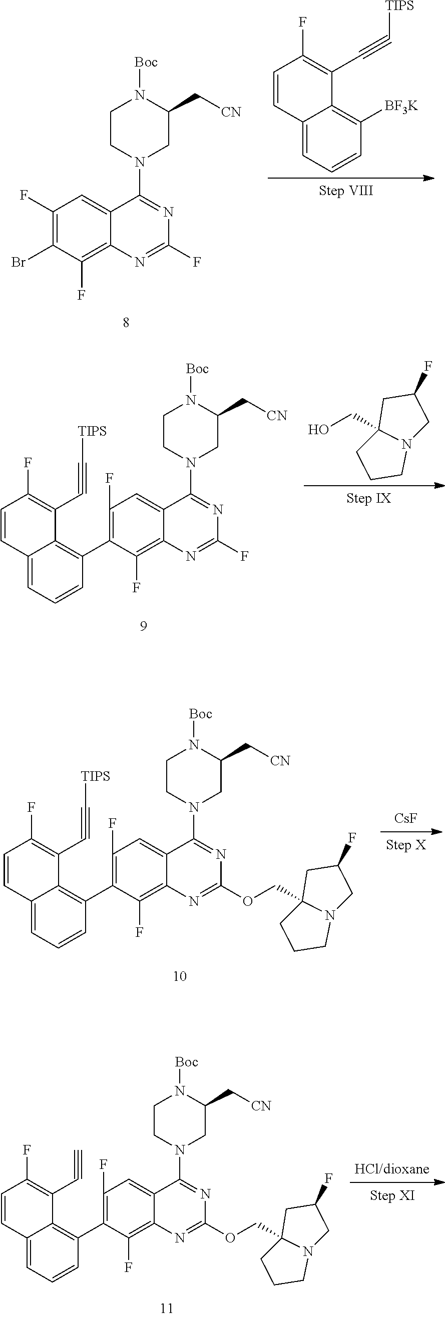

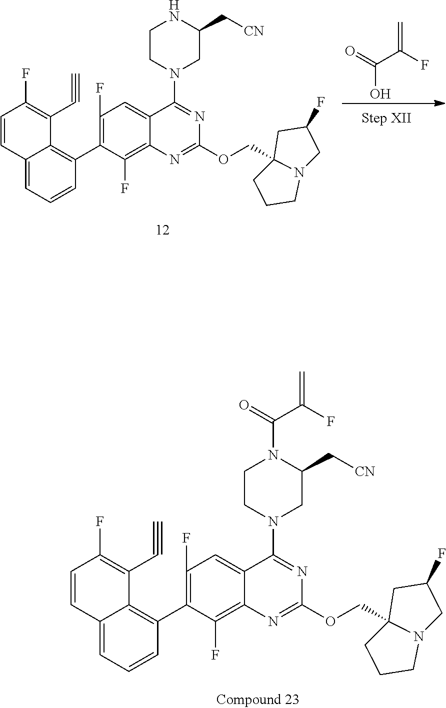

Starting from the intermediate No. 6 compound, the specific synthesis process of Compound 23 was as follows:

### Step VIII: Synthesis of tert-butyl (2S)-2-(cyanomethyl)-4-(2,6,8-trifluoro-7-(7-fluoro-8-((triisopropylsilyl) ethynyl) naphth-1-yl)-quinazolin-4-yl) piperazine-1-carboxylate

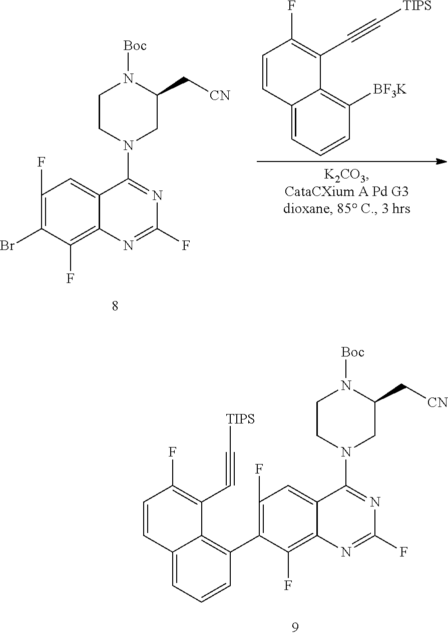

After tert-butyl (S)-4-(7-bromo-2,6,8-trifluoroquinazolin-4-yl)-2-(cyanomethyl)piperazine-1-carboxylate (200 mg, 411 umol) and potassium (7-fluoro-8-((triisopropylsilyl)ethynyl)naphth-1-yl)fluoroboronate (279 mg, 616 umol) were dissolved in dioxane (4 mL) and water (0.4 mL), methylsulfonyloxy(diadamantyl-n-butylphosphino)-2-amino-1,1-biphenyl-2-yl)palladium(II) (59.9 mg, 82.2 umol) and potassium carbonate (170 mg, 1.23 mmol) were added. The reaction liquid was reacted at 80° C. for 3 hours. The reaction was complete as monitored by LCMS. The reaction liquid was concentrated, and the crude product was subjected to column chromatography to obtain tert-butyl (2S)-2-(cyanomethyl)-4-(2,6,8-trifluoro-7-(7-fluoro-8-((triisopropylsilyl)ethynyl)naphth-1-yl)-quinazolin-4-yl)piperazine-1-carboxylate (350 mg, yield 58.1%) as a yellow solid.

LCMS: m/z 732.3 [M+H]⁺.

¹H NMR (400 MHz, CDCl₃): δ=8.03-7.89 (m, 2H), 7.60-7.52 (m, 1H), 7.50-7.33 (m, 3H), 4.78-4.62 (m, 1H), 4.40-4.11 (m, 3H), 3.74-3.65 (m, 1H), 3.62-3.17 (m, 2H), 2.92-2.65 (m, 2H), 1.25 (s, 9H), 0.94-0.81 (m, 18H), 0.63-0.51 (m, 3H) ppm.

### Step IX: Synthesis of tert-butyl (2S)-2-(cyanomethyl)-4-(6,8-difluoro-7-(7-fluoro-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate

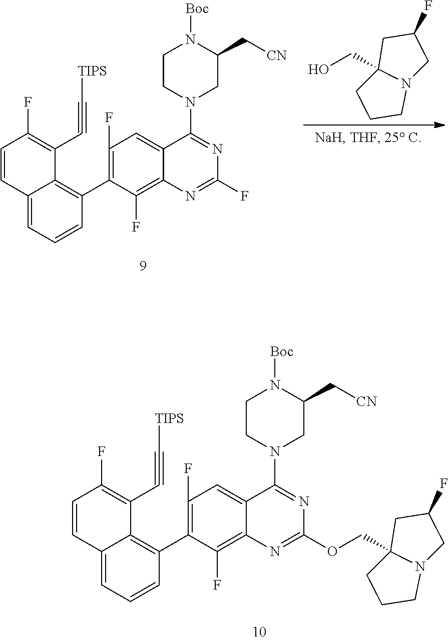

After ((2R,7aS)-2-fluoro-tetrahydro-1H-pyrrolizin-7a(5H)-yl)methanol (304 mg, 1.91 mmol) was dissolved in tetrahydrofuran (5 mL) under nitrogen protection at 0° C., sodium hydride (99.7 mg, 2.49 mmol) was added and then reacted at 20° C. for 1 hour, and tert-butyl (2S)-2-(cyanomethyl)-4-(2,6,8-trifluoro-7-(7-fluoro-8-((triisopropylsilyl)ethynyl)naphth-1-yl)-quinazolin-4-yl)piperazine-1-carboxylate (350 mg, 478 umol) was then added and reacted at 20° C. for 30 minutes. The reaction was complete as monitored by LCMS. The reaction liquid was quenched by adding water at 0° C. and extracted with ethyl acetate, and the organic phase was washed with a sodium chloride aqueous solution, dried over sodium sulfate, filtered, and concentrated to obtain tert-butyl (2S)-2-(cyanomethyl)-4-(6,8-difluoro-7-(7-fluoro-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate (270 mg, yield 64.8%) as a yellow solid.

LCMS: m/z 871.5 [M+H]⁺.

¹H NMR (400 MHz, CDCl₃): δ=8.00-7.89 (m, 2H), 7.58-7.52 (m, 1H), 7.48-7.43 (m, 1H), 7.39-7.28 (m, 2H), 5.40-5.14 (m, 1H), 4.77-4.67 (m, 1H), 4.39-4.22 (m, 2H), 4.22-4.15 (m, 2H), 3.57-3.34 (m, 2H), 3.32-3.16 (m, 4H), 3.10-2.91 (m, 2H), 2.38-2.08 (m, 4H), 2.01-1.88 (m, 4H), 1.54 (d, J=3.5 Hz, 9H), 0.92-0.81 (m, 18H), 0.63-0.49 (m, 3H) ppm.

### Step X: Synthesis of tert-butyl (2S)-2-(cyanomethyl)-4-(7-(8-ethynyl-7-fluoronaphth-1-yl)-6,8-difluoro-2-(((2R,7aS)-2-fluorotetrahydro-1H-pyrrolizin-7a(5H)-ylmethoxy)quinazolin-4-yl)piperazine-1-carboxylate

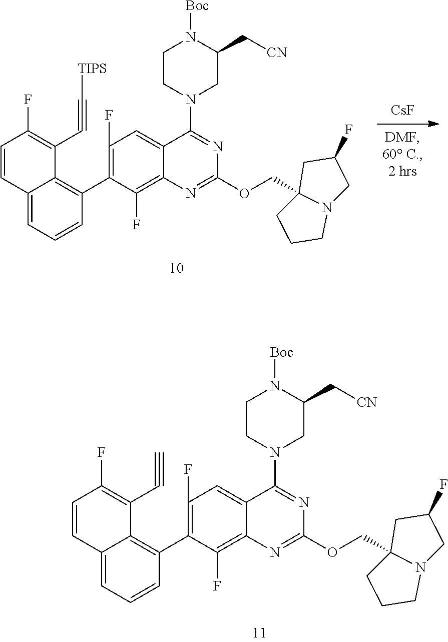

After tert-butyl (2S)-2-(cyanomethyl)-4-(6,8-difluoro-7-(7-fluoro-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate (270 mg, 309 umol) was dissolved in N,N-dimethylformamide (2.5 mL), cesium fluoride (235 mg, 1.55 mmol) was added and reacted at 60° C. for 2 hours. The reaction was complete as monitored by LCMS. Water was added to the reaction liquid, which was extracted with ethyl acetate, and the organic phase was washed with a sodium chloride aqueous solution, dried over sodium sulfate, filtered, and concentrated to obtain tert-butyl (2S)-2-(cyanomethyl)-4-(7-(8-ethynyl-7-fluoronaphth-1-yl)-6,8-difluoro-2-(((2R,7aS)-2-fluorotetrahydro-1H-pyrrolizin-7a(5H)-ylmethoxy)quinazolin-4-yl)piperazine-1-carboxylate (200 mg, 279 umol) as a yellow solid.

LCMS: m/z 715.3 [M+H]⁺.

¹H NMR (400 MHz, CDCl₃): δ=8.03-7.94 (m, 2H), 7.62-7.55 (m, 1H), 7.54-7.47 (m, 1H), 7.40-7.28 (m, 2H), 5.41-5.21 (m, 1H), 4.67 (br s, 1H), 4.39-4.01 (m, 5H), 3.62 (br d, J=12.8 Hz, 1H), 3.48-3.15 (m, 5H), 3.14-2.96 (m, 2H), 2.87-2.72 (m, 2H), 2.37-2.12 (m, 4H), 1.97-1.92 (m, 2H), 1.53 (s, 9H) ppm.

### Step XI: Synthesis of 2-((2S)-4-(7-(8-ethynyl-7-fluoronaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile

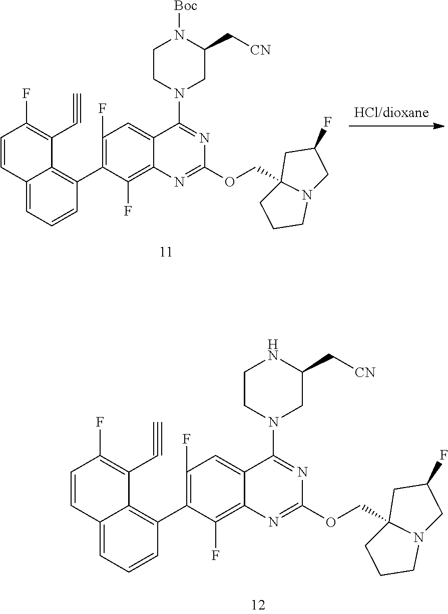 [SMILES: C#Cc1c(F)ccc2cccc(-c3c(F)cc4c(N5CCN(C(=O)OC(C)(C)C)[C@@H](CC#N)C5)nc(OC[C@@]56CCCN5C[C@H](F)C6)nc4c3F)c12 ; C#Cc1c(F)ccc2cccc(-c3c(F)cc4c(N5CCN[C@@H](CC#N)C5)nc(OC[C@@]56CCCN5C[C@H](F)C6)nc4c3F)c12]

Under nitrogen protection, tert-butyl (2S)-2-(cyanomethyl)-4-(7-(8-ethynyl-7-fluoronaphth-1-yl)-6,8-difluoro-2-(((2R,7aS)-2-fluorotetrahydro-1H-pyrrolizin-7a(5H)-ylmethoxy)quinazolin-4-yl)piperazine-1-carboxylate (200 mg, 279 umol) was dissolved in hydrochloric acid/ethyl acetate (5 mL) and then reacted at 0° C. for 30 minutes. The reaction was complete as monitored by LCMS. The reaction liquid was concentrated to obtain 2-((2S)-4-(7-(8-ethynyl-7-fluoronaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile (160 mg, crude) as a yellow solid.

LCMS: m/z 715.3 [M+H]⁺.

### Step XII: Synthesis of 2-((2S)-4-(7-(8-ethynyl-7-fluoronaphth-1-yl)-6,8-difluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

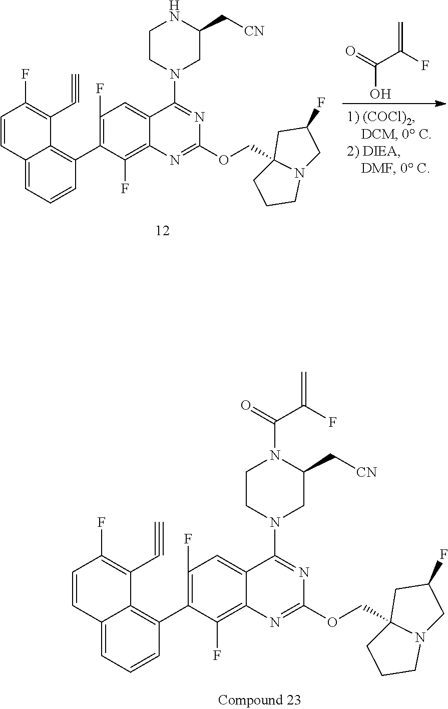

After 2-fluoroacrylic acid (93.7 mg, 1.04 mmol) was dissolved in dichloromethane (4 mL) at 0° C., oxalyl chloride (135 mg, 1.07 mmol) was added and reacted at this temperature for 10 minutes, and then, after 2-((2S)-4-(7-(8-ethynyl-7-fluoronaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile (160 mg, 260 umol) dissolved in N,N-dimethylformamide (2 mL) was added at 0° C., N,N-diisopropylethylamine (100 mg, 780 umol) was added and finally reacted at 0° C. for 30 minutes. The reaction was complete as monitored by LCMS. The reaction liquid was filtered and subjected to prep-HPLC and SFC for separation and purification to obtain 2-((2S)-4-(7-(8-ethynyl-7-fluoronaphth-1-yl)-6,8-difluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile as a white solid. The product was subjected to SFC chiral resolution to obtain two peaks 23A and 23B of the target compound. Compounds 23A and 23B were axially chiral isomers of each other.

23A: (16.2 mg, yield 8.95%), LCMS: m/z 687.5 (M+H)+.

¹H NMR (400 MHz, CD₃CN): 6=8.17-8.10 (m, 2H), 7.72-7.60 (m, 2H), 7.54 (dd, J₁=1.8, J₂=9.8 Hz, 1H), 7.47 (t, J=9.0 Hz, 1H), 5.38-5.16 (m, 3H), 5.07-4.76 (m, 1H), 4.31-4.01 (m, 5H), 3.93-3.51 (m, 2H), 3.45-3.33 (m, 1H), 3.26 (d, J=1.0 Hz, 1H), 3.19-3.03 (m, 4H), 2.96-2.83 (m, 2H), 2.24-2.14 (m, 2H), 2.10-2.03 (m, 2H), 1.89-1.83 (m, 2H) ppm.

23B: (13.9 mg, yield 7.75%), LCMS: m/z 687.5 (M+H)+.

¹H NMR (400 MHz, CD₃CN): δ=8.17-8.10 (m, 2H), 7.71-7.59 (m, 2H), 7.54-7.43 (m, 2H), 5.37-5.14 (m, 3H), 5.07-4.77 (m, 1H), 4.35-4.03 (m, 5H), 3.82-3.32 (m, 3H), 3.22-3.04 (m, 5H), 3.00-2.85 (m, 2H), 2.24-2.14 (m, 2H), 2.10-2.03 (m, 2H), 1.89-1.84 (m, 2H) ppm.

### Example 24: 2-((2S)-4-(7-(8-ethynyl-7-fluoro-3-hydroxynaphth-1-yl)-6,8-difluoro-2-((2R,7aS)-2-fluorotetrahydro-1H-pyrrolizine-7a(5H)-methoxy)quinazolin-4-yl)-1-(2-fluoroacryloyl)piperazin-2-yl)acetonitrile (Compound 24)

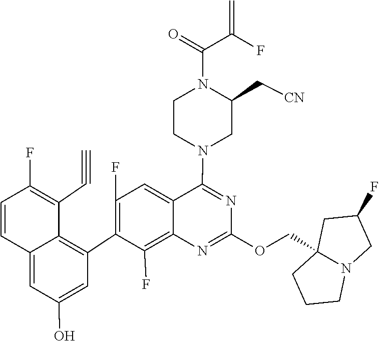 [SMILES: C#Cc1c(F)ccc2cc(O)cc(-c3c(F)cc4c(N5CCN(C(=O)C(=C)F)[C@@H](CC#N)C5)nc(OC[C@@]56CCCN5C[C@H](F)C6)nc4c3F)c12]

The specific synthesis route of Compound 24 was as follows:

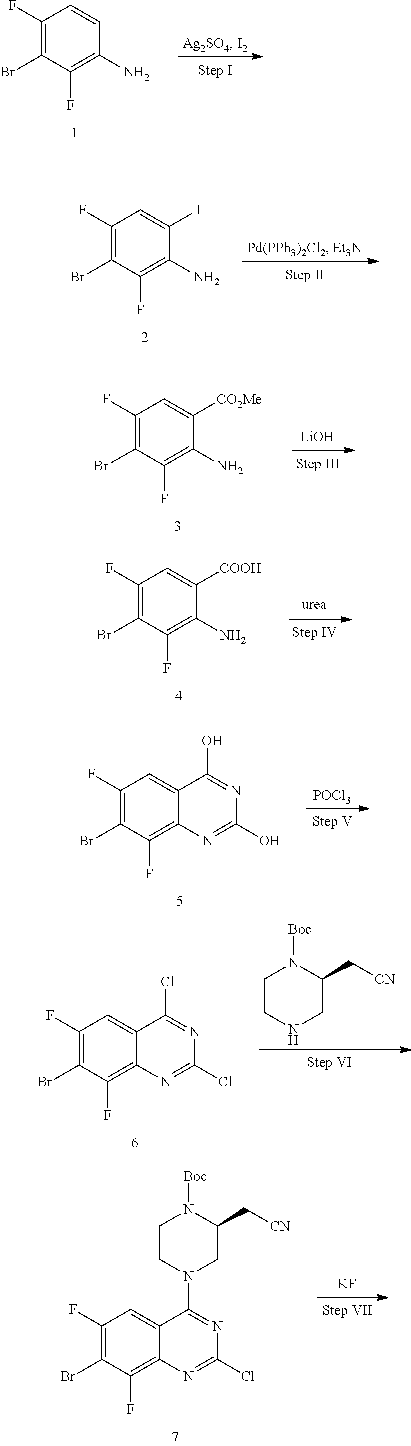

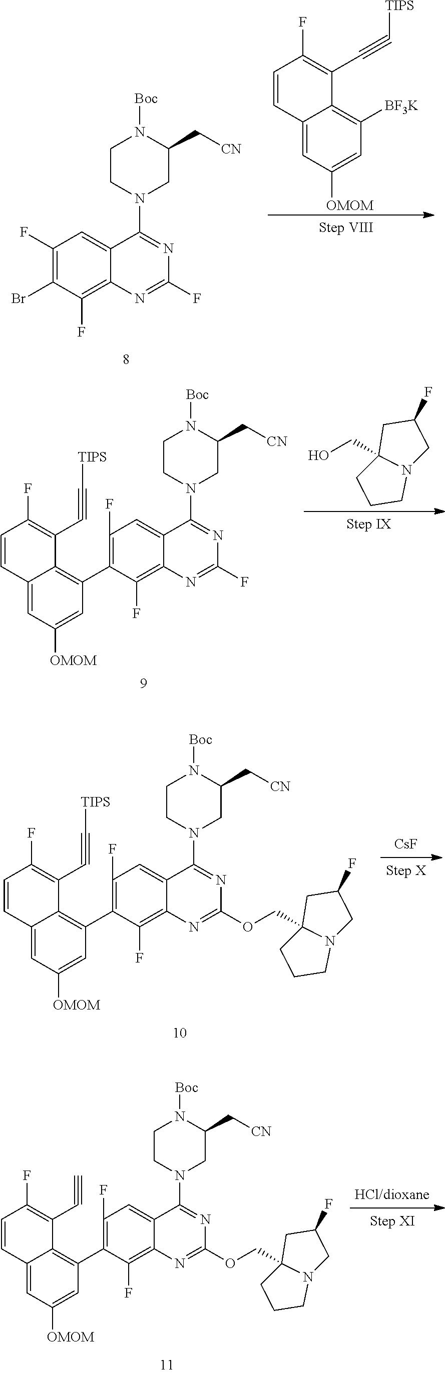

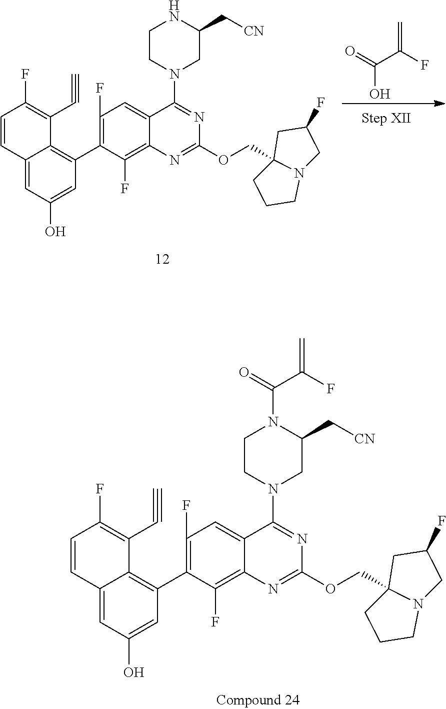

Starting from the intermediate Int-9 in the synthesis route of Compound 20, the specific synthesis steps of Compound 24 were as follows:

### Step IX: Synthesis of tert-butyl (2S′)-2-(cyanomethyl)-4-(6,8-difluoro-7-(7-fluoro-3-(methoxymethoxy)-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate

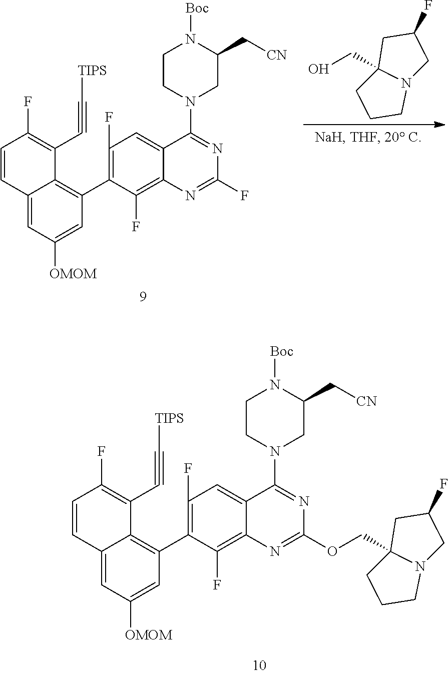

At 0° C. under nitrogen protection, ((2R,7aS)-2-fluorotetrahydro-1H-pyrrolizin-7a(5H)-yl)methanol (402 mg, 2.53 mmol) was dissolved in tetrahydrofuran (10 mL), sodium hydride (103 mg, 2.59 mmol) was added and reacted at 20° C. for 1 hour, and tert-butyl (2S)-2-(cyanomethyl)-4-(2,6,8-trifluoro-7-(7-fluoro-3-(methoxymethoxy)-8-((triisopropylsilyl)ethynyl)naphth-1-yl)quinazolin-4-yl)piperazine-1-carboxylate (500 mg, 631 umol) was then added and reacted for 30 minutes. The reaction was complete as monitored by LCMS. The reaction product was quenched by adding water at 0° C., extracted with ethyl acetate, washed with a sodium chloride aqueous solution, and dried over sodium sulfate, and after filtration, concentration and purification by column chromatography, tert-butyl (2S)-2-(cyanomethyl)-4-(6,8-difluoro-7-(7-fluoro-3-(methoxymethoxy)-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate (500 mg, yield 85.1%) was obtained.

LCMS: m/z 931.6 (M+H)+.

### Step X: Synthesis of tert-butyl (2S)-2-(cyanomethyl)-4-(7-(8-ethynyl-7-fluoro-3-(methoxymethoxy)naphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate

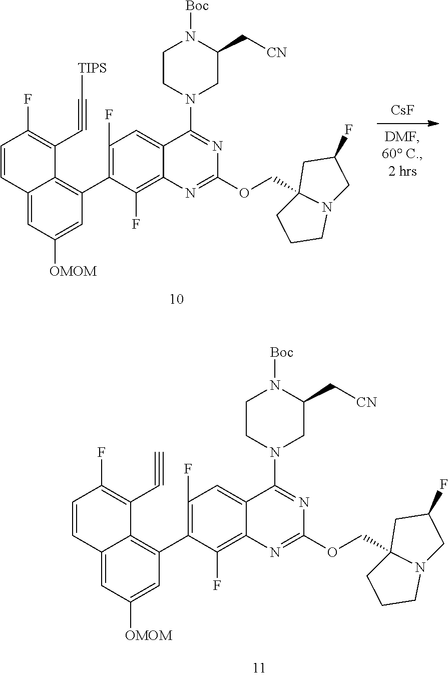

Tert-butyl (2S)-2-(cyanomethyl)-4-(6,8-difluoro-7-(7-fluoro-3-(methoxymethoxy)-8-((triisopropylsilyl)ethynyl)naphth-1-yl)-2-(((2R,7aS)-2-fluorotetrahydro-1H-pyrrolizin-7a(5H)-yl)methoxy)quinazolin-4-yl)piperazine-1-carboxylate (500 mg, 536 umol) and cesium fluoride (326 mg, 2.15 mmol) were dissolved in N,N-dimethylformamide (10 mL) and reacted at 60° C. for 1 hour. The reaction was complete as monitored by LCMS. The reaction product was quenched by adding water at 0° C. and extracted with ethyl acetate, the organic phase was washed with a saturated sodium chloride aqueous solution and dried over sodium sulfate, and after filtration, concentration and purification by column chromatography, tert-butyl (2S)-2-(cyanomethyl)-4-(7-(8-ethynyl-7-fluoro-3-(methoxymethoxy)naphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate (400 mg, yield 96.1%) was obtained.

LCMS: m/z 775.4 (M+H)+.

¹H NMR (400 MHz, CDCl₃): δ=7.82 (dd, J₁=5.8, J₂=9.1 Hz, 1H), 7.54 (d, J=2.5 Hz, 1H), 7.29 (br d, J=3.5 Hz, 2H), 7.24 (dd, J₁=2.8, J₂=4.9 Hz, 1H), 5.35 (br s, 1H), 5.27 (br d, J=1.5 Hz, 1H), 5.21 (br s, 1H), 5.14 (dd, J₁=3.2, J₂=4.2 Hz, 1H), 4.66 (br s, 1H), 4.31-4.14 (m, 4H), 4.13-3.97 (m, 1H), 3.61 (td, J₁=3.7, J₂=13.8 Hz, 1H), 3.52 (s, 3H), 3.41-3.32 (m, 1H), 3.31-3.23 (m, 2H), 3.23-3.18 (m, 2H), 2.83-2.74 (m, 2H), 2.26 (br s, 1H), 2.19 (br s, 1H), 2.05-2.00 (m, 2H), 1.85 (br d, J=6.3 Hz, 2H), 1.84-1.77 (m, 2H), 1.51 (s, 9H) ppm.

### Step XI: Synthesis of 2-((2S)-4-(7-(8-ethynyl-7-fluoro-3-hydroxynaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile

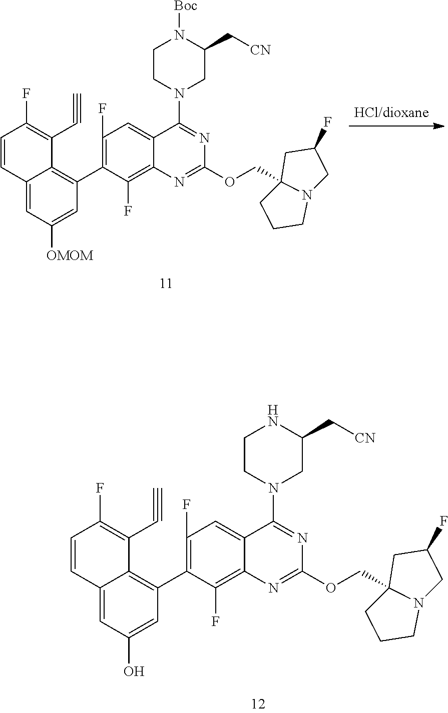

Tert-butyl (2S)-2-(cyanomethyl)-4-(7-(8-ethynyl-7-fluoro-3-(methoxymethoxy)naphth-1-yl)-6,8-difluoro-2-(((2R,7aS)-2-fluorotetrahydro-1H-pyrrolizin-7a(5H)-yl)methoxy)quinazolin-4-yl)piperazine-1-carboxylate (400 mg, 516 umol) was dissolved in dioxane hydrochloride (10 mL), and then, after nitrogen displacement three times, the mixture was reacted under nitrogen protection at 0° C. for 30 minutes. The reaction was complete as monitored by LCMS. The reaction liquid was concentrated to obtain a crude product of 2-((2S)-4-(7-(8-ethynyl-7-fluoro-3-hydroxynaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile (300 mg, crude).

LCMS: m/z 631.2 (M+H)+.

### Step XII Synthesis of 2-((2S)-4-(7-(8-ethynyl-7-fluoro-3-hydroxynaphth-1-yl)-6,8-difluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile

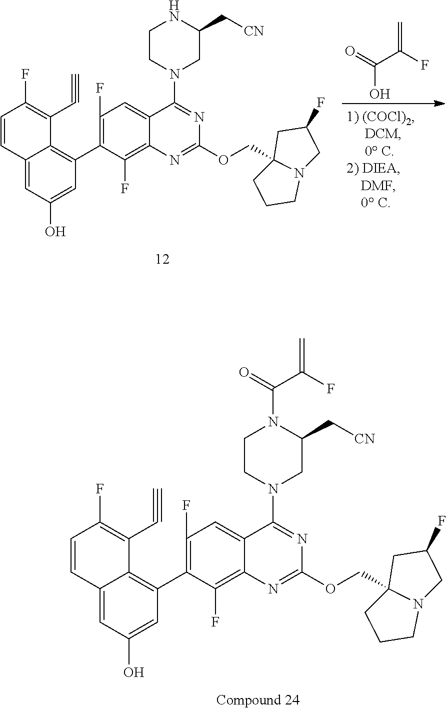

After 2-fluoroacrylic acid (171 mg, 1.90 mmol) was dissolved in dichloromethane (4 mL) at 0° C., oxalyl chloride (247 mg, 1.95 mmol) was added and reacted at this temperature for 10 minutes, a solution of 2-((2S)-4-(7-(8-ethynyl-7-fluoro-3-hydroxynaphth-1-yl)-6,8-difluoro-2-(((2R,7aS)-2-fluorotetrahydro-1H-pyrrolizin-7a(5H)-yl)methoxy)quinazolin-4-yl)piperazin-2-yl)acetonitrile (300 mg, 475 umol) and N,N-diisopropylethylamine (184 mg, 1.43 mmol) in N,N-dimethylformamide (3 mL) was then added, and the resulting mixture was stirred at 0° C. for 30 minutes. The reaction was complete as monitored by LCMS. The reaction liquid was filtered and subjected to prep-HPLC and SFC for separation and purification to obtain 2-((2S)-4-(7-(8-ethynyl-7-fluoro-3-hydroxynaphth-1-yl)-6,8-difluoro-2-(((2R,7aS)-2-fluorotetrahydro-1H-pyrrolizin-7a(5H)-yl)methoxy)quinazolin-4-yl)-1-(2-fluoroacryloyl)piperazin-2-yl)acetonitrile as a yellow solid. The product was subjected to SFC chiral resolution to obtain two peaks 24A and 24B of the target compound. Compounds 24A and 24B were axially chiral isomers of each other.

24A: (14.6 mg, yield 4.35%), LCMS: m/z 703.5 (M+H)+.

¹H NMR (400 MHz, CD₃CN): δ=7.90 (dd, J₁=5.8, J₂=9.2 Hz, 1H), 7.52 (dd, J₁=1.4, J₂=9.8 Hz, 1H), 7.41 (d, J=2.5 Hz, 1H), 7.35 (t, J=9.1 Hz, 1H), 7.22 (d, J=2.4 Hz, 1H), 5.37-5.24 (m, 2H), 5.20 (br s, 1H), 5.03-4.76 (m, 1H), 4.30-4.02 (m, 5H), 3.89-3.54 (m, 2H), 3.45-3.34 (m, 1H), 3.28-3.16 (m, 3H), 3.14-3.04 (m, 2H), 2.97-2.86 (m, 2H), 2.17-2.05 (m, 4H), 1.91-1.82 (m, 2H) ppm.

24B: (17.7 mg, yield 5.24%), LCMS: m/z 703.5 [M+H]⁺.

¹H NMR (400 MHz, CD₃CN): δ=7.91 (dd, J=5.9, J₂=9.1 Hz, 1H), 7.50 (dd, J₁=1.6, J₂=9.9 Hz, 1H), 7.41 (d, J=2.4 Hz, 1H), 7.37 (t, J=9.0 Hz, 1H), 7.21 (d, J=2.3 Hz, 1H), 5.35-5.25 (m, 2H), 5.23-5.17 (m, 1H), 5.03-4.77 (m, 1H), 4.37-4.06 (m, 5H), 3.72-3.34 (m, 3H), 3.18-3.04 (m, 5H), 2.99-2.86 (m, 2H), 2.10-2.01 (m, 4H), 1.89-1.83 (m, 2H) ppm.

### Example 25: 2-((2S)-4-(7-(7-ethynylnaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile (Compound 25)

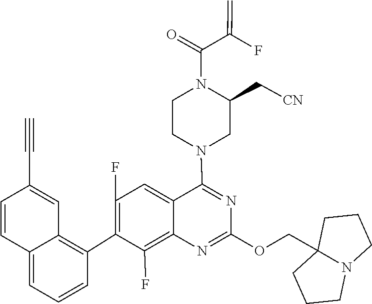 [SMILES: C#Cc1ccc2cccc(-c3c(F)cc4c(N5CCN(C(=O)C(=C)F)[C@@H](CC#N)C5)nc(OCC56CCCN5CCC6)nc4c3F)c2c1]

LCMS: m/z 651.3 [M+H]⁺.

### Example 26: 2-((2S)-4-(7-(3-ethynylnaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile (Compound 26)

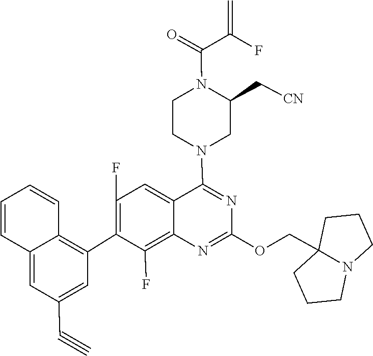 [SMILES: C#Cc1cc(-c2c(F)cc3c(N4CCN(C(=O)C(=C)F)[C@@H](CC#N)C4)nc(OCC45CCCN4CCC5)nc3c2F)c2ccccc2c1]

LCMS: m/z 651.3 [M+H]⁺.

### Example 27: 2-((2S)-4-(7-(5-ethynylnaphth-1-yl)-6,8-difluoro-2-((tetrahydro-1H-pyrrolizin-7a(5H)-yl)methoxy)quinazolin-4-yl)-1-(2-fluoroacryloyl)piperazin-2-yl)acetonitrile (Compound 27)

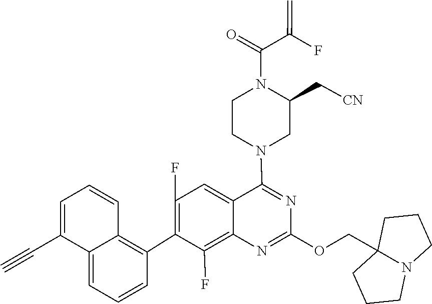 [SMILES: C#Cc1cccc2c(-c3c(F)cc4c(N5CCN(C(=O)C(=C)F)[C@@H](CC#N)C5)nc(OCC56CCCN5CCC6)nc4c3F)cccc12]

LCMS: m/z 651.3 [M+H]⁺.

### Example 28: (S)-2-(4-(7-(5-chloro-3-hydroxynaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (Compound 28)

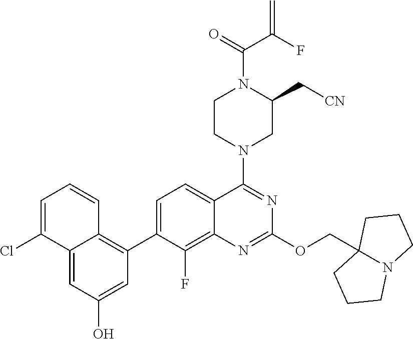 [SMILES: C=C(F)C(=O)N1CCN(c2nc(OCC34CCCN3CCC4)nc3c(F)c(-c4cc(O)cc5c(Cl)cccc45)ccc23)C[C@@H]1CC#N]

LCMS: m/z 659.5 [M+H]⁺.

### Example 29: (S)-2-(4-(7-(7-chloro-3-hydroxynaphth-1-yl)-8-fluoro-2-((tetrahydro-1H-pyrrolizin-7a(5H)-yl)methoxy)quinazolin-4-yl)-1-(2-fluoroacryloyl)piperazin-2-yl)acetonitrile (Compound 29)

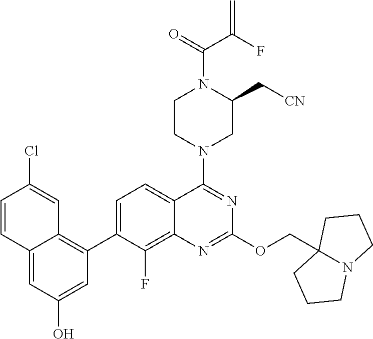 [SMILES: C=C(F)C(=O)N1CCN(c2nc(OCC34CCCN3CCC4)nc3c(F)c(-c4cc(O)cc5ccc(Cl)cc45)ccc23)C[C@@H]1CC#N]

LCMS: m/z 659.5 [M+H]⁺.

### Biological Test Evaluation

The present invention is further described and explained in conjunction with test examples. The compounds AMG510 (catalog number: HY-114277) and MRTX849 (catalog number: HY-130149) used in each test example were both purchased from MedChemExpress.

### Test Example 1: Proliferation Experiment of NCI-H358 Cells

Human lung cancer cell line NCI-H358 (ATCC, CRL-5807) was a KRAS G12C mutant cell line. NCI-H358 cells were cultured in RPMI 1640 medium containing 10% fetal bovine serum and placed in a humidified incubator with 5% CO₂ at 37° C. for growth.

According to the solution described in Cell Titer-Glo Luminescent cell Viability Assay (Promega catalog number #G7570) of Promega Corporation, the number of living cells in the culture was detected. 90 μL of cells (8,000 cells/well) were cultured in a growth medium in a Corning black transparent bottom 96-well plate and were cultured overnight in a 5% CO₂ humidified incubator at 37° C. The compound diluted serially in 100% DMSO was added to the cells by a pipette, and the cells were cultured for another 72 hours. 100 μL of the well-mixed Cell Titer-Glo reagent was added to the cells in the 96-well culture plate to lyse the cells and gently mixed. Subsequently, autofluorescence was detected on an Envision microplate detector, and the data of each compound was obtained. Finally, the data was input into a suitable software package (such as Prism) for curve fitting analysis. Based on this data, the IC₅₀ value was determined by calculating the compound concentration required to obtain a 50% inhibitory effect.

### Test Example 2: Proliferation Experiment of NCI-H23 Cells

Human lung cancer cell line NCI-H23 (ATCC, CRL-5800) was a KRAS G12C mutant cell line. NCI-H23 cells were cultured in RPMI 1640 medium containing 10% fetal bovine serum and placed in a humidified incubator with 5% CO₂ at 37° C. for growth.

According to the solution described in Cell Titer-Glo Luminescent cell Viability Assay (Promega catalog number #G7570) of Promega Corporation, the number of living cells in the culture was detected. 90 μL of cells (8,000 cells/well) were cultured in a growth medium in a Corning black transparent bottom 96-well plate and were cultured overnight in a 5% CO₂ humidified incubator at 37° C. The compound diluted serially in 100% DMSO was added to the cells by a pipette, and the cells were cultured for another 72 hours. 100 μL of the well-mixed Cell Titer-Glo reagent was added to the cells in the 96-well culture plate to lyse the cells and gently mixed. Subsequently, autofluorescence was detected on an Envision microplate detector, and the data of each compound was obtained. Finally, the data was input into a suitable software package (such as Prism) for curve fitting analysis. Based on this data, the IC₅₀ value was determined by calculating the compound concentration required to obtain a 50% inhibitory effect.

The data of the inhibitory activity of each of the compounds on KRAS G12C mutation as obtained in Test Example 1 and Test Example 2 were shown in Table 1.

TABLE 1

| Compound | Test Example 1 | Test Example 2 |
|---|---|---|
| No. | IC₅₀ (nM) | IC₅₀ (nM) |
| 2 | 0.22 | 0.65 |
| 4 | 0.44 | 1.15 |
| 5 | 1.58 | 1.27 |
| 6A | 1.83 | 1.44 |
| 7A | 0.62 | 0.60 |
| 8A | 0.24 | 0.58 |
| 11 | 0.79 | 0.86 |
| 12 | 0.66 | 1.02 |
| 17 | 0.60 | 0.53 |
| 18B | 0.50 | 0.68 |
| 19B | 0.47 | 1.71 |
| 22A | 0.47 | 0.76 |
| 24A | 0.25 | 0.52 |
| AMG510 | 14.46 | 14.96 |
| MRTX849 | 9.64 | 13.10 |

### Test Example 3: Proliferation Experiment of Ba/F3 (KRAS-G12C-Y96C) Cells

Engineered cell strain Ba/F3 (KRAS-G12C-Y96C) was a cell strain constructed by the inventors according to the methods described in Warmuth M. et al, Curr Opin Oncol. 2007 January; 19(1):55-60; and Tanaka N. et al, Cancer Discov. 2021 August; 11(8):1913-1922. The engineered cell line was cultured in RPMI 1640 medium containing 1000 fetal bovine serum and 2 mM glutamine. The cells were grown at 37° C. in a humidified incubator with 500 CO₂.

According to the solution described in Cell Titer-Glo Luminescent cell Viability Assay (Promega catalog number #G7570) of Promega Corporation, the number of living cells in the culture was detected. 90 μL of cells (3,000 cells/well) were cultured in a growth medium in a Corning black transparent bottom 96-well plate and were cultured overnight in a 50% CO₂ humidified incubator at 37° C. The compound diluted serially in 100% DMSO was added to the cells by a pipette, and the cells were cultured for another 72 hours. 100 μL of the well-mixed Cell Titer-Glo reagent was added to the cells in the 96-well culture plate to lyse the cells and gently mixed. Subsequently, autofluorescence was detected on an Envision microplate detector, and the data of each compound was obtained. Finally, the data was input into a suitable software package (such as Prism) for curve fitting analysis. Based on this data, the IC₅₀ value was determined by calculating the compound concentration required to obtain a 500% inhibitory effect.

### Test Example 4: Proliferation Experiment of Ba/F3 (KRAS-G12C-H195Q) Cells

Engineered cell strain Ba/F3 (KRAS-G12C-H95Q) was a cell strain constructed by the inventors according to the methods described in Warmuth M. et al, Curr Opin Oncol. 2007 January; 19(1):55-60; and Tanaka N. et al, Cancer Discov. 2021 August; 11(8):1913-1922. The engineered cell line was cultured in RPMI 1640 medium containing 10% fetal bovine serum and 2 mM glutamine. The cells were grown at 37° C. in a humidified incubator with 500 CO₂.

According to the solution described in Cell Titer-Glo Luminescent cell Viability Assay (Promega catalog number #G7570) of Promega Corporation, the number of living cells in the culture was detected. 90 μL of cells (3,000 cells/well) were cultured in a growth medium in a Corning black transparent bottom 96-well plate and were cultured overnight in a 5% CO₂ humidified incubator at 37° C. The compound diluted serially in 100% DMSO was added to the cells by a pipette, and the cells were cultured for another 72 hours. 100 μL of the well-mixed Cell Titer-Glo reagent was added to the cells in the 96-well culture plate to lyse the cells and gently mixed. Subsequently, autofluorescence was detected on an Envision microplate detector, and the data of each compound was obtained. Finally, the data was input into a suitable software package (such as Prism) for curve fitting analysis. Based on this data, the IC₅₀ value was determined by calculating the compound concentration required to obtain a 50% inhibitory effect.

### Test Example 5: Proliferation Experiment of Ba/F3 (KRAS-G12C-R68S) Cells

Engineered cell strain Ba/F3 (KRAS-G12C-R68S) was a cell strain constructed by the inventors according to the methods described in Warmuth M. et al, Curr Opin Oncol. 2007 January; 19(1):55-60; and Tanaka N. et al, Cancer Discov. 2021 August; 11(8):1913-1922. The engineered cell line was cultured in RPMI 1640 medium containing 10% fetal bovine serum and 2 mM glutamine. The cells were grown at 37° C. in a humidified incubator with 5% CO₂.

According to the solution described in Cell Titer-Glo Luminescent cell Viability Assay (Promega catalog number #G7570) of Promega Corporation, the number of living cells in the culture was detected. 90 μL of cells (3,000 cells/well) were cultured in a growth medium in a Corning black transparent bottom 96-well plate and were cultured overnight in a 5% CO₂ humidified incubator at 37° C. The compound diluted serially in 100% DMSO was added to the cells by a pipette, and the cells were cultured for another 72 hours. 100 μL of the well-mixed Cell Titer-Glo reagent was added to the cells in the 96-well culture plate to lyse the cells and gently mixed. Subsequently, autofluorescence was detected on an Envision microplate detector, and the data of each compound was obtained. Finally, the data was input into a suitable software package (such as Prism) for curve fitting analysis. Based on this data, the IC₅₀ value was determined by calculating the compound concentration required to obtain a 50% inhibitory effect.

### Test Example 6: Proliferation Experiment of Ba/F3 (KRAS-G12C-Q99L) Cells

Engineered cell strain Ba/F3 (KRAS-G12C-Q99L) was a cell strain constructed by the inventors according to the methods described in Warmuth M. et al, Curr Opin Oncol. 2007 January; 19(1):55-60; and Tanaka N. et al, Cancer Discov. 2021 August; 11(8):1913-1922. The engineered cell line was cultured in RPMI 1640 medium containing 10% fetal bovine serum and 2 mM glutamine. The cells were grown at 37° C. in a humidified incubator with 5% CO₂.

According to the solution described in Cell Titer-Glo Luminescent cell Viability Assay (Promega catalog number #G7570) of Promega Corporation, the number of living cells in the culture was detected. 90 μL of cells (3,000 cells/well) were cultured in a growth medium in a Corning black transparent bottom 96-well plate and were cultured overnight in a 5% CO₂ humidified incubator at 37° C. The compound diluted serially in 100% DMSO was added to the cells by a pipette, and the cells were cultured for another 72 hours. 100 μL of the well-mixed Cell Titer-Glo reagent was added to the cells in the 96-well culture plate to lyse the cells and gently mixed. Subsequently, autofluorescence was detected on an Envision microplate detector, and the data of each compound was obtained. Finally, the data was input into a suitable software package (such as Prism) for curve fitting analysis. Based on this data, the IC₅₀ value was determined by calculating the compound concentration required to obtain a 50% inhibitory effect.

The data of the inhibitory activity of each of the compounds on drug-resistant mutation as obtained in Test Examples 3 to 6 were shown in Table 2.

TABLE 2

| | Test | Test | Test | Test |
|---|---|---|---|---|
| Compound | Example 3 | Example 4 | Example 5 | Example 6 |
| No. | IC50 (nM) | IC₅₀ (nM) | IC₅₀ (nM) | IC₅₀ (nM) |
| 2 | 18.93 | 70.13 | 93.89 | 130.4 |
| 4 | 5.20 | 39.43 | 50.59 | 9.06 |
| 5 | 472.8 | 709.3 | 928.6 | 589.3 |
| 6A | 524.5 | 688.7 | 863.1 | 778.9 |
| 7A | 5.28 | 16.83 | 34.62 | 9.03 |
| 8A | 3.44 | 17.81 | 33.28 | 11.6 |
| 11 | 25.68 | 62.57 | 69.38 | 56.74 |
| 12 | 26.22 | 41.60 | 51.43 | 24.4 |
| 18B | 4.89 | 12.78 | 29.89 | 8.92 |
| 22A | 7.23 | 23.90 | 34.12 | 11.05 |
| 24A | 4.0 | 7.6 | 5.5 | 4.0 |
| 25 | >5000 | >5000 | >5000 | >5000 |
| 26 | >5000 | >5000 | >5000 | >5000 |
| 27 | >5000 | >5000 | >5000 | >5000 |
| 28 | >5000 | >5000 | >5000 | >5000 |
| 29 | >5000 | >5000 | >5000 | >5000 |
| AMG510 | 3197 | 4286 | >5000 | 3947 |
| MRTX849 | 1050 | 847.2 | 904.3 | 811.6 |

### Test Example 7: In Vitro Human Liver Microsome Stability Experiment

Eight 96-well incubation plates were prepared, named T0, T5, T15, T30, T45, T60, Blank60, and NCF60, respectively. The reaction time points corresponding to the first six incubation plates were 0, 5, 15, 30, 45 and 60 minutes, respectively. No compound to be tested or control compound was added to the Blank60 plate and samples were taken after 60 minutes of incubation. The NCF60 plate, in which a potassium phosphate buffer was used instead of an NADPH regeneration system solution (Sigma, N0505), was incubated for 60 minutes.

2 μL of a working solution of the compound to be tested or the control and 100 μL of a working solution of human liver microsomes (Corning, 452117) were added to T0, T5, T15, T30, T45, T60, and NCF60 plates, respectively, wherein the concentration of the microsome protein was 1 mg/mL. Only the working solution of microsomes was added to the Blank60 plate, and the incubation plates Blank60, T5, T15, T30, T45, and T60 except TO and NCF60 were then placed in a water bath at 37° C. for pre-incubation for about 10 minutes.

To the TO plate sample, 600 μL of a stop solution (acetonitrile:methanol (95:5, v/v) solution containing 100 ng/mL tolbutamide (Sigma, T0891)) was added first, and then an NADPH regeneration system working solution was added.

98 μL of a potassium phosphate buffer was added to each well of the NCF60 plate and incubated for 60 minutes.

After the pre-incubation in the incubation plates Blank60, T5, T15, T30, T45, and T60 was complete, 98 μL of the NADPH regeneration system working solution was added to each sample well to initiate the reaction.

After an appropriate incubation time (such as 5, 15, 30, 45, and 60 minutes), 600 μL of the stop solution was added to each sample well and control sample well in the Blank60, T5, T15, T30, T45, T60, and NCF60 plates, respectively, to stop the reaction.

All the sample plates were shaken evenly and centrifuged at 3220×g for 20 minutes. 200 μL of each test sample supernatant was taken and diluted into 200 μL of an aqueous solution containing 0.3% formic acid for LC-MS/MS analysis, and 100 μL of the control sample supernatant was taken and diluted into 300 μL of pure water for LC-MS/MS analysis. Calculations were carried out according to the following formulas:

$$C \text{?} = C \text{?} \cdot e \text{?}$$

$$when C \text{?} = \frac{1}{2} {C}_{0} ,$$

$$T \text{?} = \frac{Ln 2}{k \text{?}} = \frac{0.693}{k \text{?}}$$

$$CL \text{?} = \frac{0.696}{In vitro T \text{?}} \cdot \frac{1}{mg / microsomal protein in the reaction system}$$

$${CL}_{int ( liver )} = CL \text{?} \cdot \frac{mg of microsomes}{g of liver} \cdot \frac{g of liver}{kg of body weight}$$

$$\text{?} \text{indicates text missing or illegible when filed}$$

The experimental data of the in vitro human liver microsome stability of each of the compounds as obtained in Test Example 7 were shown in Table 3.

TABLE 3

| | | CL_{int(mic)} | CLᵢₙₜ₍ₗᵢᵥₑᵣ₎ |
|---|---|---|---|
| Compound No. | T_{1/2} (min) | (μL/min/mg) | (mL/min/kg) |
| 2 | 18.8 | 73.7 | 66.3 |
| 4 | 25.5 | 54.3 | 48.9 |
| 7A | 38.4 | 36.1 | 32.5 |
| 8A | 26.7 | 53.0 | 49.7 |
| 9 | 16.9 | 78.9 | 75.3 |
| 11 | 38.8 | 35.7 | 32.1 |
| 12 | 30.9 | 56.2 | 49.6 |
| 18B | 44.2 | 35.6 | 33.0 |
| 19B | 24.5 | 56.5 | 50.9 |
| 22A | 39.2 | 35.6 | 33.0 |
| 24A | 17.1 | 81.3 | 73.2 |

1. A compound of formula (II), or a pharmaceutically acceptable salt or stereoisomer thereof: 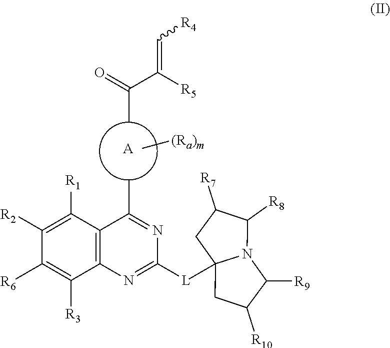
wherein
L is selected from a bond, oxygen, sulfur, —NH—, —(CH₂)ₙ—, —O(CH₂)ₙ—, —S(CH₂)ₙ—, —NH(CH₂)ₙ—, —(CH₂)ₙNH—, —(CH₂)ₙO—, —(CH₂)ₙS—, —(CH₂)ₙC(═O)—, —C(═O)O(CH₂)ₙ—, —OC(═O)(CH₂)ₙ—, —C(═O)(CH₂)ₙ—, —C(═O)NH(CH₂)ₙ—, and —NHC(═O)(CH₂)ₙ—; preferably —OCH₂—, —NHCH₂—, or —NHC(═O)—; preferably —OCH₂—, —NHCH₂—, or —NHC(═O)—;
ring A is selected from substituted or unsubstituted, saturated or unsaturated, N-containing cyclic, spiro or bridged C₄₋₁₄ compounds containing at least additionally one heteroatom selected from O, S, or N;
R₁, R₂, and R₃ are each independently selected from hydrogen, deuterium, cyano, amino, hydroxyl, halogen (such as —F, —Cl, or —Br), nitro, alkyl, alkenyl, alkynyl, alkoxy, C₁₋₃ haloalkyl, C₁₋₃ haloalkoxy (such as —OCF₃), and deuterated alkyl, wherein the amino, alkyl, alkenyl, alkynyl, alkoxy, haloalkyl, haloalkoxy, and deuterated alkyl can be optionally further substituted;
R₄ is selected from hydrogen, halogen, C₁₋₆ alkyl, C₂₋₆ alkynyl, carboxyl-substituted C₁₋₆ alkyl, amino-substituted C₁₋₆ alkyl, hydroxyl-substituted C₁₋₆ alkyl, C₁₋₆ haloalkyl, cycloalkyl, C₁-4 alkylene-C₂₋₆ heterocycloalkyl, C₂₋₆ heterocycloalkyl, aryl, heteroaryl, C₁₋₃ alkylamino-substituted C₁₋₄ alkyl, and C₁₋₃ alkylamino-substituted C₁₋₈ alkoxy;
R₅ is selected from halogen or haloalkyl;
R₆ is selected from C₆₋₁₀ aryl, C₅₋₉ heteroaryl, C₆₋₁₀ aryl-C₁₋₆ alkylene, and C₅₋₁₀ heteroaryl-C₁₋₆ alkylene, wherein the alkyl, aryl, heteroaryl, or heterocyclyl is optionally substituted with one or more substituents independently selected from halogen, cyano, amino, hydroxyl, nitro, C₁₋₆ alkyl, C₁₋₆ alkoxy, C₁₋₆ alkylamino, C₂₋₄ alkynyl, C₂₋₄ alkenyl, C₁₋₄ alkylcyano, C₃₋₆ cycloalkyl, di-C₁₋₆ alkylamino, C₁₋₆ haloalkyl, aminoacyl, C₁₋₆ alkylaminoacyl, or di-C₁₋₆ alkylaminoacyl;
R₇, R₈, R₉, and R₁₀ are each selected from hydrogen, —NH₂, —OH, —C(═O), —CN, —NO₂, halogen, —COOH, C₁₋₁₂ alkyl, C₂₋₆ alkenyl, C₂₋₆ alkynyl, C₁₋₁₂ haloalkyl, C₃₋₁₂ cycloalkyl, C₃₋₁₄ heterocycloalkyl, C₆₋₁₀ aryl, C₅₋₉ heteroaryl, —(CH₂)ₙOC(═O)—C₆₋₁₀ aryl, —(CH₂)ₙOC(═O)—C₅₋₉ heteroaryl, —(CH₂)ₙOC(═O)—C₃₋₁₄ heterocycloalkyl, —(CH₂)ₙOC(═O)N(R₁₁)₂, —N(R₁₁)₂, —NHC(═NH)NH₂, —NR₁₁C(═O)—C₆₋₁₀ aryl, —NR₁₁C(═O)—C₅₋₉ heteroaryl, —NR₁₁C(═O)—C₃₋₁₄ heterocycloalkyl, —C(═O)N(R₁₁)₂, —(CH₂)ₙNHC(═O)—C₆₋₁₀ aryl, —(CH₂)ₙNHC(═O)—C₅₋₉ heteroaryl, and —(CH₂)ₙNHC(═O)—C₃₋₁₄ heterocycloalkyl, wherein the alkyl, amino, aryl, heteroaryl, cycloalkyl, or heterocyclyl is optionally substituted with one or more substituents independently selected from C₁₋₆ alkyl, C₁₋₆ alkoxy, C₁₋₆ haloalkyl, C₁₋₆ heterocycloalkyl, —NH₂, —OH, —C(═O), —CN, —NO₂, halogen, and —COOH;
m and n are each independently 0, 1, 2, or 3; and
R₁₁ is selected from C₁₋₃ alkyl, C₁₋₃ alkoxy, C₁₋₃ haloalkyl, and hydroxyl.

2. The compound according to claim 1 or a pharmaceutically acceptable salt or stereoisomer thereof, wherein the compound is a compound of formula (II-A0), formula (II-A), formula (II-B0), or formula (II-B), or a pharmaceutically acceptable salt or stereoisomer thereof: 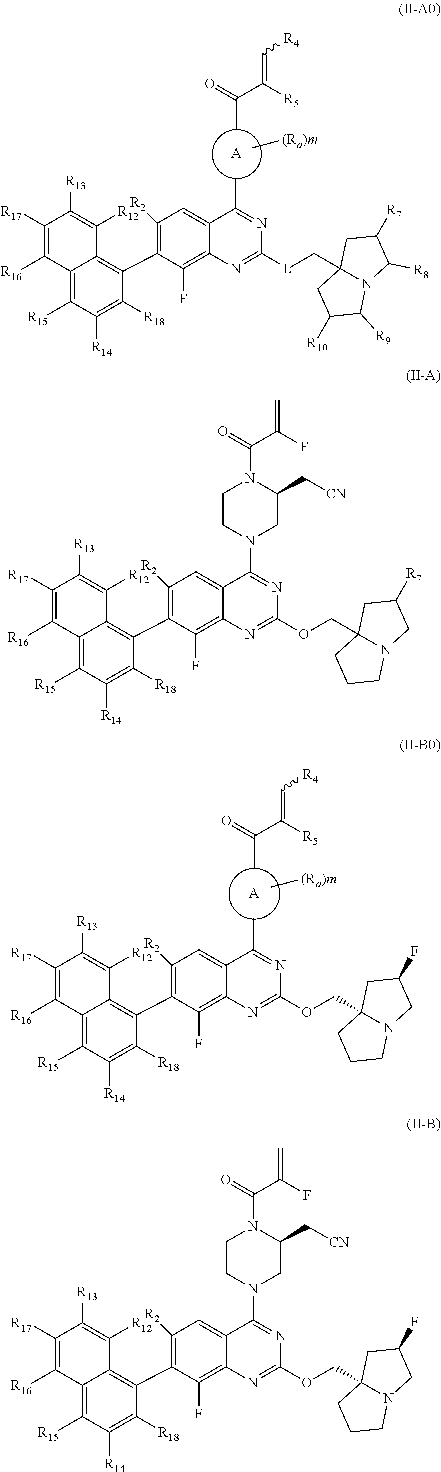
wherein R₂ has the definition given in claim 1 and is preferably hydrogen, fluorine, or chlorine; R₇ has the definition given in claim 1 and is preferably hydrogen or fluorine; R₁₂ to R₁₈ are each independently selected from hydrogen, halogen (especially fluorine and chlorine), cyano, hydroxyl, amino, C₁₋₆ alkyl (especially methyl and ethyl), and C₂₋₆ alkynyl (especially ethynyl).

3. The compound according to claim 1 or a pharmaceutically acceptable salt or stereoisomer thereof, wherein the compound is a compound of formula (II-C), formula (II-C1), or formula (II-C2): 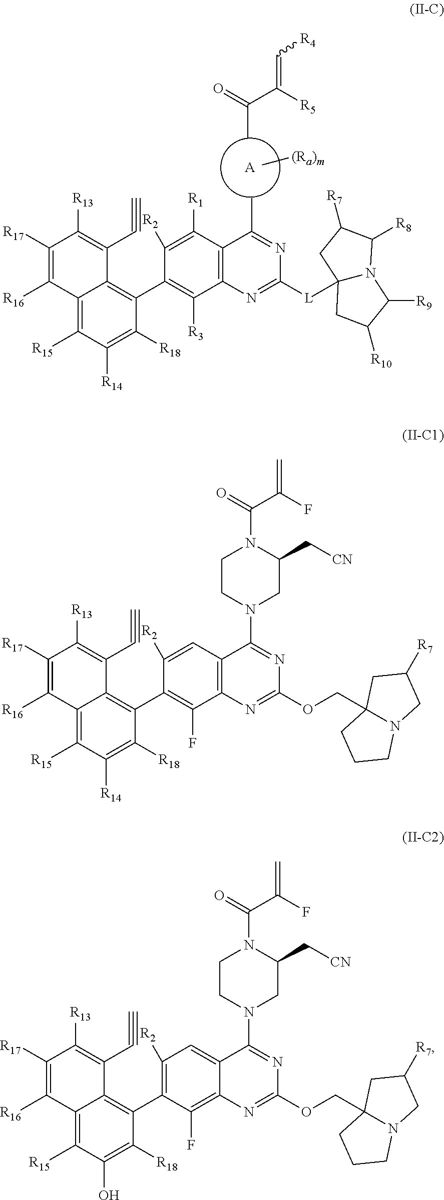
wherein R₁₃ to R₁₈ are each independently selected from hydrogen, halogen (especially fluorine and chlorine), cyano, hydroxyl, amino, C₁₋₆ alkyl (especially methyl and ethyl), and C₂₋₆ alkynyl (especially ethynyl), preferably hydrogen, fluorine, chlorine, cyano, methyl, ethyl, hydroxyl, and amino; R₇ is preferably hydrogen or fluorine.

4. The compound according to claim 1 or a pharmaceutically acceptable salt or stereoisomer thereof, which is a compound of formula (II-D) or formula (II-D0): 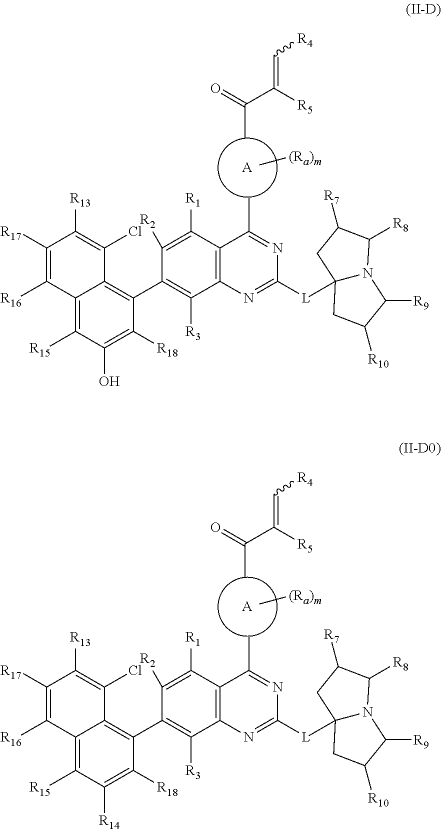
wherein
L is selected from a bond, oxygen, sulfur, —NH—, —(CH₂)ₙ—, —O(CH₂)ₙ—, —S(CH₂)ₙ—, —NH(CH₂)ₙ—, —(CH₂)ₙNH—, —(CH₂)ₙO—, —(CH₂)ₙS—, —(CH₂)ₙC(═O)—, —C(═O)O(CH₂)ₙ—, —OC(═O)(CH₂)ₙ—, —C(═O)(CH₂)ₙ—, —C(═O)NH(CH₂)ₙ—, and —NHC(═O)(CH₂)ₙ—; preferably —OCH₂—, —NHCH₂—, or —NHC(═O)—;
ring A is selected from substituted or unsubstituted, saturated or unsaturated, N-containing cyclic, spiro or bridged C₄₋₁₄ compounds containing at least additionally one heteroatom selected from O, S, or N;
R₁, R₂, and R₃ are each independently selected from hydrogen, deuterium, cyano, amino, hydroxyl, halogen (such as —F, —Cl, or —Br), nitro, alkyl, alkenyl, alkynyl, alkoxy, C₁₋₃ haloalkyl, C₁₋₃ haloalkoxy (such as —OCF₃), and deuterated alkyl, wherein the amino, alkyl, alkenyl, alkynyl, alkoxy, haloalkyl, haloalkoxy, and deuterated alkyl can be optionally further substituted;
R₄ is selected from hydrogen, halogen, C₁₋₆ alkyl, C₂₋₆ alkynyl, carboxyl-substituted C₁₋₆ alkyl, amino-substituted C₁₋₆ alkyl, hydroxyl-substituted C₁₋₆ alkyl, C₁₋₆ haloalkyl, cycloalkyl, C₁-4 alkylene-C₂₋₆ heterocycloalkyl, C₂₋₆ heterocycloalkyl, aryl, heteroaryl, C₁₋₃ alkylamino-substituted C₁₋₄ alkyl, and C₁₋₃ alkylamino-substituted C₁₋₈ alkoxy;
R₅ is selected from halogen or haloalkyl;
R₆ is selected from C₆₋₁₀ aryl, C₅₋₉ heteroaryl, C₆₋₁₀ aryl-C₁₋₆ alkylene, and C₅₋₁₀ heteroaryl-C₁₋₆ alkylene, wherein the alkyl, aryl, heteroaryl, or heterocyclyl is optionally substituted with one or more substituents independently selected from halogen, cyano, amino, hydroxyl, nitro, C₁₋₆ alkyl, C₁₋₆ alkoxy, C₁₋₆ alkylamino, C₂₋₄ alkynyl, C₂₋₄ alkenyl, C₁₋₄ alkylcyano, C₃₋₆ cycloalkyl, di-C₁₋₆ alkylamino, C₁₋₆ haloalkyl, aminoacyl, C₁₋₆ alkylaminoacyl, or di-C₁₋₆ alkylaminoacyl;
R₇, R₈, R₉, and R₁₀ are each selected from hydrogen, —NH₂, —OH, —C(═O), —CN, —NO₂, halogen, —COOH, C₁₋₁₂ alkyl, C₂₋₆ alkenyl, C₂₋₆ alkynyl, C₁₋₁₂ haloalkyl, C₃₋₁₂ cycloalkyl, C₃₋₁₄ heterocycloalkyl, C₆₋₁₀ aryl, C₅₋₉ heteroaryl, —(CH₂)ₙOC(═O)—C₆₋₁₀ aryl, —(CH₂)ₙOC(═O)—C₅₋₉ heteroaryl, —(CH₂)ₙOC(═O)—C₃₋₁₄ heterocycloalkyl, —(CH₂)ₙOC(═O)N(R₁₁)₂, —N(R₁₁)₂, —NHC(═NH)NH₂, —NR₁₁C(═O)—C₆₋₁₀ aryl, —NR₁₁C(═O)—C₅₋₉ heteroaryl, —NR₁₁C(═O)—C₃₋₁₄ heterocycloalkyl, —C(═O)N(R₁)₂, —(CH₂)ₙNHC(═O)—C₆₋₁₀ aryl, —(CH₂)ₙNHC(═O)—C₅₋₉ heteroaryl, and —(CH₂)ₙNHC(═O)—C₃₋₁₄ heterocycloalkyl, wherein the alkyl, amino, aryl, heteroaryl, cycloalkyl, or heterocyclyl is optionally substituted with one or more substituents independently selected from C₁₋₆ alkyl, C₁₋₆ alkoxy, C₁₋₆ haloalkyl, C₁₋₆ heterocycloalkyl, —NH₂, —OH, —C(═O), —CN, —NO₂, halogen, and —COOH;
m and n are each independently 0, 1, 2, or 3; and
R₁₁ is selected from C₁₋₃ alkyl, C₁₋₃ alkoxy, C₁₋₃ haloalkyl, and hydroxyl; and
R₁₃ and R₁₅ to R₁₈ are each independently selected from hydrogen, halogen (especially fluorine and chlorine), cyano, hydroxyl, amino, C₁₋₆ alkyl (especially ethyl), and C₂₋₆ alkynyl (especially ethynyl), preferably hydrogen, fluorine, chlorine, cyano, methyl, ethyl, hydroxyl, and amino.

5. The compound according to claim 4 or a pharmaceutically acceptable salt or stereoisomer thereof, wherein the compound is a compound of formula (II-D1) or formula (II-D2): 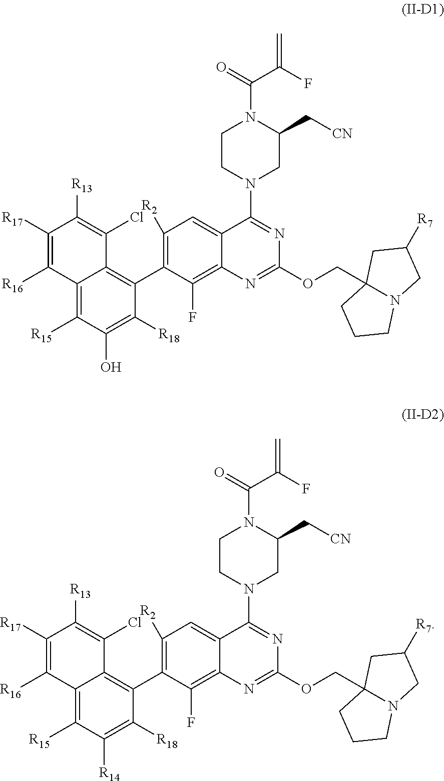
wherein R₁₃ and R₁₅ to R₁₈ are each independently selected from hydrogen, halogen (especially fluorine and chlorine), cyano, hydroxyl, amino, C₁₋₆ alkyl (especially ethyl), and C₂₋₆ alkynyl (especially ethynyl), preferably hydrogen, fluorine, chlorine, cyano, methyl, ethyl, hydroxyl, and amino; R₇ is preferably hydrogen or fluorine.

6. The compound according to claim 1 or a pharmaceutically acceptable salt thereof, wherein the compound is selected from:

| No. | Structure |
|---|---|
| 1 | 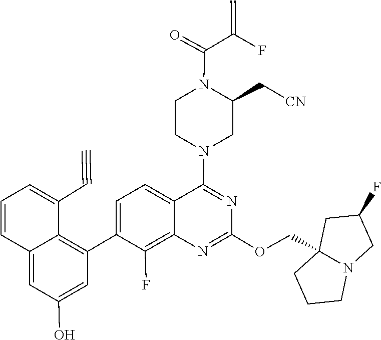 [SMILES: C#Cc1cccc2cc(O)cc(-c3ccc4c(N5CCN(C(=O)C(=C)F)[C@@H](CC#N)C5)nc(OC[C@@]56CCCN5C[C@H](F)C6)nc4c3F)c12] |
| 2 | 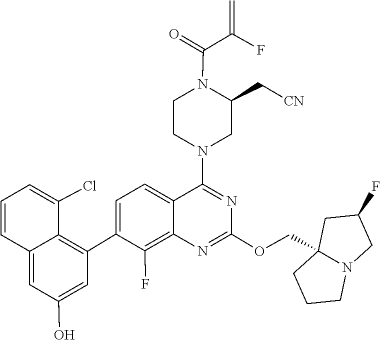 [SMILES: C=C(F)C(=O)N1CCN(c2nc(OC[C@@]34CCCN3C[C@H](F)C4)nc3c(F)c(-c4cc(O)cc5cccc(Cl)c45)ccc23)C[C@@H]1CC#N] |
| 3 | 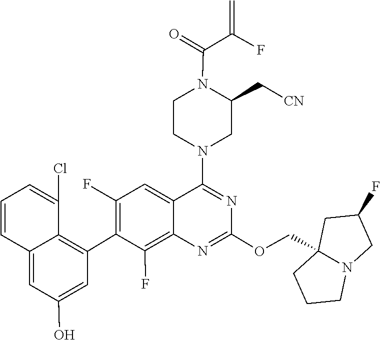 [SMILES: C=C(F)C(=O)N1CCN(c2nc(OC[C@@]34CCCN3C[C@H](F)C4)nc3c(F)c(-c4cc(O)cc5cccc(Cl)c45)c(F)cc23)C[C@@H]1CC#N] |
| 4 | 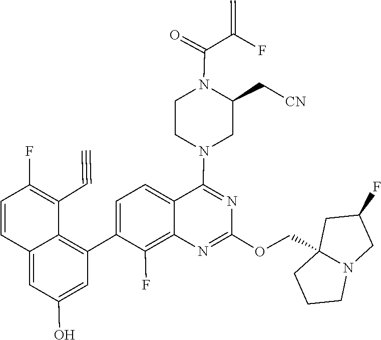 [SMILES: C#Cc1c(F)ccc2cc(O)cc(-c3ccc4c(N5CCN(C(=O)C(=C)F)[C@@H](CC#N)C5)nc(OC[C@@]56CCCN5C[C@H](F)C6)nc4c3F)c12] |
| 5 | 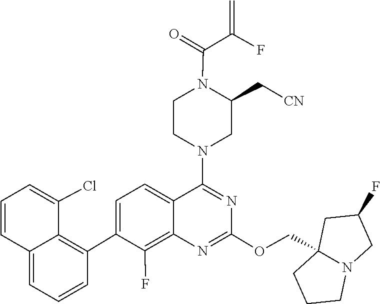 [SMILES: C=C(F)C(=O)N1CCN(c2nc(OC[C@@]34CCCN3C[C@H](F)C4)nc3c(F)c(-c4cccc5cccc(Cl)c45)ccc23)C[C@@H]1CC#N] |
| 6 | 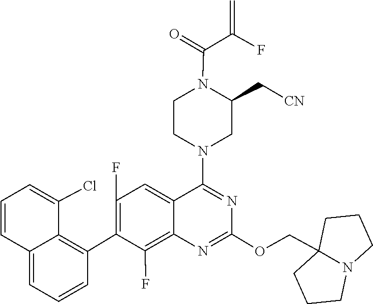 [SMILES: C=C(F)C(=O)N1CCN(c2nc(OCC34CCCN3CCC4)nc3c(F)c(-c4cccc5cccc(Cl)c45)c(F)cc23)C[C@@H]1CC#N] |
| 7 | 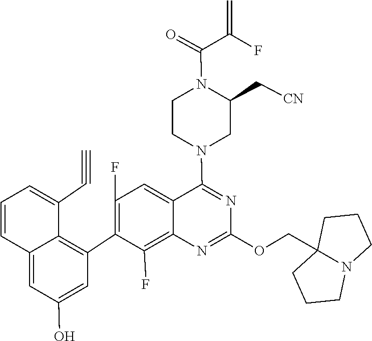 [SMILES: C#Cc1cccc2cc(O)cc(-c3c(F)cc4c(N5CCN(C(=O)C(=C)F)[C@@H](CC#N)C5)nc(OCC56CCCN5CCC6)nc4c3F)c12] |
| 8 | 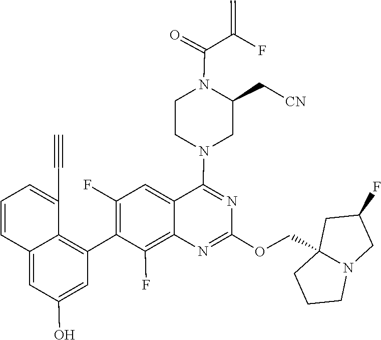 [SMILES: C#Cc1cccc2cc(O)cc(-c3c(F)cc4c(N5CCN(C(=O)C(=C)F)[C@@H](CC#N)C5)nc(OC[C@@]56CCCN5C[C@H](F)C6)nc4c3F)c12] |
| 9 | 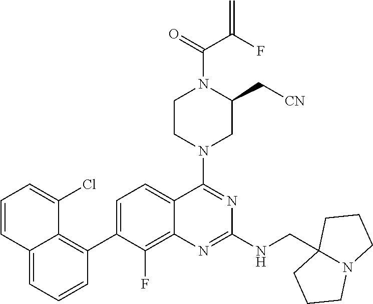 [SMILES: C=C(F)C(=O)N1CCN(c2nc(NCC34CCCN3CCC4)nc3c(F)c(-c4cccc5cccc(Cl)c45)ccc23)C[C@@H]1CC#N] |
| 10 | 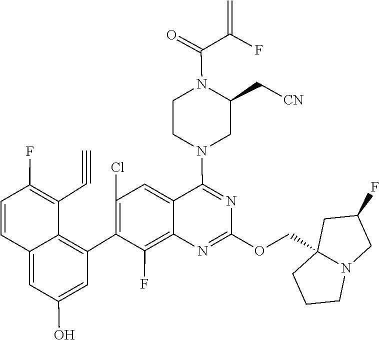 [SMILES: C#Cc1c(F)ccc2cc(O)cc(-c3c(Cl)cc4c(N5CCN(C(=O)C(=C)F)[C@@H](CC#N)C5)nc(OC[C@@]56CCCN5C[C@H](F)C6)nc4c3F)c12] |
| 11 | 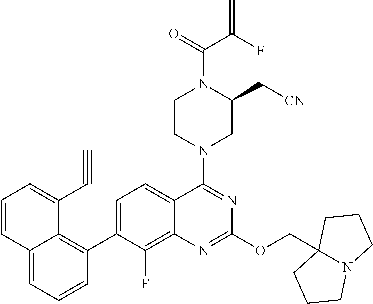 [SMILES: C#Cc1cccc2cccc(-c3ccc4c(N5CCN(C(=O)C(=C)F)[C@@H](CC#N)C5)nc(OCC56CCCN5CCC6)nc4c3F)c12] |
| 12 | 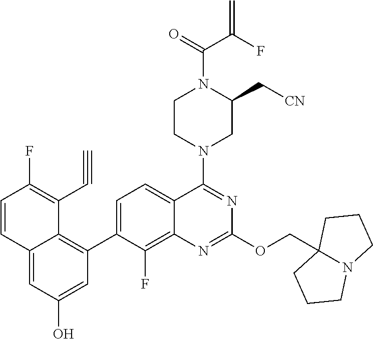 [SMILES: C#Cc1c(F)ccc2cc(O)cc(-c3ccc4c(N5CCN(C(=O)C(=C)F)[C@@H](CC#N)C5)nc(OCC56CCCN5CCC6)nc4c3F)c12] |
| 13 | 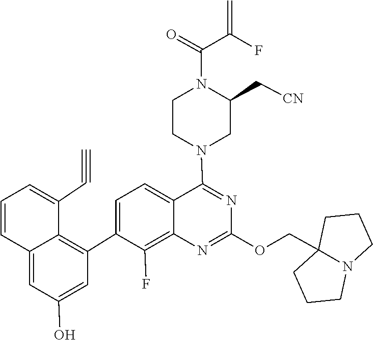 [SMILES: C#Cc1cccc2cc(O)cc(-c3ccc4c(N5CCN(C(=O)C(=C)F)[C@@H](CC#N)C5)nc(OCC56CCCN5CCC6)nc4c3F)c12] |
| 14 | 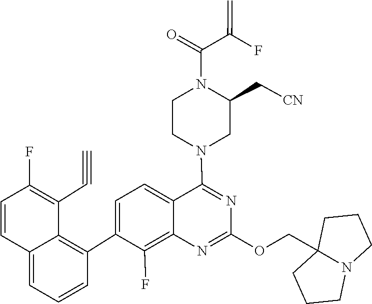 [SMILES: C#Cc1c(F)ccc2cccc(-c3ccc4c(N5CCN(C(=O)C(=C)F)[C@@H](CC#N)C5)nc(OCC56CCCN5CCC6)nc4c3F)c12] |
| 15 | 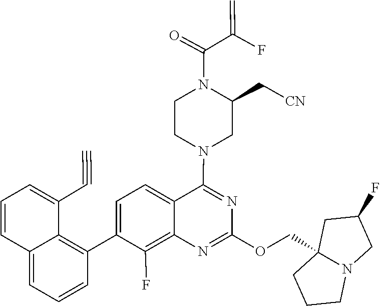 [SMILES: C#Cc1cccc2cccc(-c3ccc4c(N5CCN(C(=O)C(=C)F)[C@@H](CC#N)C5)nc(OC[C@@]56CCCN5C[C@H](F)C6)nc4c3F)c12] |
| 16 | 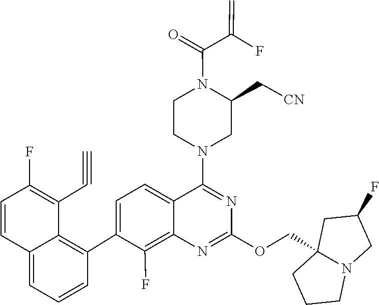 [SMILES: C#Cc1c(F)ccc2cccc(-c3ccc4c(N5CCN(C(=O)C(=C)F)[C@@H](CC#N)C5)nc(OC[C@@]56CCCN5C[C@H](F)C6)nc4c3F)c12] |
| 17 | 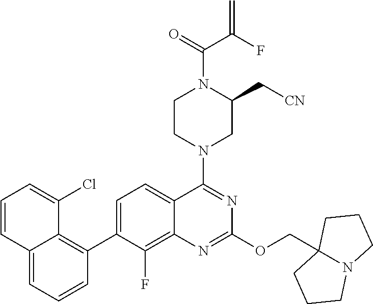 [SMILES: C=C(F)C(=O)N1CCN(c2nc(OCC34CCCN3CCC4)nc3c(F)c(-c4cccc5cccc(Cl)c45)ccc23)C[C@@H]1CC#N] |
| 18 | 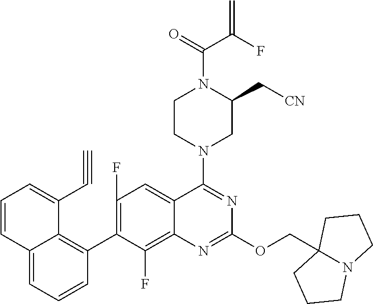 [SMILES: C#Cc1cccc2cccc(-c3c(F)cc4c(N5CCN(C(=O)C(=C)F)[C@@H](CC#N)C5)nc(OCC56CCCN5CCC6)nc4c3F)c12] |
| 19 | 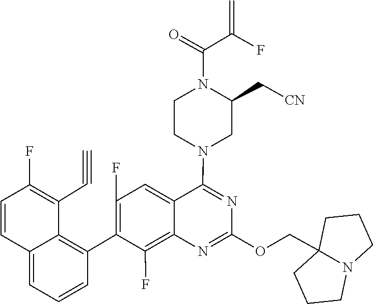 [SMILES: C#Cc1c(F)ccc2cccc(-c3c(F)cc4c(N5CCN(C(=O)C(=C)F)[C@@H](CC#N)C5)nc(OCC56CCCN5CCC6)nc4c3F)c12] |
| 20 | 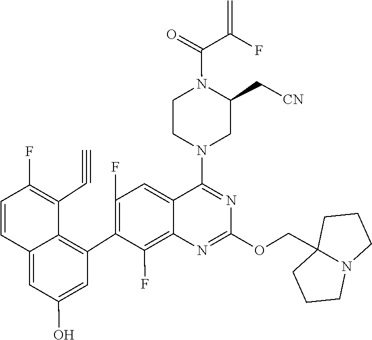 [SMILES: C#Cc1c(F)ccc2cc(O)cc(-c3c(F)cc4c(N5CCN(C(=O)C(=C)F)[C@@H](CC#N)C5)nc(OCC56CCCN5CCC6)nc4c3F)c12] |
| 21 | 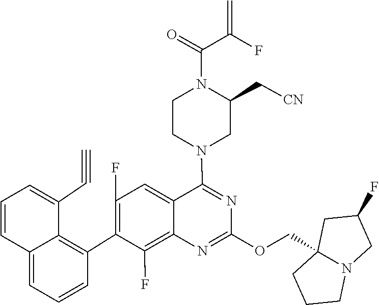 [SMILES: C#Cc1cccc2cccc(-c3c(F)cc4c(N5CCN(C(=O)C(=C)F)[C@@H](CC#N)C5)nc(OC[C@@]56CCCN5C[C@H](F)C6)nc4c3F)c12] |
| 22 | 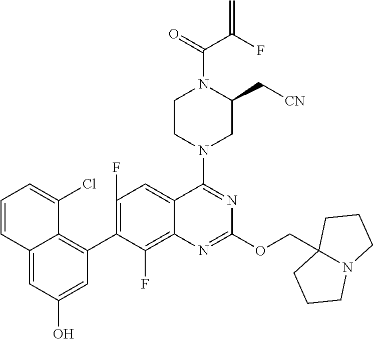 [SMILES: C=C(F)C(=O)N1CCN(c2nc(OCC34CCCN3CCC4)nc3c(F)c(-c4cc(O)cc5cccc(Cl)c45)c(F)cc23)C[C@@H]1CC#N] |
| 23 | 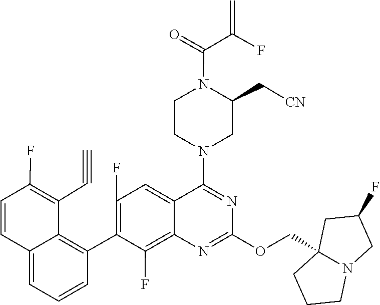 [SMILES: C#Cc1c(F)ccc2cccc(-c3c(F)cc4c(N5CCN(C(=O)C(=C)F)[C@@H](CC#N)C5)nc(OC[C@@]56CCCN5C[C@H](F)C6)nc4c3F)c12] |
| 24 | 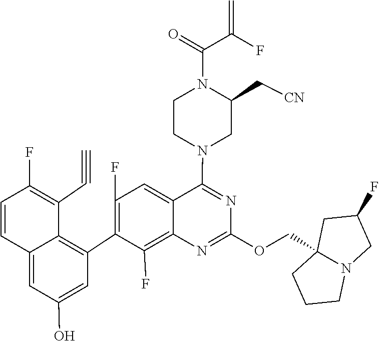 [SMILES: C#Cc1c(F)ccc2cc(O)cc(-c3c(F)cc4c(N5CCN(C(=O)C(=C)F)[C@@H](CC#N)C5)nc(OC[C@@]56CCCN5C[C@H](F)C6)nc4c3F)c12] . |

7. A pharmaceutical composition comprising:
the compound according to claim 1 or a pharmaceutically acceptable salt thereof, and
a pharmaceutically acceptable diluent or carrier.

8. A method for treating a disease that involves KRAS G12C mutation in a subject in need thereof, the method comprising:
administering the compound according to claim 1 a pharmaceutically acceptable salt thereof to a subject in need thereof.

9. The method of claim 8, wherein the disease is a cancer involving KRAS G12C mutation.

10. The method of claim 9, wherein the cancer is a lung cancer involving KRAS G12C mutation.
